SEQUENCE LISTING (100 sequences)

SEQ ID NO: 1 - protein, 22 aa, Vesiculovirus indiana
IASFFFIIGLIIGLFLVLRVGI

SEQ ID NO: 2 - protein, 37 aa, Vesiculovirus indiana
PIELVEGWFSSWKSSIASFFFIIGLIIGLFLVLRVGI

SEQ ID NO: 3 - protein, 53 aa, Vesiculovirus indiana
DDESLFFGDTGLSKNPIELVEGWFSSWKSSIASFFFIIGLIIGLFLVLRVGIH

SEQ ID NO: 4 - protein, 81 aa, Vesiculovirus indiana
GMLDSDLHLSSKAQVFEHPHIQDAASQLPDDESLFFGDTGLSKNPIELVEGWFSSWKSSIASFFFIIGLIIGLFLVLRVGI

SEQ ID NO: 5 - protein, 25 aa, Vesiculovirus indiana
HLCIKLKHTKKRQIYTDIEMNRLGK

SEQ ID NO: 6 - protein, 150 aa, Artificial Sequence
  Description of Artificial Sequence Synthetic polypeptide
IGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDI

SEQ ID NO: 7 - protein, 130 aa, Artificial Sequence
  Description of Artificial Sequence Synthetic polypeptide
IQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNL

SEQ ID NO: 8 - protein, 115 aa, Artificial Sequence
  Description of Artificial Sequence Synthetic polypeptide
IQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTT

SEQ ID NO: 9 - protein, 100 aa, Artificial Sequence
  Description of Artificial Sequence Synthetic polypeptide
IQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFING

SEQ ID NO: 10 - protein, 84 aa, Artificial Sequence
  Description of Artificial Sequence Synthetic polypeptide
IQADGWMCHASKWVTTCDFRWYGPKYITHSIRSFTPSVEQCKESIEQTKQGTWLNPGFPPQSCGYATVTDAEAVIVQVTPHHVL

SEQ ID NO: 11 - protein, 27 aa, Artificial Sequence
  Description of Artificial Sequence Synthetic peptide
GYIPEAPRDGQAYVRKDGEWVLLSTFL

SEQ ID NO: 12 - protein, 33 aa, Artificial Sequence
  Description of Artificial Sequence Synthetic polypeptide
RMKQLEDKVEELLSKQYHLENEVARLKKLVGER

SEQ ID NO: 13 - protein, 33 aa, Artificial Sequence
  Description of Artificial Sequence Synthetic polypeptide
RMKQLEDKVEELLSKNYHLENEVARLKKLVGER

SEQ ID NO: 14 - protein, 40 aa, Artificial Sequence
  Description of Artificial Sequence Synthetic polypeptide
MNPNQKIITIGSICLVVGLISLILQIGNIISIWISHSIQT

SEQ ID NO: 15 - protein, 75 aa, Artificial Sequence
  Description of Artificial Sequence Synthetic polypeptide
MNPNQKIITIGSICMVTGIVSLMLQIGNMISIWVSHSIHTGNQHQSEPISNTNFLTEKAVASVKLAGNSSLCPIN

SEQ ID NO: 16 - protein, 68 aa, Artificial Sequence
  Description of Artificial Sequence Synthetic polypeptide
KLCIEAKISNTTTDSRCPTQGEATLVEEQDTNFVCRRTFVDRGHGNGCGLFGKGSLITCAKFKCVTKL

SEQ ID NO: 17 - protein, 83 aa, Artificial Sequence
  Description of Artificial Sequence Synthetic polypeptide
IELLKTEVTNPAVLRKLCIEAKISNTTTDSRCPTQGEATLVEEQDTNFVCRRTFVDRGHGNGCGLFGKGSLITCAKFKCVTKL

SEQ ID NO: 18 - protein, 83 aa, Artificial Sequence
  Description of Artificial Sequence Synthetic polypeptide
KLCIEAKISNTTTDSRCPTQGEATLVEEQDTNFVCRRTFVDRGHGNGCGLFGKGSLITCAKFKCVTKLEGKIVQYENLKYSVI

SEQ ID NO: 19 - protein, 59 aa, Artificial Sequence
  Description of Artificial Sequence Synthetic polypeptide
EAKISNTTTDSRCPTQGEATLVEEQDTNFVCRRTFVDRGHGNGCGLFGKGSLITCAKFK

SEQ ID NO: 20 - protein, 251 aa, Artificial Sequence
  Description of Artificial Sequence Synthetic polypeptide
GSQVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYNGYDVLDNWGQGTLVTVSSGGGGSGGGGSGGGGSGGGGSDIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 21 - protein, 291 aa, Artificial Sequence
  Description of Artificial Sequence Synthetic polypeptide
GSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDGS

SEQ ID NO: 22 - protein, 299 aa, Homo sapiens
MAPWSHPSAQLQPVGGDAVSPALMVLLCLGLSLGPRTHVQAGNLSKATLWAEPGSVISRGNSVTIRCQGTLEAQEYRLVKEGSPEPWDTQNPLEPKNKARFSIPSMTEHHAGRYRCYYYSPAGWSEPSDPLELVVTGFYNKPTLSALPSPVVTSGENVTLQCGSRLRFDRFILTEEGDHKLSWTLDSQLTPSGQFQALFPVGPVTPSHRWMLRCYGSRRHILQVWSEPSDLLEIPVSGAADNLSPSQNKSDSGTASHLQDYAVENLIRMGMAGLILVVLGILIFQDWHSQRSPQAAAGR

SEQ ID NO: 23 - protein, 295 aa, Mus musculus
MTFVFTAVLCLGLNLGQETSMSEGNPHKPTLSVQPGLVVAKGKQVTISCEVTTGAREYRLYKEGGPHPWRTRNTPKTTNKAQFLIPSIEQRSGGIYRCYYKTPTGWSEHSDPLELAVTGLYSKPSLSTQPSNVVNSGETVTLQCVSTLGFNRFVLTKEGEQKWSLIQESEFINSTGQFQGLFTVGPVTPSQRWIFRCYGYHVNSPQVWSEPSDLLEIHVSEADQPLRPSPNISDPKTVSQPQNYTMENLIRMGASILVLVLLGVLLFEAQHSQRQTQHAAGRESSASFMVADTWE

SEQ ID NO: 24 - protein, 632 aa, Homo sapiens
MTPALTALLCLGLSLGPRTRMQAGPFPKPTLWAEPGSVISWGSPVTIWCQGSLEAQEYQLDKEGSPEPWDRNNPLEPKNKARFSIPSMTQHHAGRYRCHYYSSAGWSEPSDPLELVMTGFYNKPTLSALPSPVVASGGNMTLRCGSQKGYHHFVLMKEGEHQLPRTLDSQQLHSGGFQALFPVGPVTPSHRWRFTCYYYYTNTPWVWSHPSDPLEILPSGVSRKPSLLTLQGPVLAPGQSLTLQCGSDVGYDRFVLYKEGERDFLQRPGQQPQAGLSQANFTLGPVSRSYGGQYRCYGAHNLSSEWSAPSDPLDILITGQIYDTVSLSAQPGPTVASGENMTLLCQSRGYFDTFLLTKEGAAHPPLRLRSMYGAHKYQAEFPMSPVTSAHAGTYRCYGSRSSNPHLLSFPSEPLELMVSGHSGGSSLPPTGPPSTPGLGRYLEVLIGVSVAFVLLLFLLLFLLLLRQRHSKHRTSDQRKTDFQRPAGAAETEPKDRGLLRRSSPAADVQEENLYAAVKDTQSEDRVELDSQQSPHDEDPQAVTYAPVKHSSPRREMASPPSSLSGEFLDTKDRQVEEDRQMDTEAAASEASQDVTYAQLHSLTLRRKATEPPPSQEGEPPAEPSIYATLAIH

SEQ ID NO: 25 - protein, 680 aa, Mus musculus
MSCTFTALLCLGLTLSLWIPVLTGSLPKPILRVQPDSVVSRRTNVTFFCEETIGANEYRLYKDGKLYKTVTKNKQKQANKAEFSFSNVDLSNAGQYECSYSTQDESSGYSDPLELVVTGHYWTPSLSAQASPVVTSGGYVTLQCESWHNDHKFILTVEGPQKLSWTQDSQYNYSTRKYHALFSVGPVTPNQRWICRCYSYDRNRPYVWSPPSESVELLVSGNLQKPTIKAEPGSVITSKRAMTIWCQGNLDAEVYFLHNEKSQKTQSTQTLQQPGNKGRFFIPSVTQQHAGQYRCYCYSSAGWSQPSDTLELVVTGIYEYNEPRLSLLPSPVVRPGGNMTLHCASQGHYDKFILTKEDKKFANALDTEHISSSRQYQALFIIGPTTPTHTGTFRCYGYYKNTPQLWSVPSNLQQILISGLSKKPSLLTHQGHILDPGMTLTLQCFSDMNYDRFALHKVGGADIMQHSSQQTDIGFSVANFTLGYVSSSTGGQYRCYGAHNLSSEWSASSEPLDILITGQLPLTPSLSVQPNHTVHSGETVSLLCWSMDSVDTFILSKEGSAQQPLRLKSKSHDQQSQAEFSMSAVTSHLSGTYRCYGAQDSSFYLLSSASAPVELTVSGTIESSSWPPKRPNPPIPTENQDHTMENLIRMGMAVVVFIVLSILATEAWRSHRQTHCAAGN

SEQ ID NO: 26 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 27 - protein, 309 aa, Mus musculus
MDESAKTLPPPCLCFCSEKGEDMKVGYDPITPQKEEGAWFGICRDGRLLAATLLLALLSSSFTAMSLYQLAALQADLMNLRMELQSYRGSATPAAAGAPELTAGVKLLTPAAPRPHNSSRGHRNRRAFQGPEETEQDVDLSAPPAPCLPGCRHSQHDDNGMNLRNIIQDCLQLIADSDTPTIRKGTYTFVPWLLSFKRGNALEEKENKIVVRQTGYFFIYSQVLYTDPIFAMGHVIQRKKVHVFGDELSLVTLFRCIQNMPKTLPNNSCYSAGIARLEEGDEIQLAIPRENAQISRNGDDTFFGALKLL

SEQ ID NO: 28 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 29 - protein, 185 aa, Mus musculus
MAQQCFHSEYFDSLLHACKPCHLRCSNPPATCQPYCDPSVTSSVKGTYTVLWIFLGLTLVLSLALFTISFLLRKMNPEALKDEPQSPGQLDGSAQLDKADTELTRIRAGDDRIFPRSLEYTVEECTCEDCVKSKPKGDSDHFFPLPAMEEGATILVTTKTGDYGKSSVPTALQSVMGMEKPTHTR

SEQ ID NO: 30 - protein, 293 aa, Homo sapiens
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSTLGLCLCAVLCCFLVAVACFLKKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 31 - protein, 249 aa, Mus musculus
MAMAFCPKDQYWDSSRKSCVSCALTCSQRSQRTCTDFCKFINCRKEQGRYYDHLLGACVSCDSTCTQHPQQCAHFCEKRPRSQANLQPELGRPQAGEVEVRSDNSGRHQGSEHGPGLRLSSDQLTLYCTLGVCLCAIFCCFLVALASFLRRRGEPLPSQPAGPRGSQANSPHAHRPVTEACDEVTASPQPVETCSFCFPERSSPTQESAPRSLGIHGFAGTAAPQPCMRATVGGLGVLRASTGDARPAT

SEQ ID NO: 32 - protein, 184 aa, Homo sapiens
MRRGPRSLRGRDAPAPTPCVPAECFDLLVRHCVACGLLRTPRPKPAGASSPAPRTALQPQESVGAGAGEAALPLPGLLFGAPALLGLALVLALVLVGLVSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 33 - protein, 175 aa, Mus musculus
MGARRLRVRSQRSRDSSVPTQCNQTECFDPLVRNCVSCELFHTPDTGHTSSLEPGTALQPQEGSALRPDVALLVGAPALLGLILALTLVGLVSLVSWRWRQQLRTASPDTSEGVQQESLENVFVPSSETPHASAPTWPPLKEDADSALPRHSVPVPATELGSTELVTTKTAGPEQ

SEQ ID NO: 34 - protein, 128 aa, Homo sapiens
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 35 - protein, 129 aa, Mus musculus
MRAQRGLILLLLLLAVFCSTAVSLKCYNCLDPVSSCKINTTCSPNLDSCLYAVAGRQVYQQCWKLSDCNSNYIMSRLDVAGIQSKCCQWDLCNKNLDGLEEPNNAETSSLRKTALLGTSVLVAILKFCF

SEQ ID NO: 36 - protein, 381 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 37 - protein, 390 aa, Mus musculus
MIRGRAPRTRPSPPPPLLPLLSLSLLLLSPTVRGDCGPPPDIPNARPILGRHSKFAEQSKVAYSCNNGFKQVPDKSNIVVCLENGQWSSHETFCEKSCVAPERLSFASLKKEYLNMNFFPVGTIVEYECRPGFRKQPPLPGKATCLEDLVWSPVAQFCKKKSCPNPKDLDNGHINIPTGILFGSEINFSCNPGYRLVGVSSTFCSVTGNTVDWDDEFPVCTEIHCPEPPKINNGIMRGESDSYTYSQVVTYSCDKGFILVGNASIYCTVSKSDVGQWSSPPPRCIEKSKVPTKKPTINVPSTGTPSTPQKPTTESVPNPGDQPTPQKPSTVKVSATQHVPVTKTTVRHPIRTSTDKGEPNTGGDRYIYGHTCLITLTVLHVMLSLIGYLT

SEQ ID NO: 38 - protein, 153 aa, Homo sapiens [sequence omitted]

SEQ ID NO: 39 - protein, 140 aa, Mus musculus
MGLNPQLVVILLFFLECTRSHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 40 - protein, 244 aa, Homo sapiens
MDTESNRRANLALPQEPSSVPAFEVLEISPQEVSSGRLLKSASSPPLHTWLTVLKKEQEFLGVTQILTAMICLCFGTVVCSVLDISHIEGDIFSSFKAGYPFWGAIFFSISGMLSIISERRNATYLVRGSLGANTASSIAGGTGITILIINLKKSLAYIHIHSCQKFFETKCFMASFSTEIVVMMLFLTILGLGSAVSLTICGAGEELKGNKVPEDRVYEELNIYSATYSELEDPGEMSPPIDL

SEQ ID NO: 41 - protein, 235 aa, Mus musculus
MDTENRSRADLALPNPQESSSAPDIELLEASPAKAAPPKQTWRTFLKKELEFLGATQILVGLICLCFGTIVCSVLYVSDFDEEVLLLYKLGYPFWGAVLFVLSGFLSIISERKNTLYLVRGSLGANIVSSIAAGTGIAMLILNLTNNFAYMNNCKNVTEDDGCFVASFTTELVLMMLFLTILAFCSAVLFTIYRIGQELESKKVPDDRLYEELNVYSPIYSELEDKGETSSPVDS

SEQ ID NO: 42 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 43 - protein, 211 aa, Mus musculus
MKFLSARDFHPVAFLGLMLVTTTAFPTSQVRRGDFTEDTTPNRPVYTTSQVGGLITHVLWEIVEMRKELCNGNSDCMNNDDALAENNLKLPEIQRNDGCYQTGYNQEICLLKISSGLLEYHSYLEYMKNNLKDNKKDKARVLQRDTETLIHIFNQEVKDLHKIVLPTPISNALLTDKLESQKEWLRTKTIQFILKSLEEFLKVTLRSTRQT

SEQ ID NO: 44 - protein, 93 aa, Homo sapiens
MNAKVVVVLVLVLTALCLSDGKPVSLSYRCPCRFFESHVARANVKHLKILNTPNCALQIVARLKNNNRQVCIDPKLKWIQEYLEKALNKRFKM

SEQ ID NO: 45 - protein, 93 aa, Mus musculus
MDAKVVAVLALVLAALCISDGKPVSLSYRCPCRFFESHIARANVKHLKILNTPNCALQIVARLKNNNRQVCIDPKLKWIQEYLEKALNKRLKM

SEQ ID NO: 46 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 47 - protein, 537 aa, Mus musculus
MASTRAKPTLPLLLALVTVVIPGPGDAQVSIHPREAFLPQGGSVQVNCSSSCKEDLSLGLETQWLKDELESGPNWKLFELSEIGEDSSPLCFENCGTVQSSASATITVYSFPESVELRPLPAWQQVGKDLTLRCHVDGGAPRTQLSAVLLRGEEILSRQPVGGHPKDPKEITFTVLASRGDHGANFSCRTELDLRPQGLALFSNVSEARSLRTFDLPATIPKLDTPDLLEVGTQQKLFCSLEGLFPASEARIYLELGGQMPTQESTNSSDSVSATALVEVTEEFDRTLPLRCVLELADQILETQRTLTVYNFSAPVLTLSQLEVSEGSQVTVKCEAHSGSKVVLLSGVEPRPPTPQVQFTLNASSEDHKRSFFCSAALEVAGKFLFKNQTLELHVLYGPRLDETDCLGNWTWQEGSQQTLKCQAWGNPSPKMTCRRKADGALLPIGVVKSVKQEMNGTYVCHAFSSHGNVTRNVYLTVLYHSQNNWTIIILVPVLLVIVGLVMAASYVYNRQRKIRIYKLQKAQEEAIKLKGQAPPP

SEQ ID NO: 48 - protein, 1086 aa, Homo sapiens
MKDSCITVMAMALLSGFFFFAPASSYNLDVRGARSFSPPRAGRHFGYRVLQVGNGVIVGAPGEGNSTGSLYQCQSGTGHCLPVTLRGSNYTSKYLGMTLATDPTDGSILFAAVQFSTSYKTEFDFSDYVKRKDPDALLKHVKHMLLLTNTFGAINYVATEVFREELGARPDATKVLIIITDGEATDSGNIDAAKDIIRYIIGIGKHFQTKESQETLHKFASKPASEFVKILDTFEKLKDLFTELQKKIYVIEGTSKQDLTSFNMELSSSGISADLSRGHAVVGAVGAKDWAGGFLDLKADLQDDTFIGNEPLTPEVRAGYLGYTVTWLPSRQKTSLLASGAPRYQHMGRVLLFQEPQGGGHWSQVQTIHGTQIGSYFGGELCGVDVDQDGETELLLIGAPLFYGEQRGGRVFIYQRRQLGFEEVSELQGDPGYPLGRFGEAITALTDINGDGLVDVAVGAPLEEQGAVYIFNGRHGGLSPQPSQRIEGTQVLSGIQWFGRSIHGVKDLEGDGLADVAVGAESQMIVLSSRPVVDMVTLMSFSPAEIPVHEVECSYSTSNKMKEGVNITICFQIKSLIPQFQGRLVANLTYTLQLDGHRTRRRGLFPGGRHELRRNIAVTTSMSCTDFSFHFPVCVQDLISPINVSLNFSLWEEEGTPRDQRAGKDIPPILRPSLHSETWEIPFEKNCGEDKKCEANLRVSFSPARSRALRLTAFASLSVELSLSNLEEDAYWVQLDLHFPPGLSFRKVEMLKPHSQIPVSCEELPEESRLLSRALSCNVSSPIFKAGHSVALQMMFNTLVNSSWGDSVELHANVTCNNEDSDLLEDNSATTIIPILYPINILIQDQEDSTLYVSFTPKGPKIHQVKHMYQVRIQPSIHDHNIPTLEAVVGVPQPPSEGPITHQWSVQMEPPVPCHYEDLERLPDAAEPCLPGALFRCPVVFRQEILVQVIGTLELVGEIEASSMFSLCSSLSISFNSSKHFHLYGSNASLAQVVMKVDVVYEKQMLYLYVLSGIGGLLLLLLIFIVLYKVGFFKRNLKEKMEAGRGVPNGIPAEDSEQLASGQEAGDPGCLKPLHEKDSESGGGKD

SEQ ID NO: 49 - protein, 1161 aa, Mus musculus
MSFRIAGPRLLLLGLQLFAKAWSYNLDTRPTQSFLAQAGRHFGYQVLQIEDGVVVGAPGEGDNTGGLYHCRTSSEFCQPVSLHGSNHTSKYLGMTLATDAAKGSLLACDPGLSRTCDQNTYLSGLCYLFPQSLEGPMLQNRPAYQECMKGKVDLVFLFDGSQSLDRKDFEKILEFMKDVMRKLSNTSYQFAAVQFSTDCRTEFTFLDYVKQNKNPDVLLGSVQPMFLLTNTFRAINYVVAHVFKEESGARPDATKVLVIITDGEASDKGNISAAHDITRYIIGIGKHFVSVQKQKTLHIFASEPVEEFVKILDTFEKLKDLFTDLQRRIYAIEGTNRQDLTSFNMELSSSGISADLSKGHAVVGAVGAKDWAGGFLDLREDLQGATFVGQEPLTSDVRGGYLGYTVAWMTSRSSRPLLAAGAPRYQHVGQVLLFQAPEAGGRWNQTQKIEGTQIGSYFGGELCSVDLDQDGEAELLLIGAPLFFGEQRGGRVFTYQRRQSLFEMVSELQGDPGYPLGRFGAAITALTDINGDRLTDVAVGAPLEEQGAVYIFNGKPGGLSPQPSQRIQGAQVFPGIRWFGRSIHGVKDLGGDRLADVVVGAEGRVVVLSSRPVVDVVTELSFSPEEIPVHEVECSYSAREEQKHGVKLKACFRIKPLTPQFQGRLLANLSYTLQLDGHRMRSRGLFPDGSHELSGNTSITPDKSCLDFHFHFPICIQDLISPINVSLNFSLLEEEGTPRDQKGRAMQPILRPSIHTVTKEIPFEKNCGEDKKCEANLTLSSPARSGPLRLMSSASLAVEWTLSNSGEDAYWVRLDLDFPRGLSFRKVEMLQPHSRMPVSCEELTEGSSLLTKTLKCNVSSPIFKAGQEVSLQVMFNTLLNSSWEDFVELNGTVHCENENSSLQEDNSAATHIPVLYPVNILTKEQENSTLYISFTPKGPKTQQVQHVYQVRIQPSAYDHNMPTLEALVGVPWPHSEDPITYTWSVQTDPLVTCHSEDLKRPSSEAEQPCLPGVQFRCPIVFRREILIQVTGTVELSKEIKASSTLSLCSSLSVSFNSSKHFHLYGSKASEAQVLVKVDLIHEKEMLHVYVLSGIGGLVLLFLIFLALYKVGFFKRNLKEKMEADGGVPNGSPPEDTDPLAVPGEETKDMGCLEPLRESDKD

SEQ ID NO: 50 - protein, 370 aa, Homo sapiens
MGLWALLPGWVSATLLLALAALPAALAANSSGRWWGIVNVASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 51 - protein, 370 aa, Mus musculus
MGLWALLPSWVSTTLLLALTALPAALAANSSGRWWGIVNIASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSEGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 52 - protein, 23 aa, Influenza A virus
IITIGSVCMTIGMANLILQIGNI

SEQ ID NO: 53 - protein, 23 aa, Influenza A virus
LAIYSTVASSLVLVVSLGAISFW

SEQ ID NO: 54 - protein, 52 aa, Dengue virus
AYGVLFSGVSWTMKIGIGILLTWLGLNSRSTSLSMTCIAVGMVTLYLGVMVQ

SEQ ID NO: 55 - protein, 21 aa, Human immunodeficiency virus 1
FIMIVGGLVGLRIVFAVLSIV

SEQ ID NO: 56 - protein, 274 aa, Artificial Sequence
  Description of Artificial Sequence Synthetic polypeptide
MEFGLSWVFLVALFRGVQSQVQLVQSGAEVKKPGASVKVSCKVSGYTLTELSIHWVRQAPGKGLEWMGGFDPEDGETVYAQKFQGRVTMTEDTSSDTAYMELSSLRSEDTAVYYCATSFPIRGDPSYYYYYYGMDVWGQGTTVTVSSGGGGSGGGGSGGGGSGGGGSDIQMTQSPSSLSASVGDRVTITCRASQGITNYLAWFQQKPGKAPKSLIYAVSSLQSGVPSKFSGSGSGTDFTLTISSLQPEDFATYYCQQYNSYPWTFGQGTKVEIK

SEQ ID NO: 57 - protein, 268 aa, Artificial Sequence
  Description of Artificial Sequence Synthetic polypeptide
MEFGLSWVFLVALFRGVQSEVQLVESGGGVVQPGRSLRLSCAASGFTFSNYAIHWVRQAPGKGLEWVAVISYDGSNKYYADSVKGRFTISRDNSKNTLYLQMNSLRAEDTAVYYCARDFDDSSFWAFDYWGQGTLVTVSSGGGGSGGGGSGGGGSGGGGSDIQLTQSPSSLSASVGDRVTITCRASQSIRSYLNWYQQKPGKAPKLLIYAASSLQSGVPSRFSGSGSGTDFTLTISSLQPDDFATYYCQQSYSTPPATFGQGTKLEIK

SEQ ID NO: 58 - protein, 737 aa, Homo sapiens
KGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 59 - protein, 740 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHAEMGAIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 60 - protein, 335 aa, Mus sp.
MIAMLTVLLYLGLILEPRTAVQAGHLPKPIIWAEPGSVIAAYTSVITWCQGSWEAQYYHLYKEKSVNPWDTQVPLETRNKAKFNIPSMTTSYAGIYKCYYESAAGFSEHSDAMELVMTGAYENPSLSVYPSSNVTSGVSISFSCSSSIVFGRFILIQEGKHGLSWTLDSQHQANQPSYATFVLDAVTPNHNGTFRCYGYFRNEPQVWSKPSNSLDLMISETKDQSSTPTEDGLETYQKILIGVLVSFLLLFFLLLFLILIGYQYGHKKKANASVKNTQSENNAELNSWNPQNEDPQGIVYAQVKPSRLQKDTACKETQDVTYAQLCIRTQEQNNS

SEQ ID NO: 61 - protein, 247 aa, Homo sapiens
MGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVL

SEQ ID NO: 62 - protein, 133 aa, Homo sapiens
MVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 63 - protein, 205 aa, Homo sapiens
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 64 - protein, 267 aa, Artificial Sequence
  Description of Artificial Sequence Synthetic polypeptide
MPMGSLQPLATLYLLGMLVASVLADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 65 - protein, 53 aa, Mus sp.
MGRAMVARLGLGLLLLALLLPTQIYCNQTSVAPFPGNQNISASPNPSNATTRG

SEQ ID NO: 66 - protein, 243 aa, Homo sapiens
MCSRGWDSCLALELLLLPLSLLVTSIQGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARSFGVEWIASWLVVTVPTILGLLLT

SEQ ID NO: 67 - protein, 190 aa, Mus sp.
MGVRQVPWSFTWAVLQLSWQSGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMVIGIMSALVGIPVLLLLAWAL

SEQ ID NO: 68 - protein, 349 aa, Homo sapiens
MEAHNASAPFNFTLPPNFGKRPTDLALSVILVFMLFFIMLSLGCTMEFSKIKAHLWKPKGLAIALVAQYGIMPLTAFVLGKVFRLKNIEALAILVCGCSPGGNLSNVFSLAMKGDMNLSIVMTTCSTFCALGMMPLLLYIYSRGIYDGDLKDKVPYKGIVISLVLVLIPCTIGIVLKSKRPQYMRYVIKGGMIIILLCSVAVTVLSAINVGKSIMFAMTPLLIATSSLMPFIGFLLGYVLSALFCLNGRCRRTVSMETGCQNVQLCSTILNVAFPPEVIGPLFFFPLLYMIFQLGEGLLLIAIFWCYEKFKTPKDKTKMIYTAATTEETIPGALGNGTYKGEDCSPCTA

SEQ ID NO: 69 - protein, 191 aa, Mus sp.
MTCLGLRRYKAQLQLPSRTWPFVALLTLLFIPVFSEAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSDFLLWILVAVSLGLFFYSFLVTAVSLSKRIQ

SEQ ID NO: 70 - protein, 246 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPS

SEQ ID NO: 71 - protein, 848 aa, Artificial Sequence
  Description of Artificial Sequence Synthetic polypeptide
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSRGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 72 - protein, 740 aa, Artificial Sequence
  Description of Artificial Sequence Synthetic polypeptide
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 73 - protein, 978 aa, Artificial Sequence
  Description of Artificial Sequence Synthetic polypeptide
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHAEMGAIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSRIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 74 - protein, 740 aa, Artificial Sequence; identical to SEQ ID NO: 72
  Description of Artificial Sequence Synthetic polypeptide

SEQ ID NO: 75 - protein, 740 aa, Artificial Sequence; identical to SEQ ID NO: 72
  Description of Artificial Sequence Synthetic polypeptide

SEQ ID NO: 76 - protein, 801 aa, Artificial Sequence
  Description of Artificial Sequence Synthetic polypeptide
MNPNQKIITIGSICLVVGLISLILQIGNIISIWISHSIQTGGSKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLPAAARGSGLNDIFEAQKIEWHE

SEQ ID NO: 77 - protein, 836 aa, Artificial Sequence
  Description of Artificial Sequence Synthetic polypeptide
MNPNQKIITIGSICMVTGIVSLMLQIGNMISIWVSHSIHTGNQHQSEPISNTNFLTEKAVASVKLAGNSSLCPINGGSKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLPAAARGSGLNDIFEAQKIEWHE

SEQ ID NO: 78 - protein, 512 aa, Artificial Sequence
  Description of Artificial Sequence Synthetic polypeptide
MEFGLSWVFLVALFRGVQSQVQLVQSGAEVKKPGASVKVSCKVSGYTLTELSIHWVRQAPGKGLEWMGGFDPEDGETVYAQKFQGRVTMTEDTSSDTAYMELSSLRSEDTAVYYCATSFPIRGDPSYYYYYYGMDVWGQGTTVTVSSGGGGSGGGGSGGGGSGGGGSDIQMTQSPSSLSASVGDRVTITCRASQGITNYLAWFQQKPGKAPKSLIYAVSSLQSGVPSKFSGSGSGTDFTLTISSLQPEDFATYYCQQYNSYPWTFGQGTKVEIKDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 79 - protein, 376 aa, Artificial Sequence
  Description of Artificial Sequence Synthetic polypeptide
MEFGLSWVFLVALFRGVQSEVQLVESGGGVVQPGRSLRLSCAASGFTFSNYAIHWVRQAPGKGLEWVAVISYDGSNKYYADSVKGRFTISRDNSKNTLYLQMNSLRAEDTAVYYCARDFDDSSFWAFDYWGQGTLVTVSSGGGGSGGGGSGGGGSGGGGSDIQLTQSPSSLSASVGDRVTITCRASQSIRSYLNWYQQKPGKAPKLLIYAASSLQSGVPSRFSGSGSGTDFTLTISSLQPDDFATYYCQQSYSTPPATFGQGTKLEIKSRGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 80 - protein, 952 aa, Artificial Sequence
  Description of Artificial Sequence Synthetic polypeptide
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHAEMGAIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSGMLDSDLHLSSKAQVFEHPHIQDAASQLPDDESLFFGDTGLSKNPIELVEGWFSSWKSSIASFFFIIGLIIGLFLVLRVGIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNL

SEQ ID NO: 81 - protein, 948 aa, Artificial Sequence
  Description of Artificial Sequence Synthetic polypeptide
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHAEMGAIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSRIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 82 - protein, 932 aa, Artificial Sequence
  Description of Artificial Sequence Synthetic polypeptide
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHAEMGAIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSRIQADGWMCHASKWVTTCDFRWYGPKYITHSIRSFTPSVEQCKESIEQTKQGTWLNPGFPPQSCGYATVTDAEAVIVQVTPHHVLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 83 - protein, 529 aa, Artificial Sequence
  Description of Artificial Sequence Synthetic polypeptide
MTFVFTAVLCLGLNLGQETSMSEGNPHKPTLSVQPGLVVAKGKQVTISCEVTTGAREYRLYKEGGPHPWRTRNTPKTTNKAQFLIPSIEQRSGGIYRCYYKTPTGWSEHSDPLELAVTGLYSKPSLSTQPSNVVNSGETVTLQCVSTLGFNRFVLTKEGEQKWSLIQESEFINSTGQFQGLFTVGPVTPSQRWIFRCYGYHVNSPQVWSEPSDLLEIHVSEADQPLRPSPNISDPKTVSQPQNYTMENLIRMGASILVLVLLGVLLFEAQHSQRQTQHAAGRESSASFMVADTWEIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYPYDVPDYA

SEQ ID NO: 84 - protein, 876 aa, Artificial Sequence
  Description of Artificial Sequence Synthetic polypeptide
MSCTFTALLRLGLTLSLWIPVLTGSLPKPILRVQPDSVVSRRTKVTFLCEETIGANEYRLYKDGKLYKTVTKNKQKPENKAEFSFSNVDLSNAGQYRCSYSTQYKSSGYSDLLELVVTGHYWTPSLLAQASPVVTSGGYVTLQCESWHNDHKFILTVEGPQKLSWTQDSQYNYSTRKYHALFSVGPVTPNQRWICRCYSYDRNRPYVWSPPSESVELLVSGNLQKPTIKAEPGSVITSKRAMTIWCQGNLDAEVYFLHNEKSQKTQSTQTLQEPGNKGKFFIPSVTLQHAGQYRCYCYGSAGWSQPSDTLELVVTGIYEYYEPRLSVLPSPVVTAGGNMTLHCASDFPYDKFILTKEDKKFGNSLDTEHISSSGQYRALFIIGPTTPTHTGAFRCYGYYKNAPQLWSVPSALQQILISGLSKKPSLLTHQGHILDPGMTLTLQCFSDINYDRFALHKVGGADIMQHSSQQTDTGFSVANFTLGYVSSSTGGQYRCYGAHNLSSEWSASSEPLDILITGQLPLTPSLSVQPNHTVHSGETVSLLCWSMDSVDTFILSKEGSAQQPLRLKSKSHDQQSQAEFSMSAVTSHLSGTYRCYGAQDSSFYLLSSASAPVELTVSGPIETSTPPPTMSMPLGGLHMYLKIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYPYDVPDYA

SEQ ID NO: 85 - protein, 569 aa, Artificial Sequence
  Description of Artificial Sequence Synthetic polypeptide
MIAMLTVLLYLGLILEPRTAVQAGHLPKPIIWAEPGSVIAAYTSVITWCQGSWEAQYYHLYKEKSVNPWDTQVPLETRNKAKFNIPSMTTSYAGIYKCYYESAAGFSEHSDAMELVMTGAYENPSLSVYPSSNVTSGVSISFSCSSSIVFGRFILIQEGKHGLSWTLDSQHQANQPSYATFVLDAVTPNHNGTFRCYGYFRNEPQVWSKPSNSLDLMISETKDQSSTPTEDGLETYQKILIGVLVSFLLLFFLLLFLILIGYQYGHKKKANASVKNTQSENNAELNSWNPQNEDPQGIVYAQVKPSRLQKDTACKETQDVTYAQLCIRTQEQNNSIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYPYDVPDYA

SEQ ID NO: 86 - protein, 328 aa, Artificial Sequence
  Description of Artificial Sequence Synthetic polypeptide
MYTDIEMNRLGKNPNQKIITIGSICMVTGIVSLMLQIGNMISIWVSHSIHTGNQHQSEPISNTNFLTEKAVASVKLAGNSSLCPINDQLAALQADLMNLRMELQSYRGSATPAAAGAPELTAGVKLLTPAAPRPHNSSRGHRNRRAFQGPEETEQDVDLSAPPAPCLPGCRHSQHDDNGMNLRNIIQDCLQLIADSDTPTIRKGTYTFVPWLLSFKRGNALEEKENKIVVRQTGYFFIYSQVLYTDPIFAMGHVIQRKKVHVFGDELSLVTLFRCIQNMPKTLPNNSCYSAGIARLEEGDEIQLAIPRENAQISRNGDDTFFGALKLL

SEQ ID NO: 87 - protein, 308 aa, Artificial Sequence
  Description of Artificial Sequence Synthetic polypeptide
MGARRLRVRSQRSRDSSVPTQCNQTECFDPLVRNCVSCELFHTPDTGHTSSLEPGTALQPQEGSALRPDVAIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 88 - protein, 286 aa, Artificial Sequence
  Description of Artificial Sequence Synthetic polypeptide
MAQQCFHSEYFDSLLHACKPCHLRCSNPPATCQPYCDPSVTSSVKGTYTIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 89 - protein, 365 aa, Artificial Sequence
  Description of Artificial Sequence Synthetic polypeptide
MAMAFCPKDQYWDSSRKSCVSCALTCSQRSQRTCTDFCKFINCRKEQGRYYDHLLGACVSCDSTCTQHPQQCAHFCEKRPRSQANLQPELGRPQAGEVEVRSDNSGRHQGSEHGPGLRLSSDQLTLYCIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 90 - protein, 723 aa, Artificial Sequence
  Description of Artificial Sequence Synthetic polypeptide
MASTRAKPTLPLLLALVTVVIPGPGDAQVSIHPREAFLPQGGSVQVNCSSSCKEDLSLGLETQWLKDELESGPNWKLFELSEIGEDSSPLCFENCGTVQSSASATITVYSFPESVELRPLPAWQQVGKDLTLRCHVDGGAPRTQLSAVLLRGEEILSRQPVGGHPKDPKEITFTVLASRGDHGANFSCRTELDLRPQGLALFSNVSEARSLRTFDLPATIPKLDTPDLLEVGTQQKLFCSLEGLFPASEARIYLELGGQMPTQESTNSSDSVSATALVEVTEEFDRTLPLRCVLELADQILETQRTLTVYNFSAPVLTLSQLEVSEGSQVTVKCEAHSGSKVVLLSGVEPRPPTPQVQFTLNASSEDHKRSFFCSAALEVAGKFLFKNQTLELHVLYGPRLDETDCLGNWTWQEGSQQTLKCQAWGNPSPKMTCRRKADGALLPIGVVKSVKQEMNGTYVCHAFSSHGNVTRNVYLTVLYHSQNNDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 91 - protein, 330 aa, Artificial Sequence
  Description of Artificial Sequence Synthetic polypeptide
MDAKVVAVLALVLAALCISDGKPVSLSYRCPCRFFESHIARANVKHLKILNTPNCALQIVARLKNNNRQVCIDPKLKWIQEYLEKALNKRLKMIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 92 - protein, 377 aa, Artificial Sequence
  Description of Artificial Sequence Synthetic polypeptide
MGLNPQLVVILLFFLECTRSHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYSIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 93 - protein, 448 aa, Artificial Sequence
  Description of Artificial Sequence Synthetic polypeptide
MKFLSARDFHPVAFLGLMLVTTTAFPTSQVRRGDFTEDTTPNRPVYTTSQVGGLITHVLWEIVEMRKELCNGNSDCMNNDDALAENNLKLPEIQRNDGCYQTGYNQEICLLKISSGLLEYHSYLEYMKNNLKDNKKDKARVLQRDTETLIHIFNQEVKDLHKIVLPTPISNALLTDKLESQKEWLRTKTIQFILKSLEEFLKVTLRSTRQTIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 94 - protein, 596 aa, Artificial Sequence
  Description of Artificial Sequence Synthetic polypeptide
MNAPLGGIWLWLPLLLTWLTPEVNSSWWYMRATGGSSRVMCDNVPGLVSSQRQLCHRHPDVMRAISQGVAEWTAECQHQFRQHRWNCNTLDRDHSLFGRVLLRSSRESAFVYAISSAGVVFAITRACSQGEVKSCSCDPKKMGSAKDSKGIFDWGGCSDNIDYGIKFARAFVDAKERKGKDARALMNLHNNRAGRKAVKRFLKQECKCHGVSGSCTLRTCWLAMADFRKTGDYLWRKYNGAIQVVMNQDGTGFTVANERFKKPTKNDLVYFENSPDYCIRDREAGSLGTAGRVCNLTSRGMDSCEVMCCGRGYDTSHVTRMTKCGCKFHWCCAVRCQDCLEALDVHTCKAPKNADWTTATIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 95 - protein, 416 aa, Artificial Sequence
  Description of Artificial Sequence Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDRSGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 96 - protein, 546 aa, Artificial Sequence
  Description of Artificial Sequence Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 97 - protein, 485 aa, Artificial Sequence
  Description of Artificial Sequence Synthetic polypeptide
MGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 98 - protein, 502 aa, Artificial Sequence
  Description of Artificial Sequence Synthetic polypeptide
MPMGSLQPLATLYLLGMLVASVLADIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKVIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYPYDVPDYA

SEQ ID NO: 99 - protein, 220 aa, Artificial Sequence
  Description of Artificial Sequence Synthetic polypeptide
MYTDIEMNRLGKNPNQKIITIGSICMVTGIVSLMLQIGNMISIWVSHSIHTGNQHQSEPISNTNFLTEKAVASVKLAGNSSLCPINDMVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 100 - protein, 292 aa, Artificial Sequence
  Description of Artificial Sequence Synthetic polypeptide
MYTDIEMNRLGKNPNQKIITIGSICMVTGIVSLMLQIGNMISIWVSHSIHTGNQHQSEPISNTNFLTEKAVASVKLAGNSSLCPINDACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

(19) United States
(12) Patent Application Publication
(10) Pub. No.: US 2024/0360181 A1
(43) Pub. Date: Oct. 31, 2024

(54) COMPOSITIONS AND METHODS FOR MULTIVALENT SURFACE DISPLAY ON ENVELOPED PARTICLES

(71) Applicant: Achelois BioPharma, Inc., Redwood City, CA (US)

(72) Inventors: Chang-Zheng CHEN, Palo Alto, CA (US); Michael CHEN, Palo Alto, CA (US); Yiling LUO, South San Francisco, CA (US)

(21) Appl. No.: 18/560,619

(22) PCT Filed: May 19, 2022
(86) PCT No.: PCT/US2022/029995

§ 371 Date: Nov. 13, 2023

Related U.S. Application Data
(60) Provisional application No. 63/191,013, filed on May 20, 2021.

(51) Int. Cl. C07K 14/005

(52) U.S. Cl. CPC C07K 14/005; C07K 2319/02; C07K 2319/03; C07K 2319/30; C07K 2319/73; C12N 2740/15023; C12N 2740/16122; C12N 2760/16122; C12N 2760/18422; C12N 2760/20222; C12N 2770/24122

(57) ABSTRACT

Disclosed herein are recombinant fusion proteins comprising transmembrane domains, a display polypeptides, and oligomerization domains wherein when the recombinant fusion protein is expressed on the surface of an enveloped particle, the recombinant fusion protein is displayed in an oligomeric format, and enveloped particles containing the same, and methods of usc.

### CROSS-REFERENCE

This application claims the benefit of U.S. Provisional Application No. 63/191,013 filed May 20, 2021, which is incorporated herein by reference in its entirety.

### BRIEF SUMMARY

In one aspect, provided herein is a recombinant fusion protein comprising a transmembrane domain, a display polypeptide, and an oligomerization domain wherein when the recombinant fusion protein is expressed on the surface of an enveloped particle, the recombinant fusion protein is displayed in an oligomeric format. In some embodiments, the oligomerization domain comprises a dimerization domain, a trimerization domain, or a tetramerization domain. In some embodiments, the dimerization domain comprises a leucine zipper dimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain, or a foldon trimerization domain. In some embodiments, the tetramerization domain comprises an influenza neuraminidase stem domain. In some embodiments, the oligomerization domain comprises an amino acid sequence that has at least about 80%, 85%, 90%, 95%, or 99% sequence identity to an amino acid sequence of SEQ ID NOs: 6-19.

In some embodiments, when the recombinant fusion protein is expressed on the surface of the enveloped particle, the oligomerization domain is outside of the enveloped particle. In some embodiments, when the recombinant fusion protein is expressed on the surface of the enveloped particle, the oligomerization domain is outside of the enveloped particle and adjacent to a signal peptide. In some embodiments, when the recombinant fusion protein is expressed on the surface of the enveloped particle, the oligomerization domain is inside of the enveloped particle. In some embodiments, when the recombinant fusion protein is expressed on the surface of the enveloped particle, the oligomerization domain is inside of the enveloped particle and adjacent to the transmembrane domain. In some embodiments, the recombinant fusion protein comprises a signal peptide. The recombinant fusion protein of claim 13, wherein domains of the recombinant fusion protein are arranged from the N-terminus to the C-terminus in the following orders: (a) signal peptide, display polypeptide, oligomerization domain, and transmembrane domain: (b) signal peptide, display polypeptide, transmembrane domain, and oligomerization domain: or (c) signal peptide, oligomerization domain, display peptide, and transmembrane domain. In some embodiments, the recombinant fusion protein further comprises a cytosolic domain. In some embodiments, domains of the recombinant fusion protein are arranged from the N-terminus to the C-terminus in the following orders: (a) signal peptide, display polypeptide, oligomerization domain, transmembrane domain, and cytosolic domain: (b) signal peptide, display polypeptide, transmembrane domain, oligomerization domain, and cytosolic domain; or (c) signal peptide, oligomerization domain, display peptide, transmembrane domain, and cytosolic domain. In some embodiments, the enveloped particle comprises an enveloped viral-like particle, an enveloped virus, an extracellular vesicle. In some embodiments, the extracellular vesicle comprises an exosome or an ectosome. In some embodiments, the display polypeptide targets a cell surface protein that is oligomerized on a target cell for agonistic or antagonistic function. In some embodiments, the cell surface protein is dimerized and the oligomerization domain is a dimerization domain. In some embodiments, the cell surface protein is trimerized and the oligomerization domain is a trimerization domain. In some embodiments, the cell surface protein is tetramerized and the oligomerization domain is a tetramerization domain.

In some embodiments, the display polypeptide comprises a single chain variable fragment (scFv), a type I transmembrane polypeptide, a type II transmembrane polypeptide, a type III transmembrane polypeptide, a GPI-anchored polypeptide, a secreted polypeptide, or a multi-pass transmembrane polypeptide. In some embodiments, the scFv comprises an anti-BCMA scFv polypeptide, an anti-CD19 scFv polypeptide, an anti-NTD scFv polypeptide, or an anti-RBD scFv polypeptide. In some embodiments, the type I transmembrane polypeptide comprises ACE2, ICAM-1, LILRA5, LILRB3, or LILRB4. In some embodiments, the type II transmembrane polypeptide comprises DPP4, or BAFF. In some embodiments, the type III transmembrane polypeptide comprises BCMA, TACI, or BAFF Receptor. In some embodiments, the GPI-anchored polypeptide comprises CD24, CD48, CD59 or CD55. In some embodiments, the secreted polypeptide comprises IL-4, or CXCL-12. In some embodiments, the multi-pass transmembrane polypeptide comprises NTCP, or FCεRI beta. In some embodiments, the display polypeptide comprises an antibody, a receptor, a cytokine, an immune checkpoint protein, a chemokine, an adhesion factor, or a mitogen. In some embodiments, the antibody comprises a non-neutralizing antibody, a neutralizing antibody, or an antibody of multiple formats. In some embodiments, the receptor comprises ACE2, or DPP4. In some embodiments, the cytokine comprises IL-4, or IL-6. In some embodiments, the immune checkpoint protein comprises CTLA-4, or PD-1. In some embodiments, the chemokine comprises CXCL-12. In some embodiments, the adhesion factor comprises ICAM-1, or LFA-1. In some embodiments, the mitogen comprises Wnt. In some embodiments, the display polypeptide comprises an amino acid sequence that has at least about 80%, 85%, 90%, 95% or 99% sequence identity to an amino acid sequence according to SEQ ID NOs: 20-51, 56-70. In some embodiments, the transmembrane domain anchors the fusion protein to a bilayer of the enveloped particle. In some embodiments, the transmembrane domain comprises: (a) the transmembrane domain of a Vesicular Stomatitis virus glycoprotein (VSV-G): (b) the transmembrane domain of a Dengue E protein: (c) the transmembrane domain of influenza Hemagglutinin (HA): (d) the transmembrane domain of HIV surface glycoprotein GP120 or GP41: (c) the transmembrane domain of measles virus surface glycoprotein hamagglutinin (H) protein: or (f) the transmembrane domain of influenza Neuraminidase (NA). In some embodiments, the recombinant fusion protein comprises a cytosolic domain. In some embodiments, the recombinant fusion protein comprises: (a) the transmembrane domain and cytosolic domain of a Vesicular Stomatitis virus glycoprotein (VSV-G); (b) the transmembrane domain and cytosolic domain of a Dengue E protein: (c) the transmembrane domain and cytosolic domain of influenza Hemagglutinin (HA): (d) the transmembrane domain and cytosolic domain of HIV surface glycoprotein GP120 or GP41: (c) the transmembrane domain and cytosolic domain of measles virus surface glycoprotein hamagglutinin (H) protein: or (f) the transmembrane domain and cytosolic domain of influenza Neuraminidase (NA). In some embodiments. (a) the display polypeptide comprises a single chain variable fragment (scFv), a type I transmembrane polypeptide, a type II transmembrane polypeptide, a type III transmembrane polypeptide, a GPI-anchored polypeptide, a secreted polypeptide, a multi-pass transmembrane polypeptide, an antibody, a receptor, a cytokine, an immune checkpoint protein, a chemokine, an adhesion factor, or a mitogen: (b) the transmembrane domain comprises the transmembrane domain of a Vesicular Stomatitis virus glycoprotein (VSV-G), the transmembrane domain of a Dengue E protein, the transmembrane domain of influenza Hemagglutinin (HA), the transmembrane domain of HIV surface glycoprotein GP120 or GP41, the transmembrane domain of measles virus surface glycoprotein hamagglutinin (H) protein, or the transmembrane domain of influenza Neuraminidase (NA); and (c) the oligomerization domain comprises a leucine zipper dimerization domain. D4 post-fusion trimerization domain of VSV-G protein, a Dengue E protein post-fusion trimerization domain, a foldon trimerization domain, or an influenza neuraminidase stem domain. In some embodiments. (a) the display polypeptide comprises an anti-BCMA scFv polypeptide, an anti-CD19 scFv polypeptide, an anti-NTD scFv polypeptide, an anti-RBD scFv polypeptide. ACE2. ICAM-1. LILRA5. LILRB3. LILRB4, DPP4, BAFF. BCMA. TACI. BAFF Receptor. CD24. CD48. CD59. CD55. IL-4. CXCL-12. NTCP. FCεRI beta, a non-neutralizing antibody, a neutralizing antibody, an antibody of multiple formats. IL-6. CTLA-4, PD-1. LFA-1. Wnt. CD80. CD86. OX40L. CD3, or 4-1BBL: (b) the transmembrane domain comprises the transmembrane domain of a Vesicular Stomatitis virus glycoprotein (VSV-G), the transmembrane domain of a Dengue E protein, the transmembrane domain of influenza Hemagglutinin (HA), the transmembrane domain of HIV surface glycoprotein GP120 or GP41, the transmembrane domain of measles virus surface glycoprotein hamagglutinin (H) protein, or the transmembrane domain of influenza Neuraminidase (NA); and (c) the oligomerization domain comprises a leucine zipper dimerization domain, D4 post-fusion trimerization domain of VSV-G protein, a Dengue E protein post-fusion trimerization domain, a foldon trimerization domain, or an influenza neuraminidase stem domain. In some embodiments, (a) the display polypeptide comprises an amino acid sequence that has at least about 80%, 85%, 90%, 95% or 99% sequence identity to an amino acid sequence according to SEQ ID NOs: 20-51, 56-70; (b) the transmembrane domain comprises the transmembrane domain of a Vesicular Stomatitis virus glycoprotein (VSV-G), the transmembrane domain of a Dengue E protein, the transmembrane domain of influenza Hemagglutinin (HA), the transmembrane domain of HIV surface glycoprotein GP120 or GP41, the transmembrane domain of measles virus surface glycoprotein hamagglutinin (H) protein, or the transmembrane domain of influenza Neuraminidase (NA); and (c) the oligomerization domain comprises a leucine zipper dimerization domain, D4 post-fusion trimerization domain of VSV-G protein, a Dengue E protein post-fusion trimerization domain, a foldon trimerization domain, or an influenza neuraminidase stem domain. In some embodiments, (a) the display polypeptide comprises an amino acid sequence that has at least about 80%, 85%, 90%, 95% or 99% sequence identity to an amino acid sequence according to SEQ ID NOs: 20-51, 56-70; (b) the transmembrane domain comprises an amino acid sequence that has at least about 80%, 85%, 90%, 95% or 99% sequence identity to an amino acid sequence according to SEQ ID NOs: 1-4, 52-55; and (c) the oligomerization domain comprises an amino acid sequence that has at least about 80%, 85%, 90%, 95% or 99% sequence identity to an amino acid sequence of SEQ ID NOs: 6-19. In some embodiments, the cytosolic domain comprises an amino acid sequence that has at least about 80%, 85%, 90%, 95% or 99% sequence identity to SEQ ID NO: 5. In some embodiments, the transmembrane domain comprises an amino acid sequence that has at least about 80%, 85%, 90%, 95% or 99% sequence identity to an amino acid sequence according to SEQ ID NOs: 1-4, 52-55.

In some embodiments, when the recombinant fusion protein is expressed on the surface of the enveloped particle the recombinant fusion protein is expressed at a valency of about 10 copies on the surface of the enveloped particle. In some embodiments, when the recombinant fusion protein is expressed on the surface of the enveloped particle the recombinant fusion protein is expressed at a valency of about 10 to 15 copies on the surface of the enveloped particle. In some embodiments, when the recombinant fusion protein is expressed on the surface of the enveloped particle the recombinant fusion protein is expressed at a valency of at least about 25 copies on the surface of the enveloped particle. In some embodiments, when the recombinant fusion protein is expressed on the surface of the enveloped particle the recombinant fusion protein is expressed at a valency of at least about 50 copies on the surface of the enveloped particle. In some embodiments, when the recombinant fusion protein is expressed on the surface of the enveloped particle the recombinant fusion protein is expressed at a valency of at least about 100 copies on the surface of the enveloped particle. In some embodiments, when the recombinant fusion protein is expressed on the surface of the enveloped particle the recombinant fusion protein is expressed at a valency of at least about 200 copies on the surface of the enveloped particle. In some embodiments, when the recombinant fusion protein is expressed on the surface of the enveloped particle the recombinant fusion protein is expressed at a valency of at least about 400 copies on the surface of the enveloped particle. In some embodiments, when the recombinant fusion protein is expressed on the surface of the enveloped particle the recombinant fusion protein is expressed at a valency of at least about 600 copies on the surface of the enveloped particle. In some embodiments, when the recombinant fusion protein is expressed on the surface of the enveloped particle the recombinant fusion protein is expressed at least about 1000 copies on the surface of the enveloped particle. In another aspect, provided herein is an enveloped particle comprising the recombinant fusion protein. In another aspect, provided herein is an enveloped particle comprising: a first recombinant fusion protein that is displayed in an oligomeric format on the surface of the enveloped particle wherein the first recombinant fusion protein comprises a transmembrane domain, a display polypeptide, and an oligomerization domain; and a second recombinant fusion protein that is displayed in a monomeric format on the surface of the enveloped particle wherein the second recombinant fusion protein comprises a transmembrane domain and a display polypeptide. In some embodiments, the oligomerization domain comprises a dimerization domain, a trimerization domain, or a tetramerization domain. In some embodiments, the dimerization domain comprises a leucine zipper dimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain, or a foldon trimerization domain. In some embodiments, the tetramerization domain comprises an influenza neuraminidase stem domain. In some embodiments, the oligomerization domain comprises an amino acid sequence that has at least about 80%, 85%, 90%, 95% or 99% sequence identity to an amino acid sequence of SEQ ID NOs: 6-19. In some embodiments, when the first recombinant fusion protein is expressed on the surface of the enveloped particle, the oligomerization domain is outside of the enveloped particle. In some embodiments, when the first recombinant fusion protein is expressed on the surface of the enveloped particle, the oligomerization domain is outside of the enveloped particle and adjacent to a signal peptide. In some embodiments, when the first recombinant fusion protein is expressed on the surface of the enveloped particle, the oligomerization domain is inside of the enveloped particle. In some embodiments, when the first recombinant fusion protein is expressed on the surface of the enveloped particle, the oligomerization domain is inside of the enveloped particle and adjacent to the transmembrane domain.

In some embodiments, the first recombinant fusion protein comprises a signal peptide. In some embodiments, domains of the first recombinant fusion protein are arranged from the N-terminus to the C-terminus in the following orders: (a) signal peptide, display polypeptide, oligomerization domain, and transmembrane domain: (b) signal peptide, display polypeptide, transmembrane domain, and oligomerization domain: or (c) signal peptide, oligomerization domain, display peptide, and transmembrane domain. In some embodiments, the first or second recombinant fusion protein further comprises a cytosolic domain. In some embodiments, domains of the first recombinant fusion protein are arranged from the N-terminus to the C-terminus in the following orders: (a) signal peptide, display polypeptide, oligomerization domain, transmembrane domain, and cytosolic domain: (b) signal peptide, display polypeptide, transmembrane domain, oligomerization domain, and cytosolic domain; or (c) signal peptide, oligomerization domain, display peptide, transmembrane domain, and cytosolic domain. In some embodiments, the enveloped particle comprises an enveloped viral-like particle, an enveloped virus, an extracellular vesicle. In some embodiments, the extracellular vesicle comprises an exosome or an ectosome. In some embodiments, the first recombinant fusion protein and the second recombinant fusion protein have the same display polypeptide. In some embodiments, the first recombinant fusion protein and the second recombinant fusion protein have different display polypeptides. In some embodiments, the display polypeptide of the first or second recombinant fusion protein targets a cell surface protein that is oligomerized on a target cell for agonistic or antagonistic function. In some embodiments, the cell surface protein is dimerized and the oligomerization domain of the first recombinant fusion protein comprises a dimerization domain. In some embodiments, the cell surface protein is trimerized and the oligomerization domain of the first recombinant fusion protein comprises a trimerization domain. In some embodiments, the cell surface protein is tetramerized and the oligomerization domain of the first recombinant fusion protein comprises a tetramerization domain. In some embodiments, the display polypeptide of the first or second recombinant fusion protein comprises a single chain variable fragment (scFv), a type I transmembrane polypeptide, a type II transmembrane polypeptide, a type III transmembrane polypeptide, a GPI-anchored polypeptide, a secreted polypeptide, or a multi-pass transmembrane polypeptide. In some embodiments, the scFv comprises an anti-BCMA scFv polypeptide, an anti-NTD scFv polypeptide, an anti-RBD scFv polypeptide, or an anti-CD19 scFv polypeptide. In some embodiments, the type I transmembrane polypeptide comprises ACE2. ICAM-1. LILRA5. LILRB3, or LILRB4. In some embodiments, the type II transmembrane polypeptide comprises DPP4, or BAFF. In some embodiments, the type III transmembrane polypeptide comprises BCMA. TACI, or BAFF Receptor. In some embodiments, the GPI-anchored polypeptide comprises CD24. CD48, CD59 or CD55. In some embodiments, the secreted polypeptide comprises IL-4, or CXCL-12. In some embodiments, the multi-pass transmembrane polypeptide comprises NTCP, or FCεRI beta. In some embodiments, the display polypeptide of the first or second recombinant fusion protein comprises an antibody, a receptor, a cytokine, an immune checkpoint protein, a chemokine, an adhesion factor, or a mitogen. In some embodiments, the antibody comprises a non-neutralizing antibody, a neutralizing antibody, or an antibody of multiple formats. In some embodiments, the receptor comprises ACE2, or DPP4. In some embodiments, the cytokine comprises IL-4, or IL-6.

In some embodiments, the immune checkpoint protein comprises CTLA-4, or PD-1. In some embodiments, the chemokine comprises CXCL-12. In some embodiments, the adhesion factor comprises ICAM-1, or LFA-1. In some embodiments, the mitogen comprises Wnt. In some embodiments, the display polypeptide of the first or second recombinant fusion protein comprises an amino acid sequence that has at least about 80%, 85%, 90%, 95% or 99% sequence identity to an amino acid sequence according to SEQ ID NOs: 20-51, 56-70. In some embodiments, the first recombinant fusion protein and the second recombinant fusion protein have the same transmembrane domains. In some embodiments, the first recombinant fusion protein and the second recombinant fusion protein have different transmembrane domains. In some embodiments, the transmembrane domain anchors the fusion protein to a bilayer of the enveloped particle. In some embodiments, the first or second recombinant fusion protein comprises: (a) the transmembrane domain of a Vesicular Stomatitis virus glycoprotein (VSV-G); (b) the transmembrane domain of a Dengue E protein: (c) the transmembrane domain of influenza Hemagglutinin (HA): (d) the transmembrane domain of HIV surface glycoprotein GP120 or GP41: (c) the transmembrane domain of measles virus surface glycoprotein hamagglutinin (H) protein; or (f) the transmembrane domain of influenza Neuraminidase (NA). In some embodiments, the first or second recombinant fusion protein comprises: (a) the transmembrane domain and cytosolic domain of a Vesicular Stomatitis virus glycoprotein (VSV-G); (b) the transmembrane domain and cytosolic domain of a Dengue E protein: (c) the transmembrane domain and cytosolic domain of influenza Hemagglutinin (HA): (d) the transmembrane domain and cytosolic domain of HIV surface glycoprotein GP120 or GP41: (c) the transmembrane domain and cytosolic domain of measles virus surface glycoprotein hamagglutinin (H) protein: or (f) the transmembrane domain and cytosolic domain of influenza Neuraminidase (NA). In some embodiments. (a) the display polypeptide of the first or second recombinant fusion protein comprises a single chain variable fragment (scFv), a type I transmembrane polypeptide, a type II transmembrane polypeptide, a type III transmembrane polypeptide, a GPI-anchored polypeptide, a secreted polypeptide, a multi-pass transmembrane polypeptide, an antibody, a receptor, a cytokine, an immune checkpoint protein, a chemokine, an adhesion factor, or a mitogen: (b) the transmembrane domain of the first or second recombinant fusion comprises the transmembrane domain of a Vesicular Stomatitis virus glycoprotein (VSV-G), the transmembrane domain of a Dengue E protein, the transmembrane domain of influenza Hemagglutinin (HA), the transmembrane domain of HIV surface glycoprotein GP120 or GP41, the transmembrane domain of measles virus surface glycoprotein hamagglutinin (H) protein, or the transmembrane domain of influenza Neuraminidase (NA); and (c) the oligomerization domain of the first recombinant fusion protein comprises a leucine zipper dimerization domain. D4 post-fusion trimerization domain of VSV-G protein, a Dengue E protein post-fusion trimerization domain, a foldon trimerization domain, or an influenza neuraminidase stem domain. The enveloped particle of claim 60, wherein: (a) the display polypeptide of the first or second recombinant fusion protein comprises an anti-BCMA scFv polypeptide, an anti-CD19 scFv polypeptide, an anti-NTD scFv polypeptide, an anti-RBD scFv polypeptide. ACE2. ICAM-1. LILRA5. LILRB3. LILRB4. DPP4. BAFF. BCMA. TACI. BAFF Receptor. CD24, CD48. CD59. CD55. IL-4. CXCL-12. NTCP. FCεRI beta, a non-neutralizing antibody, a neutralizing antibody, an antibody of multiple formats, IL-6, CTLA-4. PD-1. LFA-1. Wnt. CD80. CD86. OX40L. CD3, or 4-1BBL: (b) the transmembrane domain of the first or second recombinant fusion comprises the transmembrane domain of a Vesicular Stomatitis virus glycoprotein (VSV-G), the transmembrane domain of a Dengue E protein, the transmembrane domain of influenza Hemagglutinin (HA), the transmembrane domain of HIV surface glycoprotein GP120 or GP41, the transmembrane domain of measles virus surface glycoprotein hamagglutinin (H) protein, or the transmembrane domain of influenza Neuraminidase (NA); and (c) the oligomerization domain of the first recombinant fusion protein comprises a leucine zipper dimerization domain. D4 post-fusion trimerization domain of VSV-G protein, a Dengue E protein post-fusion trimerization domain, a foldon trimerization domain, or an influenza neuraminidase stem domain. In some embodiments. (a) the display polypeptide of the first or second recombinant fusion protein comprises an amino acid sequence that has at least about 80%, 85%, 90%, 95% or 99% sequence identity to an amino acid sequence according to SEQ ID NOs: 20-51, 56-70; (b) the transmembrane domain of the first or second recombinant fusion comprises the transmembrane domain of a Vesicular Stomatitis virus glycoprotein (VSV-G), the transmembrane domain of a Dengue E protein, the transmembrane domain of influenza Hemagglutinin (HA), the transmembrane domain of HIV surface glycoprotein GP120 or GP41, the transmembrane domain of measles virus surface glycoprotein hamagglutinin (H) protein, or the transmembrane domain of influenza Neuraminidase (NA); and (c) the oligomerization domain of the first recombinant fusion protein comprises a leucine zipper dimerization domain, D4 post-fusion trimerization domain of VSV-G protein, a Dengue E protein post-fusion trimerization domain, a foldon trimerization domain, or an influenza neuraminidase stem domain. In some embodiments, (a) the display polypeptide of the first or second recombinant fusion protein comprises an amino acid sequence that has at least about 80%, 85%, 90%, 95% or 99% sequence identity to an amino acid sequence according to SEQ ID NOs: 20-51, 56-70; (b) the transmembrane domain of the first or second recombinant fusion comprises an amino acid sequence that has at least about 80%, 85%, 90%, 95% or 99% sequence identity to an amino acid sequence according to SEQ ID NOs: 1-4, 52-55; and (c) the oligomerization domain of the first recombinant fusion protein comprises an amino acid sequence that has at least about 80%, 85%, 90%, 95% or 99% sequence identity to an amino acid sequence of SEQ ID NOs: 6-19. In some embodiments, the cytosolic domain comprises an amino acid sequence that has at least about 80%, 85%, 90%, 95% or 99% sequence identity to SEQ ID No: 5. In some embodiments, the transmembrane domain comprises an amino acid sequence that has at least about 80%, 85%, 90%, 95% or 99% sequence identity to an amino acid sequence according to SEQ ID NOs: 1-4, 52-55.

In some embodiments, when the first or second recombinant fusion protein is expressed on the surface of the enveloped particle the first or second recombinant fusion protein is expressed at a valency of about 10 copies on the surface of the enveloped particle. In some embodiments, when the first or second recombinant fusion protein is expressed on the surface of the enveloped particle the first or second recombinant fusion protein is expressed at a valency of about 10 to 15 copies on the surface of the enveloped particle. In some embodiments, when the first or second recombinant fusion protein is expressed on the surface of the enveloped particle the first or second recombinant fusion protein is expressed at a valency of at least about 25 copies on the surface of the enveloped particle. In some embodiments, when the first or second recombinant fusion protein is expressed on the surface of the enveloped particle the first or second recombinant fusion protein is expressed at a valency of at least about 50 copies on the surface of the enveloped particle. In some embodiments, when the first or second recombinant fusion protein is expressed on the surface of the enveloped particle the first or second recombinant fusion protein is expressed at a valency of at least about 100 copies on the surface of the enveloped particle. In some embodiments, when the first or second recombinant fusion protein is expressed on the surface of the enveloped particle the first or second recombinant fusion protein is expressed at a valency of at least about 200 copies on the surface of the enveloped particle. In some embodiments, when the first or second recombinant fusion protein is expressed on the surface of the enveloped particle the first or second recombinant fusion protein is expressed at a valency of at least about 400 copies on the surface of the enveloped particle. In some embodiments, when the first or second recombinant fusion protein is expressed on the surface of the enveloped particle the first or second recombinant fusion protein is expressed at a valency of at least about 600 copies on the surface of the enveloped particle. In some embodiments, when the first or second recombinant fusion protein is expressed on the surface of the enveloped particle the first or second recombinant fusion protein is expressed on the surface of the enveloped particle at a valency of at least about 1000 copies on the surface of the enveloped particle. In some embodiments, the recombinant fusion protein comprises an amino acid sequence having at least about 75%, 80%, 85%, 90%, 95%, or 99% sequence identity to any of SEQ ID NOs: 72-78, 81-94, 96-100. In some embodiments, the first recombinant fusion protein comprises an amino acid sequence having at least about 75%, 80%, 85%, 90%, 95%, or 99% sequence identity to any of SEQ ID NOs: 72-78, 81-94, 96-100. In some embodiments, the second recombinant fusion protein comprises an amino acid sequence having at least about 75%, 80%, 85%, 90%, 95%, or 99% sequence identity to any of SEQ ID NOs: 71-100.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates vector design for a monomeric display vector expressing a fusion protein consisting of a protein linked to the VSVG transmembrane and intracellular domains.

FIG. 1B illustrates vector design for a trimeric display vector expressing a fusion protein consisting of a protein linked to the D4 post-fusion trimerization domain of VSVG, followed by the transmembrane and intracellular domains of VSVG.

FIGS. 2A-2C illustrate generation of monomeric enveloped particles.

FIGS. 3A-3C illustrate generation of trimeric enveloped particles.

FIGS. 4A-4C illustrate generation of generation of mixed monomeric and trimeric enveloped particles.

FIGS. 5A-5C illustrate various D4 configurations.

FIGS. 6A-6C illustrate various oligomerization domains and configurations.

FIG. 7. illustrates the design of monomeric display vectors.

FIG. 8A illustrates the generation of monomeric viral-like particles (VLPs) with viral genomes.

FIG. 8B illustrates the generation of monomeric VLPs without viral genomes.

FIG. 8C illustrates the generation of monomeric extracellular vesicles (EVs).

FIG. 9A illustrates quantitative western-blot analysis of monomeric ACE2-VM MVPs.

FIG. 9B illustrates western-blot analysis of monomeric ACE2-VM MVPs under reducing or non-reducing conditions.

FIG. 9C illustrates SARS CoV-2 pseudovirus neutralization analysis of monomeric ACE2-VM MVPs.

FIG. 10 illustrates the design of MVP display vector with an oligomerization domain.

FIG. 11 illustrates the design of a dimeric MVP display vector.

FIG. 12A illustrates the generation of dimeric VLPs with viral genomes.

FIG. 12B illustrates the generation of dimeric VLPs without viral genomes.

FIG. 12C illustrates the generation of dimeric EVs.

FIG. 13A illustrates quantitative western-blot analysis of dimeric ACE2-LZ MVPs.

FIG. 13B illustrates western-blot analyses of dimeric ACE2-LZ MVPs under reducing or non-reducing conditions.

FIG. 13C illustrates SARS CoV-2 pseudovirus neutralization analysis of dimeric ACE2-LZ MVPs.

FIG. 14 illustrates the design of trimeric MVP display vector using the D4 post-fusion trimerization domain from VSVG.

FIG. 15 illustrates the design of trimeric MVP display vector using the DE post-fusion trimerization domain of Dengue E protein.

FIG. 16 illustrates the design of trimeric MVP display vector using the Foldon trimerization domain of T4 Phage Fibritin.

FIG. 17A illustrates the generation of trimeric VLPs with viral genomes.

FIG. 17B illustrates the generation of trimeric VLPs without viral genomes.

FIG. 17C illustrates the generation of trimeric EVs.

FIG. 18A illustrates quantitative western-blot analysis of trimeric ACE2-D4, ACE2-DE, and ACE2-Fold MVPs.

FIG. 18B illustrates western-blot analysis of ACE2-D4, ACE2-DE, and ACE2-Fold MVPs under reducing or non-reducing conditions.

FIG. 18C illustrates SARS CoV-2 pseudovirus neutralization analysis of trimeric ACE2-D4, ACE2-DE, and ACE2-Fold MVPs.

FIG. 19A illustrates quantitative western-blot analysis of monomeric ACE2-VM MVPs, dimeric ACE2-LZ MVPs, and trimeric ACE2-D4, ACE2-DE, ACE2-Fold MVPs under reducing condition.

FIG. 19B illustrates western-blot analysis of monomeric ACE2-VM MVPs, dimeric ACE2-LZ MVPs, and trimeric ACE2-DE, ACE2-Fold MVPs under non-reducing and reducing conditions.

FIG. 19C illustrates SARS CoV-2 pseudovirus neutralization analysis of monomeric ACE2-VM MVPs, dimeric ACE2-LZ MVPs, and trimeric ACE2-D4, ACE2-DE, ACE2-Fold MVPs.

FIG. 20 illustrates design of tetrameric, NA-fusion construct for displaying Type II transmembrane proteins on MVPs.

FIG. 21A illustrates the generation of tetrameric VLPs with viral genomes.

FIG. 21B illustrates the generation of tetrameric VLPs without viral genomes.

FIG. 21C illustrates the generation of tetrameric EVs.

FIG. 22A illustrates two variants of tetrameric type II display anchors.

FIG. 22B illustrates quantitative western-blot analysis of tetrameric DPP4-MVPs with two display variants.

FIG. 22C illustrates MERS CoV-2 pseudovirus neutralization analysis of tetrameric DPP4-MVPs with two display variants.

FIG. 23A illustrates the generation of mixed oligomeric VLPs with viral genomes.

FIG. 23B illustrates the generation of mixed oligomeric VLPs without viral genomes.

FIG. 23C illustrates the generation of mixed oligomeric EVs.

FIG. 24A illustrates quantitative western-blot analysis of single and mixed MVPs targeting NTD, RBD, or both.

FIG. 24B illustrates SARS CoV-2 pseudovirus neutralization analysis of single and mixed MVPs targeting NTD, RBD, or both.

FIG. 24C illustrates fold-of-repression by single and mixed MVPs targeting NTD, RBD, or both.

FIG. 25A illustrates MVP display vectors with oligomerization domain located before or after the transmembrane anchor.

FIG. 25B illustrates quantitative western-blot analysis of trimeric ACE2-MVPs with D4 trimerization domain located before or after the transmembrane anchor.

FIG. 25C illustrates SARS CoV-2 pseudovirus neutralization analysis of ACE2-MVPs with D4 trimerization domain located before or after the transmembrane anchor.

FIG. 26A illustrates the truncated forms of D4 trimerization domain.

FIG. 26B illustrates quantitative western blot analysis of ACE2-MVPs with truncated forms of D4 trimerization domain.

FIG. 26C illustrates SARS CoV-2 pseudovirus neutralization analysis of ACE2-MVPs with the truncated forms of D4 trimerization domain.

FIG. 27A illustrates western blot analysis of trimeric MVPs displaying several types of murine LILR.

FIG. 27B illustrates quantitative western blot characterization of MVPs displaying various peptides in different oligomeric forms.

FIG. 27C illustrates quantitative western blot characterization of monomeric and trimeric MVPs displaying anti-CD19 single chain variable fragment (scFv).

FIG. 28A illustrates generation of trimeric ACE2 extracellular vesicles (ACE-D4VG EVs).

FIG. 28B illustrates particle size distribution of ACE2-D4VG EVs.

FIG. 28C illustrates quantitative western-blot analysis of ACE2-D4VG EVs.

FIG. 28D illustrates SARS CoV-2 pseudovirus neutralization analysis of ACE2-D4VG EVs.

FIG. 28E illustrates the neutralizing activity of ACE2-D4VG EVs using the SARS CoV-2 live virus neutralization assay.

FIG. 28F illustrates the cytoxicity of ACE2-D4VG EVs using the SARS CoV-2 live virus neutralization assay.

FIG. 29A illustrates vector design for trimeric display of co-stimulatory molecules on EVs.

FIG. 29B illustrates generation of EVs displaying co-stimulatory molecules.

FIG. 30A illustrates western-blot analysis of human CD86-EVs in non-reducing or reducing conditions.

FIG. 30B illustrates the effects of co-stimulatory human CD86-EVs on T cell activation.

FIG. 30C illustrates the effects of co-stimulatory human CD86-EVs on T cell differentiation.

FIG. 30D illustrates the effects of co-stimulatory human CD86-EVs on T cell proliferation.

FIG. 31A illustrates western-blot analysis of human OX40L-EVs in non-reducing or reducing conditions.

FIG. 31B illustrates the effects of co-stimulatory human OX40L-EVs on T cell activation.

FIG. 31C illustrates the effects of co-stimulatory human OX40L-EVs on T cell differentiation.

FIG. 31D illustrates the effects of co-stimulatory human OX40L-EVs on T cell proliferation.

FIG. 32A illustrates the effects of co-stimulatory human 41BBL-EVs on T cell activation.

FIG. 32B illustrates the effects of co-stimulatory human 41BBL-EVs on T cell differentiation.

FIG. 32C illustrates the effects of co-stimulatory human 41BBL-EVs on T cell proliferation.

### DETAILED DESCRIPTION OF THE INVENTION

Multivalent interactions play critical roles in a variety of biological processes. A multivalent ligand can bind to one or more multiple receptors with enhanced functional affinity. Such interactions can induce receptor clustering and lead to signal transduction. However, despite the beneficial role of multivalent interactions in normal physiological processes, multivalent interactions also underlie cell regulation that contributes to disease pathogenesis. Multivalence can exponentially enhance the functional affinity of ligand-receptor interactions and may create significant barriers for small molecule or antibody drugs.

To overcome such challenges associated with targeting disease pathogenesis characterized by multivalence, described herein are recombinant fusion proteins that comprise oligomerization domains that when expressed on the surface of enveloped particle are displayed in an oligomerized format. In some embodiments, the recombinant fusion protein is expressed on the surface of the enveloped particle at a valency of 10 or more copies. Without being bound by a particular theory, expression of the recombinant fusion proteins at a high valency on the enveloped particle can effectively mimic the multivalent target involved in the disease progression and counteract the multivalent target through antagonism or agonism. The multivalent target can be further mimicked through the use of an oligomerization domain in combination with a high copy number to counteract the multivalent target more effectively than administration of a single agent such as isolated antibodies or small molecules.

### Recombinant Fusion Proteins

Disclosed herein are recombinant fusion proteins comprising a transmembrane domain, a display polypeptide, and an oligomerization domain wherein when the recombinant fusion protein is expressed on the surface of an enveloped particle, the recombinant fusion protein is displayed in an oligomeric format. In some embodiments, the recombinant fusion protein comprises a cytosolic domain.

In some embodiments, the oligomerization domain is a dimerization domain. In some embodiments, the dimerization domain comprises a leucine zipper dimerization domain. In some embodiments, the oligomerization domain is a trimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the oligomerization domain is a tetramerization domain. In some embodiments, the tetramerization domain comprises an influenza neuraminidase stem domain.

TABLE 1
Exemplary Oligomerization Domain Sequences

| Oligomerization | | | |
|---|---|---|---|
| Domain | Valency | Amino Acid Sequences | SEQ ID No. |
| D4 Variation 1 | Trimer | IGTALQVKMPKSHKAIQADGWMCHASK | 6 |
| | | WVTTCDFRWYGPKYITHSIRSFTPSVEQ | |
| | | CKESIEQTKQGTWLNPGFPPQSCGYATV | |
| | | TD7AEAVIVQVTPHHVLVDEYTGEWVD | |
| | | SQFIN8GKCSNYICPTVHNSTTWHSDYK | |
| | | VKGLCDSNLISMDI | |
| D4 Variation 2 | Trimer | IQADGWMCHASKWVTTCDFRWYGPKY | 7 |
| | | ITHSIRSFTPSVEQCKESIEQTKQGTWLNP | |
| | | GFPPQSCGYATVTDAEAVIVQVTPHHVL | |
| | | VDEYTGEWVDSQFINGKCSNYICPTVHN | |
| | | STTWHSDYKVKGLCDSNL | |
| D4 Variation 3 | Trimer | IQADGWMCHASKWVTTCDFRWYGPKY | 8 |
| | | ITHSIRSFTPSVEQCKESIEQTKQGTWLNP | |
| | | GFPPQSCGYATVTDAEAVIVQVTPHHVL | |
| | | VDEYTGEWVDSQFINGKCSNYICPTVHN | |
| | | STT | |
| D4 Variation 4 | Trimer | IQADGWMCHASKWVTTCDFRWYGPKY | 9 |
| | | ITHSIRSFTPSVEQCKESIEQTKQGTWLNP | |
| | | GFPPQSCGYATVIDAEAVIVQVTPHHVL | |
| | | VDEYTGEWVDSQFING | |
| D4 Variation 5 | Trimer | IQADGWMCHASKWVTTCDFRWYGPKY | 10 |
| | | ITHSIRSFTPSVEQCKESIEQTKQGTWLNP | |
| | | GFPPQSCGYATVTDAEAVIVQVTPHHVL | |
| Foldon | Trimer | GYIPEAPRDGQAYVRKDGEWVLLSTFL | 11 |
| Leucine Zipper | Dimer | RMKQLEDKVEELLSKQYHLENEVARLK | 12 |
| V1 | | KLVGER | |
| Leucine Zipper | Dimer | RMKQLEDKVEELLSKNYHLENEVARLK | 13 |
| V2 | | KLVGER | |
| Neuraminidase | Tetramer | MNPNQKIITIGSICLVVGLISLILQIGNIISI | 14 |
| Stem V1 | | WISHSIQT | |
| Neuraminidase | Tetramer | MNPNQKIITIGSICMVTGIVSLMLQIGNM | 15 |
| Stem V2 | | ISIWVSHSIHTGNQHQSEPISNTNFLTEKA | |
| | | VASVKLAGNSSLCPIN | |
| Dengue E Fusion | Trimer | KLCIEAKISNTTTDSRCPTQGEATLVEEQ | 16 |
| V1 | | DTNFVCRRTFVDRGHGNGCGLFGKGSLI | |
| | | TCAKFKCVTKL | |
| Dengue E Fusion | Trimer | IELLKTEVTNPAVLRKLCIEAKISNTTTDS | 17 |
| V2 | | RCPTQGEATLVEEQDTNFVCRRTFVDRG | |
| | | HGNGCGLFGKGSLITCAKFKCVTKL | |
| Dengue E Fusion | Trimer | KLCIEAKISNTTTDSRCPTQGEATLVEEQ | 18 |
| V3 | | DTNFVCRRTFVDRGHGNGCGLFGKGSLI | |
| | | TCAKFKCVTKLEGKIVQYENLKYSVI | |
| Dengue E Fusion | Trimer | EAKISNTTTDSRCPTQGEATLVEEQDTNF | 19 |
| V4 | | VCRRTFVDRGHGNGCGLFGKGSLITCAKFK | |

In some embodiments, the oligomerization domain comprises an amino acid sequence of at least 75% sequence identity to an amino acid sequence according to SEQ ID NO: 6. In some embodiments, the oligomerization domain comprises an amino acid sequence of at least 80% sequence identity to an amino acid sequence according to SEQ ID NO: 6. In some embodiments, the oligomerization domain comprises an amino acid sequence of at least 85% sequence identity to an amino acid sequence according to SEQ ID NO: 6. In some embodiments, the oligomerization domain comprises an amino acid sequence of at least 90% sequence identity to an amino acid sequence according to SEQ ID NO: 6. In some embodiments, the oligomerization domain comprises an amino acid sequence of at least 95% sequence identity to an amino acid sequence according to SEQ ID NO: 6. In some embodiments, the oligomerization domain comprises an amino acid sequence of at least 97% sequence identity to an amino acid sequence according to SEQ ID NO: 6. In some embodiments, the oligomerization domain comprises an amino acid sequence of at least 98% sequence identity to an amino acid sequence according to SEQ ID NO: 6. In some embodiments, the oligomerization domain comprises an amino acid sequence of at least 99% sequence identity to an amino acid sequence according to SEQ ID NO: 6.

In some embodiments, the oligomerization domain comprises an amino acid sequence of at least 75% sequence homology to an amino acid sequence according to SEQ ID NO: 6. In some embodiments, the oligomerization domain comprises an amino acid sequence of at least 80% sequence homology to an amino acid sequence according to SEQ ID NO: 6. In some embodiments, the oligomerization domain comprises an amino acid sequence of at least 85% sequence homology to an amino acid sequence according to SEQ ID NO: 6. In some embodiments, the oligomerization domain comprises an amino acid sequence of at least 90% sequence homology to an amino acid sequence according to SEQ ID NO: 6. In some embodiments, the oligomerization domain comprises an amino acid sequence of at least 95% sequence homology to an amino acid sequence according to SEQ ID NO: 6. In some embodiments, the oligomerization domain comprises an amino acid sequence of at least 97% sequence homology to an amino acid sequence according to SEQ ID NO: 6. In some embodiments, the oligomerization domain comprises an amino acid sequence of at least 98% sequence homology to an amino acid sequence according to SEQ ID NO: 6. In some embodiments, the oligomerization domain comprises an amino acid sequence of at least 99% sequence homology to an amino acid sequence according to SEQ ID NO: 6.

In some embodiments, the oligomerization domain comprises an amino acid sequence according to SEQ ID NO: 6.

In some embodiments, the oligomerization domain comprises an amino acid sequence of at least 75% sequence identity to an amino acid sequence according to SEQ ID NO: 7. In some embodiments, the oligomerization domain comprises an amino acid sequence of at least 80% sequence identity to an amino acid sequence according to SEQ ID NO: 7. In some embodiments, the oligomerization domain comprises an amino acid sequence of at least 85% sequence identity to an amino acid sequence according to SEQ ID NO: 7. In some embodiments, the oligomerization domain comprises an amino acid sequence of at least 90% sequence identity to an amino acid sequence according to SEQ ID NO: 7. In some embodiments, the oligomerization domain comprises an amino acid sequence of at least 95% sequence identity to an amino acid sequence according to SEQ ID NO: 7. In some embodiments, the oligomerization domain comprises an amino acid sequence of at least 97% sequence identity to an amino acid sequence according to SEQ ID NO: 7. In some embodiments, the oligomerization domain comprises an amino acid sequence of at least 98% sequence identity to an amino acid sequence according to SEQ ID NO: 7. In some embodiments, the oligomerization domain comprises an amino acid sequence of at least 99% sequence identity to an amino acid sequence according to SEQ ID NO: 7.

In some embodiments, the oligomerization domain comprises an amino acid sequence of at least 75% sequence homology to an amino acid sequence according to SEQ ID NO: 7. In some embodiments, the oligomerization domain comprises an amino acid sequence of at least 80% sequence homology to an amino acid sequence according to SEQ ID NO: 7. In some embodiments, the oligomerization domain comprises an amino acid sequence of at least 85% sequence homology to an amino acid sequence according to SEQ ID NO: 7. In some embodiments, the oligomerization domain comprises an amino acid sequence of at least 90% sequence homology to an amino acid sequence according to SEQ ID NO: 7. In some embodiments, the oligomerization domain comprises an amino acid sequence of at least 95% sequence homology to an amino acid sequence according to SEQ ID NO: 7. In some embodiments, the oligomerization domain comprises an amino acid sequence of at least 97% sequence homology to an amino acid sequence according to SEQ ID NO: 7. In some embodiments, the oligomerization domain comprises an amino acid sequence of at least 98% sequence homology to an amino acid sequence according to SEQ ID NO: 7. In some embodiments, the oligomerization domain comprises an amino acid sequence of at least 99% sequence homology to an amino acid sequence according to SEQ ID NO: 7.

In some embodiments, the oligomerization domain comprises an amino acid sequence according to SEQ ID NO: 7.

In some embodiments, the oligomerization domain comprises an amino acid sequence of at least 75% sequence identity to an amino acid sequence according to SEQ ID NO: 8. In some embodiments, the oligomerization domain comprises an amino acid sequence of at least 80% sequence identity to an amino acid sequence according to SEQ ID NO: 8. In some embodiments, the oligomerization domain comprises an amino acid sequence of at least 85% sequence identity to an amino acid sequence according to SEQ ID NO: 8. In some embodiments, the oligomerization domain comprises an amino acid sequence of at least 90% sequence identity to an amino acid sequence according to SEQ ID NO: 8. In some embodiments, the oligomerization domain comprises an amino acid sequence of at least 95% sequence identity to an amino acid sequence according to SEQ ID NO: 8. In some embodiments, the oligomerization domain comprises an amino acid sequence of at least 97% sequence identity to an amino acid sequence according to SEQ ID NO: 8. In some embodiments, the oligomerization domain comprises an amino acid sequence of at least 98% sequence identity to an amino acid sequence according to SEQ ID NO: 8. In some embodiments, the oligomerization domain comprises an amino acid sequence of at least 99% sequence identity to an amino acid sequence according to SEQ ID NO: 8.

In some embodiments, the oligomerization domain comprises an amino acid sequence of at least 75% sequence homology to an amino acid sequence according to SEQ ID NO: 8. In some embodiments, the oligomerization domain comprises an amino acid sequence of at least 80% sequence homology to an amino acid sequence according to SEQ ID NO: 8. In some embodiments, the oligomerization domain comprises an amino acid sequence of at least 85% sequence homology to an amino acid sequence according to SEQ ID NO: 8. In some embodiments, the oligomerization domain comprises an amino acid sequence of at least 90% sequence homology to an amino acid sequence according to SEQ ID NO: 8. In some embodiments, the oligomerization domain comprises an amino acid sequence of at least 95% sequence homology to an amino acid sequence according to SEQ ID NO: 8. In some embodiments, the oligomerization domain comprises an amino acid sequence of at least 97% sequence homology to an amino acid sequence according to SEQ ID NO: 8. In some embodiments, the oligomerization domain comprises an amino acid sequence of at least 98% sequence homology to an amino acid sequence according to SEQ ID NO: 8. In some embodiments, the oligomerization domain comprises an amino acid sequence of at least 99% sequence homology to an amino acid sequence according to SEQ ID NO: 8.

In some embodiments, the oligomerization domain comprises an amino acid sequence according to SEQ ID NO: 8.

In some embodiments, the oligomerization domain comprises an amino acid sequence of at least 75% sequence identity to an amino acid sequence according to SEQ ID NO: 9. In some embodiments, the oligomerization domain comprises an amino acid sequence of at least 80% sequence identity to an amino acid sequence according to SEQ ID NO: 9. In some embodiments, the oligomerization domain comprises an amino acid sequence of at least 85% sequence identity to an amino acid sequence according to SEQ ID NO: 9. In some embodiments, the oligomerization domain comprises an amino acid sequence of at least 90% sequence identity to an amino acid sequence according to SEQ ID NO: 9. In some embodiments, the oligomerization domain comprises an amino acid sequence of at least 95% sequence identity to an amino acid sequence according to SEQ ID NO: 9. In some embodiments, the oligomerization domain comprises an amino acid sequence of at least 97% sequence identity to an amino acid sequence according to SEQ ID NO: 9. In some embodiments, the oligomerization domain comprises an amino acid sequence of at least 98% sequence identity to an amino acid sequence according to SEQ ID NO: 9. In some embodiments, the oligomerization domain comprises an amino acid sequence of at least 99% sequence identity to an amino acid sequence according to SEQ ID NO: 9.

In some embodiments, the oligomerization domain comprises an amino acid sequence of at least 75% sequence homology to an amino acid sequence according to SEQ ID NO: 9. In some embodiments, the oligomerization domain comprises an amino acid sequence of at least 80% sequence homology to an amino acid sequence according to SEQ ID NO: 9. In some embodiments, the oligomerization domain comprises an amino acid sequence of at least 85% sequence homology to an amino acid sequence according to SEQ ID NO: 9. In some embodiments, the oligomerization domain comprises an amino acid sequence of at least 90% sequence homology to an amino acid sequence according to SEQ ID NO: 9. In some embodiments, the oligomerization domain comprises an amino acid sequence of at least 95% sequence homology to an amino acid sequence according to SEQ ID NO: 9. In some embodiments, the oligomerization domain comprises an amino acid sequence of at least 97% sequence homology to an amino acid sequence according to SEQ ID NO: 9. In some embodiments, the oligomerization domain comprises an amino acid sequence of at least 98% sequence homology to an amino acid sequence according to SEQ ID NO: 9. In some embodiments, the oligomerization domain comprises an amino acid sequence of at least 99% sequence homology to an amino acid sequence according to SEQ ID NO: 9.

In some embodiments, the oligomerization domain comprises an amino acid sequence according to SEQ ID NO: 9.

In some embodiments, the oligomerization domain comprises an amino acid sequence of at least 75% sequence identity to an amino acid sequence according to SEQ ID NO: 10. In some embodiments, the oligomerization domain comprises an amino acid sequence of at least 80% sequence identity to an amino acid sequence according to SEQ ID NO: 10. In some embodiments, the oligomerization domain comprises an amino acid sequence of at least 85% sequence identity to an amino acid sequence according to SEQ ID NO: 10. In some embodiments, the oligomerization domain comprises an amino acid sequence of at least 90% sequence identity to an amino acid sequence according to SEQ ID NO: 10. In some embodiments, the oligomerization domain comprises an amino acid sequence of at least 95% sequence identity to an amino acid sequence according to SEQ ID NO: 10. In some embodiments, the oligomerization domain comprises an amino acid sequence of at least 97% sequence identity to an amino acid sequence according to SEQ ID NO: 10. In some embodiments, the oligomerization domain comprises an amino acid sequence of at least 98% sequence identity to an amino acid sequence according to SEQ ID NO: 10. In some embodiments, the oligomerization domain comprises an amino acid sequence of at least 99% sequence identity to an amino acid sequence according to SEQ ID NO: 10.

In some embodiments, the oligomerization domain comprises an amino acid sequence of at least 75% sequence homology to an amino acid sequence according to SEQ ID NO: 10. In some embodiments, the oligomerization domain comprises an amino acid sequence of at least 80% sequence homology to an amino acid sequence according to SEQ ID NO: 10. In some embodiments, the oligomerization domain comprises an amino acid sequence of at least 85% sequence homology to an amino acid sequence according to SEQ ID NO: 10. In some embodiments, the oligomerization domain comprises an amino acid sequence of at least 90% sequence homology to an amino acid sequence according to SEQ ID NO: 10. In some embodiments, the oligomerization domain comprises an amino acid sequence of at least 95% sequence homology to an amino acid sequence according to SEQ ID NO: 10. In some embodiments, the oligomerization domain comprises an amino acid sequence of at least 97% sequence homology to an amino acid sequence according to SEQ ID NO: 10. In some embodiments, the oligomerization domain comprises an amino acid sequence of at least 98% sequence homology to an amino acid sequence according to SEQ ID NO: 10. In some embodiments, the oligomerization domain comprises an amino acid sequence of at least 99% sequence homology to an amino acid sequence according to SEQ ID NO: 10.

In some embodiments, the oligomerization domain comprises an amino acid sequence according to SEQ ID NO: 10.

In some embodiments, the oligomerization domain comprises an amino acid sequence of at least 75% sequence identity to an amino acid sequence according to SEQ ID NO: 11. In some embodiments, the oligomerization domain comprises an amino acid sequence of at least 80% sequence identity to an amino acid sequence according to SEQ ID NO: 11. In some embodiments, the oligomerization domain comprises an amino acid sequence of at least 85% sequence identity to an amino acid sequence according to SEQ ID NO: 11. In some embodiments, the oligomerization domain comprises an amino acid sequence of at least 90% sequence identity to an amino acid sequence according to SEQ ID NO: 11. In some embodiments, the oligomerization domain comprises an amino acid sequence of at least 95% sequence identity to an amino acid sequence according to SEQ ID NO: 11. In some embodiments, the oligomerization domain comprises an amino acid sequence of at least 97% sequence identity to an amino acid sequence according to SEQ ID NO: 11.

In some embodiments, the oligomerization domain comprises an amino acid sequence of at least 75% sequence homology to an amino acid sequence according to SEQ ID NO: 11. In some embodiments, the oligomerization domain comprises an amino acid sequence of at least 80% sequence homology to an amino acid sequence according to SEQ ID NO: 11. In some embodiments, the oligomerization domain comprises an amino acid sequence of at least 85% sequence homology to an amino acid sequence according to SEQ ID NO: 11. In some embodiments, the oligomerization domain comprises an amino acid sequence of at least 90% sequence homology to an amino acid sequence according to SEQ ID NO: 11. In some embodiments, the oligomerization domain comprises an amino acid sequence of at least 95% sequence homology to an amino acid sequence according to SEQ ID NO: 11. In some embodiments, the oligomerization domain comprises an amino acid sequence of at least 97% sequence homology to an amino acid sequence according to SEQ ID NO: 11.

In some embodiments, the oligomerization domain comprises an amino acid sequence according to SEQ ID NO: 11.

In some embodiments, the oligomerization domain comprises an amino acid sequence of at least 75% sequence identity to an amino acid sequence according to SEQ ID NO: 12. In some embodiments, the oligomerization domain comprises an amino acid sequence of at least 80% sequence identity to an amino acid sequence according to SEQ ID NO: 12. In some embodiments, the oligomerization domain comprises an amino acid sequence of at least 85% sequence identity to an amino acid sequence according to SEQ ID NO: 12. In some embodiments, the oligomerization domain comprises an amino acid sequence of at least 90% sequence identity to an amino acid sequence according to SEQ ID NO: 12. In some embodiments, the oligomerization domain comprises an amino acid sequence of at least 95% sequence identity to an amino acid sequence according to SEQ ID NO: 12. In some embodiments, the oligomerization domain comprises an amino acid sequence of at least 97% sequence identity to an amino acid sequence according to SEQ ID NO: 12.

In some embodiments, the oligomerization domain comprises an amino acid sequence of at least 75% sequence homology to an amino acid sequence according to SEQ ID NO: 12. In some embodiments, the oligomerization domain comprises an amino acid sequence of at least 80% sequence homology to an amino acid sequence according to SEQ ID NO: 12. In some embodiments, the oligomerization domain comprises an amino acid sequence of at least 85% sequence homology to an amino acid sequence according to SEQ ID NO: 12. In some embodiments, the oligomerization domain comprises an amino acid sequence of at least 90% sequence homology to an amino acid sequence according to SEQ ID NO: 12. In some embodiments, the oligomerization domain comprises an amino acid sequence of at least 95% sequence homology to an amino acid sequence according to SEQ ID NO: 12. In some embodiments, the oligomerization domain comprises an amino acid sequence of at least 97% sequence homology to an amino acid sequence according to SEQ ID NO: 12.

In some embodiments, the oligomerization domain comprises an amino acid sequence according to SEQ ID NO: 12.

In some embodiments, the oligomerization domain comprises an amino acid sequence of at least 75% sequence identity to an amino acid sequence according to SEQ ID NO: 13. In some embodiments, the oligomerization domain comprises an amino acid sequence of at least 80% sequence identity to an amino acid sequence according to SEQ ID NO: 13. In some embodiments, the oligomerization domain comprises an amino acid sequence of at least 85% sequence identity to an amino acid sequence according to SEQ ID NO: 13. In some embodiments, the oligomerization domain comprises an amino acid sequence of at least 90% sequence identity to an amino acid sequence according to SEQ ID NO: 13. In some embodiments, the oligomerization domain comprises an amino acid sequence of at least 95% sequence identity to an amino acid sequence according to SEQ ID NO: 13. In some embodiments, the oligomerization domain comprises an amino acid sequence of at least 97% sequence identity to an amino acid sequence according to SEQ ID NO: 13.

In some embodiments, the oligomerization domain comprises an amino acid sequence of at least 75% sequence homology to an amino acid sequence according to SEQ ID NO: 13. In some embodiments, the oligomerization domain comprises an amino acid sequence of at least 80% sequence homology to an amino acid sequence according to SEQ ID NO: 13. In some embodiments, the oligomerization domain comprises an amino acid sequence of at least 85% sequence homology to an amino acid sequence according to SEQ ID NO: 13. In some embodiments, the oligomerization domain comprises an amino acid sequence of at least 90% sequence homology to an amino acid sequence according to SEQ ID NO: 13. In some embodiments, the oligomerization domain comprises an amino acid sequence of at least 95% sequence homology to an amino acid sequence according to SEQ ID NO: 13. In some embodiments, the oligomerization domain comprises an amino acid sequence of at least 97% sequence homology to an amino acid sequence according to SEQ ID NO: 13.

In some embodiments, the oligomerization domain comprises an amino acid sequence according to SEQ ID NO: 13.

In some embodiments, the oligomerization domain comprises an amino acid sequence of at least 75% sequence identity to an amino acid sequence according to SEQ ID NO: 14. In some embodiments, the oligomerization domain comprises an amino acid sequence of at least 80% sequence identity to an amino acid sequence according to SEQ ID NO: 14. In some embodiments, the oligomerization domain comprises an amino acid sequence of at least 85% sequence identity to an amino acid sequence according to SEQ ID NO: 14. In some embodiments, the oligomerization domain comprises an amino acid sequence of at least 90% sequence identity to an amino acid sequence according to SEQ ID NO: 14. In some embodiments, the oligomerization domain comprises an amino acid sequence of at least 95% sequence identity to an amino acid sequence according to SEQ ID NO: 14. In some embodiments, the oligomerization domain comprises an amino acid sequence of at least 97% sequence identity to an amino acid sequence according to SEQ ID NO: 14.

In some embodiments, the oligomerization domain comprises an amino acid sequence of at least 75% sequence homology to an amino acid sequence according to SEQ ID NO: 14. In some embodiments, the oligomerization domain comprises an amino acid sequence of at least 80% sequence homology to an amino acid sequence according to SEQ ID NO: 14. In some embodiments, the oligomerization domain comprises an amino acid sequence of at least 85% sequence homology to an amino acid sequence according to SEQ ID NO: 14. In some embodiments, the oligomerization domain comprises an amino acid sequence of at least 90% sequence homology to an amino acid sequence according to SEQ ID NO: 14. In some embodiments, the oligomerization domain comprises an amino acid sequence of at least 95% sequence homology to an amino acid sequence according to SEQ ID NO: 14. In some embodiments, the oligomerization domain comprises an amino acid sequence of at least 97% sequence homology to an amino acid sequence according to SEQ ID NO: 14.

In some embodiments, the oligomerization domain comprises an amino acid sequence according to SEQ ID NO: 14.

In some embodiments, the oligomerization domain comprises an amino acid sequence of at least 75% sequence identity to an amino acid sequence according to SEQ ID NO: 15. In some embodiments, the oligomerization domain comprises an amino acid sequence of at least 80% sequence identity to an amino acid sequence according to SEQ ID NO: 15. In some embodiments, the oligomerization domain comprises an amino acid sequence of at least 85% sequence identity to an amino acid sequence according to SEQ ID NO: 15. In some embodiments, the oligomerization domain comprises an amino acid sequence of at least 90% sequence identity to an amino acid sequence according to SEQ ID NO: 15. In some embodiments, the oligomerization domain comprises an amino acid sequence of at least 95% sequence identity to an amino acid sequence according to SEQ ID NO: 15. In some embodiments, the oligomerization domain comprises an amino acid sequence of at least 97% sequence identity to an amino acid sequence according to SEQ ID NO: 15. In some embodiments, the oligomerization domain comprises an amino acid sequence of at least 98% sequence identity to an amino acid sequence according to SEQ ID NO: 15.

In some embodiments, the oligomerization domain comprises an amino acid sequence of at least 75% sequence homology to an amino acid sequence according to SEQ ID NO: 15. In some embodiments, the oligomerization domain comprises an amino acid sequence of at least 80% sequence homology to an amino acid sequence according to SEQ ID NO: 15. In some embodiments, the oligomerization domain comprises an amino acid sequence of at least 85% sequence homology to an amino acid sequence according to SEQ ID NO: 15. In some embodiments, the oligomerization domain comprises an amino acid sequence of at least 90% sequence homology to an amino acid sequence according to SEQ ID NO: 15. In some embodiments, the oligomerization domain comprises an amino acid sequence of at least 95% sequence homology to an amino acid sequence according to SEQ ID NO: 15. In some embodiments, the oligomerization domain comprises an amino acid sequence of at least 97% sequence homology to an amino acid sequence according to SEQ ID NO: 15. In some embodiments, the oligomerization domain comprises an amino acid sequence of at least 98% sequence homology to an amino acid sequence according to SEQ ID NO: 15.

In some embodiments, the oligomerization domain comprises an amino acid sequence according to SEQ ID NO: 15.

In some embodiments, the oligomerization domain comprises an amino acid sequence of at least 75% sequence identity to an amino acid sequence according to SEQ ID NO: 16. In some embodiments, the oligomerization domain comprises an amino acid sequence of at least 80% sequence identity to an amino acid sequence according to SEQ ID NO: 16. In some embodiments, the oligomerization domain comprises an amino acid sequence of at least 85% sequence identity to an amino acid sequence according to SEQ ID NO: 16. In some embodiments, the oligomerization domain comprises an amino acid sequence of at least 90% sequence identity to an amino acid sequence according to SEQ ID NO: 16. In some embodiments, the oligomerization domain comprises an amino acid sequence of at least 95% sequence identity to an amino acid sequence according to SEQ ID NO: 16. In some embodiments, the oligomerization domain comprises an amino acid sequence of at least 97% sequence identity to an amino acid sequence according to SEQ ID NO: 16. In some embodiments, the oligomerization domain comprises an amino acid sequence of at least 98% sequence identity to an amino acid sequence according to SEQ ID NO: 16. In some embodiments, the oligomerization domain comprises an amino acid sequence of at least 99% sequence identity to an amino acid sequence according to SEQ ID NO: 16.

In some embodiments, the oligomerization domain comprises an amino acid sequence of at least 75% sequence homology to an amino acid sequence according to SEQ ID NO: 16. In some embodiments, the oligomerization domain comprises an amino acid sequence of at least 80% sequence homology to an amino acid sequence according to SEQ ID NO: 16. In some embodiments, the oligomerization domain comprises an amino acid sequence of at least 85% sequence homology to an amino acid sequence according to SEQ ID NO: 16. In some embodiments, the oligomerization domain comprises an amino acid sequence of at least 90% sequence homology to an amino acid sequence according to SEQ ID NO: 16. In some embodiments, the oligomerization domain comprises an amino acid sequence of at least 95% sequence homology to an amino acid sequence according to SEQ ID NO: 16. In some embodiments, the oligomerization domain comprises an amino acid sequence of at least 97% sequence homology to an amino acid sequence according to SEQ ID NO: 16. In some embodiments, the oligomerization domain comprises an amino acid sequence of at least 98% sequence homology to an amino acid sequence according to SEQ ID NO: 16. In some embodiments, the oligomerization domain comprises an amino acid sequence of at least 99% sequence homology to an amino acid sequence according to SEQ ID NO: 16.

In some embodiments, the oligomerization domain comprises an amino acid sequence according to SEQ ID NO: 16.

In some embodiments, the oligomerization domain comprises an amino acid sequence of at least 75% sequence identity to an amino acid sequence according to SEQ ID NO: 17. In some embodiments, the oligomerization domain comprises an amino acid sequence of at least 80% sequence identity to an amino acid sequence according to SEQ ID NO: 17. In some embodiments, the oligomerization domain comprises an amino acid sequence of at least 85% sequence identity to an amino acid sequence according to SEQ ID NO: 17. In some embodiments, the oligomerization domain comprises an amino acid sequence of at least 90% sequence identity to an amino acid sequence according to SEQ ID NO: 17. In some embodiments, the oligomerization domain comprises an amino acid sequence of at least 95% sequence identity to an amino acid sequence according to SEQ ID NO: 17. In some embodiments, the oligomerization domain comprises an amino acid sequence of at least 97% sequence identity to an amino acid sequence according to SEQ ID NO: 17. In some embodiments, the oligomerization domain comprises an amino acid sequence of at least 98% sequence identity to an amino acid sequence according to SEQ ID NO: 17. In some embodiments, the oligomerization domain comprises an amino acid sequence of at least 99% sequence identity to an amino acid sequence according to SEQ ID NO: 17.

In some embodiments, the oligomerization domain comprises an amino acid sequence of at least 75% sequence homology to an amino acid sequence according to SEQ ID NO: 17. In some embodiments, the oligomerization domain comprises an amino acid sequence of at least 80% sequence homology to an amino acid sequence according to SEQ ID NO: 17. In some embodiments, the oligomerization domain comprises an amino acid sequence of at least 85% sequence homology to an amino acid sequence according to SEQ ID NO: 17. In some embodiments, the oligomerization domain comprises an amino acid sequence of at least 90% sequence homology to an amino acid sequence according to SEQ ID NO: 17. In some embodiments, the oligomerization domain comprises an amino acid sequence of at least 95% sequence homology to an amino acid sequence according to SEQ ID NO: 17. In some embodiments, the oligomerization domain comprises an amino acid sequence of at least 97% sequence homology to an amino acid sequence according to SEQ ID NO: 17. In some embodiments, the oligomerization domain comprises an amino acid sequence of at least 98% sequence homology to an amino acid sequence according to SEQ ID NO: 17. In some embodiments, the oligomerization domain comprises an amino acid sequence of at least 99% sequence homology to an amino acid sequence according to SEQ ID NO: 17.

In some embodiments, the oligomerization domain comprises an amino acid sequence according to SEQ ID NO: 17.

In some embodiments, the oligomerization domain comprises an amino acid sequence of at least 75% sequence identity to an amino acid sequence according to SEQ ID NO: 18. In some embodiments, the oligomerization domain comprises an amino acid sequence of at least 80% sequence identity to an amino acid sequence according to SEQ ID NO: 18. In some embodiments, the oligomerization domain comprises an amino acid sequence of at least 85% sequence identity to an amino acid sequence according to SEQ ID NO: 18. In some embodiments, the oligomerization domain comprises an amino acid sequence of at least 90% sequence identity to an amino acid sequence according to SEQ ID NO: 18. In some embodiments, the oligomerization domain comprises an amino acid sequence of at least 95% sequence identity to an amino acid sequence according to SEQ ID NO: 18. In some embodiments, the oligomerization domain comprises an amino acid sequence of at least 97% sequence identity to an amino acid sequence according to SEQ ID NO: 18. In some embodiments, the oligomerization domain comprises an amino acid sequence of at least 98% sequence identity to an amino acid sequence according to SEQ ID NO: 18. In some embodiments, the oligomerization domain comprises an amino acid sequence of at least 99% sequence identity to an amino acid sequence according to SEQ ID NO: 18.

In some embodiments, the oligomerization domain comprises an amino acid sequence of at least 75% sequence homology to an amino acid sequence according to SEQ ID NO: 18. In some embodiments, the oligomerization domain comprises an amino acid sequence of at least 80% sequence homology to an amino acid sequence according to SEQ ID NO: 18. In some embodiments, the oligomerization domain comprises an amino acid sequence of at least 85% sequence homology to an amino acid sequence according to SEQ ID NO: 18. In some embodiments, the oligomerization domain comprises an amino acid sequence of at least 90% sequence homology to an amino acid sequence according to SEQ ID NO: 18. In some embodiments, the oligomerization domain comprises an amino acid sequence of at least 95% sequence homology to an amino acid sequence according to SEQ ID NO: 18. In some embodiments, the oligomerization domain comprises an amino acid sequence of at least 97% sequence homology to an amino acid sequence according to SEQ ID NO: 18. In some embodiments, the oligomerization domain comprises an amino acid sequence of at least 98% sequence homology to an amino acid sequence according to SEQ ID NO: 18. In some embodiments, the oligomerization domain comprises an amino acid sequence of at least 99% sequence homology to an amino acid sequence according to SEQ ID NO: 18. In some embodiments, the oligomerization domain comprises an amino acid sequence according to SEQ ID NO: 18.

In some embodiments, the oligomerization domain comprises an amino acid sequence of at least 75% sequence identity to an amino acid sequence according to SEQ ID NO: 19. In some embodiments, the oligomerization domain comprises an amino acid sequence of at least 80% sequence identity to an amino acid sequence according to SEQ ID NO: 19. In some embodiments, the oligomerization domain comprises an amino acid sequence of at least 85% sequence identity to an amino acid sequence according to SEQ ID NO: 19. In some embodiments, the oligomerization domain comprises an amino acid sequence of at least 90% sequence identity to an amino acid sequence according to SEQ ID NO: 19. In some embodiments, the oligomerization domain comprises an amino acid sequence of at least 95% sequence identity to an amino acid sequence according to SEQ ID NO: 19. In some embodiments, the oligomerization domain comprises an amino acid sequence of at least 97% sequence identity to an amino acid sequence according to SEQ ID NO: 19. In some embodiments, the oligomerization domain comprises an amino acid sequence of at least 98% sequence identity to an amino acid sequence according to SEQ ID NO: 19. In some embodiments, the oligomerization domain comprises an amino acid sequence of at least 99% sequence identity to an amino acid sequence according to SEQ ID NO: 19.

In some embodiments, the oligomerization domain comprises an amino acid sequence of at least 75% sequence homology to an amino acid sequence according to SEQ ID NO: 19. In some embodiments, the oligomerization domain comprises an amino acid sequence of at least 80% sequence homology to an amino acid sequence according to SEQ ID NO: 19. In some embodiments, the oligomerization domain comprises an amino acid sequence of at least 85% sequence homology to an amino acid sequence according to SEQ ID NO: 19. In some embodiments, the oligomerization domain comprises an amino acid sequence of at least 90% sequence homology to an amino acid sequence according to SEQ ID NO: 19. In some embodiments, the oligomerization domain comprises an amino acid sequence of at least 95% sequence homology to an amino acid sequence according to SEQ ID NO: 19. In some embodiments, the oligomerization domain comprises an amino acid sequence of at least 97% sequence homology to an amino acid sequence according to SEQ ID NO: 19. In some embodiments, the oligomerization domain comprises an amino acid sequence of at least 98% sequence homology to an amino acid sequence according to SEQ ID NO: 19. In some embodiments, the oligomerization domain comprises an amino acid sequence of at least 99% sequence homology to an amino acid sequence according to SEQ ID NO: 19. In some embodiments, the oligomerization domain comprises an amino acid sequence according to SEQ ID NO: 19. In some instances, the oligomerization domain comprises an amino acid sequence comprising at least a portion having at least or about 10, 20, 30, 40, 50, 60, 70, 80, 90, 100, 110, 120, 130 amino acid sequences of any sequence according to Table 1.

The term “sequence identity.” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. Typically, techniques for determining sequence identity include comparing two nucleotide or amino acid sequences and the determining their percent identity. Sequence comparisons, such as for the purpose of assessing identities, may be performed by any suitable alignment algorithm, including but not limited to the Needleman-Wunsch algorithm (see, e.g., the EMBOSS Needle aligner available at www.ebi.ac.uk/Tools/psa/emboss_needle/, optionally with default settings), the BLAST algorithm (see, e.g., the BLAST alignment tool available at blast.ncbi.nlm.nih.gov/Blast.cgi, optionally with default settings), and the Smith-Waterman algorithm (see, e.g., the EMBOSS Water aligner available at www.ebi.ac.uk/Tools/psa/emboss_water/, optionally with default settings). Optimal alignment may be assessed using any suitable parameters of a chosen algorithm, including default parameters. The “percent identity”, also referred to as “percent homology”, between two sequences may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990): Karlin and Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the sequences being compared. Default parameters are provided to optimize searches with short query sequences, for example, with the blastp program. The program also allows use of an SEG filter to mask-off segments of the query sequences as determined by the SEG program of Wootton and Federhen, Computers and Chemistry 17:149-163 (1993). High sequence identity generally includes ranges of sequence identity of approximately 80% to 100% and integer values there between.

In some embodiments, when the recombinant fusion protein is expressed on the surface of the enveloped particle, the oligomerization domain is outside of the enveloped particle. In some embodiments, when the recombinant fusion protein is expressed on the surface of the enveloped particle, the oligomerization domain is outside of the enveloped particle and adjacent to a signal peptide. In some embodiments, when the recombinant fusion protein is expressed on the surface of the enveloped particle, the oligomerization domain is inside of the enveloped particle. In some embodiments, when the recombinant fusion protein is expressed on the surface of the enveloped particle, the oligomerization domain is inside of the enveloped particle and adjacent to the transmembrane domain.

In some embodiments, the recombinant fusion protein comprises a signal peptide. In some embodiments, domains of the recombinant fusion protein are arranged from the N-terminus to the C-terminus in the following orders: (a) signal peptide, display polypeptide, oligomerization domain, and transmembrane domain: (b) signal peptide, display polypeptide, transmembrane domain, and oligomerization domain: or (c) signal peptide, oligomerization domain, display peptide, and transmembrane domain. In some embodiments, domains of the recombinant fusion protein are arranged from the N-terminus to the C-terminus in the following order: signal peptide, display polypeptide, oligomerization domain, and transmembrane domain. In some embodiments, domains of the recombinant fusion protein are arranged from the N-terminus to the C-terminus in the following order: signal peptide, display polypeptide, transmembrane domain, and oligomerization domain. In some embodiments, domains of the recombinant fusion protein are arranged from the N-terminus to the C-terminus in the following order: signal peptide, oligomerization domain, display peptide, and transmembrane domain.

In some embodiments, domains of the recombinant fusion protein are arranged from the N-terminus to the C-terminus in the following orders: (a) signal peptide, display polypeptide, oligomerization domain, transmembrane domain, and cytosolic domain: (b) signal peptide, display polypeptide, transmembrane domain, oligomerization domain, and cytosolic domain: or (c) signal peptide, oligomerization domain, display peptide, transmembrane domain, and cytosolic domain. In some embodiments, domains of the recombinant fusion protein are arranged from the N-terminus to the C-terminus in the following order: signal peptide, display polypeptide, oligomerization domain, transmembrane domain, and cytosolic domain. In some embodiments, domains of the recombinant fusion protein are arranged from the N-terminus to the C-terminus in the following order: signal peptide, display polypeptide, transmembrane domain, oligomerization domain, and cytosolic domain. In some embodiments, domains of the recombinant fusion protein are arranged from the N-terminus to the C-terminus in the following order: signal peptide, oligomerization domain, display peptide, transmembrane domain, and cytosolic domain.

In some embodiments, the enveloped particle comprises an enveloped viral-like particle, an enveloped virus, an extracellular vesicle. In some embodiments, the extracellular vesicle comprises an exosome or an ectosome.

In some embodiments, the display polypeptide targets a cell surface protein that is oligomerized on a target cell for agonistic or antagonistic function. In some embodiments, the cell surface protein is dimerized and the oligomerization domain is a dimerization domain. In some embodiments, the cell surface protein is trimerized and the oligomerization domain is a trimerization domain. In some embodiments, the cell surface protein is tetramerized and the oligomerization domain is a tetramerization domain.

In some embodiments, the display polypeptide comprises a single chain variable fragment (scFv), a type I transmembrane polypeptide, a type II transmembrane polypeptide, a type III transmembrane polypeptide, a GPI-anchored polypeptide, a secreted polypeptide, or a multi-pass transmembrane polypeptide. In some embodiments, the scFv comprises an anti-BCMA scFv polypeptide, an anti-NTD scFv polypeptide, an anti-RBD scFv polypeptide, or an anti-CD19 scFv polypeptide. In some embodiments, the type I transmembrane polypeptide comprises ACE2, ICAM-1, LILRA5, LILRB3, or LILRB4. In some embodiments, the type II transmembrane polypeptide comprises DPP4, or BAFF. In some embodiments, the type III transmembrane polypeptide comprises BCMA, TACI, or BAFF Receptor. In some embodiments, the GPI-anchored polypeptide comprises CD24, CD48, CD59 or CD55. In some embodiments, the secreted polypeptide comprises IL-4, or CXCL-12. In some embodiments, the multi-pass transmembrane polypeptide comprises NTCP, or FCεRI beta. In some embodiments, the display polypeptide comprises an antibody, a receptor, a cytokine, an immune checkpoint protein, a chemokine, an adhesion factor, or a mitogen. In some embodiments, the antibody comprises a non-neutralizing antibody, a neutralizing antibody, or an antibody of multiple formats. In some embodiments, the receptor comprises ACE2, or DPP4. In some embodiments, the cytokine comprises IL-4, or IL-6. In some embodiments, the immune checkpoint protein comprises CTLA-4, or PD-1. In some embodiments, the chemokine comprises CXCL-12. In some embodiments, the adhesion factor comprises ICAM-1, or LFA-1. In some embodiments, the mitogen comprises Wnt. In some embodiments, the display polypeptide comprises an anti-BCMA scFv polypeptide, an anti-CD19 scFv polypeptide, an anti-NTD scFv polypeptide, an anti-RBD scFv polypeptide, ACE2, ICAM-1, LILRA5, LILRB3, LILRB4, DPP4, BAFF, BCMA, TACI, BAFF Receptor, CD24, CD48, CD59, CD55, IL-4, CXCL-12, NTCP, FC&RI beta, a non-neutralizing antibody, a neutralizing antibody, an antibody of multiple formats, IL-6, CTLA-4, PD-1, LFA-1, Wnt, CD80, CD86, OX40L, CD3, or 4-1BBL.

TABLE 2
Exemplary Display Polypeptides

| Display | | NCBI | | |
|---|---|---|---|---|
| Polypeptide | Classification | Ref No. | Amino Acid Sequence (N to C) | SEQ ID NO: |
| anti- | scFv | n/a | GSQVQLVQSGAEVKKPGSSVKVSCKASG | 20 |
| BCMA | | | GTFSNYWMHWVRQAPGQGLEWMGATY | |
| scFv | | | RGHSDTYYNQKFKGRVTITADKSTSTAY | |
| | | | MELSSLRSEDTAVYYCARGAIYNGYDVL | |
| | | | DNWGQGTLVTVSSGGGGSGGGGSGGGG | |
| | | | SGGGGSDIQMTQSPSSLSASVGDRVTITC | |
| | | | SASQDISNYLNWYQQKPGKAPKLLIYYTS | |
| | | | NLHSGVPSRFSGSGSGTDFTLTISSLQPED | |
| | | | FATYYCQQYRKLPWTFGQGTKLEIKR | |
| anti- | scFv | n/a | GSDIQMTQTTSSLSASLGDRVTISCRASQ | 21 |
| CD19 | | | DISKYLNWYQQKPDGTVKLLIYHTSRLH | |
| | | | SGVPSRFSGSGSGTDYSLTISNLEQEDIAT | |
| | | | YFCQQGNTLPYTFGGGTKLEITGGGGSG | |
| | | | GGGSGGGGSEVKLQESGPGLVAPSQSLS | |
| | | | VTCTVSGVSLPDYGVSWIRQPPRKGLEW | |
| | | | LGVIWGSETTYYNSALKSRLTIIKDNSKS | |
| | | | QVFLKMNSLQTDDTAIYYCAKHYYYGG | |
| | | | SYAMDYWGQGTSVTVSSTTTPAPRPPTP | |
| | | | APTIASQPLSLRPEACRPAAGGAVHTRGL | |
| | | | DFACDGs | |
| Human | type I | NP_067073.1 | MAPWSHPSAQLQPVGGDAVSPALMVLL | 22 |
| LILRA | transmembrane | | CLGLSLGPRTHVQAGNLSKATLWAEPGS | |
| 5 | polypeptide | | VISRGNSVTIRCQGTLEAQEYRLVKEGSP | |
| | | | EPWDTQNPLEPKNKARFSIPSMTEHHAG | |
| | | | RYRCYYYSPAGWSEPSDPLELVVTGFYN | |
| | | | KPTLSALPSPVVTSGENVTLQCGSRLRFD | |
| | | | RFILTEEGDHKLSWTLDSQLTPSGQFQAL | |
| | | | FPVGPVTPSHRWMLRCYGSRRHILQVWS | |
| | | | EPSDLLEIPVSGAADNLSPSQNKSDSGTAS | |
| | | | HLQDYAVENLIRMGMAGLILVVLGILIFQ | |
| | | | DWHSQRSPQAAAGR | |
| Murine | type I | NP_001074708.2 | MTFVFTAVLCLGLNLGQETSMSEGNPHK | 23 |
| LILRA | transmembrane | | PTLSVQPGLVVAKGKQVTISCEVTTGARE | |
| 5 | polypeptide | | YRLYKEGGPHPWRTRNTPKTTNKAQFLI | |
| | | | PSIEQRSGGIYRCYYKTPTGWSEHSDPLE | |
| | | | LAVTGLYSKPSLSTQPSNVVNSGETVTLQ | |
| | | | CVSTLGFNRFVLTKEGEQKWSLIQESEFI | |
| | | | NSTGQFQGLFTVGPVTPSQRWIFRCYGY | |
| | | | HVNSPQVWSEPSDLLEIHVSEADQPLRPS | |
| | | | PNISDPKTVSQPQNYTMENLIRMGASILV | |
| | | | LVLLGVLLFEAQHSQRQTQHAAGRESSA | |
| | | | SFMVADTWE | |
| Human | type I | NP_001074919.2 | MTPALTALLCLGLSLGPRTRMQAGPFPKP | 24 |
| LILRB | transmembrane | | TLWAEPGSVISWGSPVTIWCQGSLEAQE | |
| 3 | polypeptide | | YQLDKEGSPEPWDRNNPLEPKNKARFSIP | |
| | | | SMTQHHAGRYRCHYYSSAGWSEPSDPLE | |
| | | | LVMTGFYNKPTLSALPSPVVASGGNMTL | |
| | | | RCGSQKGYHHFVLMKEGEHQLPRTLDSQ | |
| | | | QLHSGGFQALFPVGPVTPSHRWRFTCYY | |
| | | | YYTNTPWVWSHPSDPLEILPSGVSRKPSL | |
| | | | LTLQGPVLAPGQSLTLQCGSDVGYDRFV | |
| | | | LYKEGERDFLQRPGQQPQAGLSQANFTL | |
| | | | GPVSRSYGGQYRCYGAHNLSSEWSAPSD | |
| | | | PLDILITGQIYDTVSLSAQPGPTVASGENM | |
| | | | TLLCQSRGYFDTFLLTKEGAAHPPLRLRS | |
| | | | MYGAHKYQAEFPMSPVTSAHAGTYRCY | |
| | | | GSRSSNPHLLSFPSEPLELMVSGHSGGSSL | |
| | | | PPTGPPSTPGLGRYLEVLIGVSVAFVLLLF | |
| | | | LLLFLLLLRQRHSKHRTSDQRKTDFQRPA | |
| | | | GAAETEPKDRGLLRRSSPAADVQEENLY | |
| | | | AAVKDTQSEDRVELDSQQSPHDEDPQAV | |
| | | | TYAPVKHSSPRREMASPPSSLSGEFLDTK | |
| | | | DRQVEEDRQMDTEAAASEASQDVTYAQ | |
| | | | LHSLTLRRKATEPPPSQEGEPPAEPSIYAT | |
| | | | LAIH | |
| Murine | type I | NP_032874.1 | MSCTFTALLCLGLTLSLWIPVLTGSLPKPI | 25 |
| LILRB | transmembrane | | LRVQPDSVVSRRTNVTFFCEETIGANEYR | |
| 3 | polypeptide | | LYKDGKLYKTVTKNKQKQANKAEFSFS | |
| | | | NVDLSNAGQYECSYSTQDESSGYSDPLEL | |
| | | | VVTGHYWTPSLSAQASPVVTSGGYVTLQ | |
| | | | CESWHNDHKFILTVEGPQKLSWTQDSQY | |
| | | | NYSTRKYHALFSVGPVTPNQRWICRCYS | |
| | | | YDRNRPYVWSPPSESVELLVSGNLQKPTI | |
| | | | KAEPGSVITSKRAMTIWCQGNLDAEVYF | |
| | | | LHNEKSQKTQSTQTLQQPGNKGRFFIPSV | |
| | | | TQQHAGQYRCYCYSSAGWSQPSDTLELV | |
| | | | VTGIYEYNEPRLSLLPSPVVRPGGNMTLH | |
| | | | CASQGHYDKFILTKEDKKFANALDTEHIS | |
| | | | SSRQYQALFIIGPTTPTHTGTFRCYGYYK | |
| | | | NTPQLWSVPSNLQQILISGLSKKPSLLTHQ | |
| | | | GHILDPGMTLTLQCFSDMNYDRFALHKV | |
| | | | GGADIMQHSSQQTDIGFSVANFTLGYVSS | |
| | | | STGGQYRCYGAHNLSSEWSASSEPLDILI | |
| | | | TGQLPLTPSLSVQPNHTVHSGETVSLLCW | |
| | | | SMDSVDTFILSKEGSAQQPLRLKSKSHDQ | |
| | | | QSQAEFSMSAVTSHLSGTYRCYGAQDSS | |
| | | | FYLLSSASAPVELTVSGTIESSSWPPKRPN | |
| | | | PPIPTENQDHTMENLIRMGMAVVVFIVLS | |
| | | | ILATEAWRSHRQTHCAAGN | |
| Human | type II | Q9Y275 | MDDSTEREQSRLTSCLKKREEMKLKECV | 26 |
| BAFF | transmembrane | | SILPRKESPSVRSSKDGKLLAATLLLALLS | |
| | | | CCLTVVSFYQVAALQGDLASLRAELQGH | |
| | | | HAEKLPAGAGAPKAGLEEAPAVTAGLKI | |
| | | | FEPPAPGEGNSSQNSRNKRAVQGPEETVT | |
| | | | QDCLQLIADSETPTIQKGSYTFVPWLLSF | |
| | | | KRGSALEEKENKILVKETGYFFIYGQVLY | |
| | | | TDKTYAMGHLIQRKKVHVFGDELSLVTL | |
| | | | FRCIQNMPETLPNNSCYSAGIAKLEEGDE | |
| | | | LQLAIPRENAQISLDGDVTFFGALKLL | |
| Murine | type II | Q9WU72 | MDESAKTLPPPCLCFCSEKGEDMKVGYD | 27 |
| BAFF | transmembrane | | PITPQKEEGAWFGICRDGRLLAATLLLAL | |
| | | | LSSSFTAMSLYQLAALQADLMNLRMELQ | |
| | | | SYRGSATPAAAGAPELTAGVKLLTPAAP | |
| | | | RPHNSSRGHRNRRAFQGPEETEQDVDLS | |
| | | | APPAPCLPGCRHSQHDDNGMNLRNIIQD | |
| | | | CLQLIADSDTPTIRKGTYTFVPWLLSFKR | |
| | | | GNALEEKENKIVVRQTGYFFIYSQVLYTD | |
| | | | PIFAMGHVIQRKKVHVFGDELSLVTLFRC | |
| | | | IQNMPKTLPNNSCYSAGIARLEEGDEIQL | |
| | | | AIPRENAQISRNGDDTFFGALKLL | |
| Human | type III | Q02223 | MLQMAGQCSQNEYFDSLLHACIPCQLRC | 28 |
| BCMA | transmembrane | | SSNTPPLTCQRYCNASVTNSVKGTNAILW | |
| | polypeptide | | TCLGLSLIISLAVFVLMFLLRKINSEPLKD | |
| | | | EFKNTGSGLLGMANIDLEKSRTGDEIILPR | |
| | | | GLEYTVEECTCEDCIKSKPKVDSDHCFPL | |
| | | | PAMEEGATILVTTKTNDYCKSLPAALSAT | |
| | | | EIEKSISAR | |
| Murine | type III | O88472 | MAQQCFHSEYFDSLLHACKPCHLRCSNP | 29 |
| BCMA | transmembrane | | PATCQPYCDPSVTSSVKGTYTVLWIFLGL | |
| | polypeptide | | TLVLSLALFTISFLLRKMNPEALKDEPQSP | |
| | | | GQLDGSAQLDKADTELTRIRAGDDRIFPR | |
| | | | SLEYTVEECTCEDCVKSKPKGDSDHFFPL | |
| | | | PAMEEGATILVTTKTGDYGKSSVPTALQS | |
| | | | VMGMEKPTHTR | |
| Human | type III | O14836 | MSGLGRSRRGGRSRVDQEERFPQGLWTG | 30 |
| TACI | transmembrane | | VAMRSCPEEQYWDPLLGTCMSCKTICNH | |
| | polypeptide | | QSQRTCAAFCRSLSCRKEQGKFYDHLLR | |
| | | | DCISCASICGQHPKQCAYFCENKLRSPVN | |
| | | | LPPELRRQRSGEVENNSDNSGRYQGLEH | |
| | | | RGSEASPALPGLKLSADQVALVYSTLGLC | |
| | | | LCAVLCCFLVAVACFLKKRGDPCSCQPR | |
| | | | SRPRQSPAKSSQDHAMEAGSPVSTSPEPV | |
| | | | ETCSFCFPECRAPTQESAVTPGTPDPTCA | |
| | | | GRWGCHTRTTVLQPCPHIPDSGLGIVCVP | |
| | | | AQEGGPGA | |
| Murine | type III | Q9ET35 | MAMAFCPKDQYWDSSRKSCVSCALTCS | 31 |
| TACI | transmembrane | | QRSQRTCTDFCKFINCRKEQGRYYDHLL | |
| | polypeptide | | GACVSCDSTCTQHPQQCAHFCEKRPRSQ | |
| | | | ANLQPELGRPQAGEVEVRSDNSGRHQGS | |
| | | | EHGPGLRLSSDQLTLYCTLGVCLCAIFCC | |
| | | | FLVALASFLRRRGEPLPSQPAGPRGSQAN | |
| | | | SPHAHRPVTEACDEVTASPQPVETCSFCF | |
| | | | PERSSPTQESAPRSLGIHGFAGTAAPQPC | |
| | | | MRATVGGLGVLRASTGDARPAT | |
| Human | type III | Q5H8V1 | MRRGPRSLRGRDAPAPTPCVPAECFDLL | 32 |
| BAFF | transmembrane | | VRHCVACGLLRTPRPKPAGASSPAPRTAL | |
| Receptor | polypeptide | | QPQESVGAGAGEAALPLPGLLFGAPALL | |
| | | | GLALVLALVLVGLVSWRRRQRRLRGASS | |
| | | | AEAPDGDKDAPEPLDKVIILSPGISDATAP | |
| | | | AWPPPGEDPGTTPPGHSVPVPATELGSTE | |
| | | | LVTTKTAGPEQQ | |
| Murine | type III | Q9D8D0 | MGARRLRVRSQRSRDSSVPTQCNQTECF | 33 |
| BAFF | transmembrane | | DPLVRNCVSCELFHTPDTGHTSSLEPGTA | |
| Recept | polypeptide | | LQPQEGSALRPDVALLVGAPALLGLILAL | |
| or | | | TLVGLVSLVSWRWRQQLRTASPDTSEGV | |
| | | | QQESLENVFVPSSETPHASAPTWPPLKED | |
| | | | ADSALPRHSVPVPATELGSTELVTTKTAG | |
| | | | PEQ | |
| Human | GPI-anchored | NP_000602.1 | MGIQGGSVLFGLLLVLAVFCHSGHSLQC | 34 |
| CD59 | polypeptide | | YNCPNPTADCKTAVNCSSDFDACLITKA | |
| | | | GLQVYNKCWKFEHCNFNDVTTRLRENE | |
| | | | LTYYCCKKDLCNFNEQLENGGTSLSEKT | |
| | | | VLLLVTPFLAAAWSLHP | |
| Murine | GPI-anchored | NP_862906.1 | MRAQRGLILLLLLLAVFCSTAVSLKCYNC | 35 |
| CD59 | polypeptide | | LDPVSSCKINTTCSPNLDSCLYAVAGRQV | |
| | | | YQQCWKLSDCNSNYIMSRLDVAGIQSKC | |
| | | | CQWDLCNKNLDGLEEPNNAETSSLRKTA | |
| | | | LLGTSVLVAILKFCF | |
| Human | GPI-anchored | NP_000565.1 | MTVARPSVPAALPLLGELPRLLLLVLLCL | 36 |
| CD55 | polypeptide | | PAVWGDCGLPPDVPNAQPALEGRTSFPE | |
| | | | DTVITYKCEESFVKIPGEKDSVICLKGSQ | |
| | | | WSDIEEFCNRSCEVPTRLNSASLKQPYITQ | |
| | | | NYFPVGTVVEYECRPGYRREPSLSPKLTC | |
| | | | LQNLKWSTAVEFCKKKSCPNPGEIRNGQI | |
| | | | DVPGGILFGATISFSCNTGYKLFGSTSSFC | |
| | | | LISGSSVQWSDPLPECREIYCPAPPQIDNGI | |
| | | | IQGERDHYGYRQSVTYACNKGFTMIGEH | |
| | | | SIYCTVNNDEGEWSGPPPECRGKSLTSKV | |
| | | | PPTVQKPTTVNVPTTEVSPTSQKTTTKTT | |
| | | | TPNAQATRSTPVSRTTKHFHETTPNKGSG | |
| | | | TTSGTTRLLSGHTCFTLTGLLGTLVTMGL | |
| | | | LT | |
| Murine | GPI-anchored | NP_034146.2 | MIRGRAPRTRPSPPPPLLPLLSLSLLLLSPT | 37 |
| CD55 | polypeptide | | VRGDCGPPPDIPNARPILGRHSKFAEQSK | |
| | | | VAYSCNNGFKQVPDKSNIVVCLENGQWS | |
| | | | SHETFCEKSCVAPERLSFASLKKEYLNMN | |
| | | | FFPVGTIVEYECRPGFRKQPPLPGKATCLE | |
| | | | DLVWSPVAQFCKKKSCPNPKDLDNGHIN | |
| | | | IPTGILFGSEINFSCNPGYRLVGVSSTFCSV | |
| | | | TGNTVDWDDEFPVCTEIHCPEPPKINNGI | |
| | | | MRGESDSYTYSQVVTYSCDKGFILVGNA | |
| | | | SIYCTVSKSDVGQWSSPPPRCIEKSKVPTK | |
| | | | KPTINVPSTGTPSTPQKPTTESVPNPGDQP | |
| | | | TPQKPSTVKVSATQHVPVTKTTVRHPIRT | |
| | | | STDKGEPNTGGDRYIYGHTCLITLTVLHV | |
| | | | MLSLIGYLT | |
| Human | secreted | P05112 | MGLTSQLLPPLFFLLACAGNFVHGHKCDI | 38 |
| IL-4 | polypeptide | | TLQEIIKTLNSLTEQKTLCTELTVTDIFAA | |
| | | | SKNTTEKETFCRAATVLRQFYSHHEKDT | |
| | | | RCLGATAQQFHRHKQLIRFLKRLDRNLW | |
| | | | GLAGLNSCPVKEANQSTLENFLERLKTIM | |
| | | | REKYSKCSS | |
| Murine | secreted | P07750 | MGLNPQLVVILLFFLECTRSHIHGCDKNH | 39 |
| IL-4 | polypeptide | | LREIIGILNEVTGEGTPCTEMDVPNVLTAT | |
| | | | KNTTESELVCRASKVLRIFYLKHGKTPCL | |
| | | | KKNSSVLMELQRLFRAFRCLDSSISCTMN | |
| | | | ESKSTSLKDFLESLKSIMQMDYS | |
| Human | multi-pass | Q01362 | MDTESNRRANLALPQEPSSVPAFEVLEISP | 40 |
| FCϵRI | transmembrane | | QEVSSGRLLKSASSPPLHTWLTVLKKEQE | |
| beta | polypeptide | | FLGVTQILTAMICLCFGTVVCSVLDISHIE | |
| | | | GDIFSSFKAGYPFWGAIFFSISGMLSIISER | |
| | | | RNATYLVRGSLGANTASSIAGGTGITILII | |
| | | | NLKKSLAYIHIHSCQKFFETKCFMASFSTE | |
| | | | IVVMMLFLTILGLGSAVSLTICGAGEELK | |
| | | | GNKVPEDRVYEELNIYSATYSELEDPGE | |
| | | | MSPPIDL | |
| Murine | multi-pass | P20490 | MDTENRSRADLALPNPQESSSAPDIELLE | 41 |
| FCϵRI | transmembrane | | ASPAKAAPPKQTWRTFLKKELEFLGATQI | |
| beta | polypeptide | | LVGLICLCFGTIVCSVLYVSDFDEEVLLLY | |
| | | | KLGYPFWGAVLFVLSGFLSIISERKNTLY | |
| | | | LVRGSLGANIVSSIAAGTGIAMLILNLTNN | |
| | | | FAYMNNCKNVTEDDGCFVASFTTELVL | |
| | | | MMLFLTILAFCSAVLFTIYRIGQELESKKV | |
| | | | PDDRLYEELNVYSPIYSELEDKGETSSPV | |
| | | | DS | |
| Human | cytokine | P05231 | MNSFSTSAFGPVAFSLGLLLVLPAAFPAP | 42 |
| IL-6 | | | VPPGEDSKDVAAPHRQPLTSSERIDKQIR | |
| | | | YILDGISALRKETCNKSNMCESSKEALAE | |
| | | | NNLNLPKMAEKDGCFQSGFNEETCLVKII | |
| | | | TGLLEFEVYLEYLQNRFESSEEQARAVQ | |
| | | | MSTKVLIQFLQKKAKNLDAITTPDPTTNA | |
| | | | SLLTKLQAQNQWLQDMTTHLILRSFKEF | |
| | | | LQSSLRALRQM | |
| Murine | cytokine | P08505 | MKFLSARDFHPVAFLGLMLVTTTAFPTS | 43 |
| IL-6 | | | QVRRGDFTEDTTPNRPVYTTSQVGGLITH | |
| | | | VLWEIVEMRKELCNGNSDCMNNDDALA | |
| | | | ENNLKLPEIQRNDGCYQTGYNQEICLLKI | |
| | | | SSGLLEYHSYLEYMKNNLKDNKKDKAR | |
| | | | VLQRDTETLIHIFNQEVKDLHKIVLPTPIS | |
| | | | NALLTDKLESQKEWLRTKTIQFILKSLEEF | |
| | | | LKVTLRSTRQT | |
| Human | chemokine | P48061 | MNAKVVVVLVLVLTALCLSDGKPVSLSY | 44 |
| CXCL- | | | RCPCRFFESHVARANVKHLKILNTPNCAL | |
| 12 | | | QIVARLKNNNRQVCIDPKLKWIQEYLEK | |
| | | | ALNKRFKM | |
| Murine | chemokine | P40224 | MDAKVVAVLALVLAALCISDGKPVSLSY | 45 |
| CXCL- | | | RCPCRFFESHIARANVKHLKILNTPNCAL | |
| 12 | | | QIVARLKNNNRQVCIDPKLKWIQEYLEK | |
| | | | ALNKRLKM | |
| Human | adhesion factor | NP_000192.2 | MAPSSPRPALPALLVLLGALFPGPGNAQT | 46 |
| ICAM- | | | SVSPSKVILPRGGSVLVTCSTSCDQPKLLG | |
| 1 | | | IETPLPKKELLLPGNNRKVYELSNVQEDS | |
| | | | QPMCYSNCPDGQSTAKTFLTVYWTPERV | |
| | | | ELAPLPSWQPVGKNLTLRCQVEGGAPRA | |
| | | | NLTVVLLRGEKELKREPAVGEPAEVTTT | |
| | | | VLVRRDHHGANFSCRTELDLRPQGLELF | |
| | | | ENTSAPYQLQTFVLPATPPQLVSPRVLEV | |
| | | | DTQGTVVCSLDGLFPVSEAQVHLALGDQ | |
| | | | RLNPTVTYGNDSFSAKASVSVTAEDEGT | |
| | | | QRLTCAVILGNQSQETLQTVTIYSFPAPN | |
| | | | VILTKPEVSEGTEVTVKCEAHPRAKVTLN | |
| | | | GVPAQPLGPRAQLLLKATPEDNGRSFSCS | |
| | | | ATLEVAGQLIHKNQTRELRVLYGPRLDE | |
| | | | RDCPGNWTWPENSQQTPMCQAWGNPLP | |
| | | | ELKCLKDGTFPLPIGESVTVTRDLEGTYL | |
| | | | CRARSTQGEVTRKVTVNVLSPRYEIVIITV | |
| | | | VAAAVIMGTAGLSTYLYNRQRKIKKYRL | |
| | | | QQAQKGTPMKPNTQATPP | |
| Murine | adhesion factor | NM_010493.3 | MASTRAKPTLPLLLALVTVVIPGPGDAQV | 47 |
| ICAM- | | | SIHPREAFLPQGGSVQVNCSSSCKEDLSL | |
| 1 | | | GLETQWLKDELESGPNWKLFELSEIGEDS | |
| | | | SPLCFENCGTVQSSASATITVYSFPESVEL | |
| | | | RPLPAWQQVGKDLTLRCHVDGGAPRTQ | |
| | | | LSAVLLRGEEILSRQPVGGHPKDPKEITFT | |
| | | | VLASRGDHGANFSCRTELDLRPQGLALFS | |
| | | | NVSEARSLRTFDLPATIPKLDTPDLLEVGT | |
| | | | QQKLFCSLEGLFPASEARIYLELGGQMPT | |
| | | | QESTNSSDSVSATALVEVTEEFDRTLPLR | |
| | | | CVLELADQILETQRTLTVYNFSAPVLTLS | |
| | | | QLEVSEGSQVTVKCEAHSGSKVVLLSGV | |
| | | | EPRPPTPQVQFTLNASSEDHKRSFFCSAA | |
| | | | LEVAGKFLFKNQTLELHVLYGPRLDETD | |
| | | | CLGNWTWQEGSQQTLKCQAWGNPSPK | |
| | | | MTCRRKADGALLPIGVVKSVKQEMNGT | |
| | | | YVCHAFSSHGNVTRNVYLTVLYHSQNN | |
| | | | WTIIILVPVLLVIVGLVMAASYVYNRQRK | |
| | | | IRIYKLQKAQEEAIKLKGQAPPP | |
| Human | adhesion factor | NM_001114380.2 | MKDSCITVMAMALLSGFFFFAPASSYNL | 48 |
| LFA-1 | | | DVRGARSFSPPRAGRHFGYRVLQVGNGV | |
| | | | IVGAPGEGNSTGSLYQCQSGTGHCLPVTL | |
| | | | RGSNYTSKYLGMTLATDPTDGSILFAAV | |
| | | | QFSTSYKTEFDFSDYVKRKDPDALLKHV | |
| | | | KHMLLLTNTFGAINYVATEVFREELGAR | |
| | | | PDATKVLIIITDGEATDSGNIDAAKDIIRYI | |
| | | | IGIGKHFQTKESQETLHKFASKPASEFVKI | |
| | | | LDTFEKLKDLFTELQKKIYVIEGTSKQDL | |
| | | | TSFNMELSSSGISADLSRGHAVVGAVGA | |
| | | | KDWAGGFLDLKADLQDDTFIGNEPLTPE | |
| | | | VRAGYLGYTVTWLPSRQKTSLLASGAPR | |
| | | | YQHMGRVLLFQEPQGGGHWSQVQTIHG | |
| | | | TQIGSYFGGELCGVDVDQDGETELLLIGA | |
| | | | PLFYGEQRGGRVFIYQRRQLGFEEVSELQ | |
| | | | GDPGYPLGRFGEAITALTDINGDGLVDVA | |
| | | | VGAPLEEQGAVYIFNGRHGGLSPQPSQRI | |
| | | | EGTQVLSGIQWFGRSIHGVKDLEGDGLA | |
| | | | DVAVGAESQMIVLSSRPVVDMVTLMSFS | |
| | | | PAEIPVHEVECSYSTSNKMKEGVNITICFQ | |
| | | | IKSLIPQFQGRLVANLTYTLQLDGHRTRR | |
| | | | RGLFPGGRHELRRNIAVTTSMSCTDFSFH | |
| | | | FPVCVQDLISPINVSLNFSLWEEEGTPRDQ | |
| | | | RAGKDIPPILRPSLHSETWEIPFEKNCGED | |
| | | | KKCEANLRVSFSPARSRALRLTAFASLSV | |
| | | | ELSLSNLEEDAYWVQLDLHFPPGLSFRKV | |
| | | | EMLKPHSQIPVSCEELPEESRLLSRALSCN | |
| | | | VSSPIFKAGHSVALQMMFNTLVNSSWGD | |
| | | | SVELHANVTCNNEDSDLLEDNSATTIIPIL | |
| | | | YPINILIQDQEDSTLYVSFTPKGPKIHQVK | |
| | | | HMYQVRIQPSIHDHNIPTLEAVVGVPQPP | |
| | | | SEGPITHQWSVQMEPPVPCHYEDLERLPD | |
| | | | AAEPCLPGALFRCPVVFRQEILVQVIGTLE | |
| | | | LVGEIEASSMFSLCSSLSISFNSSKHFHLY | |
| | | | GSNASLAQVVMKVDVVYEKQMLYLYVL | |
| | | | SGIGGLLLLLLIFIVLYKVGFFKRNLKEKM | |
| | | | EAGRGVPNGIPAEDSEQLASGQEAGDPG | |
| | | | CLKPLHEKDSESGGGKD | |
| Murine | adhesion factor | NM_001253873.1 | MSFRIAGPRLLLLGLQLFAKAWSYNLDT | 49 |
| LFA-1 | | | RPTQSFLAQAGRHFGYQVLQIEDGVVVG | |
| | | | APGEGDNTGGLYHCRTSSEFCQPVSLHGS | |
| | | | NHTSKYLGMTLATDAAKGSLLACDPGLS | |
| | | | RTCDQNTYLSGLCYLFPQSLEGPMLQNR | |
| | | | PAYQECMKGKVDLVFLFDGSQSLDRKDF | |
| | | | EKILEFMKDVMRKLSNTSYQFAAVQFST | |
| | | | DCRTEFTFLDYVKQNKNPDVLLGSVQPM | |
| | | | FLLTNTFRAINYVVAHVFKEESGARPDAT | |
| | | | KVLVIITDGEASDKGNISAAHDITRYIIGIG | |
| | | | KHFVSVQKQKTLHIFASEPVEEFVKILDTF | |
| | | | EKLKDLFTDLQRRIYAIEGTNRQDLTSFN | |
| | | | MELSSSGISADLSKGHAVVGAVGAKDW | |
| | | | AGGFLDLREDLQGATFVGQEPLTSDVRG | |
| | | | GYLGYTVAWMTSRSSRPLLAAGAPRYQ | |
| | | | HVGQVLLFQAPEAGGRWNQTQKIEGTQI | |
| | | | GSYFGGELCSVDLDQDGEAELLLIGAPLF | |
| | | | FGEQRGGRVFTYQRRQSLFEMVSELQGD | |
| | | | PGYPLGRFGAAITALTDINGDRLTDVAVG | |
| | | | APLEEQGAVYIFNGKPGGLSPQPSQRIQG | |
| | | | AQVFPGIRWFGRSIHGVKDLGGDRLADV | |
| | | | VVGAEGRVVVLSSRPVVDVVTELSFSPEE | |
| | | | IPVHEVECSYSAREEQKHGVKLKACFRIK | |
| | | | PLTPQFQGRLLANLSYTLQLDGHRMRSR | |
| | | | GLFPDGSHELSGNTSITPDKSCLDFHFHFP | |
| | | | ICIQDLISPINVSLNFSLLEEEGTPRDQKGR | |
| | | | AMQPILRPSIHTVTKEIPFEKNCGEDKKCE | |
| | | | ANLTLSSPARSGPLRLMSSASLAVEWTLS | |
| | | | NSGEDAYWVRLDLDFPRGLSFRKVEMLQ | |
| | | | PHSRMPVSCEELTEGSSLLTKTLKCNVSS | |
| | | | PIFKAGQEVSLQVMENTLLNSSWEDFVEL | |
| | | | NGTVHCENENSSLQEDNSAATHIPVLYPV | |
| | | | NILTKEQENSTLYISFTPKGPKTQQVQHV | |
| | | | YQVRIQPSAYDHNMPTLEALVGVPWPHS | |
| | | | EDPITYTWSVQTDPLVTCHSEDLKRPSSE | |
| | | | AEQPCLPGVQFRCPIVFRREILIQVTGTVE | |
| | | | LSKEIKASSTLSLCSSLSVSFNSSKHFHLY | |
| | | | GSKASEAQVLVKVDLIHEKEMLHVYVLS | |
| | | | GIGGLVLLFLIFLALYKVGFFKRNLKEKM | |
| | | | EADGGVPNGSPPEDTDPLAVPGEETKDM | |
| | | | GCLEPLRESDKD | |
| Human | mitogen | NP_005421.1 | MGLWALLPGWVSATLLLALAALPAALA | 50 |
| Wnt | | | ANSSGRWWGIVNVASSTNLLTDSKSLQL | |
| | | | VLEPSLQLLSRKQRRLIRQNPGILHSVSGG | |
| | | | LQSAVRECKWQFRNRRWNCPTAPGPHLF | |
| | | | GKIVNRGCRETAFIFAITSAGVTHSVARSC | |
| | | | SEGSIESCTCDYRRRGPGGPDWHWGGCS | |
| | | | DNIDFGRLFGREFVDSGEKGRDLRFLMN | |
| | | | LHNNEAGRTTVFSEMRQECKCHGMSGSC | |
| | | | TVRTCWMRLPTLRAVGDVLRDREDGAS | |
| | | | RVLYGNRGSNRASRAELLRLEPEDPAHK | |
| | | | PPSPHDLVYFEKSPNFCTYSGRLGTAGTA | |
| | | | GRACNSSSPALDGCELLCCGRGHRTRTQ | |
| | | | RVTERCNCTFHWCCHVSCRNCTHTRVLH | |
| | | | ECL | |
| Murine | mitogen | NP_067254.1 | MGLWALLPSWVSTTLLLALTALPAALAA | 51 |
| Wnt | | | NSSGRWWGIVNIASSTNLLTDSKSLQLVL | |
| | | | EPSLQLLSRKQRRLIRQNPGILHSVSGGLQ | |
| | | | SAVRECKWQFRNRRWNCPTAPGPHLFG | |
| | | | KIVNRGCRETAFIFAITSAGVTHSVARSCS | |
| | | | EGSIESCTCDYRRRGPGGPDWHWGGCSD | |
| | | | NIDFGRLFGREFVDSGEKGRDLRFLMNL | |
| | | | HNNEAGRTTVFSEMRQECKCHGMSGSCT | |
| | | | VRTCWMRLPTLRAVGDVLRDRFDGASR | |
| | | | VLYGNRGSNRASRAELLRLEPEDPAHKPP | |
| | | | SPHDLVYFEKSPNFCTYSGRLGTAGTAGR | |
| | | | ACNSSSPALDGCELLCCGRGHRTRTQRV | |
| | | | TERCNCTFHWCCHVSCRNCTHTRVLHECL | |
| CV26 | ScFv | — | MEFGLSWVFLVALFRGVQSQVQLVQSG | 56 |
| scFv | | | AEVKKPGASVKVSCKVSGYTLTELSIHW | |
| | | | VRQAPGKGLEWMGGFDPEDGETVYAQK | |
| | | | FQGRVTMTEDTSSDTAYMELSSLRSEDT | |
| | | | AVYYCATSFPIRGDPSYYYYYYGMDVW | |
| | | | GQGTTVTVSSGGGGSGGGGSGGGGSGG | |
| | | | GGSDIQMTQSPSSLSASVGDRVTITCRAS | |
| | | | QGITNYLAWFQQKPGKAPKSLIYAVSSLQ | |
| | | | SGVPSKFSGSGSGTDFTLTISSLQPEDFAT | |
| | | | YYCQQYNSYPWTFGQGTKVEIK | |
| C018 | ScFv | — | MEFGLSWVFLVALFRGVQSEVQLVESGG | 57 |
| scFv | | | GVVQPGRSLRLSCAASGFTFSNYAIHWV | |
| | | | RQAPGKGLEWVAVISYDGSNKYYADSV | |
| | | | KGRFTISRDNSKNTLYLQMNSLRAEDTA | |
| | | | VYYCARDFDDSSFWAFDYWGQGTLVTV | |
| | | | SSGGGGSGGGGSGGGGSGGGGSDIQLTQ | |
| | | | SPSSLSASVGDRVTITCRASQSIRSYLNWY | |
| | | | QQKPGKAPKLLIYAASSLQSGVPSRFSGS | |
| | | | GSGTDFTLTISSLQPDDFATYYCQQSYSTP | |
| | | | PATFGQGTKLEIK | |
| human | Type II | — | KGTDDATADSRKTYTLTDYLKNTYRLKL | 58 |
| DPP4 | transmembrane | | YSLRWISDHEYLYKQENNILVFNAEYGN | |
| ecto | polypeptide | | SSVFLENSTFDEFGHSINDYSISPDGQFILL | |
| | | | EYNYVKQWRHSYTASYDIYDLNKRQLIT | |
| | | | EERIPNNTQWVTWSPVGHKLAYVWNND | |
| | | | IYVKIEPNLPSYRITWTGKEDIIYNGITDW | |
| | | | VYEEEVFSAYSALWWSPNGTFLAYAQFN | |
| | | | DTEVPLIEYSFYSDESLQYPKTVRVPYPK | |
| | | | AGAVNPTVKFFVVNTDSLSSVTNATSIQI | |
| | | | TAPASMLIGDHYLCDVTWATQERISLQW | |
| | | | LRRIQNYSVMDICDYDESSGRWNCLVAR | |
| | | | QHIEMSTTGWVGRFRPSEPHFTLDGNSFY | |
| | | | KIISNEEGYRHICYFQIDKKDCTFITKGTW | |
| | | | EVIGIEALTSDYLYYISNEYKGMPGGRNL | |
| | | | YKIQLSDYTKVTCLSCELNPERCQYYSVS | |
| | | | FSKEAKYYQLRCSGPGLPLYTLHSSVND | |
| | | | KGLRVLEDNSALDKMLQNVQMPSKKLD | |
| | | | FIILNETKFWYQMILPPHFDKSKKYPLLLD | |
| | | | VYAGPCSQKADTVFRLNWATYLASTENII | |
| | | | VASFDGRGSGYQGDKIMHAINRRLGTFE | |
| | | | VEDQIEAARQFSKMGFVDNKRIAIWGWS | |
| | | | YGGYVTSMVLGSGSGVFKCGIAVAPVSR | |
| | | | WEYYDSVYTERYMGLPTPEDNLDHYRN | |
| | | | STVMSRAENFKQVEYLLIHGTADDNVHF | |
| | | | QQSAQISKALVDVGVDFQAMWYTDEDH | |
| | | | GIASSTAHQHIYTHMSHFIKQCFSLP | |
| Human | Type I | — | MSSSSWLLLSLVAVTAAQSTIEEQAKTFL | 59 |
| ACE2 | transmembrane | | DKFNHEAEDLFYQSSLASWNYNTNITEE | |
| ecto | polypeptide | | NVQNMNNAGDKWSAFLKEQSTLAQMYP | |
| | | | LQEIQNLTVKLQLQALQQNGSSVLSEDKS | |
| | | | KRLNTILNTMSTIYSTGKVCNPDNPQECL | |
| | | | LLEPGLNEIMANSLDYNERLWAWESWRS | |
| | | | EVGKQLRPLYEEYVVLKNEMARANHYE | |
| | | | DYGDYWRGDYEVNGVDGYDYSRGQLIE | |
| | | | DVEHTFEEIKPLYEHLHAYVRAKLMNAY | |
| | | | PSYISPIGCLPAHLLGDMWGRFWTNLYSL | |
| | | | TVPFGQKPNIDVTDAMVDQAWDAQRIFK | |
| | | | EAEKFFVSVGLPNMTQGFWENSMLTDPG | |
| | | | NVQKAVCHPTAWDLGKGDFRILMCTKV | |
| | | | TMDDFLTAHAEMGAIQYDMAYAAQPFL | |
| | | | LRNGANEGFHEAVGEIMSLSAATPKHLK | |
| | | | SIGLLSPDFQEDNETEINFLLKQALTIVGT | |
| | | | LPFTYMLEKWRWMVFKGEIPKDQWMK | |
| | | | KWWEMKREIVGVVEPVPHDETYCDPAS | |
| | | | LFHVSNDYSFIRYYTRTLYQFQFQEALCQ | |
| | | | AAKHEGPLHKCDISNSTEAGQKLFNMLR | |
| | | | LGKSEPWTLALENVVGAKNMNVRPLLN | |
| | | | YFEPLFTWLKDQNKNSFVGWSTDWSPY | |
| | | | ADQSIKVRISLKSALGDKAYEWNDNEMY | |
| | | | LFRSSVAYAMRQYFLKVKNQMILFGEED | |
| | | | VRVANLKPRISFNFFVTAPKNVSDIIPRTE | |
| | | | VEKAIRMSRSRINDAFRLNDNSLEFLGIQP | |
| | | | TLGPPNQPPVS | |
| Mouse | Type I | — | MIAMLTVLLYLGLILEPRTAVQAGHLPKP | 60 |
| LILRB | transmembrane | | IIWAEPGSVIAAYTSVITWCQGSWEAQYY | |
| 4 ecto | polypeptide | | HLYKEKSVNPWDTQVPLETRNKAKFNIP | |
| | | | SMTTSYAGIYKCYYESAAGFSEHSDAME | |
| | | | LVMTGAYENPSLSVYPSSNVTSGVSISFS | |
| | | | CSSSIVFGRFILIQEGKHGLSWTLDSQHQA | |
| | | | NQPSYATFVLDAVTPNHNGTFRCYGYFR | |
| | | | NEPQVWSKPSNSLDLMISETKDQSSTPTE | |
| | | | DGLETYQKILIGVLVSFLLLFFLLLFLILIG | |
| | | | YQYGHKKKANASVKNTQSENNAELNSW | |
| | | | NPQNEDPQGIVYAQVKPSRLQKDTACKE | |
| | | | TQDVTYAQLCIRTQEQNNS | |
| Human | Type I | — | MGLSNILFVMAFLLSGAAPLKIQAYFNET | 61 |
| CD86 | transmembrane | | ADLPCQFANSQNQSLSELVVFWQDQENL | |
| ecto | polypeptide | | VLNEVYLGKEKFDSVHSKYMGRTSFDSD | |
| | | | SWTLRLHNLQIKDKGLYQCIIHHKKPTG | |
| | | | MIRIHQMNSELSVLANFSQPEIVPISNITEN | |
| | | | VYINLTCSSIHGYPEPKKMSVLLRTKNSTI | |
| | | | EYDGIMQKSQDNVTELYDVSISLSVSFPD | |
| | | | VTSNMTIFCILETDKTRLLSSPFSIELEDPQ | |
| | | | PPPDHIPWITAVL | |
| Human | Type II | — | MVSHRYPRIQSIKVQFTEYKKEKGFILTS | 62 |
| OX40L | transmembrane | | QKEDEIMKVQNNSVIINCDGFYLISLKGY | |
| ecto | polypeptide | | FSQEVNISLHYQKDEEPLFQLKKVRSVNS | |
| | | | LMVASLTYKDKVYLNVTTDNTSLDDFH | |
| | | | VNGGELILIHQNPGEFCVL | |
| Human | Type II | — | ACPWAVSGARASPGSAASPRLREGPELSP | 63 |
| 4- | transmembrane | | DDPAGLLDLRQGMFAQLVAQNVLLIDGP | |
| 1BBL | polypeptide | | LSWYSDPGLAGVSLTGGLSYKEDTKELV | |
| ecto | | | VAKAGVYYVFFQLELRRVVAGEGSGSVS | |
| | | | LALHLQPLRSAAGAAALALTVDLPPASSE | |
| | | | ARNSAFGFQGRLLHLSAGQRLGVHLHTE | |
| | | | ARARHAWQLTQGATVLGLFRVTPEIPAG | |
| | | | LPSPRSE | |
| Anti- | scFv | — | MPMGSLQPLATLYLLGMLVASVLADIKL | 64 |
| CD3 | | | QQSGAELARPGASVKMSCKTSGYTFTRY | |
| scFv | | | TMHWVKQRPGQGLEWIGYINPSRGYTN | |
| | | | YNQKFKDKATLTTDKSSSTAYMQLSSLT | |
| | | | SEDSAVYYCARYYDDHYCLDYWGQGTT | |
| | | | LTVSSVEGGSGGSGGSGGSGGVDDIQLT | |
| | | | QSPAIMSASPGEKVTMTCRASSSVSYMN | |
| | | | WYQQKSGTSPKRWIYDTSKVASGVPYRF | |
| | | | SGSGSGTSYSLTISSMEAEDAATYYCQQ | |
| | | | WSSNPLTFGAGTKLELK | |
| mouse | GPI-Anchored | — | MGRAMVARLGLGLLLLALLLPTQIYCNQ | 65 |
| CD24 | | | TSVAPFPGNQNISASPNPSNATTRG | |
| ecto | | | | |
| hu | GPI-Anchored | — | MCSRGWDSCLALELLLLPLSLLVTSIQGH | 66 |
| CD48 | | | LVHMTVVSGSNVTLNISESLPENYKQLT | |
| ecto | | | WFYTFDQKIVEWDSRKSKYFESKFKGRV | |
| | | | RLDPQSGALYISKVQKEDNSTYIMRVLK | |
| | | | KTGNEQEWKIKLQVLDPVPKPVIKIEKIE | |
| | | | DMDDNCYLKLSCVIPGESVNYTWYGDK | |
| | | | RPFPKELQNSVLETTLMPHNYSRCYTCQ | |
| | | | VSNSVSSKNGTVCLSPPCTLARSFGVEWI | |
| | | | ASWLVVTVPTILGLLLT | |
| mouse | Immune | — | MGVRQVPWSFTWAVLQLSWQSGWLLE | 67 |
| PD-1 | Checkpoint | | VPNGPWRSLTFYPAWLTVSEGANATFTC | |
| ecto | Protein | | SLSNWSEDLMLNWNRLSPSNQTEKQAAF | |
| | | | CNGLSQPVQDARFQIIQLPNRHDFHMNIL | |
| | | | DTRRNDSGIYLCGAISLHPKAKIEESPGAE | |
| | | | LVVTERILETSTRYPSPSPKPEGRFQGMVI | |
| | | | GIMSALVGIPVLLLLAWAL | |
| human | Multi-pass | — | MEAHNASAPFNFTLPPNFGKRPTDLALSV | 68 |
| NTCP | Transmembrane | | ILVFMLFFIMLSLGCTMEFSKIKAHLWKP | |
| | polypeptide | | KGLAIALVAQYGIMPLTAFVLGKVFRLK | |
| | | | NIEALAILVCGCSPGGNLSNVFSLAMKGD | |
| | | | MNLSIVMTTCSTFCALGMMPLLLYIYSRG | |
| | | | IYDGDLKDKVPYKGIVISLVLVLIPCTIGIV | |
| | | | LKSKRPQYMRYVIKGGMIIILLCSVAVTV | |
| | | | LSAINVGKSIMFAMTPLLIATSSLMPFIGF | |
| | | | LLGYVLSALFCLNGRCRRTVSMETGCQN | |
| | | | VQLCSTILNVAFPPEVIGPLFFFPLLYMIFQ | |
| | | | LGEGLLLIAIFWCYEKFKTPKDKTKMIYT | |
| | | | AATTEETIPGALGNGTYKGEDCSPCTA | |
| Mouse | Immune | — | MTCLGLRRYKAQLQLPSRTWPFVALLTL | 69 |
| CTLA- | Checkpoint | | LFIPVFSEAIQVTQPSVVLASSHGVASFPC | |
| 4 ecto | Protein | | EYSPSHNTDEVRVTVLRQTNDQMTEVCA | |
| | | | TTFTEKNTVGFLDYPFCSGTFNESRVNLTI | |
| | | | QGLRAVDTGLYLCKVELMYPPPYFVGM | |
| | | | GNGTQIYVIDPEPCPDSDFLLWILVAVSL | |
| | | | GLFFYSFLVTAVSLSKRIQ | |
| human | Type I | — | MGHTRRQGTSPSKCPYLNFFQLLVLAGL | 70 |
| CD80 | transmembrane | | SHFCSGVIHVTKEVKEVATLSCGHNVSVE | |
| ecto | polypeptide | | ELAQTRIYWQKEKKMVLTMMSGDMNIW | |
| | | | PEYKNRTIFDITNNLSIVILALRPSDEGTYE | |
| | | | CVVLKYEKDAFKREHLAEVTLSVKADFP | |
| | | | TPSISDFEIPTSNIRRIICSTSGGFPEPHLSW | |
| | | | LENGEELNAINTTVSQDPETELYAVSSKL | |
| | | | DFNMTTNHSFMCLIKYGHLRVNQTFNW | |
| | | | NTTKQEHFPDNLLPS | |

In some embodiments, the display polypeptide comprises an amino acid sequence according to any amino acid sequence of Table 2, or a sequence that is substantially similar to a sequence of Table 2 (e.g. about 75%, 76%, 77%, 78%, 79%, 80%, 81%, 82%, 83%, 84%, 85%, 86%, 87%, 88%, 89$, 90%, 91%, 92%, 93%, 94%, 95%, 96%, 97%, 98%, or about 99% sequence identity). In some instances, the display polypeptide comprises an amino acid sequence comprising at least a portion having at least or about 50, 60, 70, 80, 90, 100, 150, 200, 250, 300, 350, 400, 450, 500, 550, 600, 650, 700, 750, 800, 850, 900, 950, or 1000 amino acid sequences of any sequence according to Table 2.

In some embodiments, the transmembrane domain comprises the transmembrane domain of a Vesicular Stomatitis virus glycoprotein (VSV-G). In some embodiments, the recombinant fusion protein comprises the transmembrane domain and cytosolic domain of a Vesicular Stomatitis virus glycoprotein (VSV-G). In some embodiments, the transmembrane domain comprises the transmembrane domain of a Dengue E protein. In some embodiments, the recombinant fusion protein comprises the transmembrane domain and cytosolic domain of a Dengue E protein. In some embodiments, the transmembrane domain comprises the transmembrane domain of influenza Hemagglutinin (HA). In some embodiments, the recombinant fusion protein comprises the transmembrane domain and cytosolic domain of influenza Hemagglutinin (HA). In some embodiments, the transmembrane domain comprises the transmembrane domain of HIV surface glycoprotein GP120 or GP41. In some embodiments, recombinant fusion protein comprises the transmembrane domain and cytosolic domain of HIV surface glycoprotein GP120 or GP41. In some embodiments, the transmembrane domain comprises the transmembrane domain of measles virus surface glycoprotein hamagglutinin (H) protein. In some embodiments, the recombinant fusion protein comprises the transmembrane domain and cytosolic domain of measles virus surface glycoprotein hamagglutinin (H) protein. In some embodiments, the transmembrane domain comprises the transmembrane domain of influenza Neuraminidase (NA). In some embodiments, the recombinant fusion protein comprises the transmembrane domain and cytosolic domain of influenza Neuraminidase (NA).

TABLE 3
Exemplary Transmembrane Domain Sequences

| Transmembrane Domain | Amino Acid Sequence | SEQ ID NO: |
|---|---|---|
| VSV-G | IASFFFIIGLIIGLFLVLRVGI | 1 |
| Transmembrane | | |
| (TM) V1 | | |
| VSV-G | PIELVEGWFSSWKSSIASFFFIIGLIIGLFLVLRVGKI | 2 |
| Transmembrane | | |
| (TM) V2 | | |
| VSV-G | DDESLFFGDTGLSKNPIELVEGWFSSWKSSIASFFFII | 3 |
| Transmembrane | GLIIGLFLVLRVGIH | |
| (TM) V3 | | |
| VSV-G | GMLDSDLHLSSKAQVFEHPHIQDAASQLPDDESLFFGD | 4 |
| Transmembrane | TGLSKNPIELVEGWFSSWKSSIASFFFIIGLIIGLFLV | |
| (TM) V4 | LRVGI | |
| VSV-G | HLCIKLKHTKKRQIYTDIEMNRLGK | 5 |
| Cytosolic Tail | | |
| (CT) | | |
| Influenza | IITIGSVCMTIGMANLILQIGNI | 52 |
| Neuraminidase | | |
| TM (N1) | | |
| Influenza | LAIYSTVASSLVLVVSLGAISFW | 53 |
| Hemagglutinin | | |
| TM (H1) | | |
| Dengue E | AYGVLFSGVSWTMKIGIGILLTWLGLNSRSTSLSMTCI | 54 |
| Protein TM | AVGMVTLYLGVMVQ | |
| HIV gp TM | FIMIVGGLVGLRIVFAVLSIV | 55 |

In some embodiments, the transmembrane domain comprises an amino acid sequence of at least 75% sequence identity to an amino acid sequence according to SEQ ID NO: 1. In some embodiments, the transmembrane domain comprises an amino acid sequence of at least 80% sequence identity to an amino acid sequence according to SEQ ID NO: 1. In some embodiments, the transmembrane domain comprises an amino acid sequence of at least 85% sequence identity to an amino acid sequence according to SEQ ID NO: 1. In some embodiments, the transmembrane domain comprises an amino acid sequence of at least 90% sequence identity to an amino acid sequence according to SEQ ID NO: 1. In some embodiments, the transmembrane domain comprises an amino acid sequence of at least 95% sequence identity to an amino acid sequence according to SEQ ID NO: 1.

In some embodiments, the transmembrane domain comprises an amino acid sequence of at least 75% sequence homology to an amino acid sequence according to SEQ ID NO: 1. In some embodiments, the transmembrane domain comprises an amino acid sequence of at least 80% sequence homology to an amino acid sequence according to SEQ ID NO: 1. In some embodiments, the transmembrane domain comprises an amino acid sequence of at least 85% sequence homology to an amino acid sequence according to SEQ ID NO: 1. In some embodiments, the transmembrane domain comprises an amino acid sequence of at least 90% sequence homology to an amino acid sequence according to SEQ ID NO: 1. In some embodiments, the transmembrane domain comprises an amino acid sequence of at least 95% sequence homology to an amino acid sequence according to SEQ ID NO: 1. In some embodiments, the transmembrane domain comprises an amino acid sequence according to SEQ ID NO: 1. In some embodiments, the transmembrane domain comprises an amino acid sequence of at least 75% sequence identity to an amino acid sequence according to SEQ ID NO: 2. In some embodiments, the transmembrane domain comprises an amino acid sequence of at least 80% sequence identity to an amino acid sequence according to SEQ ID NO: 2. In some embodiments, the transmembrane domain comprises an amino acid sequence of at least 85% sequence identity to an amino acid sequence according to SEQ ID NO: 2. In some embodiments, the transmembrane domain comprises an amino acid sequence of at least 90% sequence identity to an amino acid sequence according to SEQ ID NO: 2. In some embodiments, the transmembrane domain comprises an amino acid sequence of at least 95% sequence identity to an amino acid sequence according to SEQ ID NO: 2. In some embodiments, the transmembrane domain comprises an amino acid sequence of at least 97% sequence identity to an amino acid sequence according to SEQ ID NO: 2.

In some embodiments, the transmembrane domain comprises an amino acid sequence of at least 75% sequence homology to an amino acid sequence according to SEQ ID NO: 2. In some embodiments, the transmembrane domain comprises an amino acid sequence of at least 80% sequence homology to an amino acid sequence according to SEQ ID NO: 2. In some embodiments, the transmembrane domain comprises an amino acid sequence of at least 85% sequence homology to an amino acid sequence according to SEQ ID NO: 2. In some embodiments, the transmembrane domain comprises an amino acid sequence of at least 90% sequence homology to an amino acid sequence according to SEQ ID NO: 2. In some embodiments, the transmembrane domain comprises an amino acid sequence of at least 95% sequence homology to an amino acid sequence according to SEQ ID NO: 2. In some embodiments, the transmembrane domain comprises an amino acid sequence of at least 97% sequence homology to an amino acid sequence according to SEQ ID NO: 2.

In some embodiments, the transmembrane domain comprises an amino acid sequence according to SEQ ID NO: 2.

In some embodiments, the transmembrane domain comprises an amino acid sequence of at least 75% sequence identity to an amino acid sequence according to SEQ ID NO: 3. In some embodiments, the transmembrane domain comprises an amino acid sequence of at least 80% sequence identity to an amino acid sequence according to SEQ ID NO: 3. In some embodiments, the transmembrane domain comprises an amino acid sequence of at least 85% sequence identity to an amino acid sequence according to SEQ ID NO: 3. In some embodiments, the transmembrane domain comprises an amino acid sequence of at least 90% sequence identity to an amino acid sequence according to SEQ ID NO: 3. In some embodiments, the transmembrane domain comprises an amino acid sequence of at least 95% sequence identity to an amino acid sequence according to SEQ ID NO: 3. In some embodiments, the transmembrane domain comprises an amino acid sequence of at least 97% sequence identity to an amino acid sequence according to SEQ ID NO: 3. In some embodiments, the transmembrane domain comprises an amino acid sequence of at least 98% sequence identity to an amino acid sequence according to SEQ ID NO: 3.

In some embodiments, the transmembrane domain comprises an amino acid sequence of at least 75% sequence homology to an amino acid sequence according to SEQ ID NO: 3. In some embodiments, the transmembrane domain comprises an amino acid sequence of at least 80% sequence homology to an amino acid sequence according to SEQ ID NO: 3. In some embodiments, the transmembrane domain comprises an amino acid sequence of at least 85% sequence homology to an amino acid sequence according to SEQ ID NO: 3. In some embodiments, the transmembrane domain comprises an amino acid sequence of at least 90% sequence homology to an amino acid sequence according to SEQ ID NO: 3. In some embodiments, the transmembrane domain comprises an amino acid sequence of at least 95% sequence homology to an amino acid sequence according to SEQ ID NO: 3. In some embodiments, the transmembrane domain comprises an amino acid sequence of at least 97% sequence homology to an amino acid sequence according to SEQ ID NO: 3. In some embodiments, the transmembrane domain comprises an amino acid sequence of at least 98% sequence homology to an amino acid sequence according to SEQ ID NO: 3.

In some embodiments, the transmembrane domain comprises an amino acid sequence according to SEQ ID NO: 3.

In some embodiments, the transmembrane domain comprises an amino acid sequence of at least 75% sequence identity to an amino acid sequence according to SEQ ID NO: 4. In some embodiments, the transmembrane domain comprises an amino acid sequence of at least 80% sequence identity to an amino acid sequence according to SEQ ID NO: 4. In some embodiments, the transmembrane domain comprises an amino acid sequence of at least 85% sequence identity to an amino acid sequence according to SEQ ID NO: 4. In some embodiments, the transmembrane domain comprises an amino acid sequence of at least 90% sequence identity to an amino acid sequence according to SEQ ID NO: 4. In some embodiments, the transmembrane domain comprises an amino acid sequence of at least 95% sequence identity to an amino acid sequence according to SEQ ID NO: 4. In some embodiments, the transmembrane domain comprises an amino acid sequence of at least 97% sequence identity to an amino acid sequence according to SEQ ID NO: 4. In some embodiments, the transmembrane domain comprises an amino acid sequence of at least 98% sequence identity to an amino acid sequence according to SEQ ID NO: 4.

In some embodiments, the transmembrane domain comprises an amino acid sequence of at least 75% sequence homology to an amino acid sequence according to SEQ ID NO: 4. In some embodiments, the transmembrane domain comprises an amino acid sequence of at least 80% sequence homology to an amino acid sequence according to SEQ ID NO: 4. In some embodiments, the transmembrane domain comprises an amino acid sequence of at least 85% sequence homology to an amino acid sequence according to SEQ ID NO: 4. In some embodiments, the transmembrane domain comprises an amino acid sequence of at least 90% sequence homology to an amino acid sequence according to SEQ ID NO: 4. In some embodiments, the transmembrane domain comprises an amino acid sequence of at least 95% sequence homology to an amino acid sequence according to SEQ ID NO: 4. In some embodiments, the transmembrane domain comprises an amino acid sequence of at least 97% sequence homology to an amino acid sequence according to SEQ ID NO: 4. In some embodiments, the transmembrane domain comprises an amino acid sequence of at least 98% sequence homology to an amino acid sequence according to SEQ ID NO: 4.

In some embodiments, the transmembrane domain comprises an amino acid sequence according to SEQ ID NO: 4.

In some embodiments, the transmembrane domain comprises an amino acid sequence of at least 75% sequence identity to an amino acid sequence according to SEQ ID NO: 5. In some embodiments, the transmembrane domain comprises an amino acid sequence of at least 80% sequence identity to an amino acid sequence according to SEQ ID NO: 5. In some embodiments, the transmembrane domain comprises an amino acid sequence of at least 85% sequence identity to an amino acid sequence according to SEQ ID NO: 5. In some embodiments, the transmembrane domain comprises an amino acid sequence of at least 90% sequence identity to an amino acid sequence according to SEQ ID NO: 5. In some embodiments, the transmembrane domain comprises an amino acid sequence of at least 95% sequence identity to an amino acid sequence according to SEQ ID NO: 5. In some embodiments, the transmembrane domain comprises an amino acid sequence of at least 96% sequence identity to an amino acid sequence according to SEQ ID NO: 5.

In some embodiments, the cytosolic domain comprises an amino acid sequence having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or about 99% sequence identity to SEQ ID No: 5. In some embodiments, the cytosolic domain comprises an amino acid sequence of SEQ ID No: 5.

In some embodiments, the transmembrane domain comprises an amino acid sequence of at least 75% sequence homology to an amino acid sequence according to SEQ ID NO: 5. In some embodiments, the transmembrane domain comprises an amino acid sequence of at least 80% sequence homology to an amino acid sequence according to SEQ ID NO: 5. In some embodiments, the transmembrane domain comprises an amino acid sequence of at least 85% sequence homology to an amino acid sequence according to SEQ ID NO: 5. In some embodiments, the transmembrane domain comprises an amino acid sequence of at least 90% sequence homology to an amino acid sequence according to SEQ ID NO: 5. In some embodiments, the transmembrane domain comprises an amino acid sequence of at least 95% sequence homology to an amino acid sequence according to SEQ ID NO: 5. In some embodiments, the transmembrane domain comprises an amino acid sequence of at least 96% sequence homology to an amino acid sequence according to SEQ ID NO: 5.

In some embodiments, the transmembrane domain comprises an amino acid sequence according to SEQ ID NO: 5.

In some embodiments, the transmembrane domain comprises an amino acid sequence of at least 75% sequence identity to an amino acid sequence according to SEQ ID NO: 52. In some embodiments, the transmembrane domain comprises an amino acid sequence of at least 80% sequence identity to an amino acid sequence according to SEQ ID NO: 52. In some embodiments, the transmembrane domain comprises an amino acid sequence of at least 85% sequence identity to an amino acid sequence according to SEQ ID NO: 52. In some embodiments, the transmembrane domain comprises an amino acid sequence of at least 90% sequence identity to an amino acid sequence according to SEQ ID NO: 52. In some embodiments, the transmembrane domain comprises an amino acid sequence of at least 95% sequence identity to an amino acid sequence according to SEQ ID NO: 52.

In some embodiments, the transmembrane domain comprises an amino acid sequence of at least 75% sequence homology to an amino acid sequence according to SEQ ID NO: 52. In some embodiments, the transmembrane domain comprises an amino acid sequence of at least 80% sequence homology to an amino acid sequence according to SEQ ID NO: 52. In some embodiments, the transmembrane domain comprises an amino acid sequence of at least 85% sequence homology to an amino acid sequence according to SEQ ID NO: 52. In some embodiments, the transmembrane domain comprises an amino acid sequence of at least 90% sequence homology to an amino acid sequence according to SEQ ID NO: 52. In some embodiments, the transmembrane domain comprises an amino acid sequence of at least 95% sequence homology to an amino acid sequence according to SEQ ID NO: 52. In some embodiments, the transmembrane domain comprises an amino acid sequence according to SEQ ID NO: 52. In some embodiments, the transmembrane domain comprises an amino acid sequence of at least 75% sequence identity to an amino acid sequence according to SEQ ID NO: 53. In some embodiments, the transmembrane domain comprises an amino acid sequence of at least 80% sequence identity to an amino acid sequence according to SEQ ID NO: 53. In some embodiments, the transmembrane domain comprises an amino acid sequence of at least 85% sequence identity to an amino acid sequence according to SEQ ID NO: 53. In some embodiments, the transmembrane domain comprises an amino acid sequence of at least 90% sequence identity to an amino acid sequence according to SEQ ID NO: 53. In some embodiments, the transmembrane domain comprises an amino acid sequence of at least 95% sequence identity to an amino acid sequence according to SEQ ID NO: 53. In some embodiments, the transmembrane domain comprises an amino acid sequence of at least 97% sequence identity to an amino acid sequence according to SEQ ID NO: 53.

In some embodiments, the transmembrane domain comprises an amino acid sequence of at least 75% sequence homology to an amino acid sequence according to SEQ ID NO: 53. In some embodiments, the transmembrane domain comprises an amino acid sequence of at least 80% sequence homology to an amino acid sequence according to SEQ ID NO: 53. In some embodiments, the transmembrane domain comprises an amino acid sequence of at least 85% sequence homology to an amino acid sequence according to SEQ ID NO: 53. In some embodiments, the transmembrane domain comprises an amino acid sequence of at least 90% sequence homology to an amino acid sequence according to SEQ ID NO: 53. In some embodiments, the transmembrane domain comprises an amino acid sequence of at least 95% sequence homology to an amino acid sequence according to SEQ ID NO: 53. In some embodiments, the transmembrane domain comprises an amino acid sequence of at least 97% sequence homology to an amino acid sequence according to SEQ ID NO: 53.

In some embodiments, the transmembrane domain comprises an amino acid sequence according to SEQ ID NO: 53.

In some embodiments, the transmembrane domain comprises an amino acid sequence of at least 75% sequence identity to an amino acid sequence according to SEQ ID NO: 54. In some embodiments, the transmembrane domain comprises an amino acid sequence of at least 80% sequence identity to an amino acid sequence according to SEQ ID NO: 54. In some embodiments, the transmembrane domain comprises an amino acid sequence of at least 85% sequence identity to an amino acid sequence according to SEQ ID NO: 54. In some embodiments, the transmembrane domain comprises an amino acid sequence of at least 90% sequence identity to an amino acid sequence according to SEQ ID NO: 54. In some embodiments, the transmembrane domain comprises an amino acid sequence of at least 95% sequence identity to an amino acid sequence according to SEQ ID NO: 54. In some embodiments, the transmembrane domain comprises an amino acid sequence of at least 97% sequence identity to an amino acid sequence according to SEQ ID NO: 54. In some embodiments, the transmembrane domain comprises an amino acid sequence of at least 98% sequence identity to an amino acid sequence according to SEQ ID NO: 54.

In some embodiments, the transmembrane domain comprises an amino acid sequence of at least 75% sequence homology to an amino acid sequence according to SEQ ID NO: 54. In some embodiments, the transmembrane domain comprises an amino acid sequence of at least 80% sequence homology to an amino acid sequence according to SEQ ID NO: 54. In some embodiments, the transmembrane domain comprises an amino acid sequence of at least 85% sequence homology to an amino acid sequence according to SEQ ID NO: 54. In some embodiments, the transmembrane domain comprises an amino acid sequence of at least 90% sequence homology to an amino acid sequence according to SEQ ID NO: 54. In some embodiments, the transmembrane domain comprises an amino acid sequence of at least 95% sequence homology to an amino acid sequence according to SEQ ID NO: 54. In some embodiments, the transmembrane domain comprises an amino acid sequence of at least 97% sequence homology to an amino acid sequence according to SEQ ID NO: 54. In some embodiments, the transmembrane domain comprises an amino acid sequence of at least 98% sequence homology to an amino acid sequence according to SEQ ID NO: 54. In some embodiments, the transmembrane domain comprises an amino acid sequence according to SEQ ID NO: 54. In some embodiments, the transmembrane domain comprises an amino acid sequence of at least 75% sequence identity to an amino acid sequence according to SEQ ID NO: 55. In some embodiments, the transmembrane domain comprises an amino acid sequence of at least 80% sequence identity to an amino acid sequence according to SEQ ID NO: 55. In some embodiments, the transmembrane domain comprises an amino acid sequence of at least 85% sequence identity to an amino acid sequence according to SEQ ID NO: 55. In some embodiments, the transmembrane domain comprises an amino acid sequence of at least 90% sequence identity to an amino acid sequence according to SEQ ID NO: 55. In some embodiments, the transmembrane domain comprises an amino acid sequence of at least 95% sequence identity to an amino acid sequence according to SEQ ID NO: 55. In some embodiments, the transmembrane domain comprises an amino acid sequence of at least 96% sequence identity to an amino acid sequence according to SEQ ID NO: 55.

In some embodiments, the transmembrane domain comprises an amino acid sequence of at least 75% sequence homology to an amino acid sequence according to SEQ ID NO: 55. In some embodiments, the transmembrane domain comprises an amino acid sequence of at least 80% sequence homology to an amino acid sequence according to SEQ ID NO: 55. In some embodiments, the transmembrane domain comprises an amino acid sequence of at least 85% sequence homology to an amino acid sequence according to SEQ ID NO: 55. In some embodiments, the transmembrane domain comprises an amino acid sequence of at least 90% sequence homology to an amino acid sequence according to SEQ ID NO: 55. In some embodiments, the transmembrane domain comprises an amino acid sequence of at least 95% sequence homology to an amino acid sequence according to SEQ ID NO: 55. In some embodiments, the transmembrane domain comprises an amino acid sequence of at least 96% sequence homology to an amino acid sequence according to SEQ ID NO: 55. In some embodiments, the transmembrane domain comprises an amino acid sequence according to SEQ ID NO: 55. In some embodiments, the transmembrane domain comprises an amino acid sequence disclosed in Table 3, or an amino acid sequence that is substantially identical to an amino acid sequence in Table 3 (e.g. about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or about 99% sequence identity). In some instances, the transmembrane domain comprises an amino acid sequence comprising at least a portion having at least or about 10, 20, 30, 40, 50, 60, 70, 80, 90, 100, 110, 120, 130 amino acid sequences of any sequence according to Table 3.

In some embodiments, when the recombinant fusion protein is expressed on the surface of the enveloped particle the recombinant fusion protein is expressed at a valency of about 10 copies on the surface of the enveloped particle. In some embodiments, when the recombinant fusion protein is expressed on the surface of the enveloped particle the recombinant fusion protein is expressed at a valency of about 10 to 15 copies on the surface of the enveloped particle. In some embodiments, when the recombinant fusion protein is expressed on the surface of the enveloped particle the recombinant fusion protein is expressed at a valency of at least about 25 copies on the surface of the enveloped particle. In some embodiments, when the recombinant fusion protein is expressed on the surface of the enveloped particle the recombinant fusion protein is expressed at a valency of at least about 50 copies on the surface of the enveloped particle. In some embodiments, when the recombinant fusion protein is expressed on the surface of the enveloped particle the recombinant fusion protein is expressed at a valency of at least about 100 copies on the surface of the enveloped particle. In some embodiments, when the recombinant fusion protein is expressed on the surface of the enveloped particle the recombinant fusion protein is expressed at a valency of at least about 200 copies on the surface of the enveloped particle. In some embodiments, when the recombinant fusion protein is expressed on the surface of the enveloped particle the recombinant fusion protein is expressed at a valency of at least about 400 copies on the surface of the enveloped particle. In some embodiments, when the recombinant fusion protein is expressed on the surface of the enveloped particle the recombinant fusion protein is expressed at a valency of at least about 600 copies on the surface of the enveloped particle. In some embodiments, when the recombinant fusion protein is expressed on the surface of the enveloped particle the recombinant fusion protein is expressed at least about 1000 copies on the surface of the enveloped particle.

In some embodiments, the recombinant fusion protein comprises an amino acid sequence having at least about 75% sequence identity to any of SEQ ID NOs: 72-78, 81-94, 96-100. In some embodiments, the recombinant fusion protein comprises an amino acid sequence having at least about 76% sequence identity to any of SEQ ID NOs: 72-78, 81-94, 96-100. In some embodiments, the recombinant fusion protein comprises an amino acid sequence having at least about 77% sequence identity to any of SEQ ID NOs: 72-78, 81-94, 96-100. In some embodiments, the recombinant fusion protein comprises an amino acid sequence having at least about 78% sequence identity to any of SEQ ID NOs: 72-78, 81-94, 96-100. In some embodiments, the recombinant fusion protein comprises an amino acid sequence having at least about 79% sequence identity to any of SEQ ID NOs: 72-78, 81-94, 96-100. In some embodiments, the recombinant fusion protein comprises an amino acid sequence having at least about 80% sequence identity to any of SEQ ID NOs: 72-78, 81-94, 96-100. In some embodiments, the recombinant fusion protein comprises an amino acid sequence having at least about 81% sequence identity to any of SEQ ID NOs: 72-78, 81-94, 96-100. In some embodiments, the recombinant fusion protein comprises an amino acid sequence having at least about 82% sequence identity to any of SEQ ID NOs: 72-78, 81-94, 96-100. In some embodiments, the recombinant fusion protein comprises an amino acid sequence having at least about 83% sequence identity to any of SEQ ID NOs: 72-78, 81-94, 96-100. In some embodiments, the recombinant fusion protein comprises an amino acid sequence having at least about 84% sequence identity to any of SEQ ID NOs: 72-78, 81-94, 96-100. In some embodiments, the recombinant fusion protein comprises an amino acid sequence having at least about 85% sequence identity to any of SEQ ID NOs: 72-78, 81-94, 96-100. In some embodiments, the recombinant fusion protein comprises an amino acid sequence having at least about 86% sequence identity to any of SEQ ID NOs: 72-78, 81-94, 96-100. In some embodiments, the recombinant fusion protein comprises an amino acid sequence having at least about 87% sequence identity to any of SEQ ID NOs: 72-78, 81-94, 96-100. In some embodiments, the recombinant fusion protein comprises an amino acid sequence having at least about 88% sequence identity to any of SEQ ID NOs: 72-78, 81-94, 96-100. In some embodiments, the recombinant fusion protein comprises an amino acid sequence having at least about 89% sequence identity to any of SEQ ID NOs: 72-78, 81-94, 96-100. In some embodiments, the recombinant fusion protein comprises an amino acid sequence having at least about 90% sequence identity to any of SEQ ID NOs: 72-78, 81-94, 96-100. In some embodiments, the recombinant fusion protein comprises an amino acid sequence having at least about 91% sequence identity to any of SEQ ID NOs: 72-78, 81-94, 96-100. In some embodiments, the recombinant fusion protein comprises an amino acid sequence having at least about 92% sequence identity to any of SEQ ID NOs: 72-78, 81-94, 96-100. In some embodiments, the recombinant fusion protein comprises an amino acid sequence having at least about 93% sequence identity to any of SEQ ID NOs: 72-78, 81-94, 96-100. In some embodiments, the recombinant fusion protein comprises an amino acid sequence having at least about 94% sequence identity to any of SEQ ID NOs: 72-78, 81-94, 96-100. In some embodiments, the recombinant fusion protein comprises an amino acid sequence having at least about 95% sequence identity to any of SEQ ID NOs: 72-78, 81-94, 96-100. In some embodiments, the recombinant fusion protein comprises an amino acid sequence having at least about 96% sequence identity to any of SEQ ID NOs: 72-78, 81-94, 96-100. In some embodiments, the recombinant fusion protein comprises an amino acid sequence having at least about 97% sequence identity to any of SEQ ID NOs: 72-78, 81-94, 96-100. In some embodiments, the recombinant fusion protein comprises an amino acid sequence having at least about 98% sequence identity to any of SEQ ID NOs: 72-78, 81-94, 96-100. In some embodiments, the recombinant fusion protein comprises an amino acid sequence having at least about 99% sequence identity to any of SEQ ID NOs: 72-78, 81-94, 96-100. In some embodiments, the recombinant fusion protein comprises an amino acid sequence of any of SEQ ID NOs: 72-78, 81-94, 96-100.

### Enveloped Particles

Disclosed herein are enveloped particles comprising the recombinant fusion proteins described herein. In some embodiments, the enveloped particle comprises a viral-like particle. In some embodiments, the enveloped particle comprises an enveloped virus. In some embodiments, the enveloped particle comprises an extracellular vesicle. In some embodiments, the extracellular vesicle comprises an exosome or an ectosome.

Disclosed herein are enveloped particles comprising: (a) a first recombinant fusion protein that is displayed in an oligomeric format on the surface of the enveloped particle wherein the first recombinant fusion protein comprises a transmembrane domain, a display polypeptide, and an oligomerization domain; and (b) a second recombinant fusion protein that is displayed in a monomeric format on the surface of the enveloped particle wherein the second recombinant fusion protein comprises a transmembrane domain and a display polypeptide.

In some embodiments, the oligomerization domain is a dimerization domain. In some embodiments, the dimerization domain comprises a leucine zipper dimerization domain. In some embodiments, the oligomerization domain is a trimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the oligomerization domain is a tetramerization domain. In some embodiments, the tetramerization domain comprises an influenza neuraminidase stem domain.

In some embodiments, the oligomerization domain comprises an amino acid sequence disclosed in Table 1, or an amino acid sequence that is substantially identical to an amino acid sequence in Table 1 (e.g. about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or about 99% sequence identity). In some instances, the oligomerization domain comprises an amino acid sequence comprising at least a portion having at least or about 10, 20, 30, 40, 50, 60, 70, 80, 90, 100, 110, 120, 130 amino acid sequences of any sequence according to Table 1.

In some embodiments, when the first recombinant fusion protein is expressed on the surface of the enveloped particle, the oligomerization domain is outside of the enveloped particle. In some embodiments, when the first recombinant fusion protein is expressed on the surface of the enveloped particle, the oligomerization domain is outside of the enveloped particle and adjacent to a signal peptide. In some embodiments, when the first recombinant fusion protein is expressed on the surface of the enveloped particle, the oligomerization domain is inside of the enveloped particle. In some embodiments, when the first recombinant fusion protein is expressed on the surface of the enveloped particle, the oligomerization domain is inside of the enveloped particle and adjacent to the transmembrane domain. In some embodiments, the first recombinant fusion protein comprises a signal peptide. In some embodiments, domains of the first recombinant fusion protein are arranged from the N-terminus to the C-terminus in the following orders: (a) signal peptide, display polypeptide, oligomerization domain, and transmembrane domain: (b) signal peptide, display polypeptide, transmembrane domain, and oligomerization domain: or (c) signal peptide, oligomerization domain, display peptide, and transmembrane domain. In some embodiments, domains of the first recombinant fusion protein are arranged from the N-terminus to the C-terminus in the following orders: (a) signal peptide, display polypeptide, oligomerization domain, transmembrane domain, and cytosolic domain: (b) signal peptide, display polypeptide, transmembrane domain, oligomerization domain, and cytosolic domain: or (c) signal peptide, oligomerization domain, display peptide, transmembrane domain, and cytosolic domain. In some embodiments, the enveloped particle comprises an enveloped viral-like particle, an enveloped virus, an extracellular vesicle. In some embodiments, the extracellular vesicle comprises an exosome or an ectosome. In some embodiments, the first recombinant fusion protein and the second recombinant fusion protein have the same display polypeptide. In some embodiments, the first recombinant fusion protein and the second recombinant fusion protein have different display polypeptides. In some embodiments, the display polypeptide targets a cell surface protein that is oligomerized on a target cell for agonistic or antagonistic function. In some embodiments, the cell surface protein is dimerized and the oligomerization domain is a dimerization domain. In some embodiments, the cell surface protein is trimerized and the oligomerization domain is a trimerization domain. In some embodiments, the cell surface protein is tetramerized and the oligomerization domain is a tetramerization domain.

In some embodiments, the display polypeptide comprises a single chain variable fragment (scFv), a type I transmembrane polypeptide, a type II transmembrane polypeptide, a type III transmembrane polypeptide, a GPI-anchored polypeptide, a secreted polypeptide, or a multi-pass transmembrane polypeptide. In some embodiments, the scFv comprises an anti-BCMA scFv polypeptide, an anti-NTD scFv polypeptide, an anti-RBD scFv polypeptide, or an anti-CD19 scFv polypeptide. In some embodiments, the type I transmembrane polypeptide comprises ACE2, ICAM-1. LILRA5, LILRB3, or LILRB4. In some embodiments, the type II transmembrane polypeptide comprises DPP4, or BAFF. In some embodiments, the type III transmembrane polypeptide comprises BCMA, TACI, or BAFF Receptor. In some embodiments, the GPI-anchored polypeptide comprises CD24, CD48, CD59 or CD55. In some embodiments, the secreted polypeptide comprises IL-4, or CXCL-12. In some embodiments, the multi-pass transmembrane polypeptide comprises NTCP, or FCεRI beta. In some embodiments, the display polypeptide comprises an antibody, a receptor, a cytokine, an immune checkpoint protein, a chemokine, an adhesion factor, or a mitogen. In some embodiments, the antibody comprises a non-neutralizing antibody, a neutralizing antibody, or an antibody of multiple formats. In some embodiments, the receptor comprises ACE2, or DPP4. In some embodiments, the cytokine comprises IL-4, or IL-6. In some embodiments, the immune checkpoint protein comprises CTLA-4, or PD-1. In some embodiments, the chemokine comprises CXCL-12. In some embodiments, the adhesion factor comprises ICAM-1, or LFA-1. In some embodiments, the mitogen comprises Wnt. In some embodiments, the display polypeptide comprises an anti-BCMA scFv polypeptide, an anti-CD19 scFv polypeptide, an anti-NTD scFv polypeptide, an anti-RBD scFv polypeptide, ACE2, ICAM-1, LILRA5, LILRB3, LILRB4, DPP4, BAFF, BCMA, TACI, BAFF Receptor, CD24, CD48, CD59, CD55, IL-4, CXCL-12, NTCP, FCεRI beta, a non-neutralizing antibody, a neutralizing antibody, an antibody of multiple formats, IL-6, CTLA-4, PD-1, LFA-1, Wnt, CD80, CD86, OX40L, CD3, or 4-1BBL.

In some embodiments, the display polypeptide comprises an amino acid sequence according to any amino acid sequence of Table 2, or a sequence that is substantially similar to a sequence of Table 2 (e.g. about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or about 99% sequence identity). In some instances, the display polypeptide comprises an amino acid sequence comprising at least a portion having at least or about 50, 60, 70, 80, 90, 100, 150, 200, 250, 300, 350, 400, 450, 500, 550, 600, 650, 700, 750, 800, 850, 900, 950, or 1000 amino acid sequences of any sequence according to Table 2.

In some embodiments, the first recombinant fusion protein and the second recombinant fusion protein have the same transmembrane domains. In some embodiments, the first recombinant fusion protein and the second recombinant fusion protein have different transmembrane domains. In some embodiments, the transmembrane domain anchors the fusion protein to a bilayer of the enveloped particle. In some embodiments, the transmembrane domain comprises the transmembrane domain of a Vesicular Stomatitis virus glycoprotein (VSV-G). In some embodiments, the recombinant fusion protein comprises the transmembrane domain and cytosolic domain of a Vesicular Stomatitis virus glycoprotein (VSV-G). In some embodiments, the first or second recombinant fusion protein comprises a cytosolic domain comprising an amino acid sequence at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or about 99% identical to that set forth in SEQ ID No: 5. In some embodiments, the first or second recombinant fusion protein comprises the cytosolic domain comprising an amino acid sequence of SEQ ID No: 5. In some embodiments, the transmembrane domain comprises the transmembrane domain of a Dengue E protein. In some embodiments, the recombinant fusion protein comprises the transmembrane domain and cytosolic domain of a Dengue E protein. In some embodiments, the transmembrane domain comprises the transmembrane domain of influenza Hemagglutinin (HA). In some embodiments, the recombinant fusion protein comprises the transmembrane domain and cytosolic domain of influenza Hemagglutinin (HA). In some embodiments, the transmembrane domain comprises the transmembrane domain of HIV surface glycoprotein GP120 or GP41. In some embodiments, the recombinant fusion protein comprises the transmembrane domain and cytosolic domain of HIV surface glycoprotein GP120 or GP41. In some embodiments, the transmembrane domain comprises the transmembrane domain of measles virus surface glycoprotein hamagglutinin (H) protein. In some embodiments, the recombinant fusion protein comprises the transmembrane domain and cytosolic domain of measles virus surface glycoprotein hamagglutinin (H) protein. In some embodiments, the transmembrane domain comprises the transmembrane domain of influenza Neuraminidase (NA). In some embodiments, the recombinant fusion protein comprises the transmembrane domain and cytosolic domain of influenza Neuraminidase (NA). In some embodiments, the transmembrane domain comprises an amino acid sequence at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or about 99% identical to that set forth in SEQ ID NOs: 1-4, and 52-55.

In some embodiments, the transmembrane domain comprises an amino acid sequence disclosed in Table 3, or an amino acid sequence that is substantially identical to an amino acid sequence in Table 3 (e.g. about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or about 99% sequence identity). In some instances, the transmembrane domain comprises an amino acid sequence comprising at least a portion having at least or about 10, 20, 30, 40, 50, 60, 70, 80, 90, 100, 110, 120, 130 amino acid sequences of any sequence according to Table 3.

In some embodiments, when the first recombinant fusion protein is expressed on the surface of the enveloped particle the first recombinant fusion protein is expressed at a valency of about 10 copies on the surface of the enveloped particle. In some embodiments, when the first recombinant fusion protein is expressed on the surface of the enveloped particle the first recombinant fusion protein is expressed at a valency of about 10 to 15 copies on the surface of the enveloped particle. In some embodiments, when the first recombinant fusion protein is expressed on the surface of the enveloped particle the first recombinant fusion protein is expressed at a valency of at least about 25 copies on the surface of the enveloped particle. In some embodiments, when the first recombinant fusion protein is expressed on the surface of the enveloped particle the first recombinant fusion protein is expressed at a valency of at least about 50 copies on the surface of the enveloped particle. In some embodiments, when the first recombinant fusion protein is expressed on the surface of the enveloped particle the first recombinant fusion protein is expressed at a valency of at least about 100 copies on the surface of the enveloped particle. In some embodiments, when the first recombinant fusion protein is expressed on the surface of the enveloped particle the first recombinant fusion protein is expressed at a valency of at least about 200 copies on the surface of the enveloped particle. In some embodiments, when the first recombinant fusion protein is expressed on the surface of the enveloped particle the first recombinant fusion protein is expressed at a valency of at least about 400 copies on the surface of the enveloped particle. In some embodiments, when the first recombinant fusion protein is expressed on the surface of the enveloped particle the first recombinant fusion protein is expressed at a valency of at least about 600 copies on the surface of the enveloped particle. In some embodiments, when the first recombinant fusion protein is expressed on the surface of the enveloped particle the first recombinant fusion protein is expressed on the surface of the enveloped particle at a valency of at least about 1000 copies on the surface of the enveloped particle.

In some embodiments, when the second recombinant fusion protein is expressed on the surface of the enveloped particle the second recombinant fusion protein is expressed at a valency of about 10 copies on the surface of the enveloped particle. In some embodiments, when the second recombinant fusion protein is expressed on the surface of the enveloped particle the second recombinant fusion protein is expressed at a valency of about 10 to 15 copies on the surface of the enveloped particle. In some embodiments, when the second recombinant fusion protein is expressed on the surface of the enveloped particle the second recombinant fusion protein is expressed at a valency of at least about 25 copies on the surface of the enveloped particle. In some embodiments, when the second recombinant fusion protein is expressed on the surface of the enveloped particle the second recombinant fusion protein is expressed at a valency of at least about 50 copies on the surface of the enveloped particle. In some embodiments, when the second recombinant fusion protein is expressed on the surface of the enveloped particle the second recombinant fusion protein is expressed at a valency of at least about 100 copies on the surface of the enveloped particle. In some embodiments, when the second recombinant fusion protein is expressed on the surface of the enveloped particle the second recombinant fusion protein is expressed at a valency of at least about 200 copies on the surface of the enveloped particle. In some embodiments, when the second recombinant fusion protein is expressed on the surface of the enveloped particle the second recombinant fusion protein is expressed at a valency of at least about 400 copies on the surface of the enveloped particle. In some embodiments, when the second recombinant fusion protein is expressed on the surface of the enveloped particle the second recombinant fusion protein is expressed at a valency of at least about 600 copies on the surface of the enveloped particle. In some embodiments, when the second recombinant fusion protein is expressed on the surface of the enveloped particle the second recombinant fusion protein is expressed on the surface of the enveloped particle at a valency of at least about 1000 copies on the surface of the enveloped particle.

In some embodiments, the first recombinant fusion protein comprises an amino acid sequence having at least about 75% sequence identity to any of SEQ ID NOs: 72-78, 81-94, 96-100. In some embodiments, the first recombinant fusion protein comprises an amino acid sequence having at least about 76% sequence identity to any of SEQ ID NOs: 72-78, 81-94, 96-100. In some embodiments, the first recombinant fusion protein comprises an amino acid sequence having at least about 77% sequence identity to any of SEQ ID NOs: 72-78, 81-94, 96-100. In some embodiments, the first recombinant fusion protein comprises an amino acid sequence having at least about 78% sequence identity to any of SEQ ID NOs: 72-78, 81-94, 96-100. In some embodiments, the first recombinant fusion protein comprises an amino acid sequence having at least about 79% sequence identity to any of SEQ ID NOs: 72-78, 81-94, 96-100. In some embodiments, the first recombinant fusion protein comprises an amino acid sequence having at least about 80% sequence identity to any of SEQ ID NOs: 72-78, 81-94, 96-100. In some embodiments, the first recombinant fusion protein comprises an amino acid sequence having at least about 81% sequence identity to any of SEQ ID NOs: 72-78, 81-94, 96-100. In some embodiments, the first recombinant fusion protein comprises an amino acid sequence having at least about 82% sequence identity to any of SEQ ID NOs: 72-78, 81-94, 96-100. In some embodiments, the first recombinant fusion protein comprises an amino acid sequence having at least about 83% sequence identity to any of SEQ ID NOs: 72-78, 81-94, 96-100. In some embodiments, the first recombinant fusion protein comprises an amino acid sequence having at least about 84% sequence identity to any of SEQ ID NOs: 72-78, 81-94, 96-100. In some embodiments, the first recombinant fusion protein comprises an amino acid sequence having at least about 85% sequence identity to any of SEQ ID NOs: 72-78, 81-94, 96-100. In some embodiments, the first recombinant fusion protein comprises an amino acid sequence having at least about 86% sequence identity to any of SEQ ID NOs: 72-78, 81-94, 96-100. In some embodiments, the first recombinant fusion protein comprises an amino acid sequence having at least about 87% sequence identity to any of SEQ ID NOs: 72-78, 81-94, 96-100. In some embodiments, the first recombinant fusion protein comprises an amino acid sequence having at least about 88% sequence identity to any of SEQ ID NOs: 72-78, 81-94, 96-100. In some embodiments, the first recombinant fusion protein comprises an amino acid sequence having at least about 89% sequence identity to any of SEQ ID NOs: 72-78, 81-94, 96-100. In some embodiments, the first recombinant fusion protein comprises an amino acid sequence having at least about 90% sequence identity to any of SEQ ID NOs: 72-78, 81-94, 96-100. In some embodiments, the first recombinant fusion protein comprises an amino acid sequence having at least about 91% sequence identity to any of SEQ ID NOs: 72-78, 81-94, 96-100. In some embodiments, the first recombinant fusion protein comprises an amino acid sequence having at least about 92% sequence identity to any of SEQ ID NOs: 72-78, 81-94, 96-100. In some embodiments, the first recombinant fusion protein comprises an amino acid sequence having at least about 93% sequence identity to any of SEQ ID NOs: 72-78, 81-94, 96-100. In some embodiments, the first recombinant fusion protein comprises an amino acid sequence having at least about 94% sequence identity to any of SEQ ID NOs: 72-78, 81-94, 96-100. In some embodiments, the first recombinant fusion protein comprises an amino acid sequence having at least about 95% sequence identity to any of SEQ ID NOs: 72-78, 81-94, 96-100. In some embodiments, the first recombinant fusion protein comprises an amino acid sequence having at least about 96% sequence identity to any of SEQ ID NOs: 72-78, 81-94, 96-100. In some embodiments, the first recombinant fusion protein comprises an amino acid sequence having at least about 97% sequence identity to any of SEQ ID NOs: 72-78, 81-94, 96-100. In some embodiments, the first recombinant fusion protein comprises an amino acid sequence having at least about 98% sequence identity to any of SEQ ID NOs: 72-78, 81-94, 96-100. In some embodiments, the first recombinant fusion protein comprises an amino acid sequence having at least about 99% sequence identity to any of SEQ ID NOs: 72-78, 81-94, 96-100. In some embodiments, the first recombinant fusion protein comprises an amino acid sequence of any of SEQ ID NOs: 72-78, 81-94, 96-100.

In some embodiments, the second recombinant fusion protein comprises an amino acid sequence having at least about 75% sequence identity to any of SEQ ID NOs: 71-100. In some embodiments, the second recombinant fusion protein comprises an amino acid sequence having at least about 76% sequence identity to any of SEQ ID NOs: 71-100. In some embodiments, the second recombinant fusion protein comprises an amino acid sequence having at least about 77% sequence identity to any of SEQ ID NOs: 71-100. In some embodiments, the second recombinant fusion protein comprises an amino acid sequence having at least about 78% sequence identity to any of SEQ ID NOs: 71-100. In some embodiments, the second recombinant fusion protein comprises an amino acid sequence having at least about 79% sequence identity to any of SEQ ID NOs: 71-100. In some embodiments, the second recombinant fusion protein comprises an amino acid sequence having at least about 80% sequence identity to any of SEQ ID NOs: 71-100. In some embodiments, the second recombinant fusion protein comprises an amino acid sequence having at least about 81% sequence identity to any of SEQ ID NOs: 71-100. In some embodiments, the second recombinant fusion protein comprises an amino acid sequence having at least about 82% sequence identity to any of SEQ ID NOs: 71-100. In some embodiments, the second recombinant fusion protein comprises an amino acid sequence having at least about 83% sequence identity to any of SEQ ID NOs: 71-100. In some embodiments, the second recombinant fusion protein comprises an amino acid sequence having at least about 84% sequence identity to any of SEQ ID NOs: 71-100. In some embodiments, the second recombinant fusion protein comprises an amino acid sequence having at least about 85% sequence identity to any of SEQ ID NOs: 71-100. In some embodiments, the second recombinant fusion protein comprises an amino acid sequence having at least about 86% sequence identity to any of SEQ ID NOs: 71-100. In some embodiments, the second recombinant fusion protein comprises an amino acid sequence having at least about 87% sequence identity to any of SEQ ID NOs: 71-100. In some embodiments, the second recombinant fusion protein comprises an amino acid sequence having at least about 88% sequence identity to any of SEQ ID NOs: 71-100. In some embodiments, the second recombinant fusion protein comprises an amino acid sequence having at least about 89% sequence identity to any of SEQ ID NOs: 71-100. In some embodiments, the second recombinant fusion protein comprises an amino acid sequence having at least about 90% sequence identity to any of SEQ ID NOs: 71-100. In some embodiments, the second recombinant fusion protein comprises an amino acid sequence having at least about 91% sequence identity to any of SEQ ID NOs: 71-100. In some embodiments, the second recombinant fusion protein comprises an amino acid sequence having at least about 92% sequence identity to any of SEQ ID NOs: 71-100. In some embodiments, the second recombinant fusion protein comprises an amino acid sequence having at least about 93% sequence identity to any of SEQ ID NOs: 71-100. In some embodiments, the second recombinant fusion protein comprises an amino acid sequence having at least about 94% sequence identity to any of SEQ ID NOs: 71-100. In some embodiments, the second recombinant fusion protein comprises an amino acid sequence having at least about 95% sequence identity to any of SEQ ID NOs: 71-100. In some embodiments, the second recombinant fusion protein comprises an amino acid sequence having at least about 96% sequence identity to any of SEQ ID NOs: 71-100. In some embodiments, the second recombinant fusion protein comprises an amino acid sequence having at least about 97% sequence identity to any of SEQ ID NOs: 71-100. In some embodiments, the second recombinant fusion protein comprises an amino acid sequence having at least about 98% sequence identity to any of SEQ ID NOs: 71-100. In some embodiments, the second recombinant fusion protein comprises an amino acid sequence having at least about 99% sequence identity to any of SEQ ID NOs: 71-100. In some embodiments, the second recombinant fusion protein comprises an amino acid sequence of any of SEQ ID NOs: 71-100.

### Compositions for Generation of Enveloped Particles

Disclosed herein are compositions comprising a nucleic acid sequence that encodes any of the recombinant fusion proteins described herein.

Disclosed herein are compositions comprising a nucleic acid sequence that encodes any of the first recombinant fusion proteins described herein and any of the second recombinant fusion protein described herein.

In some embodiments, the composition further comprises a second nucleic acid sequence that encodes one or more packaging viral proteins. In some embodiments, the one or more packaging viral proteins is a lentiviral protein, a retroviral protein, an adenoviral protein, or combinations thereof. In some embodiments, the one or more packaging viral proteins comprises gag, pol, pre, tat, rev, or combinations thereof.

In some embodiments, the compositions further comprising a third nucleic acid sequence that encodes a reporter, a therapeutic molecule, or combinations thereof. In some embodiments, the reporter is a fluorescent protein or luciferase. In some embodiments, the fluorescent protein is green fluorescent protein. In some embodiments, the therapeutic molecule is an immune modulating protein, a cellular signal modulating molecule, a proliferation modulating molecule, a cell death modulating molecule, or combinations thereof.

Exemplary reporters include, but are not limited to, acetohydroxyacid synthase (AHAS), alkaline phosphatase (AP), beta galactosidase (LacZ), beta glucuronidase (GUS), chloramphenicol acetyltransferase (CAT), green fluorescent protein (GFP), red fluorescent protein (RFP), yellow fluorescent protein (YFP), cyan fluorescent protein (CFP), cerulean fluorescent protein, citrine fluorescent protein, orange fluorescent protein, cherry fluorescent protein, turquoise fluorescent protein, blue fluorescent protein, horseradish peroxidase (HRP), luciferase (Luc), nopaline synthase (NOS), octopine synthase (OCS), luciferase, and derivatives thereof. Methods to determine modulation of a reporter gene are well known in the art, and include, but are not limited to, fluorometric methods (e.g. fluorescence spectroscopy, Fluorescence Activated Cell Sorting (FACS), fluorescence microscopy), and antibiotic resistance determination. In some embodiments, the reporter is a fluorescent protein. In some embodiments, the fluorescent protein is green fluorescent protein. In some embodiments, the reporter protein emits green fluorescence, yellow fluorescence, or red fluorescence. In some embodiments, the reporter is an enzyme. In some embodiments, the enzyme is β-galactosidase, alkaline phosphatase, β-lactamase, or luciferase.

In some embodiments, the nucleic acid sequence that encodes the recombinant fusion protein and the second nucleic acid sequence and the third nucleic acid sequence are within a same vector.

In some embodiments, the nucleic acid sequence that encodes the first recombinant fusion protein and the second recombinant fusion protein and the second nucleic acid sequence and the third nucleic acid sequence are within a same vector. In some embodiments, the nucleic acid sequence that encodes the recombinant fusion protein and the second nucleic acid sequence and the third nucleic acid sequence are within different vectors.

In some embodiments, the nucleic acid sequence that encodes the first recombinant fusion protein and the second recombinant fusion protein and the second nucleic acid sequence and the third nucleic acid sequence are within different vectors. In some embodiments, the vector is a lentivirus vector, an adenovirus vector, or an adeno-associated virus vector.

Various vectors, in some embodiments, are used herein. In some embodiments, the vector is a eukaryotic or prokaryotic vector. In some embodiments, the vector is a viral vector. In some embodiments, the vector is a lentivirus vector, an adenovirus vector, or an adeno-associated virus vector. Exemplary vectors include, without limitation, mammalian expression vectors: pSF-CMV-NEO-NH2-PPT-3XFLAG, pSF-CMV-NEO—COOH-3XFLAG, pSF-CMV—PURO-NH2-GST-TEV, pSF-OXB20-COOH-TEV-FLAG (R)-6His, pCEP4 pDEST27, pSF-CMV-Ub-KrYFP, pSF-CMV-FMDV-daGFP, pEFla-mCherry-NI Vector, pEFla-tdTomato Vector, pSF-CMV-FMDV-Hygro, pSF-CMV-PGK-Puro, pMCP-tag (m), and pSF-CMV—PURO-NH2-CMYC: bacterial expression vectors: pSF-OXB20-BetaGal.pSF-OXB20-Fluc, pSF-OXB20, and pSF-Tac: plant expression vectors: pRI 101-AN DNA and pCambia2301; and yeast expression vectors: pTYB21 and pKLAC2, and insect vectors: pAc5.1/V5-His A and pDEST8.

### Methods for Targeting an Oligomerized Cell Surface Protein

Disclosed herein are methods of agonizing or antagonizing a cell that has an oligomerized cell surface protein comprising contacting the cell with an enveloped particle comprising a first recombinant fusion protein that is displayed in an oligomeric format on the surface of the enveloped particle wherein the first recombinant fusion protein comprises a transmembrane domain, a display polypeptide, and an oligomerization domain.

In some embodiments, the enveloped particle further comprises a second recombinant fusion protein that is displayed in a monomeric format on the surface of the enveloped particle wherein the second recombinant fusion protein comprises a transmembrane domain and a display polypeptide.

In some embodiments, the oligomerization domain is a dimerization domain. In some embodiments, the dimerization domain comprises a leucine zipper dimerization domain. In some embodiments, the oligomerization domain is a trimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the oligomerization domain is a tetramerization domain. In some embodiments, the tetramerization domain comprises an influenza neuraminidase stem domain.

In some embodiments, the oligomerization domain comprises an amino acid sequence disclosed in Table 1, or an amino acid sequence that is substantially identical to an amino acid sequence in Table 1 (e.g. 80%, 85%, 90%, 95%, 96%, 97%, 98%, 99% sequence identity). In some instances, the oligomerization domain comprises an amino acid sequence comprising at least a portion having at least or about 10, 20, 30, 40, 50, 60, 70, 80, 90, 100, 110, 120, 130 amino acid sequences of any sequence according to Table 1.

In some embodiments, when the first recombinant fusion protein is expressed on the surface of the enveloped particle, the oligomerization domain is outside of the enveloped particle. In some embodiments, when the first recombinant fusion protein is expressed on the surface of the enveloped particle, the oligomerization domain is outside of the enveloped particle and adjacent to a signal peptide. In some embodiments, when the first recombinant fusion protein is expressed on the surface of the enveloped particle, the oligomerization domain is inside of the enveloped particle.

In some embodiments, when the first recombinant fusion protein is expressed on the surface of the enveloped particle, the oligomerization domain is inside of the enveloped particle and adjacent to the transmembrane domain.

In some embodiments, the first recombinant fusion protein comprises a signal peptide. In some embodiments, domains of the first recombinant fusion protein are arranged from the N-terminus to the C-terminus in the following orders: (a) signal peptide, display polypeptide, oligomerization domain, transmembrane domain, and cytosolic domain: (b) signal peptide, display polypeptide, transmembrane domain, oligomerization domain, and cytosolic domain: or (c) signal peptide, oligomerization domain, display peptide, transmembrane domain, and cytosolic domain.

In some embodiments, the enveloped particle comprises an enveloped viral-like particle, an enveloped virus, an extracellular vesicle. In some embodiments, the extracellular vesicle comprises an exosome or an ectosome.

In some embodiments, the first recombinant fusion protein and the second recombinant fusion protein have the same display polypeptide. In some embodiments, the first recombinant fusion protein and the second recombinant fusion protein have different display polypeptides.

In some embodiments, the display polypeptide targets the cell surface protein that is oligomerized on a target cell for agonistic or antagonistic function. In some embodiments, the cell surface protein is dimerized and the oligomerization domain is a dimerization domain. In some embodiments, the cell surface protein is trimerized and the oligomerization domain is a trimerization domain. In some embodiments, the cell surface protein is tetramerized and the oligomerization domain is a tetramerization domain.

In some embodiments, the display polypeptide comprises a single chain variable fragment (scFv), a type I transmembrane polypeptide, a type II transmembrane polypeptide, a type III transmembrane polypeptide, a GPI-anchored polypeptide, a secreted polypeptide, or a multi-pass transmembrane polypeptide. In some embodiments, the scFv comprises an anti-BCMA scFv polypeptide, an anti-NTD scFv polypeptide, an anti-RBD scFv polypeptide, or an anti-CD19 scFv polypeptide. In some embodiments, the type I transmembrane polypeptide comprises ACE2, ICAM-1, LILRA5, LILRB3, or LILRB4. In some embodiments, the type II transmembrane polypeptide comprises DPP4, or BAFF. In some embodiments, the type III transmembrane polypeptide comprises BCMA, TACI, or BAFF Receptor. In some embodiments, the GPI-anchored polypeptide comprises CD24, CD48, CD59 or CD55. In some embodiments, the secreted polypeptide comprises IL-4, or CXCL-12. In some embodiments, the multi-pass transmembrane polypeptide comprises NTCP, or FCεRI beta. In some embodiments, the display polypeptide comprises an antibody, a receptor, a cytokine, an immune checkpoint protein, a chemokine, an adhesion factor, or a mitogen. In some embodiments, the antibody comprises a non-neutralizing antibody, a neutralizing antibody, or an antibody of multiple formats. In some embodiments, the receptor comprises ACE2, or DPP4. In some embodiments, the cytokine comprises IL-4, or IL-6. In some embodiments, the immune checkpoint protein comprises CTLA-4, or PD-1. In some embodiments, the chemokine comprises CXCL-12. In some embodiments, the adhesion factor comprises ICAM-1, or LFA-1. In some embodiments, the mitogen comprises Wnt. In some embodiments, the display polypeptide comprises an anti-BCMA scFv polypeptide, an anti-CD19 scFv polypeptide, an anti-NTD scFv polypeptide, an anti-RBD scFv polypeptide, ACE2, ICAM-1, LILRA5, LILRB3, LILRB4, DPP4, BAFF, BCMA, TACI, BAFF Receptor, CD24, CD48, CD59, CD55, IL-4, CXCL-12, NTCP, FCεRI beta, a non-neutralizing antibody, a neutralizing antibody, an antibody of multiple formats, IL-6, CTLA-4, PD-1, LFA-1, Wnt, CD80, CD86, OX40L, CD3, or 4-1BBL.

In some embodiments, the display polypeptide comprises an amino acid sequence according to any amino acid sequence of Table 2, or a sequence that is substantially similar to a sequence of Table 2 (e.g. 90%, 91%, 92%, 93%, 94%, 95%, 96%, 97%, 98%, 99% sequence identity). In some instances, the display polypeptide comprises an amino acid sequence comprising at least a portion having at least or about 50, 60, 70, 80, 90, 100, 150, 200, 250, 300, 350, 400, 450, 500, 550, 600, 650, 700, 750, 800, 850, 900, 950, or 1000 amino acid sequences of any sequence according to Table 2.

In some embodiments, the first recombinant fusion protein and the second recombinant fusion protein have the same transmembrane domains. In some embodiments, the first recombinant fusion protein and the second recombinant fusion protein have different transmembrane domains. In some embodiments, the transmembrane domain anchors the fusion protein to a bilayer of the enveloped particle.

In some embodiments, the transmembrane domain comprises the transmembrane domain of a Vesicular Stomatitis virus glycoprotein (VSV-G). In some embodiments, the recombinant fusion protein comprises the transmembrane domain and cytosolic domain of a Vesicular Stomatitis virus glycoprotein (VSV-G). In some embodiments, the transmembrane domain comprises the transmembrane domain of a Dengue E protein. In some embodiments, the recombinant fusion protein comprises the transmembrane domain and cytosolic domain of a Dengue E protein. In some embodiments, the transmembrane domain comprises the transmembrane domain of influenza Hemagglutinin (HA). In some embodiments, the recombinant fusion protein comprises the transmembrane domain and cytosolic domain of influenza Hemagglutinin (HA). In some embodiments, the transmembrane domain comprises the transmembrane domain of HIV surface glycoprotein GP120 or GP41. In some embodiments, the recombinant fusion protein comprises the transmembrane domain and cytosolic domain of HIV surface glycoprotein GP120 or GP41. In some embodiments, the transmembrane domain comprises the transmembrane domain of measles virus surface glycoprotein hamagglutinin (H) protein. In some embodiments, the recombinant fusion protein comprises the transmembrane domain and cytosolic domain of measles virus surface glycoprotein hamagglutinin (H) protein. In some embodiments, the transmembrane domain comprises the transmembrane domain of influenza Neuraminidase (NA). In some embodiments, the recombinant fusion protein comprises the transmembrane domain and cytosolic domain of influenza Neuraminidase (NA).

In some embodiments, the transmembrane domain comprises an amino acid sequence disclosed in Table 3, or an amino acid sequence that is substantially identical to an amino acid sequence in Table 3 (e.g. 80%, 85%, 90%, 95%, 96%, 97%, 98%, 99% sequence identity). In some instances, the transmembrane domain comprises an amino acid sequence comprising at least a portion having at least or about 10, 20, 30, 40, 50, 60, 70, 80, 90, 100, 110, 120, 130 amino acid sequences of any sequence according to Table 3.

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising.” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

A list of genes or biomarkers are disclosed herein: B-cell maturation antigen (BCMA), cluster of differentiation 19 (CD19), leukocyte immunoglobulin-like receptor subfamily A member 5 (LILRA5), leukocyte immunoglobulin-like receptor subfamily B member 3 (LILRB3), B-cell activating factor (BAFF), transmembrane activator and CAML interactor (TACI), B-cell activating factor receptor (BAFFR), CD59 glycoprotein preproprotein (CD59), complement decay-accelerating factor (CD55), interleukin 4 (IL-4), high-affinity IgE receptor beta (FCεRI beta), interleukin 6 (IL-6), C-X-C motif chemokine ligand 12 (CXCL-12), intercellular adhesion molecule 1 (ICAM-1), lymphocyte function-associated antigen 1 (LFA-1), dipeptidyl-peptidase 4 (DPP-4), angiotensin-converting Enzyme 2 (ACE2), leukocyte Immunoglobulin like receptor B4 (LILRB4), cluster of differentiation 86 (CD86), OX40 ligand (OX40L), 4-1BB ligand (4-1BBL), cluster of differentiation 3 (CD3), cluster of differentiation 24 (CD24), cluster of differentiation 48 (CD48), programmed cell death protein 1 (PD-1), sodium/bile acid cotransporter (NTCP), cytotoxic T-lymphocyte-associated protein 4 (CTLA-4), and cluster of differentiation 80 (CD80).

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1. Design of Enveloped Particle Display Vector

To mimic multivalent protein expression on cell surface, two different types of enveloped particle display vectors for efficient protein display on viral-like particle (VLP) and extracellular vesicles (EV) are designed.

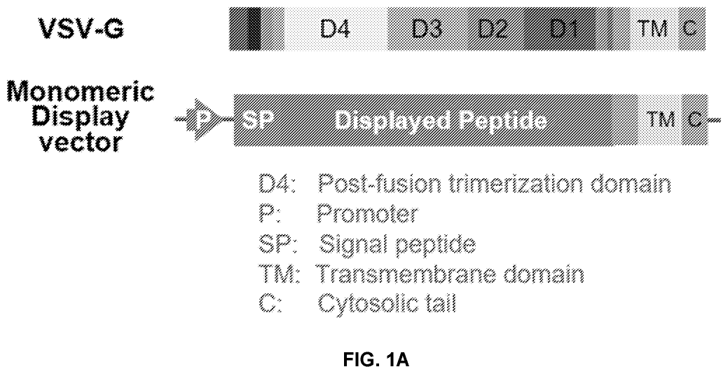

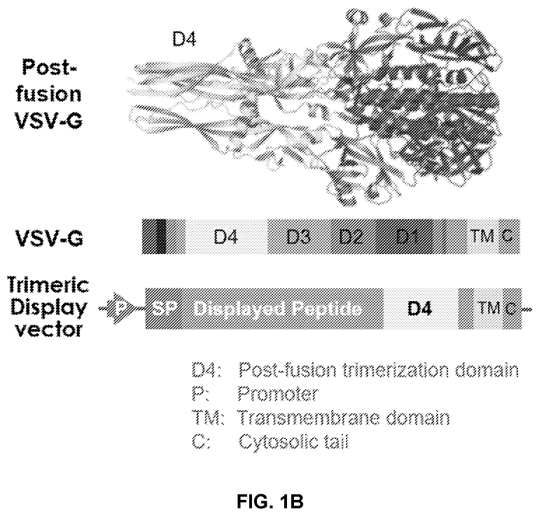

A monomeric display vector expressing a fusion protein consisting of a protein linked to the VSVG transmembrane and intracellular domains is designed as shown in FIG. 1A to display hundreds of copies of monomeric proteins on the surface of VLPs and EVs. Aside from the use of monomeric formats that are suited to form high avidity interactions with similarly displayed patterned proteins on cell surfaces, enveloped particles are made to match oligomeric display formats to enhance avidity at the level of individual oligomeric binding partners. To this end, a trimeric display vector expressing a fusion protein consisting of a protein linked to the D4 post-fusion trimerization domain of VSVG, followed by the transmembrane and intracellular domains of VSVG is designed as shown in FIG. 1B. The vector is used to display hundreds of copies of trimeric proteins on the surface of VLPs and EVs and are well suited to form high avidity interactions with similarly oligomeric proteins on the surface of cells.

### Example 2. Generation of Monomeric Enveloped Particles

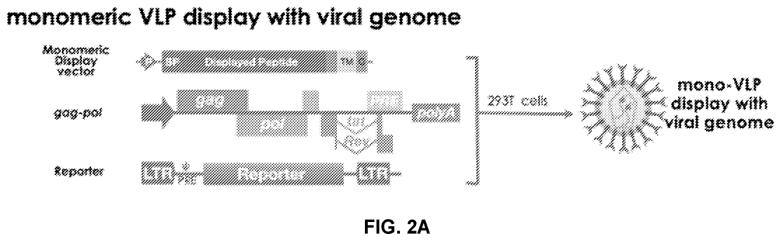

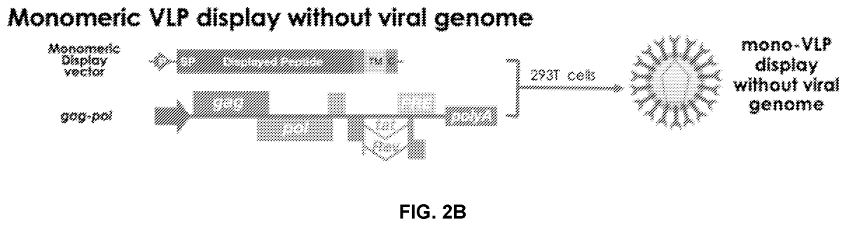

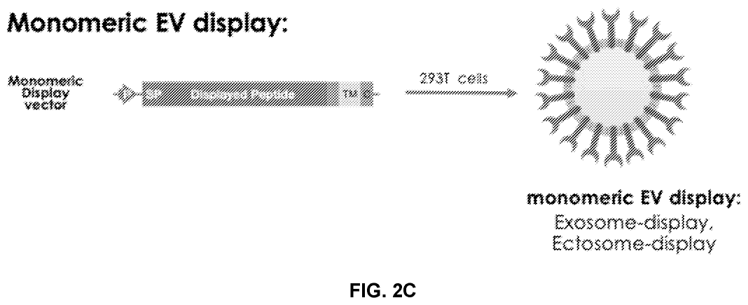

Multivalent proteins are displayed as monomers on the surface of a viral-like particle (VLP) and an extracellular vesicle using a monomeric display vector. The monomeric VLP-based enveloped particle is produced with viral RNA genomes in which the monomeric peptide display construct with a lentiviral packaging construct expresses essential packaging components including Gag-Pol and Rev proteins and a viral genome transfer encoding a GFP/luciferase reporter as shown in FIG. 2A. The monomeric VLP-based enveloped particle without RNA genome is produced by co-transfecting displaying vector with only a lentiviral packaging construct but not the viral genome transfer vector as shown in FIG. 2B. The monomeric EV-based enveloped particle which includes exosomes and ectosomes is produced by transfecting only monomeric peptide displaying vector in 293T cells as shown in FIG. 2C.

Codon-optimized display peptide sequences are synthesized (Twist) and cloned into a display construct to create fusion peptides consisting of the extracellular domain of display peptide and a display anchoring protein. To generate enveloped particles displaying monomeric peptides the extracellular domains were fused to a synthetic VSV-G sequence encoding the transmembrane and cytoplasmic tail domains.

Enveloped particles based on VLPs or EVs can be produced from transfected 293T cells. To produce lentiviral-VLP based MVPs with viral genomes, surface display construct, lentiviral packaging vector (i.e. psPAX2), and lentiviral genome transfer vector are co-transfected into 293T cells. To produce lentiviral-VLP based MVPs without viral genomes, display peptides displaying construct and lentiviral packaging vector (i.e. psPAX2) are co-transfected into 293T cells. Finally, to produce EV-based MVPs, only display peptide displaying construct is transfected into 293T cells.

Specifically, in preparation for transfection, 7.5×10⁶ HEK293T cells (ATCC CRL-3216) are seeded overnight in 10-cm dishes containing DMEM media with glucose, L-glutamine and sodium pyruvate (Corning) supplemented with 10% fetal bovine serum (Sigma) and 1% Penicillin Streptomycin (Life Technologies), referred to as “293T Growth Media.” Cells should reach ˜90% confluence the next day at time of transfection. The following day, transfection DNA mixture along with polyethylenimine (PEI) in OPTI-MEM reduced serum medium (gibco) are prepared. Transfection mixture is incubated at room temperature for 15 minutes before being added to cells, which are then incubated at 37° C. in 5% CO2. 6 hours post-transfection, 293T Growth Media is changed to 293T Growth Media supplemented with 0.1% sodium butyrate (referred to as “Transfection Media”) before being returned to incubation. After incubating for 24 hours at 37° C. with 5% CO2 in Transfection Media, supernatant containing pseudovirus is collected, centrifuged at 1680 rpm for 5 minutes to remove cellular debris and mixed with 1× polyethylene glycol 8000 solution (PEG, Hampton Research), before being stored at 4° C. for 24 hours to allow fractionation. Cells are replenished with fresh Transfection Media, and a second pseudovirus supernatant collection are performed at 48 hours. Supernatant collections are then pooled, PEG precipitated and purified by size exclusion chromatography using Sephacryl S-300 High Resolution Beads (Sigma Aldrich).

### Example 3. Generation of Trimeric Enveloped Particles

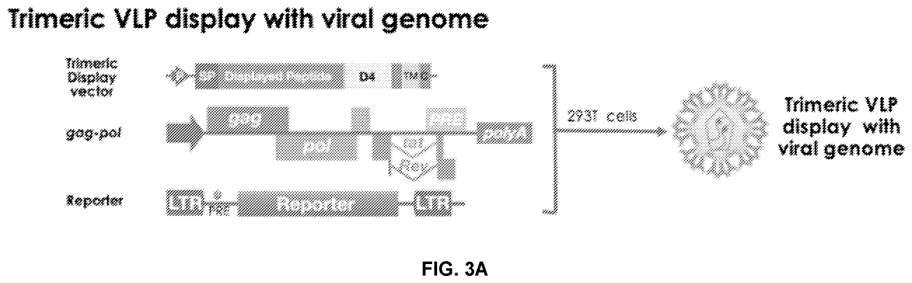

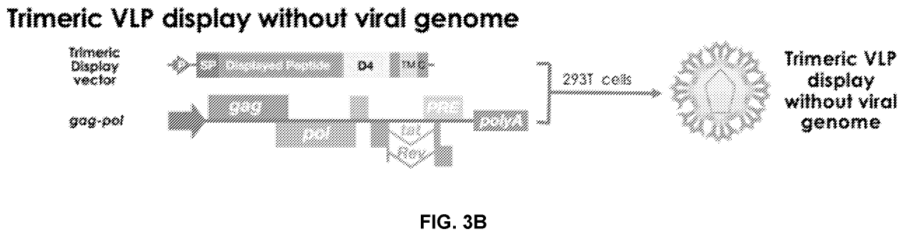

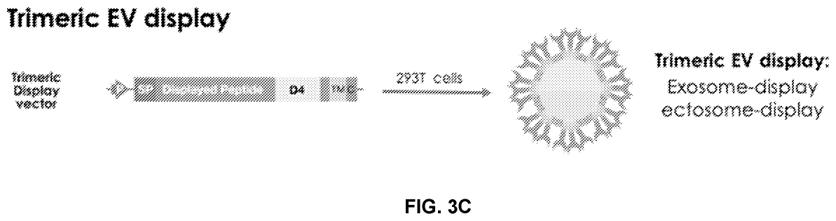

Multivalent proteins are displayed as trimers on the surface of a viral-like particle (VLP) and an extracellular vesicle using a trimeric display vector. The trimeric VLP-based enveloped particle is produced with viral RNA genomes in which the trimeric peptide display construct with a lentiviral packaging construct expresses essential packaging components including Gag-Pol and Rev proteins and a viral genome transfer encoding a GFP/luciferase reported as shown in FIG. 3A. The trimeric VLP-based enveloped particle without RNA genome is produced by co-transfecting displaying vector together with only a lentiviral packaging construct but not the viral genome transfer vector as shown in FIG. 3B. The trimeric EV-based enveloped particle which includes exosomes and ectosomes is produced by transfecting only the trimeric peptide displaying vector in 293T cells as shown in FIG. 3C.

Codon-optimized display peptide sequences are synthesized (Twist) and cloned into a display construct to create fusion peptides consisting of the extracellular domain of display peptide and a display anchoring protein. To generate enveloped particles displaying trimeric peptides the extracellular domains were fused to a synthetic VSV-G sequence encoding the D4 post-fusion trimerization domain and the transmembrane and cytoplasmic tail domains. Unless described explicitly otherwise, the D4 trimerization domain used in the trimeric display vector comprises the amino acid sequence of SEQ ID No. 7, which is designated as D4 Variation 2 (D4V2).

Methods for production of VLP-based or EV-based enveloped particles are according to methods as described in Example 2.

### Example 4. Generation of Mixed Monomeric and Trimeric Enveloped Particles

Enveloped particles displaying mixed monomeric and trimeric proteins are generated by co-transfecting monomeric and trimeric peptide display constructs. Such design is used to increase the density of the displayed peptide or to create combinatorial of distinct displayed peptides. Mixed monomeric and trimeric enveloped particles are built with VLPs and EVs by co-transfecting monomeric and trimeric display vectors.

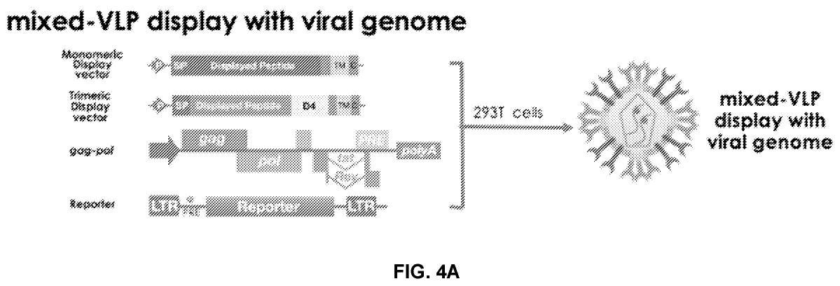

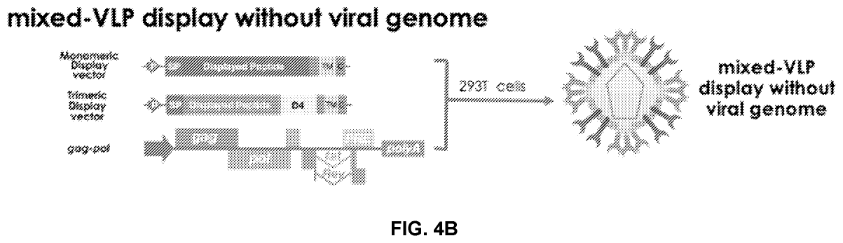

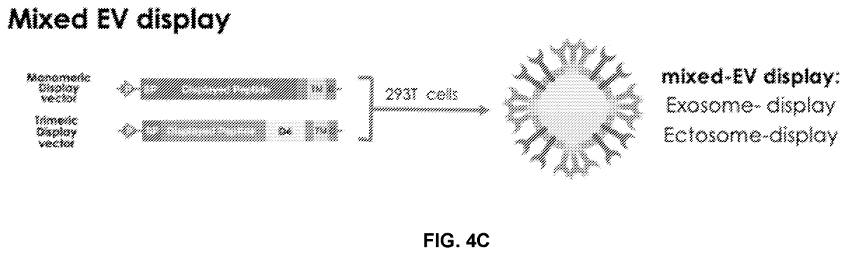

To produce mixed VLPs with viral RNA genomes, the mixed monomeric and trimeric display peptides fusion constructs are co-transfected with a lentiviral packaging construct expressing essential packaging components, such as Gag-Pol and Rec proteins, and viral genome transfer vector encoding a GFP/luciferase reported as shown in FIG. 4A. The mixed VLP-based enveloped particle without RNA genome are produced by co-transfecting the mixed monomeric and trimeric display vector together with only a lentiviral packaging construct but not the viral genome transfer vector as shown in FIG. 4B. The mixed EV-based enveloped particle which includes mixed exosome-enveloped particle and ectosome-enveloped particle is produced by transfecting the mixed monomeric and trimeric display peptide constructs into 293T cells as shown in FIG. 4C.

Methods for production of VLP-based or EV-based enveloped particles are according to methods as described in Example 2.

### Example 5. Protein Display Configurations on Enveloped Particles

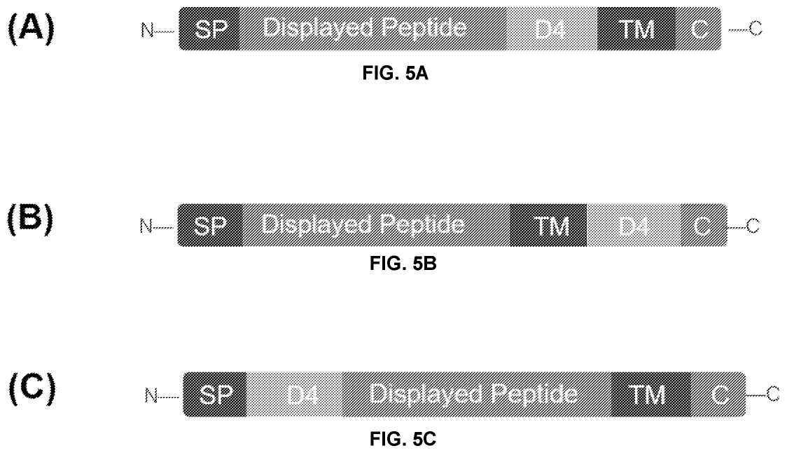

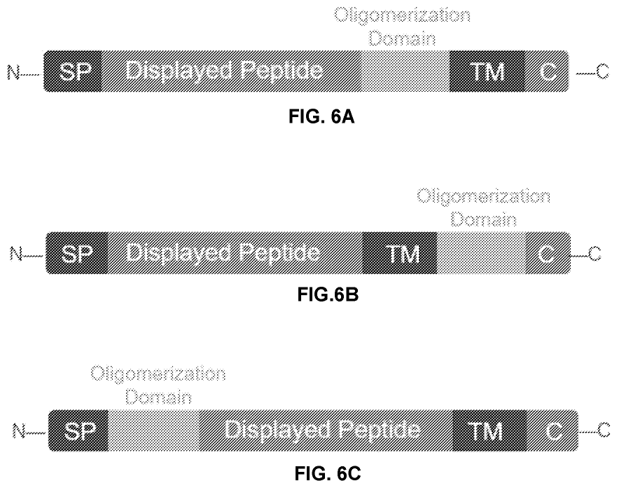

Enveloped particles are genetically programmed to display peptides of interest in various configurations by modifying the display vector as shown in FIGS. 5A-5C and FIGS. 6A-C. The VSVG D4 trimerization domain can be placed at various positions of the fusion peptide: (1) extracellular and juxtaposed to the transmembrane domain: (2) intracellular and juxtaposed to the transmembrane domain: (3) extracellular and after the signal peptide (FIG. 5A-5C). Furthermore, various oligomerization domains may be used for distinct surface display patterns that are suitable for the function of displayed molecules (FIG. 6A-6C). In addition to the VSVG D4 trimerization domain, the Dengue E protein fusion domain or a foldon domain are used to create trimeric display patterns on the surface of VLPs and EVs. Leucine zipper domains and the influenza neuraminidase stem domain are used to create dimeric and tetrameric display patterns on the surface of VLPs and EVs, respectively. Exemplary oligomerization domains and valence are summarized in Table 4. With these display configurations, it is possible to program combinatorial MVPs with mixed monomeric, dimeric, trimeric, and tetrameric display patterns optimized to their function in target cell regulation or virus neutralization.

TABLE 4
Exemplary oligomerization domains and valence

| Oligomerization Domain | Valence |
|---|---|
| VSV-G protein D4 | Trimer |
| Dengue E protein fusion protein | Trimer |
| Foldon | Trimer |
| Leucine Zipper | Dimer |
| Influenza Neuraminidase stem | Tetramer |

### Example 6. Characterization of Proteins Displayed on Enveloped Particles

The concentration of VLP- or EV-based enveloped particles are measured by P24 ELISA or tunable resistive pulse sensing (TRPS, qNano), respectively. Then, copies of displayed peptides on enveloped particles are determined by quantitative Western-blot analyses. Finally, the oligomerization patterns of displayed peptides on the enveloped particles were discerned by non-reducing PAGE analyses. Enveloped particles are expected to display at least 10 copies of protein molecules on surface of VLPs and EVs with monomeric or trimeric configurations.

### Lentiviral Particle Quantification by p24 ELISA and Tunable Resistive Pulse Sensing

P24 concentrations in pseudovirus samples of pseudotyped coronaviruses, influenza viruses and antibody-based antivirus particles are determined using an HIV p24 SimpleStep ELISA kit (Abcam) per the manufacturer's protocol. Concentrations of lentiviral pseudovirus particles are extrapolated from the assumption that each lentiviral particle contains approximately 2000 molecules of p24, or 1.25×10⁴ pseudovirus particles per picogram of p24 protein.

Pseudovirus concentrations determined via p24 ELISA are corroborated by tunable resistive pulse sensing (TRPS, qNano, IZON). Purified pseudovirus collections are diluted in 0.2 μm filtered PBS with 0.03% Tween-20 (Thermo Fisher Scientific) prior to qNano analysis. Concentration and size distributions of pseudotyped particles are then determined using an NP200 nanopore at a 45.5 mm stretch, and applied voltages between 0.5 and 0.7V are used to achieve a stable current of 130 nA through the nanopore. Measurements for each pseudovirus sample are taken at pressures of 3, 5 and 8 mbar, and considered valid if at least 500 events were recorded, particle rate was linear and root mean squared signal noise was maintained below 10 pA. Pseudovirus concentrations are then determined by comparison to a standardized multi-pressure calibration using CPC200 (mode diameter: 200 nm) (IZON) carboxylated polystyrene beads diluted 1:200 in 0.2 μM filtered PBS from their original concentration of 7.3×10¹¹ particles per/mL. Measurements are analyzed using IZON Control Suite 3.4 software to determine original sample concentrations.

### Quantify Lentiviral VLP-Based MVPs

P24 concentrations of the MVP samples are determined by using the Abcam HIV P24 SimpleStep ELISA kit following manufacturer's instruction. The concentrations of lentiviral pseudovirion particles are derived based on the assumption that each lentiviral particle contains about ˜2000 molecules of P24 or 1.25×10⁴ viral particles/picogram of P24 protein.

### Quantify EV-Based MVPs

The sizes and concentrations of extracellular vesicle-based MVPs are determined by tunable resistive pulse sensing (TRPS, qNano, IZON). Purified pseudovirus collections are diluted in 0.2 μm filtered PBS with 0.03% Tween-20 (Thermo Fisher Scientific) prior to qNano analysis. Concentration and size distributions of MVP-ICs are then determined using an NP200 nanopore at a 45.5 mm stretch, and applied voltages between 0.5 and 0.7V were used to achieve a stable current of 130 nA through the nanopore. Measurements for each pseudovirus sample are taken at pressures of 3, 5 and 8 mbar, and considered valid if at least 500 events were recorded, particle rate was linear and root mean squared signal noise was maintained below 10 pA. MVPs concentrations are then determined by comparison to a standardized multi-pressure calibration using CPC200 (mode diameter: 200 nm) (IZON) carboxylated polystyrene beads diluted 1:200 in 0.2 μM filtered PBS from their original concentration of 7.3×10¹¹ particles per/mL. Measurements are analyzed using IZON Control Suite 3.4 software to determine original sample concentrations.

### Western Blot Analysis of MVPs

Expression of fusion proteins on MVPs are confirmed via western blot analysis of purified particles. Samples of purified MVPs are lysed at 4° C. for 10 minutes with cell lysis buffer (Cell Signaling) before being mixed with NuPage LDS sample buffer (Thermo Fisher Scientific) and boiled at 95° C. for 5 minutes. Differences in oligomerization are determined by running samples in reducing and non-reducing conditions. Under reducing conditions, 5% 2-Mercaptoethanol (Thermo Fisher Scientific) are added to samples to dissociate oligomerized MVP-ICs. Protein samples are then separated on NuPAGE 4-12% Bis-Tris gels (Thermo Fisher Scientific) and transferred onto a polyvinylidene fluoride (PVDF) membrane (Life Technologies). PVDF membranes are blocked with TRIS-buffered saline with Tween-20 (TBST) and 5% skim milk (Research Products International) for 1 hour, prior to overnight incubation with primary antibody diluted in 5% milk. For display fusion constructs expressing VSVG-tag, an anti-VSV-G epitope tag rabbit polyclonal antibody (BioLegend, Poly29039) are used at a 1:2000 dilution. The following day, the PVDF membrane are washed 3 times with 1×TBST and stained with a goat-anti-rabbit secondary antibody (IRDye 680) at a 1:5000 dilution for 60 minutes in 5% milk. Post-secondary antibody staining, the PVDF membrane are again washed 3 times with TBST before imaging on a Licor Odyssey scanner.

Alternatively, western blot analyses are performed using an automated Simple Western size-based protein assay (Protein Simple) following the manufacturer's protocols. Unless otherwise mentioned, all reagents used here are from Protein Simple. Concentrated samples are lysed as described above, before being diluted 1:10 in 0.1× sample buffer for loading on capillaries. Displayed fusion protein expression levels are identified using the same primary rabbit polyclonal antibody at a 1:400 dilution and an HRP conjugated anti-rabbit secondary antibody (Protein Simple). Chemiluminescence signal analysis and absolute quantitation are performed using Compass software (Protein Simple).

### Quantitative Western Blot Analyses

Quantitative western blot analyses are performed to determine the copies of fusion protein displayed per particle. P24 ELISA or TRPS (qNano) assays are used to determine the MVP sample concentrations. Purified MVP samples are processed and analyzed via western blot under reducing conditions as described above. A reference decoy-MVP with a known display copy number are used to generate a standard curve, from which copy numbers of displayed protein on respective particles are determined.

### Surface Displaying Constructs.

Codon-optimized immune checkpoint sequences were synthesized (Twist) and cloned into a display construct to create fusion peptides consisting of the extracellular domain of an immune checkpoint and a display anchoring protein. To generate MVPs displaying monomeric immune checkpoints, the extracellular domains of immune checkpoints were fused to a synthetic VSV-G sequence encoding the transmembrane and cytoplasmic tail domains. To generate MVPs displaying oligomerized immune checkpoints, the extracellular domains of immune checkpoints were fused to a synthetic VSV-G sequence encoding the desired oligomerization domain and the transmembrane and cytoplasmic tail domains.

### IC₅₀ Pseudovirus Neutralization Assay.

H1573/ACE2 target cells were used in SARS CoV-2 pseudovirus assays. H1650 cells were used in MERS CoV pseudovirus assays. Respective target cells were seeded in 96-well, flat-bottom, clear, tissue-culture treated plates (Thermo Fisher Scientific) at 25,000 cells/well with 6 μg/mL polybrene (Sigma) in the corresponding culture medium supplemented with 10% fetal bovine serum (Sigma) and 1% Penicillin Streptomycin (Thermo Fisher). RPMI media with glucose, HEPES Buffer, L-Glutamine, sodium bicarbonate and sodium pyruvate (Gibco) served as base medium for H1573/ACE2 cells and H1650 cells, while 293T Growth Media was used as base medium for 293T/17 cells. Pseudovirus was then added to wells at TCID50 concentrations, along with titrated neutralizing decoy MVPs in 9×2-fold or 3-fold serial dilutions, yielding a 10-point dilution curve. In delayed pseudovirus neutralization assays, pseudovirus was added to wells in TCID50 concentrations and incubated with cells for 60 minutes prior to the addition of titrated neutralizing decoy MVPs. Plates containing cells, pseudovirus and decoy MVPs were then centrifuged at 800×g, 25° C. for 60 minutes to maximize infection efficiency. 48 hours post-infection, cells were lysed using Firefly Luciferase Lysis Buffer (Biotium) and lysis was transferred to 96-well, white assay plates (Costar) before luciferase activity was analyzed via GLOMAX multi-detection system (Promega). Titrated infection data was then plotted and fitted to a 4-parameter, logistic curve (GraphPad Prism 9.0.0) to calculate the half maximal inhibitory concentration (IC₅₀) of various decoy MVPs neutralizing their respective pseudoviruses.

### Effects of Co-Stim MVPs on T Cells Activation, Proliferation and Differentiation

Purified mouse spleen T cells or human peripheral blood T cells were used to examine the effects of co-stim MVPs on T cell activation, proliferation, apoptosis, and differentiation. The 96-well plate was firstly coated with 50 μL of 1 μg/mL anti-mouse CD38 antibody (BioLegend, clone 145-2C11, Ultra-LEAF format) or 5 μg/mL anti-human CD3 antibody (BioLegend, clone UCHT1, Ultra-LEAF format) at 37° C. for 2 hrs before adding different ratio of corresponding co-stimulatory MVPs. If αCD3tri-MVP was added instead of anti-mouse CD3ε antibody or anti-human CD3 antibody, all the MVPs would mix with cells without pre-coating the plate. At day 2 or 3 posted MVP-COSTIMs treatment, cells were analyzed by FACS to determine the expression of early activation markers CD69 and CD25. T cells were split every 2-3 days to keep the concentration at around 50-150k/well in the 96-well plate. Cell counts were monitored after each cell splitting using Celigo instrument (brightfield channel and fluorescence algorism for alive cells counting) for 8-10 days to determine the effects of MVP-COSTIMs on mouse T cell proliferation or up to 15-18 days to determine the effects of MVP-COSTIMs on human T cell proliferation. The composition of effector and memory cells was quantified by FACS analyses of CD62L and CD45RO expression to determine the effect of MVP-COSTIMs on human T cell differentiation.

### Example 7. Generation and Characterization of Monomeric Multivalent Particles (MVPs)

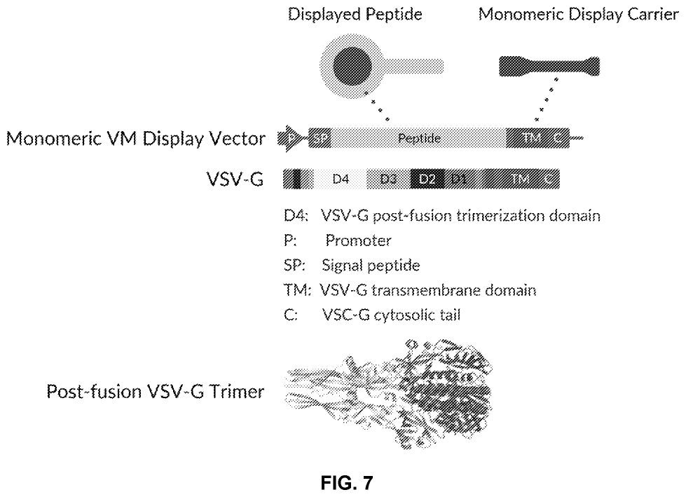

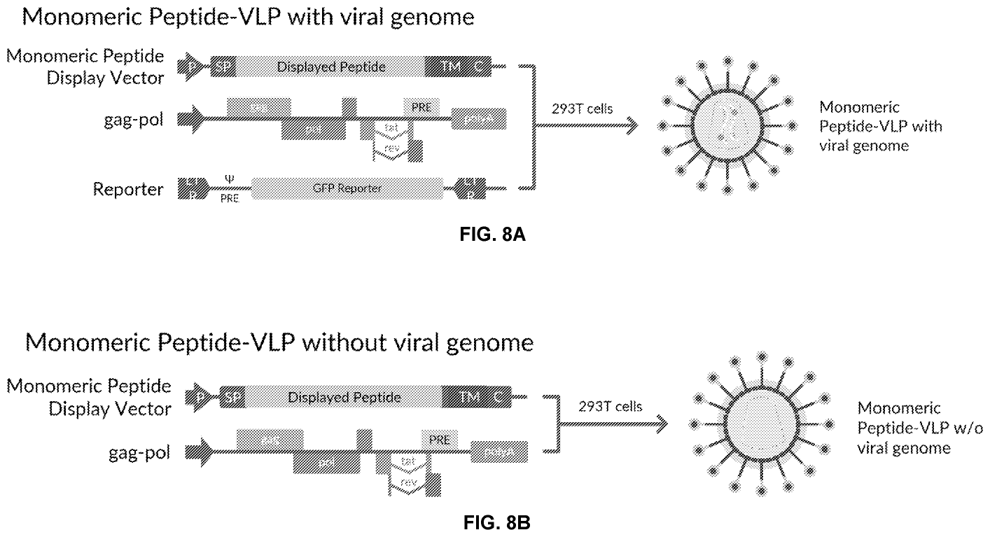

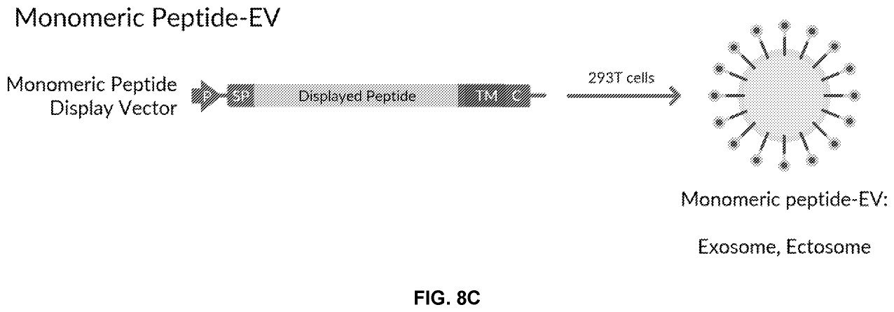

The design of a monomeric display vector is illustrated in FIG. 7. The fusion protein construct encodes a promoter, signal peptide, the desired display peptide, followed by the transmembrane and cytosolic tail domains of VSV-G protein (FIG. 7). A monomeric display vector was designed to express a fusion protein consisting of a protein linked to the VSVG transmembrane and intracellular domains (FIG. 7). The other structural domains of VSVG including the D4 post-fusion trimerization domain were eliminated to facility the display of monomeric proteins. By design, multivalent proteins can be displayed as monomers on the surface of viral-like particles (VLPs) and extracellular vesicles (EVs) such as exosomes and ectosomes using this monomeric display vector (FIGS. 8A-8C). To produce monomeric VLP-based MVPs with viral RNA genomes. HEK 293T cells were co-transfected with the monomeric peptide fusion construct with a lentiviral packaging construct expressing essential packaging components, such as Gag-Pol and rev proteins, and a viral genome transfer vector encoding a GFP/luciferase reporter (FIG. 8A). Alternatively, monomeric VLP-MVPs without RNA genome were produced by co-transfecting the HEK 293T cells with a peptide display vector together with a lentiviral packaging construct but no viral genome transfer vector (FIG. 8B). Finally, monomeric EV-based MVPs, including exosome-MVPs and ectosome-MVPs, were produced by transfecting only the monomeric peptide displaying vector in 293T cells (FIG. 8C).

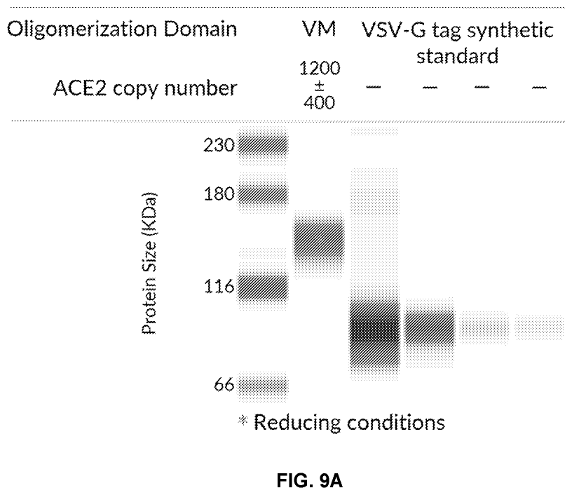

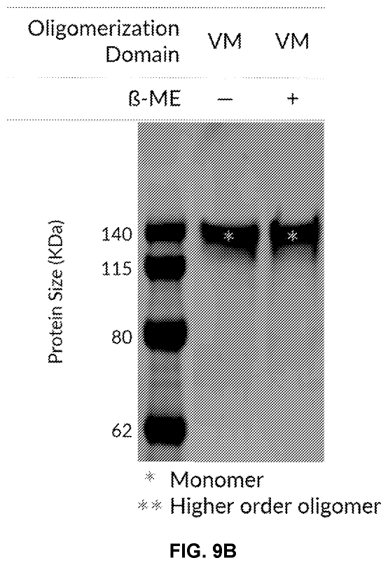

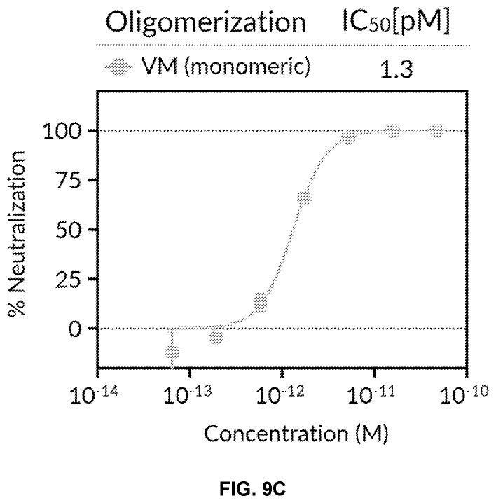

The monomeric display vector (FIG. 7) was used to produce VLPs displaying angiotensin-converting enzyme 2 (ACE2), the entry receptor for SARS CoV-2, termed ACE2-VM MVPs, according to FIG. 8A. The concentration of purified ACE2-VM MVPs was determined via P24 ELISA. Quantitative western blot analysis revealed that ACE2-VM MVPs displayed 1200±400 copies of ACE2 per particle (FIG. 9A). Furthermore, western blot analysis of ACE2-VM MVPs comparing fusion proteins under reducing and non-reducing conditions demonstrated no difference in bands, indicating that ACE2/VM fusion proteins were expressed as monomers on the surface of MVPs (FIG. 9B). To confirm that ACE2-VM MVPs displayed functional ACE2, the ACE2-VM MPVs were analyzed in pseudovirus neutralization assays against SARS CoV-2 (FIG. 9C). ACE2/VM MVPs neutralized SARS CoV-2 pseudovirus at a potent IC₅₀ of 1.3 pM (FIG. 9C), demonstrating that ACE2-VM MVPs displayed functional ACE2 that allowed for multivalent binding and neutralization of SARS CoV-2 pseudovirus. These results demonstrated that monomeric fusion peptides can effectively display thousands of copies of functional peptide on the surface of VLPs.

### Example 8. Generation and Characterization of Dimeric MVPs

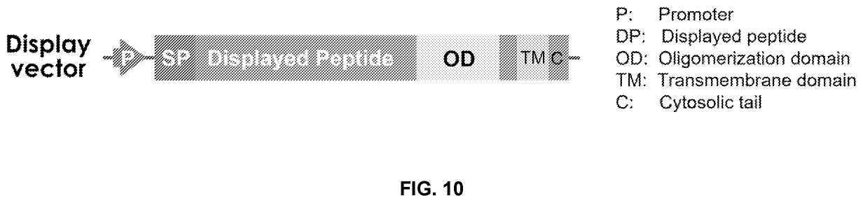

A number of proteins are clustered on a cell surface in oligomeric forms. MVPs with matching oligomeric display formats can further enhance their avidity at the level of individual oligomeric binding partners. To mimic multivalent oligomeric protein expression on cell surface, display vectors were designed to express and efficiently target displayed peptides to enveloped vesicles, such as VLPs and exosomes (FIG. 10). The display vectors were designed to express a fusion protein comprising a signal peptide, a displayed peptide, an oligomerization domain, and a transmembrane and cytosolic tail for membrane anchoring and targeting (FIG. 10). Viral surface proteins may contain unique signal sequences required for efficient display on VLPs and EVs. Various oligomerization domains were selected to test their effects on display efficiency and oligomerization format on MVPs, as listed in Table 5.

TABLE 5
Exemplary Oligomerization Domains

| OD | | | Display |
|---|---|---|---|
| (Oligomerization | | | Anchor |
| Domain) | Description | Oligomerization | Type |
| VM | VSV-G protein | Monomer | Type I |
| | transmembrane | | transmembrane |
| | region and cytosolic | | |
| | tail | | |
| LZ | GCN4 Leucine | Dimer | Type I |
| | zipper domain | | transmembrane |
| DE | Dengue E protein | Trimer | Type I |
| | post-fusion | | transmembrane |
| | trimerization domain | | |
| Fold | T4 Phage Fibritin | Trimer | Type I |
| | Foldon | | transmembrane |
| D4 | VSV-G D4 post- | Trimer | Type I |
| | fusion trimerization | | transmembrane |
| | domain | | |
| NA | Influenza | Tetramer | Type II |
| | Neuraminidase stem | | transmembrane |

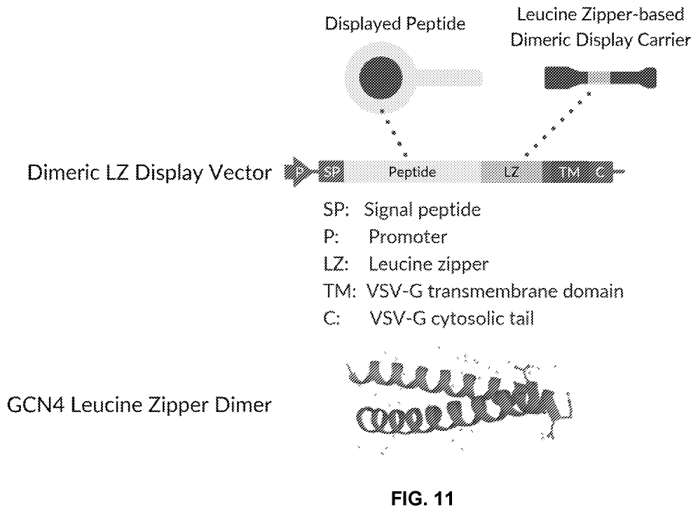

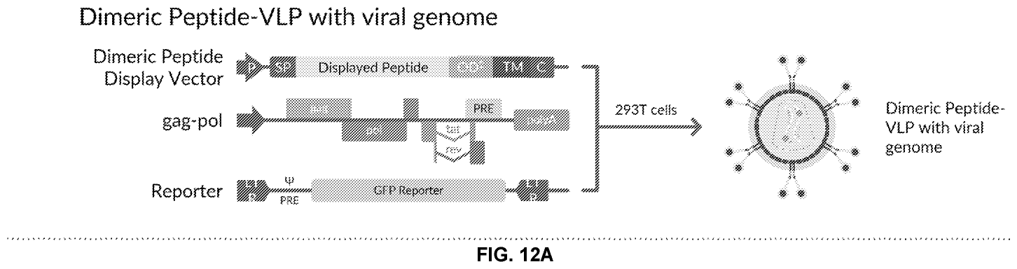

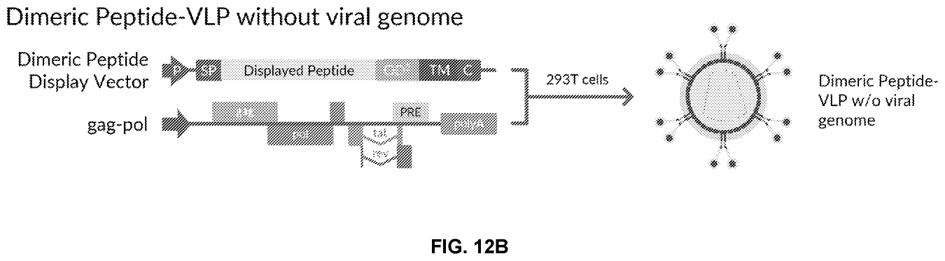

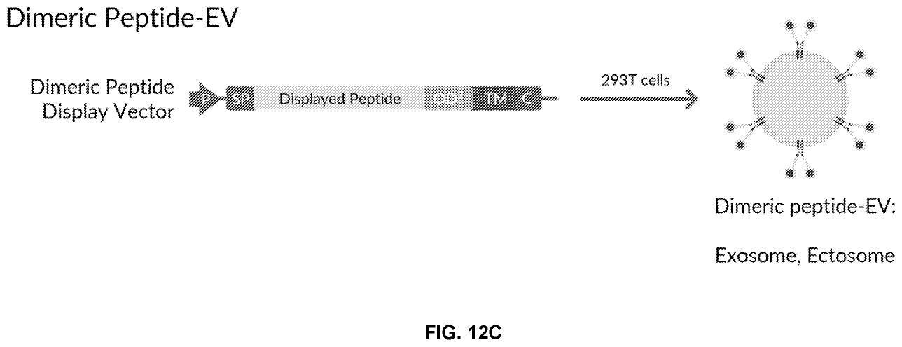

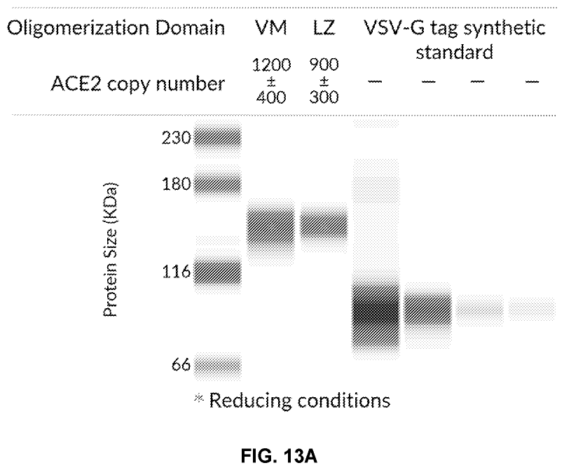

A dimeric display construct was designed that encoded a promoter, signal peptide and the desired display peptide fused to the GCN4 Leucine Zipper dimerization domain (LZ), followed by the transmembrane and cytosolic tail domains of VSV-G protein (FIG. 11). To produce dimeric VLP-based MVPs with viral RNA genomes. 293T cells were co-transfected with a dimeric peptide fusion construct with the leucine zipper dimerization domain alongside a lentiviral packaging construct expressing essential packaging components, such as Gag-Pol and Rev proteins, and a viral genome transfer vector encoding a GFP/luciferase reporter (FIG. 12A). Alternatively, dimeric VLP-based MVPs without RNA genome were produced by co-transfecting 293T cells with a dimeric peptide display vector together with a lentiviral packaging construct but without viral genome transfer vector (FIG. 12B). Finally, dimeric EV-based MVPs, including exosome-MVPs and ectosome-MVPs, were produced by transfecting 293T cells with only the dimeric peptide displaying vector in (FIG. 12C).

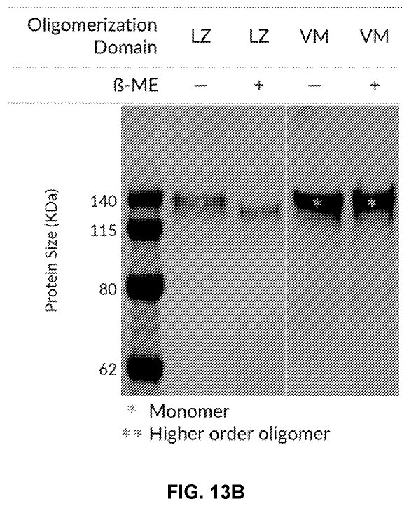

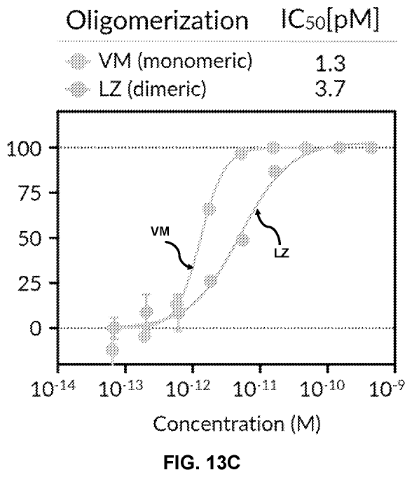

The dimeric display vector illustrated in FIG. 11 was used to produce VLPs displaying Angiotensin-converting enzyme 2 (ACE2), the entry receptor for SARS CoV-2, termed ACE2-LZ MVPs (FIG. 12A). The concentration of purified ACE2-LZ MVPs was determined via P24 ELISA. Quantitative western blot analysis revealed that ACE2/LZ MVPs display approximately 900±300 copies of ACE2 per particle, compared with 1200±400 copies per particle of monomeric ACE/VM MVPs (FIG. 13A). Quantitative western blot analysis under reducing and non-reducing conditions indicated that ACE2-LZ fusion peptides were expressed in both dimeric and monomeric forms on ACE2-LZ MVPs (FIG. 13B), unlike ACE2-VM fusion peptides, which were only monomeric. To confirm that ACE2-LZ MVPs displayed functional ACE2, the ACE2-LZ MVPs were analyzed in pseudovirus neutralization assays against SARS CoV-2. ACE2-LZ MVPs neutralized SARS CoV-2 pseudovirus at a potent IC₅₀ of 3.7 pM (FIG. 13C). This marginal decrease in potency as compared to monomeric ACE2/VM MVPs was likely due to decreased overall ACE2 copy number. These results demonstrated that ACE2-LZ MVPs displayed hundreds of functional, dimeric ACE2 fusion peptides that allowed for multivalent binding and neutralization of SARS CoV-2 pseudovirus. In general, these results demonstrated that dimeric fusion peptides can effectively display hundreds or more copies of functional peptide as dimers on the surface of MVPs.

### Example 9. Generation and Characterization of Trimeric MVPs

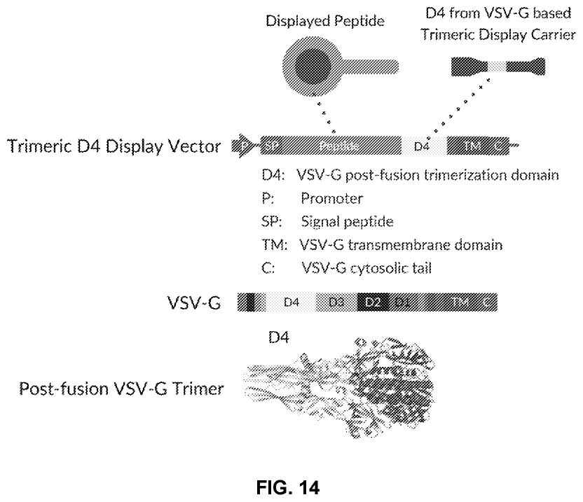

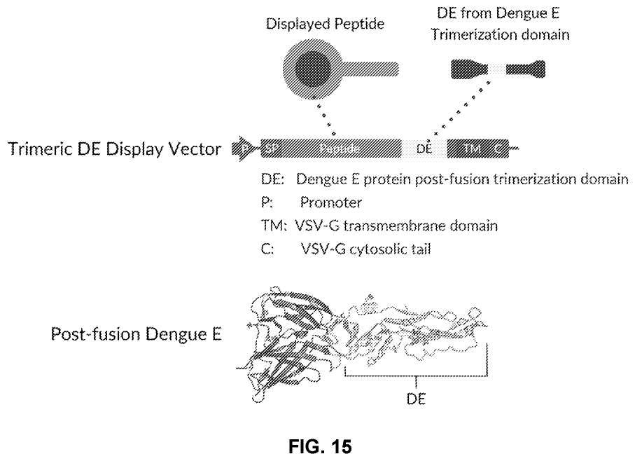

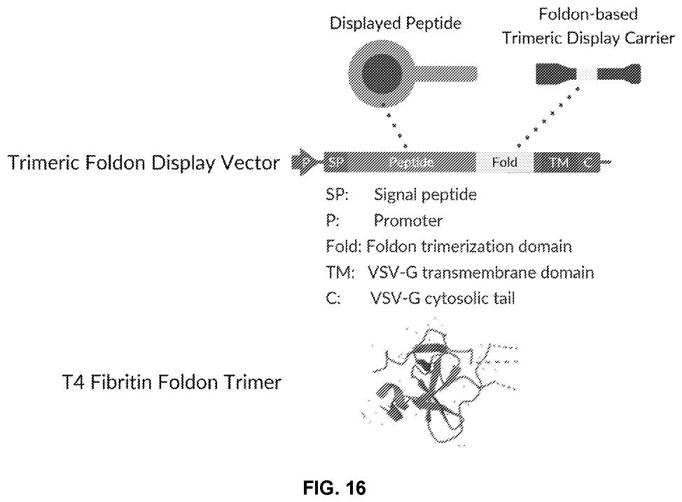

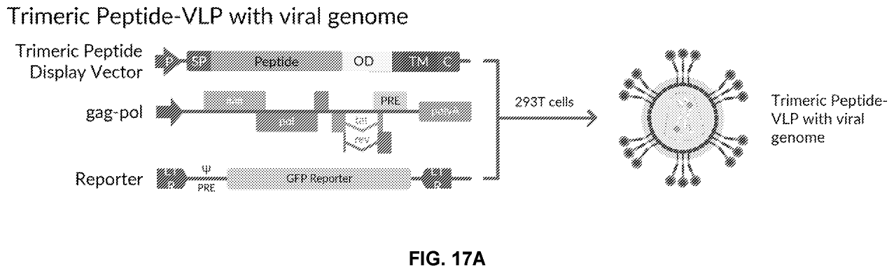

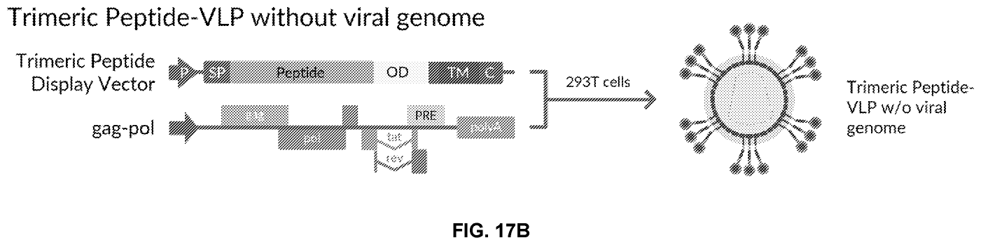

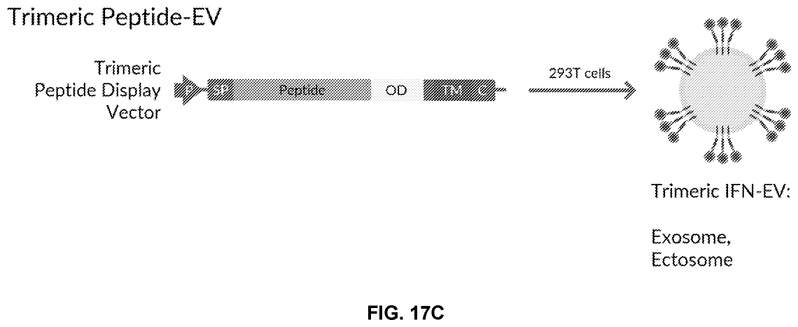

To generate trimeric MVPs, several trimeric display constructs were designed by fusing the displayed peptide to the D4 post-fusion trimerization domain of VSV-G (D4V2. FIG. 14), the Dengue E protein post-fusion trimerization domain (FIG. 15), or the T4 Phage Fibritin Foldon domain (FIG. 16), followed by the transmembrane and intracellular domains of VSV-G. These trimeric display vectors also encoded a promoter and a signal peptide. By design, multivalent proteins can be displayed as trimers on the surface of viral-like particle (VLP) and extracellular vesicle (EV), such as exosomes and ectosomes, using a trimeric display vector. To produce trimeric VLP-based MVPs with viral RNA genomes. HEK 293T cells were co-transfected with a trimeric peptide fusion construct utilizing one of several trimerization domains—D4DE, or Foldon—alongside a lentiviral packaging construct expressing essential packaging components, such as Gag-Pol and Rev proteins, and a viral genome transfer vector encoding a GFP/luciferase reporter (FIG. 17A). Alternatively, trimeric VLP-based MVPs without RNA genome were produced by co-transfecting HEK 293T cells with display vector together with only a lentiviral packaging construct but not the viral genome transfer vector (FIG. 17B). Finally, trimeric EV-based MVPs, including exosome-MVPs and ectosome-MVPs, were produced by transfecting HEK 293T cells with only the trimeric peptide display vector (FIG. 17C).

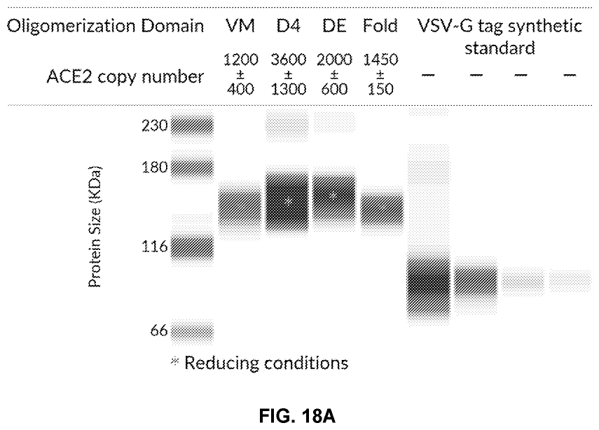

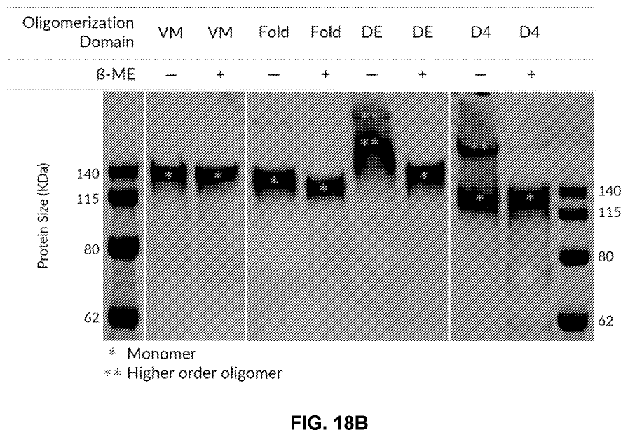

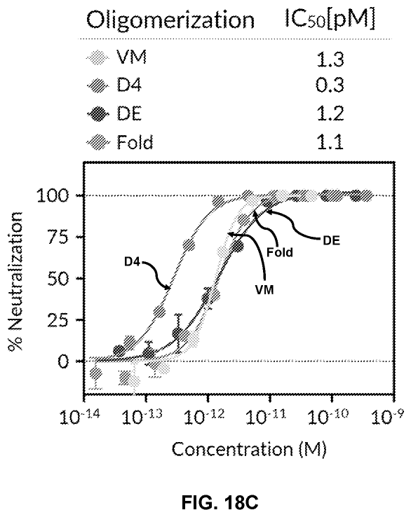

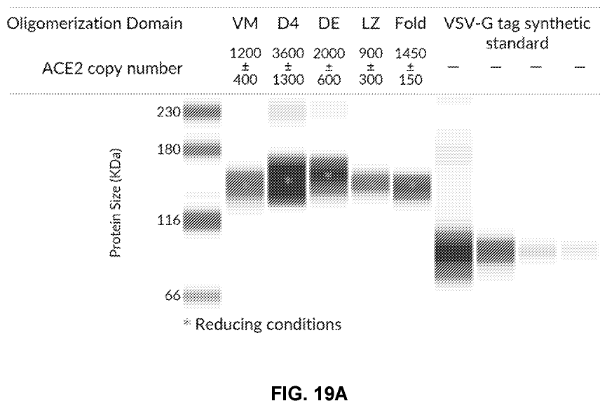

Trimeric ACE2 decoy MVPs were generated using trimeric fusion constructs derived from several trimerization domains, the D4 post-fusion trimerization domain of VSV-G (FIG. 14), the Dengue E post-fusion trimerization domain (FIG. 15), and the T4 Phage Foldon domain (FIG. 16). ACE2 MVPs displaying each of these trimeric ACE2 fusion constructs were termed ACE2-D4. ACE2-DE and ACE2-Fold MVPs, respectively. Purified ACE2-D4. ACE2-DE and ACE2-Fold MVPs were quantified via P24 ELISA. Quantitative western blot analysis of each type of trimeric MVP revealed that ACE2-D4, ACE2-DE and ACE2-Fold MVPs displayed 3600±1300, 2000±600 and 1450±150 copies of ACE2 per particle, respectively (FIG. 18A), demonstrating that inclusion of trimerization domains in fusion constructs significantly increased display copy numbers as compared to monomeric fusion constructs which displayed 1200±400 copies of ACE2 per particle (FIGS. 9A, 18A). To verify that trimerization constructs displayed ACE2 in trimers, monomeric and trimeric ACE2 MVPs were analyzed by western blots under reducing and non-reducing conditions (FIG. 18B). It was found that while ACE2-VM MVPs demonstrated only a single, monomeric band under non-reducing conditions. ACE2/D4. ACE2/DE and ACE2/Fold showed larger, higher order oligomeric bands, including trimeric bands (FIG. 18B). Of these three. ACE2/D4 and ACE2/DE MVPs demonstrated a larger proportion of oligomerized fusion peptides at much higher copies/particle (FIG. 18B). Finally, trimeric ACE2 MVPs were analyzed in pseudovirus neutralization assays against SARS CoV-2 to confirm functional ACE2 display. The results indicated that ACE2-D4. ACE2-DE and ACE2-Fold MVPs neutralized SARS CoV-2 pseudovirus at ICso of 0.3, 1.2 and 1.1 pM (FIG. 18C), respectively, and were up to 10-fold more potent than monomeric ACE2-VM MVPs (FIGS. 9C, 18C). This increase in neutralizing potency can be attributed to both the increase in overall ACE2 valency on trimeric MVPs, as well as the boosting of local multivalence between viral spike trimers and trimeric ACE2 fusion peptides, allowing trimeric ACE2 MVPs to bind and neutralize virions more effectively. These results demonstrated that ACE2 MVPs expressing trimeric fusion peptides displayed thousands of copies of functional, trimerized ACE2 per particle. Trimeric fusion display constructs incorporating trimerization domains such as D4, DE or Foldon can be used to display thousands of functional, trimeric peptides on MVPs. ACE2-D4 MVPs had the highest copies of trimeric/oligomeric ACE2 displayed per particles and were the most potent in virus neutralization, illustrating that optimized valency and oligomerization formats of ACE2 on MVPs may lead to more potent neutralizing antivirals against SARS CoV-2.

### Example 10. Comparison of Monomeric, Dimeric and Trimeric MVPs

Table 6 lists a summary of the effects of the oligomerization domains on the level of ACE2 display on MVPs and the neutralization potency of ACE2-MVPs.

TABLE 6
Comparison of ACE2-MVPs with
Different Oligomerization Domains

| Oligomerization | Displayed | Oligo- | Copy | Pseudovirus |
|---|---|---|---|---|
| Domain | Peptide | merization | Number | IC₅₀ (pM) |
| VM | ACE2 | Monomer | 1200 ± 400 | 1.3 |
| LZ | ACE2 | Dimer | 900 ± 300 | 3.7 |
| DE | ACE2 | Trimer | 2000 ± 600 | 1.2 |
| Fold | ACE2 | Trimer | 1450 ± 150 | 1.1 |
| D4 | ACE2 | Trimer | 3600 ± 1300 | 0.3 |

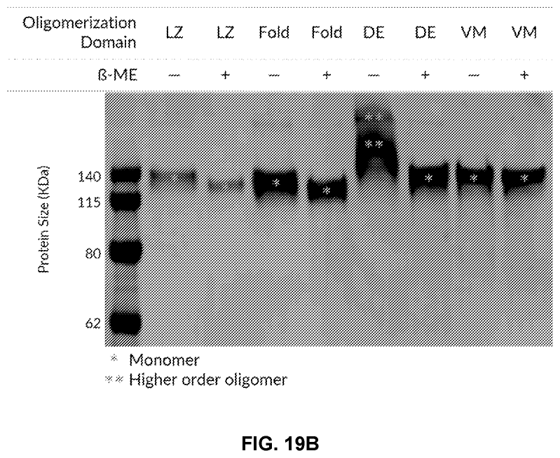

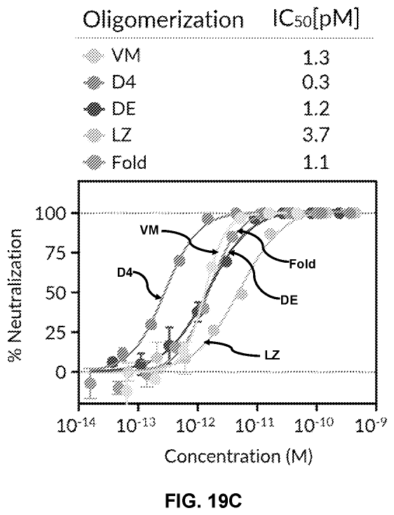

Decoy-MVPs with distinct oligomerized ACE2 display had varied copies ACE2 molecules/particle through quantitative western-blot analyses of copy numbers (Table 6, FIG. 19A). For example, trimeric ACE2-D4 MVP had the highest level of ACE2 protein displayed at ˜3600±1300 copies/particle, whereas monomeric ACE2-VM MVP and dimeric ACE2-LZ MVP displayed about 1000 copies ACE2/particle. Among the ACE2-MVPs with D4, or DE, or Fold trimeric domains, ACE2-D4 and ACE2-DE MVPs utilizing trimeric domains from viral surface proteins displayed over 2000 copies ACE2/particle, whereas ACE2-Fold MVPs utilizing a trimeric domain from bacteria phage protein displayed about 1450 copies ACE2/particle. These results illustrated that ACE2 surface display levels on VLPs can be enhanced by incorporating trimerization domains from viral surface proteins into the display vector, such as D4 of VSVG and DE of Dengue E surface protein (FIG. 19A). Moreover, D4 and DE promoted more robust trimerization or oligomerization of ACE2 display on VLPs (FIGS. 18B, 19B) by western-blot analyses of ACE2-D4, ACE2-DE, and ACE2-Fold MVPs under reducing or non-reducing conditions, suggesting that these trimeric domains enhanced surface presentation of displayed peptides. Trimeric configuration may be one of the most common features shared by many viral surface or spike proteins. Finally, the neutralizing potencies of ACE2-MVPs determined by SARS CoV-2 pseudovirus neutralization analyses were also impacted by the oligomerization format of displayed ACE2 (FIG. 19C). ACE2-D4 MVP had the highest neutralizing potency, which correlated with its high ACE2 valency and trimeric or oligomeric configuration. All ACE2-MVPs had IC₅₀s in the low picomolar range, demonstrating that they were all potent neutralizing molecules against SARS CoV-2 in pseudovirus neutralization assays.

### Example 11. Generation and Characterization of Tetrameric MVPs

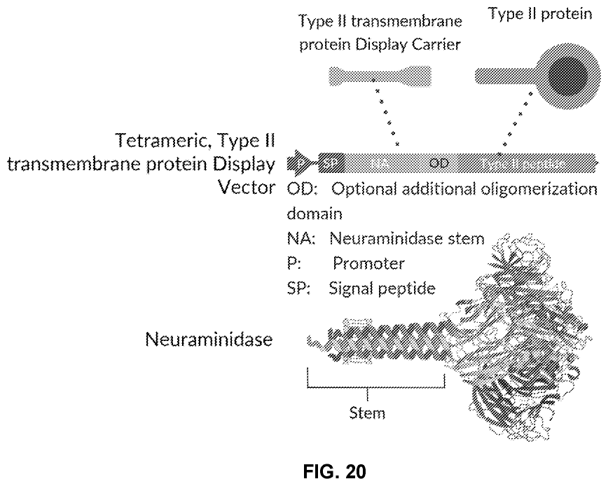

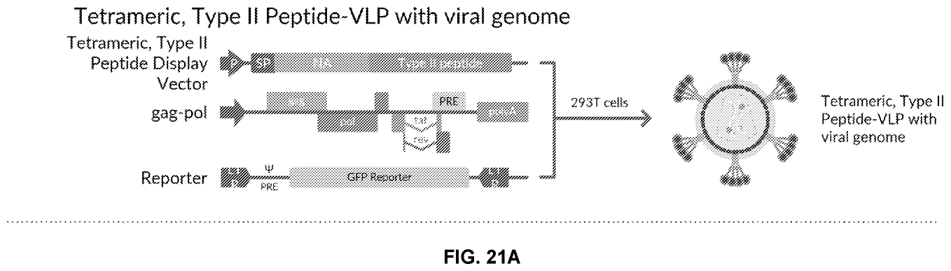

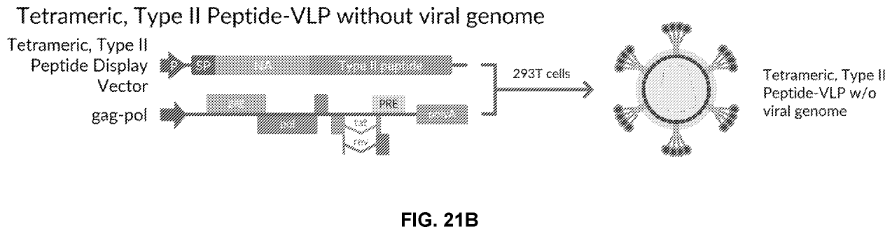

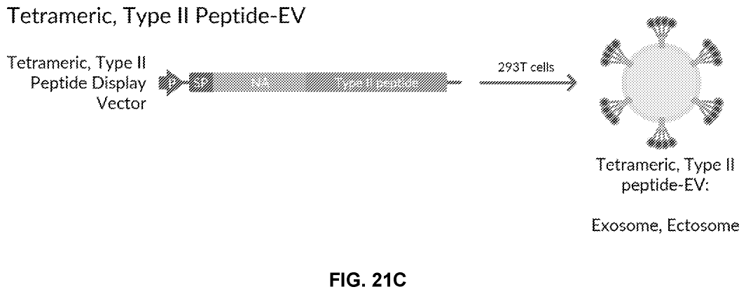

A tetrameric display construct was designed by fusing the stem domain of Influenza Neuraminidase to the Type II display peptide and it also encoded a promoter, a signal peptide (FIG. 20). By design, multivalent proteins can be displayed as tetramers on the surface of VLPs and EVs, such as exosomes and ectosomes, using a tetrameric display vector. To produce tetrameric, VLP-based MVPs with viral RNA genomes, HEK 293T cells were co-transfected the tetrameric, NA-based Type II peptide fusion construct with a lentiviral packaging construct expressing essential packaging components, such as Gag-Pol and Rev proteins, and a viral genome transfer vector encoding a GFP/luciferase reporter (FIG. 21A). Alternatively, tetrameric, VLP-based MVPs without RNA genomes were produced by co-transfecting HEK 293T cells with the tetrameric peptide display vector together with a lentiviral packaging construct but no viral genome transfer vector (FIG. 21B). Finally, tetrameric, EV-based MVPs, including exosome-MVPs and ectosome-MVPs, were produced by transfecting HEK 293T cells with only the tetrameric peptide displaying vector in 293T cells (FIG. 21C).

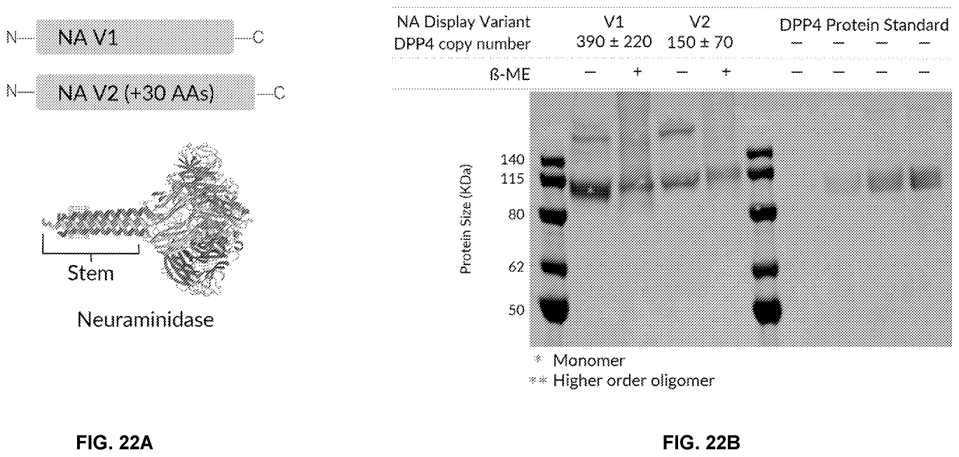

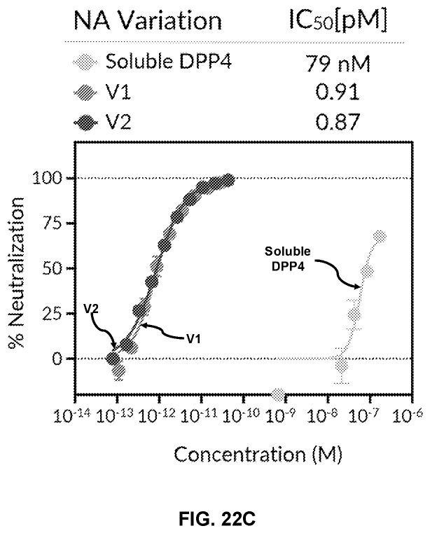

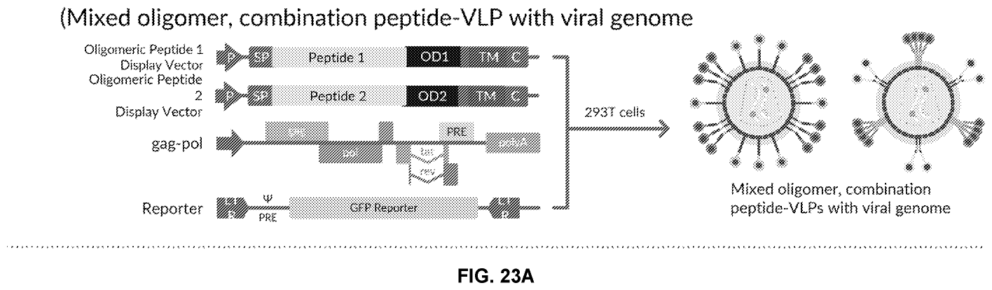

Tetrameric MVPs were generated and characterized displaying Dipeptidyl Peptidase 4 (DPP4), the entry receptor for MERS CoV, which were termed DPP4-NA MVPs. Two variations of the tetrameric fusion peptide were generated, both derived from the Influenza Neuraminidase stem domain (FIG. 22A). The concentration of purified DPP4-NA MVPs was determined via P24 ELISA. Quantitative western blot analysis showed that DPP4/NA MVPs displaying versions 1 and 2 of the DPP4/NA display constructs displayed approximately 390±220 and 150±70 copies of DPP4 per particle, respectively (FIG. 22B). In addition, western blot analysis of both versions of DPP4/NA MVPs under reducing and non-reducing conditions demonstrated that both versions resulted in multimeric DPP4 display on MVPs (FIG. 22B). Finally, to determine whether the fusion peptides effectively display functional DPP4 on MVPs, both versions of DPP4/NA MVPs were tested against MERS CoV in pseudovirus neutralization assays. Version 1 and 2 of DPP4/NA MVPs neutralized MERS CoV pseudovirus at IC₅₀s of 0.91 pM and 0.87 pM, respectively, more than 5-logs more potent than soluble DPP4 protein (FIG. 22C). These results demonstrated that DPP4-NA MVPs displayed hundreds of copies of multimeric, functional DPP4. Moreover, Neuraminidase stem-based fusion peptides were effective, Type II transmembrane proteins capable of displaying numerous, tetrameric copies of peptide on MVP surfaces.

### Example 12. Generation and Characterization of Mixed Oligomeric MVPs

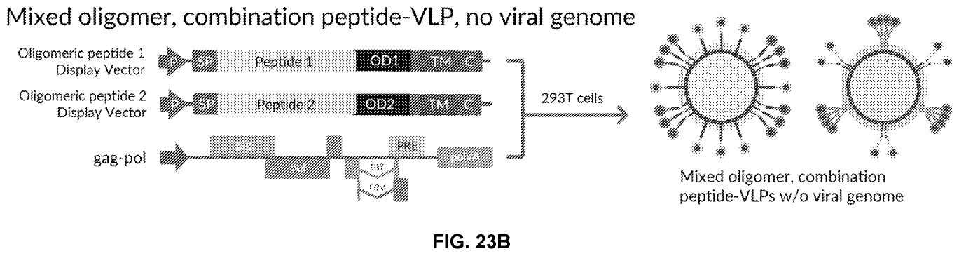

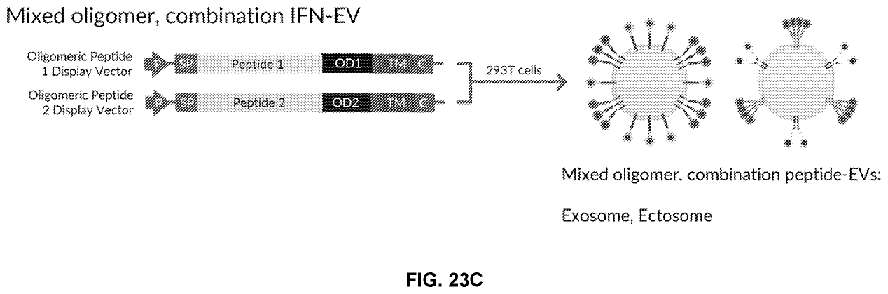

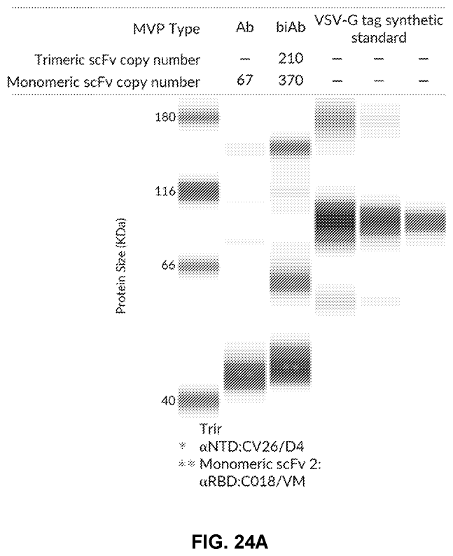

With the display vectors disclosed herein. MVPs displaying multiple peptides in mixed oligomeric formats were generated by co-transfection with distinct peptide display constructs containing differing oligomerization domains. Such design can be used to increase the display density of a displayed peptide or to create combinatorial display patterns of distinct display peptides. Mixed oligomeric MVPs can be built with VLPs and EVs, such as exosomes and ectosomes, by co-transfection with monomeric and oligomeric display vectors. To produce mixed VLP-based MVPs with viral RNA genomes. 293T cells were co-transfected with multiple distinct peptide display constructs encoding distinct peptides on various oligomerization domains, alongside with a lentiviral packaging construct expressing essential packaging components, such as Gag-Pol and Rev proteins, and a viral genome transfer vector encoding a GFP/luciferase reporter (FIG. 23A). Alternatively, mixed VLP-based MVPs without RNA genome were produced by co-transfecting the 293T cells with the mixed oligomeric display vectors together with only a lentiviral packaging construct but not the viral genome transfer vector (FIG. 23B). Finally, mixed EV-based MVPs, including mixed exosome-MVPs and ectosome-MVPs, were produced by transfecting the 293T cells with the mixed oligomeric fusion constructs alone (FIG. 23C). Mixed MVPs can be generated by co-transfection with display vectors utilizing distinct oligomerization domains disclosed herein (FIG. 10).

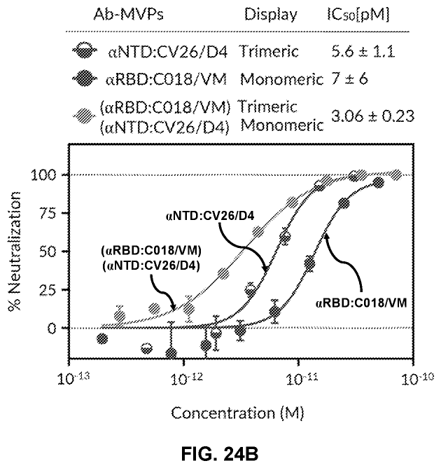

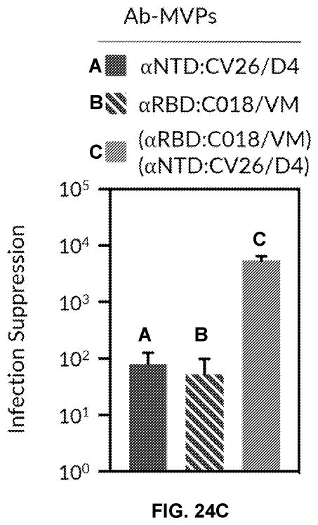

An analysis was carried out to determine whether the neutralizing potency of MVPs displaying spike-recognizing antibodies can be further enhanced with multi-specificity. For example. MVPs can be genetically programmed to display combinations of distinct scFv antibodies recognizing multiple binding sites on the SARS CoV-2 spike protein. This design, if successful, may further enhance the neutralizing potency of such MVPs against pandemic viruses and mitigate the effects of typical spike escape mutagenesis on neutralizing antibodies. To this end. (αRBD: C018/VM) (αNTD: CV26/D4) bi-specific MVPs (bi-MVPs) were designed and generated that co-displayed two scFv fusion peptides—monomeric αRBD: C018/VM and trimeric αNTD: CV26/D4—recognizing the RBD and NTD regions of the CoV-2 spike protein. The expression level and oligomerization of monomeric αRBD: C18/VM and trimeric αNTD: CV26/D4 on MVPs were confirmed by quantitative western-blot analyses (FIG. 24A). These bi-MVPs were tested against their respective target viruses in pseudovirus neutralization assays. The results demonstrated that mixed (αRBD: C018/VM) (αNTD: CV26/D4) bi-MVPs had an IC₅₀ of 3.06±0.23 pM in pseudovirus neutralization assays, a more than 2-fold increase in potency as compared to mono-specific MVPs displaying either αNTD: CV26/D4 or αRBD: C018/VM alone (FIG. 24B). Furthermore, these mixed biAb-MVPs suppressed pseudovirus infection by approximately 10.000-fold as determined by the decrease in pseudovirus infection luciferase signal, a 100-fold increase in suppression compared to each respective mono-specific MVPs (FIG. 24C). These results indicated the potential to genetically program MVPs to display distinct peptides to create bi-specific functionalities.

### Example 13. MVPs with Different Oligomerization Domain Configurations

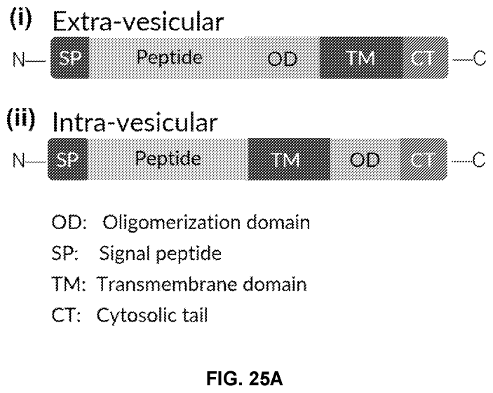

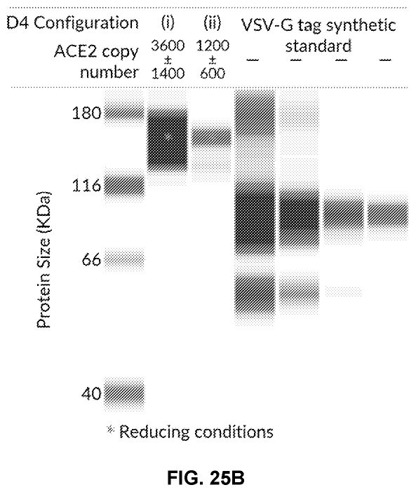

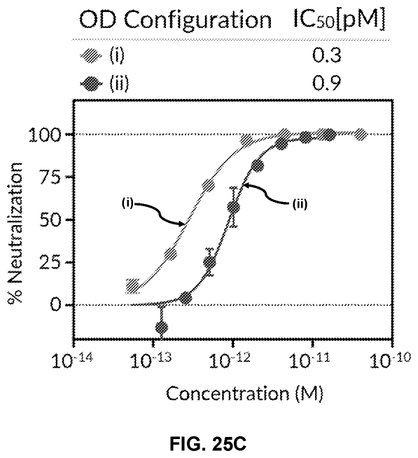

MVPs can be genetically programmed to display peptides in various configurations by modifying the display vector. The oligomerization domain can be placed at various positions in the fusion peptide: (i) extracellular and juxtaposed to the transmembrane domain and (ii) intracellular and juxtaposed to the transmembrane domain (FIG. 25A). To this end, decoy-MVPs were generated and characterized that displayed ACE2 on trimeric fusion peptides utilizing the D4 trimerization domain in various configurations (FIG. 25A). Quantitative western-blot analyses indicated that MVPs utilizing D4 in extra-vesicular configuration (i) displayed 3600±1400 copies of ACE2 per particle, nearly 3 times as many copies of ACE2 as MVPs utilizing display constructs with D4 in (ii) intra-vesicular configuration (FIG. 25B). Furthermore, configuration (i) extra-vesicular D4 MVPs neutralized SARS CoV-2 pseudovirus at an ICso of 0.3 pM, a similar 3-fold increase in potency as compared to otherwise identical configuration (ii) intra-vesicular D4 MVPs displaying ACE2 (FIG. 25C). These results demonstrated that D4 display peptides with various intra and extra-vesicular D4 configurations can effectively produce MVPs displaying high copy numbers of oligomeric peptides. Moreover, the exact configuration of oligomerization domains in fusion peptides can be adapted to fit many purposes-minimizing antigenicity, tailoring fusion peptides to display peptide structural differences-all while maintaining high copy number, oligomeric display on MVPs.

### Example 14. MVPs with Truncated Forms of D4 Oligomerization Domains

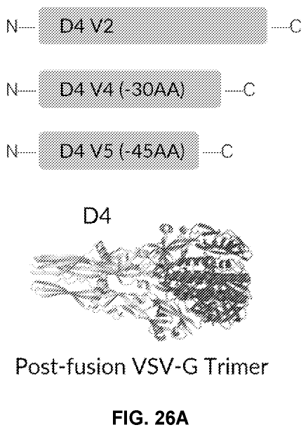

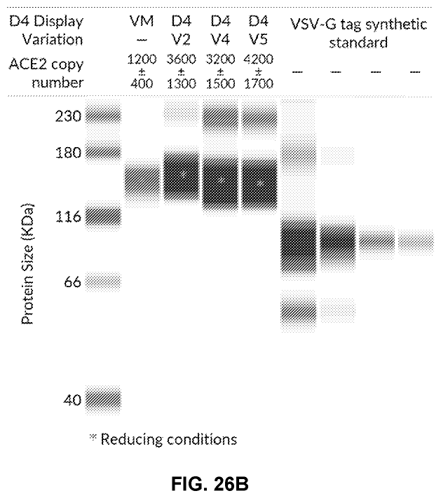

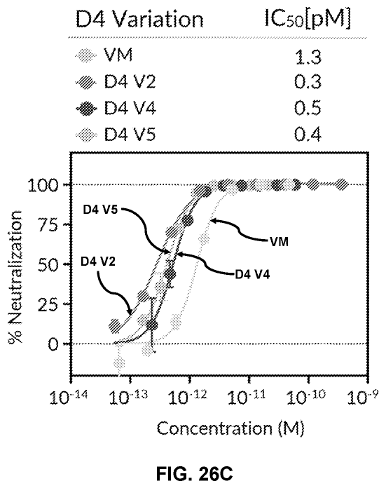

The D4 domain used in the original trimeric display vector (Designated as D4v2. SEQ ID No: 7) was modified by truncating from its n-terminus and c-terminus based on the 3-D structure of the D4 domain (FIGS. 14, 26A). The goal was to find the minimal D4 domain required for effective peptide display. To this end. ACE-D4 display vector was generated utilizing truncated D4v4 (SEQ ID No: 9), and D4v5 (SEQ ID No: 10) as the oligomerization domain (FIG. 26A) and produced variations of ACE2-D4 MVPs using these display vectors. As determined by quantitative western blot analyses. ACE2-D4v4 and ACE2-D4v5 MVPs displayed 3200±1500 and 4200±1700 copies of ACE2 per particle, respectively (FIG. 26B). These results demonstrated that truncation of D4 may further increase fusion peptide display. Both D4 variations resulted in higher ACE2 copies per particle than the monomeric control ACE2 decoy MVPs. Furthermore. ACE2-D4v2. ACE2-D4v4, and ACE2-D4v5 MVPs neutralized SARS CoV-2 pseudovirus at IC₅₀s of 0.3, 0.5 and 0.4 pM in pseudovirus neutralization assays (FIG. 26C). These results demonstrated that the D4 trimerization domain can be further optimized to increase display peptide copy number on MVPs, without compromising displayed peptide functionality.

### Example 14. MVPs with Patterned Display of Various Peptides

As listed in Table 7 and Table 8, the display methods described above were employed to generate patterned display of diverse classes of proteins with distinct structural and functional features on MVPs,

TABLE 7
MVP Peptides of Different Structural Classes

| Structural Class | Examples |
|---|---|
| Type I Transmembrane | ACE2, ICAM-1, LILR |
| Type II Transmembrane | DPP4, BAFF |
| Type III Transmembrane | BCMA, TACI, BAFF-R |
| GPI-anchored | CD24, CD48 |
| Secreted | IL-4, CXCL-12 |
| Multi-pass Transmembrane | NTCP, FcεRI-β |

TABLE 8
MVP Peptides of Different Functional Classes

| Functional Class | Examples |
|---|---|
| Antibodies | Non-neutralizing, |
| | neutralizing, |
| | multiple formats |
| Receptors | ACE2, DPP4 |
| Cytokines | IL-4, IL-6 |
| Immune Checkpoints | CTLA-4, PD-1 |
| Chemokines | CXCL-12 |
| Adhesion Factors | ICAM-1 |
| Mitogens | Wnt |

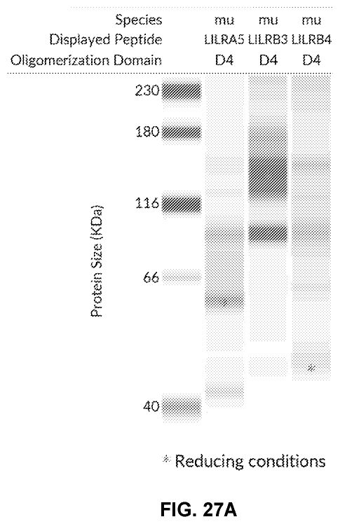

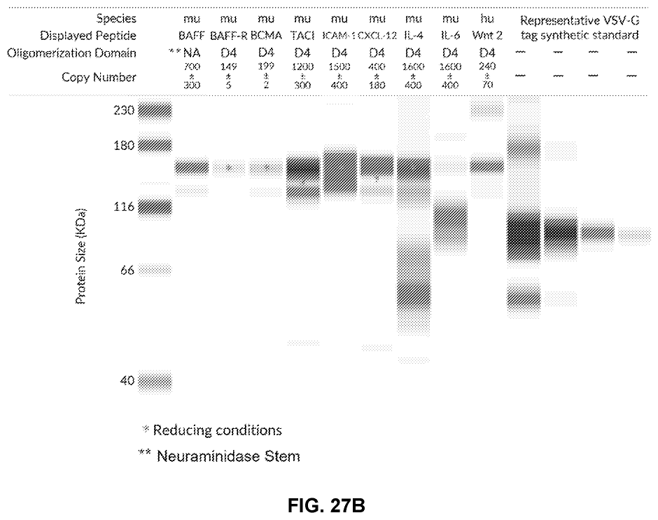

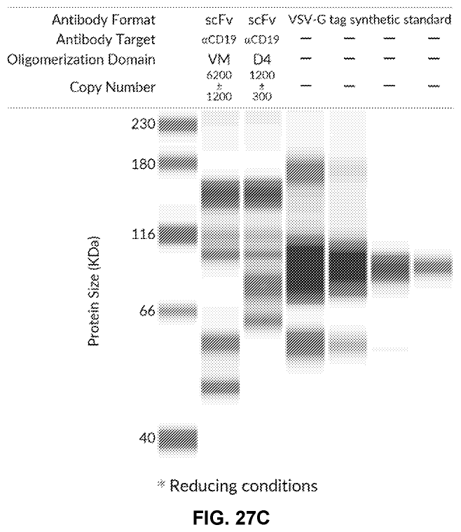

Proteins of distinct structural classes, such as extracellular domains of type I, II, and III transmembrane proteins, GPI-anchored proteins, secreted proteins, and multi-pass transmembrane proteins, were demonstrated to be effectively displayed on the surface of VLPs in monomeric or trimeric formats (Table 7, FIG. 27A-C). The display methods disclosed herein were employed to generate patterned display of diverse classes of proteins with distinct functional properties on VLPs and EVs. Single-chain antibodies, the extracellular domain of viral entry receptors, immune checkpoint, cytokines, mitogens, chemokines, and adhesion factors were demonstrated to be effectively displayed on the surface of VLPs in various oligomeric formats (Table 8, FIG. 27A-C). For example, decoy-MVPs were generated as SARS CoV-2 neutralizing therapeutics displaying thousands copies of monomeric or trimeric ACE2 extracellular domain, which is a type I transmembrane protein (FIGS. 9A-9C, 13A-13C, 18A-18C, and 19A-19C). Both monomeric and trimeric ACE2-MVPs were demonstrated to effectively neutralize SARS-CoV-2 virus in pseudovirus neutralization assays with sub-picomolar IC₅₀s (FIGS. 9C, 13C, 18C, and 19C). Furthermore, decoy-MVPs were also effectively generated as MERS CoV neutralizing therapeutics displaying hundreds of copies of DPP4 extracellular domain, which is a type II transmembrane protein (FIGS. 22A-22C). DPP4 decoy MVPs were demonstrated to effectively neutralize MERS in a pseudovirus neutralization assay with sub-picomolar IC₅₀s (FIGS. 22A-22C). Beyond the viral entry receptors, immune checkpoint, cytokines, mitogens, chemokines, and adhesion factors were effectively displayed on the surface of VLPs at high copy numbers (FIGS. 27A-27C). Western blot characterization of MVPs showed display of several types of murine LILR, LILRA5, LILRB3 and LILRB4, a type I transmembrane protein, on D4 trimeric display constructs (FIG. 27A). Quantitative western blot characterization of MVPs demonstrated display of various peptides, including cytokines (IL-4. IL-6), adhesion factors (ICAM-1), chemokines (CXCL-12), mitogens (Wnt 2). Type II transmembrane proteins (BAFF), and Type III transmembrane proteins (BAFF-R. BCMA. TACI) on various oligomerization domains (FIG. 27B). Peptide copy numbers are dependent on oligomerization domain and displayed peptide sequence. Finally. MVPs were designed and generated to display monomeric and trimeric single chain variable fragments (scFv) targeting CD19, a specific marker for B-cells or some types of B-cell leukemias, termed αCD19-VM and αCD19-D4 MVPs, respectively (FIG. 27C). αCD19-VM and αCD19-D4 MVPs displayed 6200±1200 and 1200±300 copies of scFv per particle through quantitative western blot analyses, respectively (FIG. 27C). Bi-function MVPs can be generated displaying aCD 19 and together with various receptors, or ligands, or cytokines to target the MVPs to normal or leukemia B cells. Collectively, these results demonstrated the MVP display technology can be broadly applied to generate MVPs displaying proteins of distinct structural and functional class of proteins.

### Example 15. Generation of EV-Based MVPs

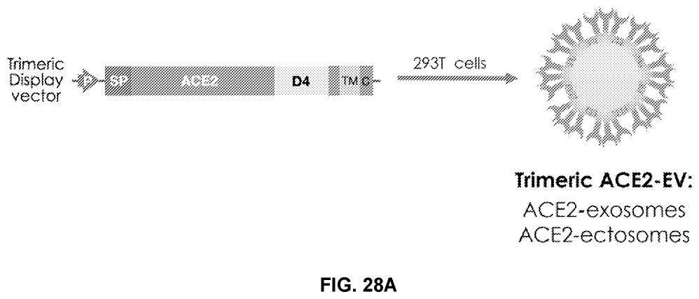

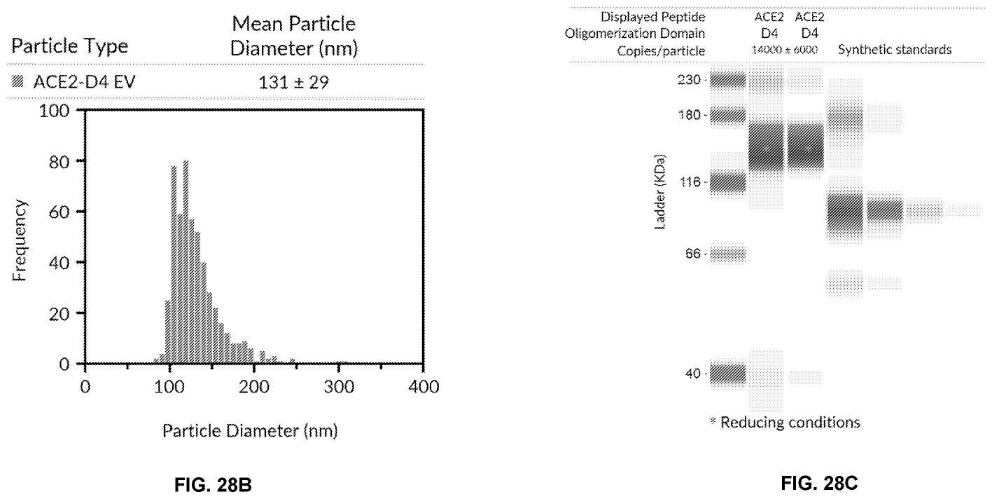

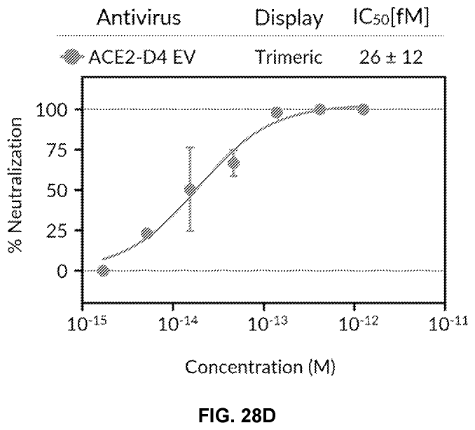

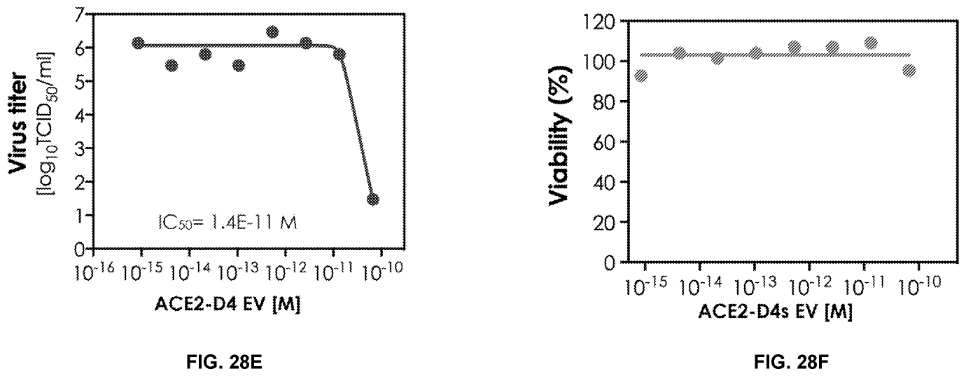

By transfecting 293T cells with only the trimeric ACE2-D4 displaying vector (FIG. 28A), extracellular vesicles (EVs) displaying multiple copies of ACE2 were generated, termed ACE2-D4 EVs. The concentration and particle size distribution of ACE2-D4-EVs were determined by Tunable Resistive Pulse Sensing analysis (TRPS) using a qNano instrument (FIG. 28B). The sizes of ACE2-D4-EVs were in the range of 131±29 nm (FIG. 28B). Each ACE2-D4-EVs particle displayed approximately 14000±6000 copies of ACE2-D4VG fusion molecules (FIG. 28C) through quantitative Western-blot analyses. The neutralizing activity of ACE2-D4-EVs was determined in a SARS CoV-2 pseudovirus infection assay using 293T/ACE2 cells as target cells and ACE2-D4-EV displaying trimeric H2A/ACE2 were highly potent inhibitors, neutralizing CoV-2 pseudovirus at IC₅₀s of 26=12 fM (FIG. 28D). The neutralizing activity (FIG. 28E) and cytoxicity (FIG. 28F) of ACE2-D4-EVs were determined in a SARS CoV-2 live virus neutralization assay. Trimeric ACE2-D4-EVs neutralized live CoV-2 virus at an IC₅₀ of 14 pM in a post-infection live SARS CoV-2 microneutralization assay and reduced viral titer by over five logs (FIG. 28E) without noticeable cytotoxicity (FIG. 28F). These results demonstrated that oligomerized ACE2-D4-EVs were highly potent against live CoV-2 virus infection.

### Example 16. Generation of Co-Stimulatory EV-Based MVPs

This example illustrates the design and production of co-stimulatory EVs. T cell activation by antigen presenting cells (APCs) requires two kinds of stimuli and involves multivalent interactions between large numbers of molecules on both cell types. T cell receptor (TCR) interaction with peptide-major histocompatibility complex (MHC) molecules on the membrane of APCs provides the first antigen-specific signal, while the interaction between co-stimulatory molecules expressed on the membrane of APC and the T cell provide the second co-stimulatory signal. The costimulatory signals are important for triggering T cell activation and differentiation and to prevent T cell anergy. For example, the CD28, 4-1BB, OX40, ICOS and GITR pathways provide co-stimulatory signals for T-cell activation, while the CTLA-4, PD-1, BTLA, and CD160 pathways provide co-inhibitory signals countering T-cell activation. The CD28-B7 family and the tumor necrosis factor superfamily (TNFSF) and TNF receptor superfamily (TNFRSF) represent two major families of co-signal receptors and their ligands in T-cell activation. Most co-signal ligands are expressed or induced on antigen-presenting cells (APCs), such as dendritic cells (DCs), B cells, and macrophages, while most co-signal receptors on T cells are constitutively expressed or induced after TCR activation. Extracellular vesicles (EVs) displaying oligomerized, multivalent co-stimulatory molecules—termed Co-Stim EVs—may be used to mimic the costimulatory signals provided by APCs to T cells.

To this end, Co-Stim EVs were designed and generated for co-stimulatory signal molecules, including CD28, 4-1BB and OX40, by displaying their corresponding ligands on EVs. These ligands are CD80/CD86, 4-1BB ligand (4-1BBL), and OX40 ligand (OX40L), respectively (Table 9).

TABLE 9
List of Co-Stimulatory
Molecules on T cells and APCs

| Co-Stimulatory | |
|---|---|
| molecules (T cells) | Ligand (APC) |
| CD28 | CD80/CD86 |
| 4-1BB | 4-1BBL |
| OX40 | OX40L |

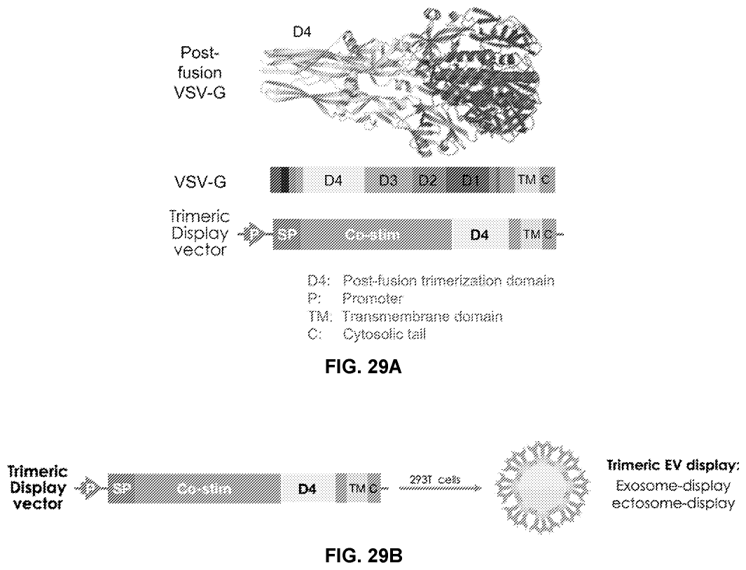

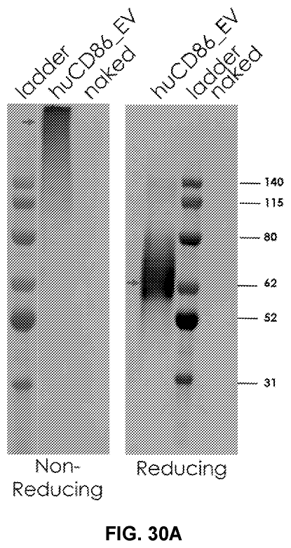

Vectors were designed to express fusion proteins comprising the extracellular domain of the co-stimulatory molecule, the D4 trimerization domain from VSVG, and the transmembrane and cytosolic domains of VSVG to display these co-stimulatory molecules on EVs in oligomerized multivalent formats (FIG. 29A). Co-Stim EVs were then produced by transfecting these display vectors into 293T cells and purified through PEG precipitation and high-speed centrifugation (FIG. 29B). Co-Stim EVs were characterized and their function was examined in T cell activation, differentiation, and proliferation.

### Characterization and Function of CD86-EVs

Along with CD80, CD86 provides costimulatory signals necessary for T cell activation and survival. CD86 belongs to the B7 family of the immunoglobulin superfamily. Both CD80 and CD86 bind as ligands to the costimulatory molecule CD28 on the surface of all naïve T cells, and to the inhibitory receptor cytotoxic T-lymphocyte antigen-4 (CTLA-4). The interaction between CD86 expressed on the surface of an APC with CD28 on the surface of T cell is important for T cell activation. This interaction is helpful for T lymphocytes to receive the full activation signal, which in turn leads to T cell differentiation and division, production of Interleukin 2 (IL-2) and cell expansion.

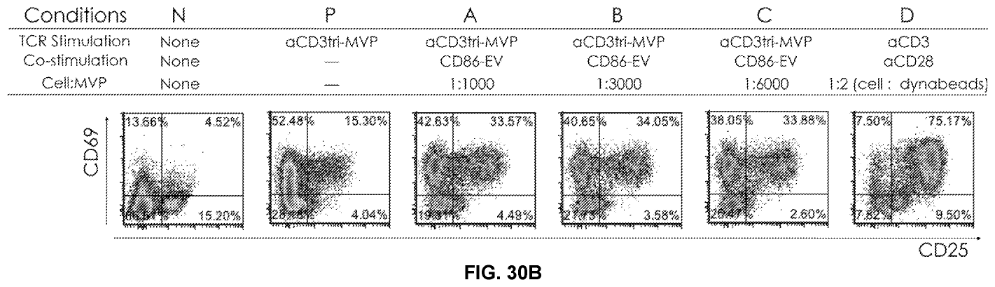

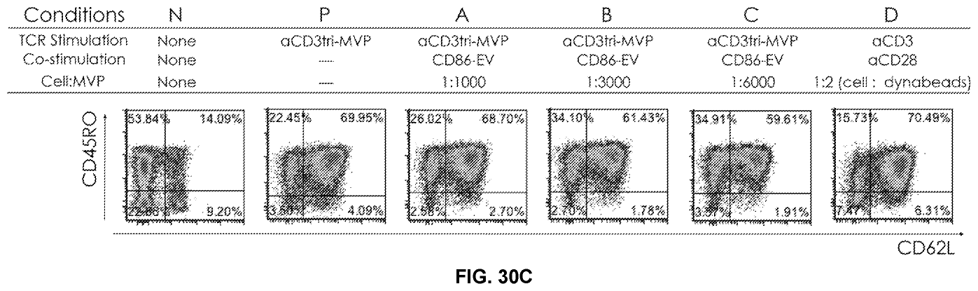

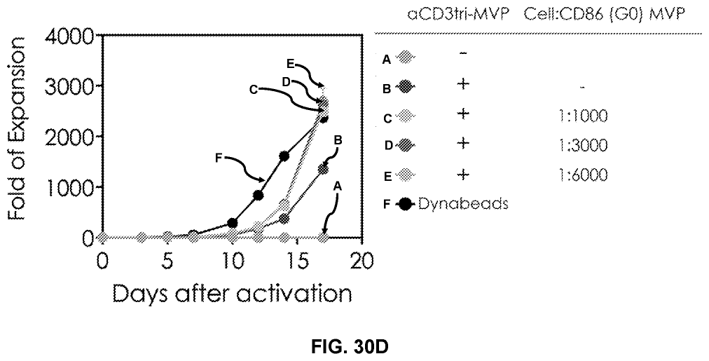

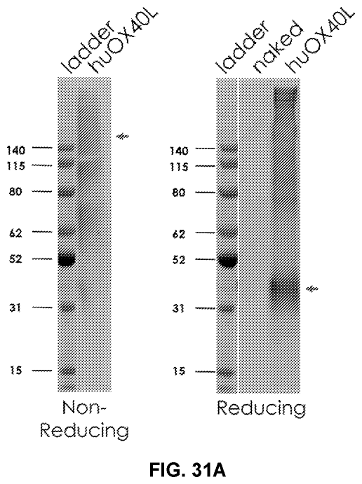

To this end, human CD86-EVs (huCD86-EVs) were generated and the display of CD86 on EVs was confirmed by western-blot analyses under non-reducing and reducing conditions, using antibody specific for human CD86 (FIG. 30A). The results demonstrated robust, oligomeric expression of human CD86 on EVs, as evidenced by the presence of larger, oligomeric bands under non-reducing conditions (FIG. 30A). The concentration of huCD86-EVs was determined by TRPS (qNano, IZON). Furthermore, it was examined whether CD86-EVs can provide co-stimulatory signal to T cells (FIG. 30B). T cells were activated with anti-CD3 particles (αCD3tri-MVP) in the presence of CD86-EVs at increasing EV to T cell ratios, 1000:1, 3000:1, 6000:1 (FIG. 30B). T cells were also activated using αCD3tri-MVPs alone without any co-stimulatory signals, or with co-stimulatory anti-CD3/CD28 dynabeads (FIG. 30B). FACS analysis was carried out starting on day 2 post-T cell stimulation to evaluate the expression of early T cell activation markers CD69 and CD25 on stimulated T-cells (FIG. 30B). The results indicated that CD69 expression levels rapidly increased on T cells, peaking on days 1 and 2 and declining on days 4 and 5. CD25 was up-regulated a slightly later than CD69, and was detected beginning on day 2. It further showed that T cells activated with αCD3tri-MVPs alone, without any costimulatory signal, consisted of roughly 15% CD69 and CD25 double-positive cells, whereas T cells activated with αCD3tri-MVPs and varied ratios of co-stimulatory huCD86-EVs had ˜34% CD69 and CD25 double-positive cells, demonstrating that huCD86-EVs provide robust co-stimulation to T cells (FIG. 30B). For comparison, T cells activated with anti-CD3/CD28 dynabeads consisted of roughly 75% CD69 and CD25 double-positive cells (FIG. 30B). Each CD3/CD28 dynabead can provide both T cell receptor activation and co-stimulatory signals, whereas &CD3tri-MVPs and huCD86-EVs provide two distinct signals, which may contribute to the difference in activation.

It was also examined whether huCD86-EVs can affect T cell differentiation, in particular the development of the central memory phenotype, as indicated by double CD62L and CD45RO expression via FACS analysis. The results indicated that higher concentrations of huCD86-EVs suppressed the development of T cells with CD62L and CD45RO double-positive central memory phenotype (FIG. 30C).

T cell activation induces T cell proliferation. Numerous factors, including ligand density, culture conditions, and starting cell population variability affect direct comparison between T cells expansion. In this example, the starting cell number was set at 50,000 in constant culture conditions with the same amount of IL-2 (600 u/mL) to examine the effects of huCD86-EVs on T cell proliferation. The results indicated that co-stimulatory huCD86-EVs further boosted T cell proliferation to levels comparable to proliferation demonstrated by T cells activated by anti-CD3/28 dynabeads, as compared to T cells activated with αCD3tri-MVP only (FIG. 30D).

Collectively, these results demonstrated that huCD86-EVs displayed high levels of functional co-stimulatory CD86 in oligomerized format and provided potent co-stimulatory signals to T cells during activation.

### Characterization and Function of OX40L-EVs

OX40L is the ligand for OX40 (also known as CD134 or TNFRSF4) and is expressed on many antigen-presenting cells (APCs) such as DC2s (a subtype of dendritic cells), macrophages, and activated B lymphocytes. Co-stimulatory signals from OX40 to a conventional T cell promote division and survival, augmenting expansion of effector and memory populations. OX40L, when expressed in conjunction with 4-1BBL, can provide a synergistic, co-stimulatory signal to an antigen reacting, naïve CD4 positive T cell, prolonging T cell proliferation and increasing production of several cytokines.

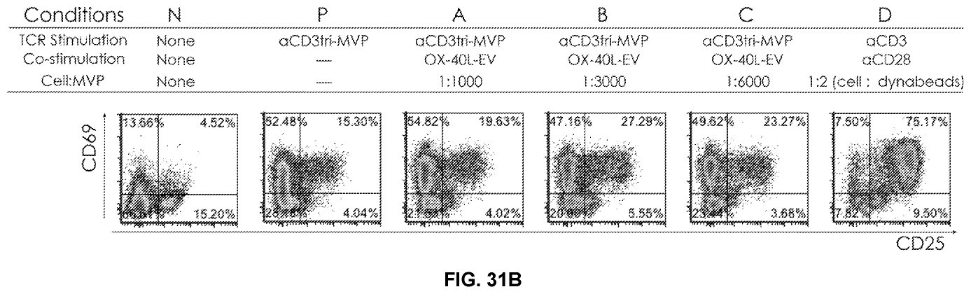

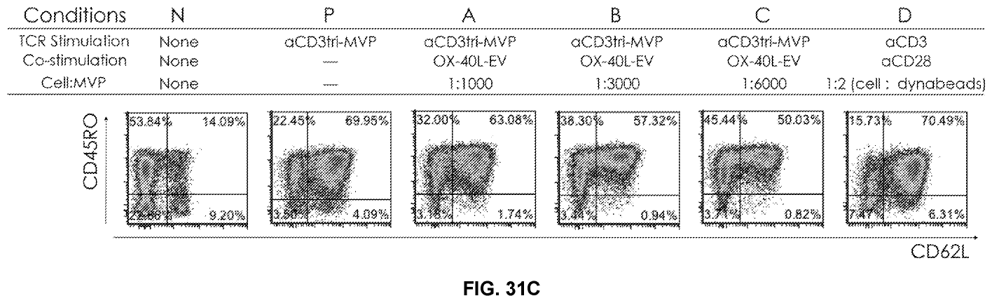

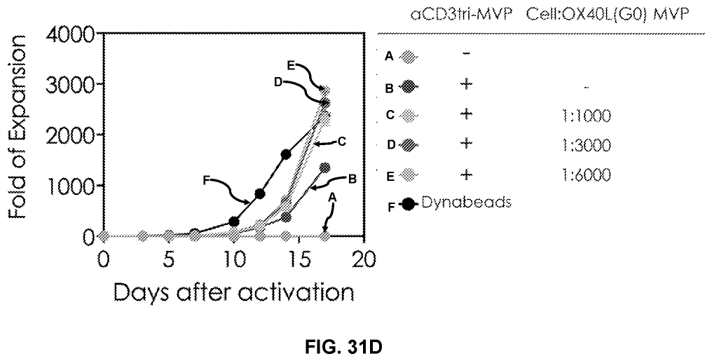

To this end, human OX40L-EVs (huOX40L-EVs) were generated and the display of OX40L on the EVs was confirmed by western-blot analyses under non-reducing and reducing conditions using antibody specific for human OX40L (FIG. 31A). Western blot analysis demonstrated robust expression of oligomeric, human OX40L on EVs (FIG. 31A). The oligomerization of OX40L on EVs cannot be effectively disrupted under the reducing conditions, as evidenced by larger oligomeric bands visible under reducing and non-reducing conditions (FIG. 31A). The concentration of purified huOX40L-EVs was determined via TRPS (qNano, IZON). Then, it was examined whether huOX40L-EVs can provide co-stimulatory signals to T cells (FIG. 31B). T cells were activated with anti-CD3 particles (αCD3tri-MVP) in the presence of co-stimulatory huOX40L-EVs at various EV to cell ratios (FIG. 31B). Activation was compared to T cells activated with αCD3tri-MVPs alone with no co-stimulatory signals, or activated with anti-CD3/CD28 Dynabeads (FIG. 31B). FACS analyses were then carried out at day-2 post-T cell stimulation to evaluate the expression of early T cell activation markers CD69 and CD25 on stimulated T-cells (FIG. 31B). CD69 levels rapidly increased on stimulated T cells, peaking on day 1 and 2 and declining beginning on days 4 and 5. CD25 was up-regulated later than CD69, and was detected beginning on day 2. The results indicated that T cells activated with αCD3tri-MVPs alone consisted of roughly 15% CD69 and CD25 double-positive cells, whereas T cells activated with αCD3tri-MVPs and varied ratios of co-stimulatory huOX40L-EVs consisted of roughly 27-29% CD69 and CD25 double-positive cells, demonstrating that huOX40L-EVs provide significant co-stimulatory signals to T cells (FIG. 31B). T cells activated with standard co-stimulatory anti-CD3/CD28 Dynabeads consisted of 75% CD69 and CD25 double-positive cells (FIG. 31B). Each CD3/CD28 Dynabead can provide both T cell receptor activation and co-stimulatory signals, whereas &CD3tri-MVPs and huOX40L-EVs provide two signals separately, which may contribute to the difference in activation.

Next, it was examined whether huOX40L-EVs can affect T cell differentiation, in particular the development of the central memory T cell phenotype, as indicated by double CD62L and CD45RO expression via FACS analysis. The results indicated that higher levels of huOX40L-EVs suppressed the development of central memory phenotype, CD62L and CD45RO double-positive T cells (FIG. 31C).

T cell activation induces T cell proliferation. Numerous factors, including ligand density, culture conditions and starting cell population variability can affect direct comparisons between T cells expansion. In this example, the starting cell number was set at 50,000 in the constant culture conditions with the same concentration of IL-2 (600 u/mL) and the effects of huOX40L-EVs on T cell proliferation were examine. The results indicated that huOX40L-EVs can further boost T cell proliferation to levels comparable to those of T cells activated by anti-CD3/28 dynabeads, compared to T cells activated with αCD3tri-MVP alone (FIG. 31D).

Collectively, these results demonstrated that huOX40Ls-EVs display high levels of co-stimulatory molecules in oligomerized format and provided potent co-stimulatory signals to T cells during activation.

### Characterization and Function of 41BB-EVs

4-1BBL is a type 2 transmembrane glycoprotein receptor found on antigen presenting cells (APCs) that binds to 4-1BB (also known as CD137). The interaction between 4-1BB and 4-1BBL provides co-stimulatory signals to a variety of T cells, which have antitumor effects in some model systems. While CD28 co-stimulatory signaling contributes to initial T cell expansion, 4-1BBL co-stimulation contributes to memory CD8 T cell survival. Co-stimulation using 4-1BB ligand (4-1BBL) or agonistic anti-4-1BB antibodies can prolong T-cell responses, avoid activation-induced cell death and can be used to discover cancer immunotherapies.

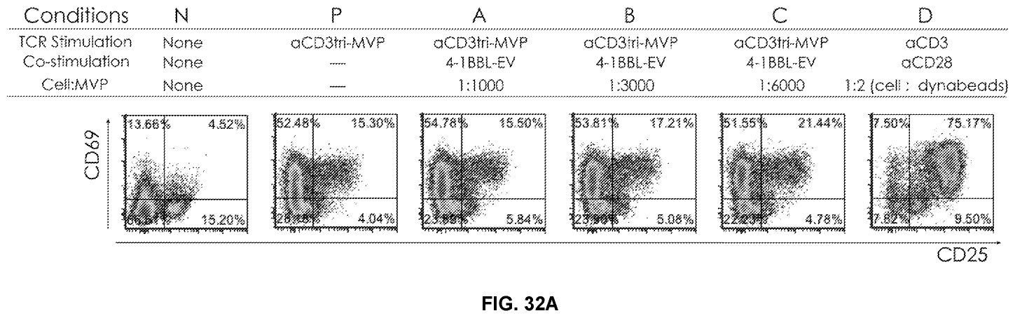

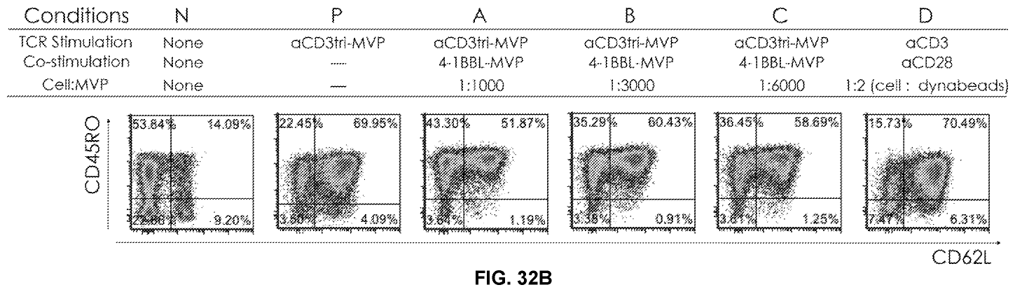

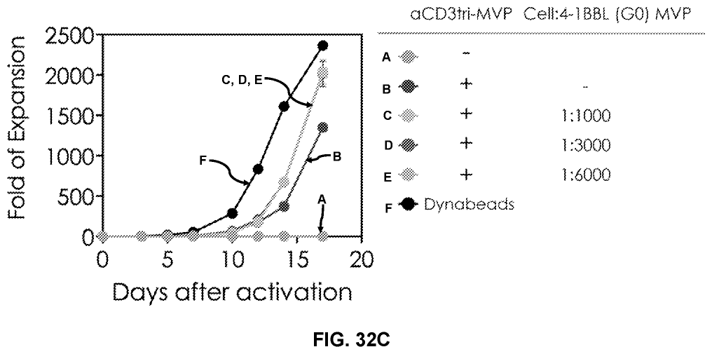

To this end, human 41BBL-EVs (hu41BBL-EVs) displaying oligomeric 4-1BBL were generated and their concentration was determined via TRPS (qNano, IZON). It was then examined whether hu41BBL-EVs can provide co-stimulatory signals to T cells (FIG. 32A). T cells were activated with anti-CD3 particles (αCD3tri-MVP) in the presence of co-stimulatory hu41BBL-EVs at various EV to T cell ratios (FIG. 32A). T cells were also activated with αCD3tri-MVPs alone without a co-stimulatory signal, or with anti-CD3/CD28 Dynabeads (FIG. 32A). FACS analysis was then carried out at day 2 post-T cell stimulation to evaluate the expression of early T cell activation markers CD69 and CD25 on stimulated T-cells (FIG. 32A). The results indicated that T cells activated with αCD3tri-MVPs alone consisted of roughly 15% CD69 and CD25 double-positive cells, whereas T cells activated with αCD3tri-MVPs and varied ratios of co-stimulatory hu41BBL-EVs had as much as 21% CD69 and CD25 double-positive cells (FIG. 32A), demonstrating that hu41BBL-EVs provided significant co-stimulatory signals to T cells. T cells activated by anti-CD3/CD28 Dynabeads consist of roughly 75% of CD69 and CD25 double-positive cells (FIG. 32A). Each CD3/CD28 dynabead can provide both T cell receptor activation and co-stimulatory signals, whereas αCD3tri-MVPs and hu41BBL-EVs provide two signals separately, which may contribute to the difference. It was further examined whether hu41BBL-EVs can affect T cell differentiation, in particular the development of the central memory T cell phenotype, as indicated by double CD62L and CD45RO expression via FACS analysis. The results indicated that higher levels of hu41BBL-EVs suppressed the development of T cells with CD62L and CD45RO double-positive central memory phenotype (FIG. 32B).

T cell activation induces T cell proliferation. Numerous factors, including ligand density, culture conditions and starting cell population variability can affect direct comparisons between T cells expansion. In this example, the starting cell numbers were set at 50,000 in the constant culture conditions with the same concentration of IL-2 (600 u/mL) and the effects of hu41BBL-EVs on T cell proliferation were examined. The results showed that hu41BBL-EVs can further boost T cell proliferation to levels slightly lower than those achieved by T cells activated by anti-CD3/28 Dynabeads (FIG. 32C).

Collectively, these results demonstrated that huOX40Ls-EVs displayed high levels of co-stimulatory molecules in oligomerized format and provided potent co-stimulatory signals to T cells during activation.

### Example 17. Exemplary Recombinant Fusion Protein Sequences

Table 10 lists exemplary recombinant fusion protein sequences which include one or more of a display peptide, an oligomerization domain, transmembrane domain, and a cytosolic domain.

TABLE 10
Exemplary Recombinant Fusion Protein Sequences

| Name | Display Peptide | OD¹ | TM² | CD³ | Amino Acid Sequences | SEQ ID No: |
|---|---|---|---|---|---|---|
| ACE2/ | ACE2 | None | VM⁴ | — | MSSSSWLLLSLVAVTAAQSTIEEQAKTFL | 71 |
| VM | ectodomain | | | | DKFNHEAEDLFYQSSLASWNYNTNITEEN | |
| | | | | | VQNMNNAGDKWSAFLKEQSTLAQMYPL | |
| | | | | | QEIQNLTVKLQLQALQQNGSSVLSEDKSK | |
| | | | | | RLNTILNTMSTIYSTGKVCNPDNPQECLL | |
| | | | | | LEPGLNEIMANSLDYNERLWAWESWRSE | |
| | | | | | VGKQLRPLYEEYVVLKNEMARANHYED | |
| | | | | | YGDYWRGDYEVNGVDGYDYSRGQLIED | |
| | | | | | VEHTFEEIKPLYEHLHAYVRAKLMNAYP | |
| | | | | | SYISPIGCLPAHLLGDMWGRFWTNLYSLT | |
| | | | | | VPFGQKPNIDVTDAMVDQAWDAQRIFKE | |
| | | | | | AEKFFVSVGLPNMTQGFWENSMLTDPGN | |
| | | | | | VQKAVCHPTAWDLGKGDFRILMCTKVT | |
| | | | | | MDDFLTAHHEMGHIQYDMAYAAQPFLL | |
| | | | | | RNGANEGFHEAVGEIMSLSAATPKHLKSI | |
| | | | | | GLLSPDFQEDNETEINFLLKQALTIVGTLP | |
| | | | | | FTYMLEKWRWMVFKGEIPKDQWMKKW | |
| | | | | | WEMKREIVGVVEPVPHDETYCDPASLFH | |
| | | | | | VSNDYSFIRYYTRTLYQFQFQEALCQAAK | |
| | | | | | HEGPLHKCDISNSTEAGQKLFNMLRLGKS | |
| | | | | | EPWTLALENVVGAKNMNVRPLLNYFEPL | |
| | | | | | FTWLKDQNKNSFVGWSTDWSPYADQSIK | |
| | | | | | VRISLKSALGDKAYEWNDNEMYLFRSSV | |
| | | | | | AYAMRQYFLKVKNQMILFGEEDVRVAN | |
| | | | | | LKPRISFNFFVTAPKNVSDIIPRTEVEKAIR | |
| | | | | | MSRSRINDAFRLNDNSLEFLGIQPTLGPPN | |
| | | | | | QPPVSSRGMLDSDLHLSSKAQVFEHPHIQ | |
| | | | | | DAASQLPDDESLFFGDTGLSKNPIELVEG | |
| | | | | | WFSSWKSSIASFFFIIGLIIGLFLVLRVGIHL | |
| | | | | | CIKLKHTKKRQIYTDIEMNRLGK | |
| ACE2/LZ | ACE2 | LZ⁵ | VM⁴ | — | MSSSSWLLLSLVAVTAAQSTIEEQAKTFL | 72 |
| | ectodomain | | | | DKFNHEAEDLFYQSSLASWNYNTNITEEN | |
| | | | | | VQNMNNAGDKWSAFLKEQSTLAQMYPL | |
| | | | | | QEIQNLTVKLQLQALQQNGSSVLSEDKSK | |
| | | | | | RLNTILNTMSTIYSTGKVCNPDNPQECLL | |
| | | | | | LEPGLNEIMANSLDYNERLWAWESWRSE | |
| | | | | | VGKQLRPLYEEYVVLKNEMARANHYED | |
| | | | | | YGDYWRGDYEVNGVDGYDYSRGQLIED | |
| | | | | | VEHTFEEIKPLYEHLHAYVRAKLMNAYP | |
| | | | | | SYISPIGCLPAHLLGDMWGRFWTNLYSLT | |
| | | | | | VPFGQKPNIDVTDAMVDQAWDAQRIFKE | |
| | | | | | AEKFFVSVGLPNMTQGFWENSMLTDPGN | |
| | | | | | VQKAVCHPTAWDLGKGDFRILMCTKVT | |
| | | | | | MDDFLTAHHEMGHIQYDMAYAAQPFLL | |
| | | | | | RNGANEGFHEAVGEIMSLSAATPKHLKSI | |
| | | | | | GLLSPDFQEDNETEINFLLKQALTIVGTLP | |
| | | | | | FTYMLEKWRWMVFKGEIPKDQWMKKW | |
| | | | | | WEMKREIVGVVEPVPHDETYCDPASLFH | |
| | | | | | VSNDYSFIRYYTRTLYQFQFQEALCQAAK | |
| | | | | | HEGPLHKCDISNSTEAGQKLFNMLRLGKS | |
| | | | | | EPWTLALENVVGAKNMNVRPLLNYFEPL | |
| | | | | | FTWLKDQNKNSFVGWSTDWSPYADQSIK | |
| | | | | | VRISLKSALGDKAYEWNDNEMYLFRSSV | |
| | | | | | AYAMRQYFLKVKNQMILFGEEDVRVAN | |
| | | | | | LKPRISFNFFVTAPKNVSDIIPRTEVEKAIR | |
| | | | | | MSRSRINDAFRLNDNSLEFLGIQPTLGPPN | |
| | | | | | QPPVS | |
| ACE2/D4 | ACE2 | ⁶D4 | VM⁴ | - | MSSSSWLLLSLVAVTAAQSTIEEQAKTFL | 73 |
| | ectodomain | | | | DKFNHEAEDLFYQSSLASWNYNTNITEEN | |
| | | | | | VQNMNNAGDKWSAFLKEQSTLAQMYPL | |
| | | | | | QEIQNLTVKLQLQALQQNGSSVLSEDKSK | |
| | | | | | RLNTILNTMSTIYSTGKVCNPDNPQECLL | |
| | | | | | LEPGLNEIMANSLDYNERLWAWESWRSE | |
| | | | | | VGKQLRPLYEEYVVLKNEMARANHYED | |
| | | | | | YGDYWRGDYEVNGVDGYDYSRGQLIED | |
| | | | | | VEHTFEEIKPLYEHLHAYVRAKLMNAYP | |
| | | | | | SYISPIGCLPAHLLGDMWGRFWTNLYSLT | |
| | | | | | VPFGQKPNIDVTDAMVDQAWDAQRIFKE | |
| | | | | | AEKFFVSVGLPNMTQGFWENSMLTDPGN | |
| | | | | | VQKAVCHPTAWDLGKGDFRILMCTKVT | |
| | | | | | MDDFLTAHAEMGAIQYDMAYAAQPFLL | |
| | | | | | RNGANEGFHEAVGEIMSLSAATPKHLKSI | |
| | | | | | GLLSPDFQEDNETEINFLLKQALTIVGTLP | |
| | | | | | FTYMLEKWRWMVFKGEIPKDQWMKKW | |
| | | | | | WEMKREIVGVVEPVPHDETYCDPASLFH | |
| | | | | | VSNDYSFIRYYTRTLYQFQFQEALCQAAK | |
| | | | | | HEGPLHKCDISNSTEAGQKLFNMLRLGKS | |
| | | | | | EPWTLALENVVGAKNMNVRPLLNYFEPL | |
| | | | | | FTWLKDQNKNSFVGWSTDWSPYADQSIK | |
| | | | | | VRISLKSALGDKAYEWNDNEMYLFRSSV | |
| | | | | | AYAMRQYFLKVKNQMILFGEEDVRVAN | |
| | | | | | LKPRISFNFFVTAPKNVSDIIPRTEVEKAIR | |
| | | | | | MSRSRINDAFRLNDNSLEFLGIQPTLGPPN | |
| | | | | | QPPVSSRIQADGWMCHASKWVTTCDFR | |
| | | | | | WYGPKYITHSIRSFTPSVEQCKESIEQTKQ | |
| | | | | | GTWLNPGFPPQSCGYATVIDAEAVIVQV | |
| | | | | | TPHHVLVDEYTGEWVDSQFINGKCSNYIC | |
| | | | | | PTVHNSTTWHSDYKVKGLCDSNLGMLDS | |
| | | | | | DLHLSSKAQVFEHPHIQDAASQLPDDESL | |
| | | | | | FFGDTGLSKNPIELVEGWFSSWKSSIASFF | |
| | | | | | FIIGLIIGLFLVLRVGIHLCIKLKHTKKRQI | |
| | | | | | YTDIEMNRLGK | |
| ACE2/ | ACE2 | DE⁷ | VM⁴ | — | MSSSSWLLLSLVAVTAAQSTIEEQAKTFL | 74 |
| DE | ectodomain | | | | DKFNHEAEDLFYQSSLASWNYNTNITEEN | |
| | | | | | VQNMNNAGDKWSAFLKEQSTLAQMYPL | |
| | | | | | QEIQNLTVKLQLQALQQNGSSVLSEDKSK | |
| | | | | | RLNTILNTMSTIYSTGKVCNPDNPQECLL | |
| | | | | | LEPGLNEIMANSLDYNERLWAWESWRSE | |
| | | | | | VGKQLRPLYEEYVVLKNEMARANHYED | |
| | | | | | YGDYWRGDYEVNGVDGYDYSRGQLIED | |
| | | | | | VEHTFEEIKPLYEHLHAYVRAKLMNAYP | |
| | | | | | SYISPIGCLPAHLLGDMWGRFWTNLYSLT | |
| | | | | | VPFGQKPNIDVTDAMVDQAWDAQRIFKE | |
| | | | | | AEKFFVSVGLPNMTQGFWENSMLTDPGN | |
| | | | | | VQKAVCHPTAWDLGKGDFRILMCTKVT | |
| | | | | | MDDFLTAHHEMGHIQYDMAYAAQPFLL | |
| | | | | | RNGANEGFHEAVGEIMSLSAATPKHLKSI | |
| | | | | | GLLSPDFQEDNETEINFLLKQALTIVGTLP | |
| | | | | | FTYMLEKWRWMVFKGEIPKDQWMKKW | |
| | | | | | WEMKREIVGVVEPVPHDETYCDPASLFH | |
| | | | | | VSNDYSFIRYYTRTLYQFQFQEALCQAAK | |
| | | | | | HEGPLHKCDISNSTEAGQKLFNMLRLGKS | |
| | | | | | EPWTLALENVVGAKNMNVRPLLNYFEPL | |
| | | | | | FTWLKDQNKNSFVGWSTDWSPYADQSIK | |
| | | | | | VRISLKSALGDKAYEWNDNEMYLFRSSV | |
| | | | | | AYAMRQYFLKVKNQMILFGEEDVRVAN | |
| | | | | | LKPRISFNFFVTAPKNVSDIIPRTEVEKAIR | |
| | | | | | MSRSRINDAFRLNDNSLEFLGIQPTLGPPN | |
| | | | | | QPPVS | |
| ACE2/ | ACE2 | Fold⁸ | VM⁴ | — | MSSSSWLLLSLVAVTAAQSTIEEQAKTFL | 75 |
| Fold | ectodomain | | | | DKFNHEAEDLFYQSSLASWNYNTNITEEN | |
| | | | | | VQNMNNAGDKWSAFLKEQSTLAQMYPL | |
| | | | | | QEIQNLTVKLQLQALQQNGSSVLSEDKSK | |
| | | | | | RLNTILNTMSTIYSTGKVCNPDNPQECLL | |
| | | | | | LEPGLNEIMANSLDYNERLWAWESWRSE | |
| | | | | | VGKQLRPLYEEYVVLKNEMARANHYED | |
| | | | | | YGDYWRGDYEVNGVDGYDYSRGQLIED | |
| | | | | | VEHTFEEIKPLYEHLHAYVRAKLMNAYP | |
| | | | | | SYISPIGCLPAHLLGDMWGRFWTNLYSLT | |
| | | | | | VPFGQKPNIDVTDAMVDQAWDAQRIFKE | |
| | | | | | AEKFFVSVGLPNMTQGFWENSMLTDPGN | |
| | | | | | VQKAVCHPTAWDLGKGDFRILMCTKVT | |
| | | | | | MDDFLTAHHEMGHIQYDMAYAAQPFLL | |
| | | | | | RNGANEGFHEAVGEIMSLSAATPKHLKSI | |
| | | | | | GLLSPDFQEDNETEINFLLKQALTIVGTLP | |
| | | | | | FTYMLEKWRWMVFKGEIPKDQWMKKW | |
| | | | | | WEMKREIVGVVEPVPHDETYCDPASLFH | |
| | | | | | VSNDYSFIRYYTRTLYQFQFQEALCQAAK | |
| | | | | | HEGPLHKCDISNSTEAGQKLFNMLRLGKS | |
| | | | | | EPWTLALENVVGAKNMNVRPLLNYFEPL | |
| | | | | | FTWLKDQNKNSFVGWSTDWSPYADQSIK | |
| | | | | | VRISLKSALGDKAYEWNDNEMYLFRSSV | |
| | | | | | AYAMRQYFLKVKNQMILFGEEDVRVAN | |
| | | | | | LKPRISFNFFVTAPKNVSDIIPRTEVEKAIR | |
| | | | | | MSRSRINDAFRLNDNSLEFLGIQPTLGPPN | |
| | | | | | QPPVS | |
| DPP4/ | DPP4 | ⁹NA- | — | — | MNPNQKIITIGSICLVVGLISLILQIGNIISIW | 76 |
| NA-V1 | ectodomain | V1 | | | ISHSIQTGGSKGTDDATADSRKTYTLTDY | |
| | | | | | LKNTYRLKLYSLRWISDHEYLYKQENNIL | |
| | | | | | VFNAEYGNSSVFLENSTFDEFGHSINDYSI | |
| | | | | | SPDGQFILLEYNYVKQWRHSYTASYDIYD | |
| | | | | | LNKRQLITEERIPNNTQWVTWSPVGHKL | |
| | | | | | AYVWNNDIYVKIEPNLPSYRITWTGKEDII | |
| | | | | | YNGITDWVYEEEVFSAYSALWWSPNGTF | |
| | | | | | LAYAQFNDTEVPLIEYSFYSDESLQYPKT | |
| | | | | | VRVPYPKAGAVNPTVKFFVVNTDSLSSV | |
| | | | | | TNATSIQITAPASMLIGDHYLCDVTWATQ | |
| | | | | | ERISLQWLRRIQNYSVMDICDYDESSGRW | |
| | | | | | NCLVARQHIEMSTTGWVGRFRPSEPHFTL | |
| | | | | | DGNSFYKIISNEEGYRHICYFQIDKKDCTF | |
| | | | | | ITKGTWEVIGIEALTSDYLYYISNEYKGM | |
| | | | | | PGGRNLYKIQLSDYTKVTCLSCELNPERC | |
| | | | | | QYYSVSFSKEAKYYQLRCSGPGLPLYTLH | |
| | | | | | SSVNDKGLRVLEDNSALDKMLQNVQMP | |
| | | | | | SKKLDFIILNETKFWYQMILPPHFDKSKK | |
| | | | | | YPLLLDVYAGPCSQKADTVFRLNWATYL | |
| | | | | | ASTENIIVASFDGRGSGYQGDKIMHAINR | |
| | | | | | RLGTFEVEDQIEAARQFSKMGFVDNKRIA | |
| | | | | | IWGWSYGGYVTSMVLGSGSGVFKCGIAV | |
| | | | | | APVSRWEYYDSVYTERYMGLPTPEDNLD | |
| | | | | | HYRNSTVMSRAENFKQVEYLLIHGTADD | |
| | | | | | NVHFQQSAQISKALVDVGVDFQAMWYT | |
| | | | | | DEDHGIASSTAHQHIYTHMSHFIKQCFSLP | |
| | | | | | AAARGSGLNDIFEAQKIEWHE | |
| DPP4/ | DPP4 | ¹⁰NA- | — | — | MNPNQKIITIGSICMVTGIVSLMLQIGNMI | 77 |
| NA-V2 | ectodomain | V2 | | | SIWVSHSIHTGNQHQSEPISNTNFLTEKAV | |
| | | | | | ASVKLAGNSSLCPINGGSKGTDDATADSR | |
| | | | | | KTYTLTDYLKNTYRLKLYSLRWISDHEY | |
| | | | | | LYKQENNILVFNAEYGNSSVFLENSTFDE | |
| | | | | | FGHSINDYSISPDGQFILLEYNYVKQWRH | |
| | | | | | SYTASYDIYDLNKRQLITEERIPNNTQWV | |
| | | | | | TWSPVGHKLAYVWNNDIYVKIEPNLPSY | |
| | | | | | RITWTGKEDIIYNGITDWVYEEEVFSAYS | |
| | | | | | ALWWSPNGTFLAYAQFNDTEVPLIEYSFY | |
| | | | | | SDESLQYPKTVRVPYPKAGAVNPTVKFF | |
| | | | | | VVNTDSLSSVTNATSIQITAPASMLIGDHY | |
| | | | | | LCDVTWATQERISLQWLRRIQNYSVMDI | |
| | | | | | CDYDESSGRWNCLVARQHIEMSTTGWV | |
| | | | | | GRFRPSEPHFTLDGNSFYKIISNEEGYRHI | |
| | | | | | CYFQIDKKDCTFITKGTWEVIGIEALTSDY | |
| | | | | | LYYISNEYKGMPGGRNLYKIQLSDYTKV | |
| | | | | | TCLSCELNPERCQYYSVSFSKEAKYYQLR | |
| | | | | | CSGPGLPLYTLHSSVNDKGLRVLEDNSAL | |
| | | | | | DKMLQNVQMPSKKLDFIILNETKFWYQM | |
| | | | | | ILPPHFDKSKKYPLLLDVYAGPCSQKADT | |
| | | | | | VFRLNWATYLASTENIIVASFDGRGSGYQ | |
| | | | | | GDKIMHAINRRLGTFEVEDQIEAARQFSK | |
| | | | | | MGFVDNKRIAIWGWSYGGYVTSMVLGS | |
| | | | | | GSGVFKCGIAVAPVSRWEYYDSVYTERY | |
| | | | | | MGLPTPEDNLDHYRNSTVMSRAENFKQV | |
| | | | | | EYLLIHGTADDNVHFQQSAQISKALVDV | |
| | | | | | GVDFQAMWYTDEDHGIASSTAHQHIYTH | |
| | | | | | MSHFIKQCFSLPAAARGSGLNDIFEAQKIE | |
| | | | | | WHE | |
| αNTD: | Anti-spike | ⁶D4 | VM⁴ | — | MEFGLSWVFLVALFRGVQSQVQLVQSGA | 78 |
| CV26/ | NTD CV26 | | | | EVKKPGASVKVSCKVSGYTLTELSIHWV | |
| D4 | scFv | | | | RQAPGKGLEWMGGFDPEDGETVYAQKF | |
| | | | | | QGRVTMTEDTSSDTAYMELSSLRSEDTA | |
| | | | | | VYYCATSFPIRGDPSYYYYYYGMDVWG | |
| | | | | | QGTTVTVSSGGGGSGGGGSGGGGSGGGG | |
| | | | | | SDIQMTQSPSSLSASVGDRVTITCRASQGI | |
| | | | | | TNYLAWFQQKPGKAPKSLIYAVSSLQSG | |
| | | | | | VPSKFSGSGSGTDFTLTISSLQPEDFATYY | |
| | | | | | CQQYNSYPWTFGQGTKVEIKDIIQADGW | |
| | | | | | MCHASKWVTTCDFRWYGPKYITHSIRSF | |
| | | | | | TPSVEQCKESIEQTKQGTWLNPGFPPQSC | |
| | | | | | GYATVTDAEAVIVQVTPHHVLVDEYTGE | |
| | | | | | WVDSQFINGKCSNYICPTVHNSTTWHSD | |
| | | | | | YKVKGLCDSNLGMLDSDLHLSSKAQVFE | |
| | | | | | HPHIQDAASQLPDDESLFFGDTGLSKNPIE | |
| | | | | | LVEGWFSSWKSSIASFFFIIGLIIGLFLVLR | |
| | | | | | VGIHLCIKLKHTKKRQIYTDIEMNRLGK | |
| αRBD: | Anti-spike | None | VM⁴ | D4_ | MEFGLSWVFLVALFRGVQSEVQLVESGG | 79 |
| C018/ | RBD C018 | | | | GVVQPGRSLRLSCAASGFTFSNYAIHWVR | |
| VM | scFv | | | | QAPGKGLEWVAVISYDGSNKYYADSVK | |
| | | | | | GRFTISRDNSKNTLYLQMNSLRAEDTAV | |
| | | | | | YYCARDFDDSSFWAFDYWGQGTLVTVSS | |
| | | | | | GGGGSGGGGSGGGGSGGGGSDIQLTQSP | |
| | | | | | SSLSASVGDRVTITCRASQSIRSYLNWYQ | |
| | | | | | QKPGKAPKLLIYAASSLQSGVPSRFSGSGS | |
| | | | | | GTDFTLTISSLQPDDFATYYCQQSYSTPPA | |
| | | | | | TFGQGTKLEIKSRGMLDSDLHLSSKAQVF | |
| | | | | | EHPHIQDAASQLPDDESLFFGDTGLSKNPI | |
| | | | | | ELVEGWFSSWKSSIASFFFIIGLIIGLFLVL | |
| | | | | | RVGIHLCIKLKHTKKRQIYTDIEMNRLGK | |
| ACE2/ | ACE2 | ⁶D4 | VM⁴ | D4_ | MSSSSWLLLSLVAVTAAQSTIEEQAKTFL | 80 |
| D4_Cyt | ectodomain | | | Cyt¹¹ | DKFNHEAEDLFYQSSLASWNYNTNITEEN | |
| | | | | | VQNMNNAGDKWSAFLKEQSTLAQMYPL | |
| | | | | | QEIQNLTVKLQLQALQQNGSSVLSEDKSK | |
| | | | | | RLNTILNTMSTIYSTGKVCNPDNPQECLL | |
| | | | | | LEPGLNEIMANSLDYNERLWAWESWRSE | |
| | | | | | VGKQLRPLYEEYVVLKNEMARANHYED | |
| | | | | | YGDYWRGDYEVNGVDGYDYSRGQLIED | |
| | | | | | VEHTFEEIKPLYEHLHAYVRAKLMNAYP | |
| | | | | | SYISPIGCLPAHLLGDMWGRFWTNLYSLT | |
| | | | | | VPFGQKPNIDVTDAMVDQAWDAQRIFKE | |
| | | | | | AEKFFVSVGLPNMTQGFWENSMLTDPGN | |
| | | | | | VQKAVCHPTAWDLGKGDFRILMCTKVT | |
| | | | | | MDDFLTAHAEMGAIQYDMAYAAQPFLL | |
| | | | | | RNGANEGFHEAVGEIMSLSAATPKHLKSI | |
| | | | | | GLLSPDFQEDNETEINFLLKQALTIVGTLP | |
| | | | | | FTYMLEKWRWMVFKGEIPKDQWMKKW | |
| | | | | | WEMKREIVGVVEPVPHDETYCDPASLFH | |
| | | | | | VSNDYSFIRYYTRTLYQFQFQEALCQAAK | |
| | | | | | HEGPLHKCDISNSTEAGQKLFNMLRLGKS | |
| | | | | | EPWTLALENVVGAKNMNVRPLLNYFEPL | |
| | | | | | FTWLKDQNKNSFVGWSTDWSPYADQSIK | |
| | | | | | VRISLKSALGDKAYEWNDNEMYLFRSSV | |
| | | | | | AYAMRQYFLKVKNQMILFGEEDVRVAN | |
| | | | | | LKPRISFNFFVTAPKNVSDIIPRTEVEKAIR | |
| | | | | | MSRSRINDAFRLNDNSLEFLGIQPTLGPPN | |
| | | | | | QPPVSSGMLDSDLHLSSKAQVFEHPHIQD | |
| | | | | | AASQLPDDESLFFGDTGLSKNPIELVEGW | |
| | | | | | FSSWKSSIASFFFIIGLIIGLFLVLRVGIIQA | |
| | | | | | DGWMCHASKWVTTCDFRWYGPKYITHS | |
| | | | | | IRSFTPSVEQCKESIEQTKQGTWLNPGFPP | |
| | | | | | QSCGYATVIDAEAVIVQVTPHHVLVDEY | |
| | | | | | TGEWVDSQFINGKCSNYICPTVHNSTTW | |
| | | | | | HSDYKVKGLCDSNL | |
| ACE2/ | ACE2 | ¹²D4- | VM⁴ | — | MSSSSWLLLSLVAVTAAQSTIEEQAKTFL | 81 |
| D4-V4 | ectodomain | V4 | | | DKFNHEAEDLFYQSSLASWNYNTNITEEN | |
| | | | | | VQNMNNAGDKWSAFLKEQSTLAQMYPL | |
| | | | | | QEIQNLTVKLQLQALQQNGSSVLSEDKSK | |
| | | | | | RLNTILNTMSTIYSTGKVCNPDNPQECLL | |
| | | | | | LEPGLNEIMANSLDYNERLWAWESWRSE | |
| | | | | | VGKQLRPLYEEYVVLKNEMARANHYED | |
| | | | | | YGDYWRGDYEVNGVDGYDYSRGQLIED | |
| | | | | | VEHTFEEIKPLYEHLHAYVRAKLMNAYP | |
| | | | | | SYISPIGCLPAHLLGDMWGRFWTNLYSLT | |
| | | | | | VPFGQKPNIDVTDAMVDQAWDAQRIFKE | |
| | | | | | AEKFFVSVGLPNMTQGFWENSMLTDPGN | |
| | | | | | VQKAVCHPTAWDLGKGDFRILMCTKVT | |
| | | | | | MDDFLTAHAEMGAIQYDMAYAAQPFLL | |
| | | | | | RNGANEGFHEAVGEIMSLSAATPKHLKSI | |
| | | | | | GLLSPDFQEDNETEINFLLKQALTIVGTLP | |
| | | | | | FTYMLEKWRWMVFKGEIPKDQWMKKW | |
| | | | | | WEMKREIVGVVEPVPHDETYCDPASLFH | |
| | | | | | VSNDYSFIRYYTRTLYQFQFQEALCQAAK | |
| | | | | | HEGPLHKCDISNSTEAGQKLFNMLRLGKS | |
| | | | | | EPWTLALENVVGAKNMNVRPLLNYFEPL | |
| | | | | | FTWLKDQNKNSFVGWSTDWSPYADQSIK | |
| | | | | | VRISLKSALGDKAYEWNDNEMYLFRSSV | |
| | | | | | AYAMRQYFLKVKNQMILFGEEDVRVAN | |
| | | | | | LKPRISFNFFVTAPKNVSDIIPRTEVEKAIR | |
| | | | | | MSRSRINDAFRLNDNSLEFLGIQPTLGPPN | |
| | | | | | QPPVSSRIQADGWMCHASKWVTTCDFR | |
| | | | | | WYGPKYITHSIRSFTPSVEQCKESIEQTKQ | |
| | | | | | GTWLNPGFPPQSCGYATVIDAEAVIVQV | |
| | | | | | TPHHVLVDEYTGEWVDSQFINGGMLDSD | |
| | | | | | LHLSSKAQVFEHPHIQDAASQLPDDESLF | |
| | | | | | FGDTGLSKNPIELVEGWFSSWKSSIASFFF | |
| | | | | | IIGLIIGLFLVLRVGIHLCIKLKHTKKRQIY | |
| | | | | | TDIEMNRLGK | |
| ACE2/ | ACE2 | ¹³D4- | VM⁴ | — | MSSSSWLLLSLVAVTAAQSTIEEQAKTFL | 82 |
| D4-V5 | ectodomain | V5 | | | DKFNHEAEDLFYQSSLASWNYNTNITEEN | |
| | | | | | VQNMNNAGDKWSAFLKEQSTLAQMYPL | |
| | | | | | QEIQNLTVKLQLQALQQNGSSVLSEDKSK | |
| | | | | | RLNTILNTMSTIYSTGKVCNPDNPQECLL | |
| | | | | | LEPGLNEIMANSLDYNERLWAWESWRSE | |
| | | | | | VGKQLRPLYEEYVVLKNEMARANHYED | |
| | | | | | YGDYWRGDYEVNGVDGYDYSRGQLIED | |
| | | | | | VEHTFEEIKPLYEHLHAYVRAKLMNAYP | |
| | | | | | SYISPIGCLPAHLLGDMWGRFWTNLYSLT | |
| | | | | | VPFGQKPNIDVTDAMVDQAWDAQRIFKE | |
| | | | | | AEKFFVSVGLPNMTQGFWENSMLTDPGN | |
| | | | | | VQKAVCHPTAWDLGKGDFRILMCTKVT | |
| | | | | | MDDFLTAHAEMGAIQYDMAYAAQPFLL | |
| | | | | | RNGANEGFHEAVGEIMSLSAATPKHLKSI | |
| | | | | | GLLSPDFQEDNETEINFLLKQALTIVGTLP | |
| | | | | | FTYMLEKWRWMVFKGEIPKDQWMKKW | |
| | | | | | WEMKREIVGVVEPVPHDETYCDPASLFH | |
| | | | | | VSNDYSFIRYYTRTLYQFQFQEALCQAAK | |
| | | | | | HEGPLHKCDISNSTEAGQKLFNMLRLGKS | |
| | | | | | EPWTLALENVVGAKNMNVRPLLNYFEPL | |
| | | | | | FTWLKDQNKNSFVGWSTDWSPYADQSIK | |
| | | | | | VRISLKSALGDKAYEWNDNEMYLFRSSV | |
| | | | | | AYAMRQYFLKVKNQMILFGEEDVRVAN | |
| | | | | | LKPRISFNFFVTAPKNVSDIIPRTEVEKAIR | |
| | | | | | MSRSRINDAFRLNDNSLEFLGIQPTLGPPN | |
| | | | | | QPPVSSRIQADGWMCHASKWVTTCDFR | |
| | | | | | WYGPKYITHSIRSFTPSVEQCKESIEQTKQ | |
| | | | | | GTWLNPGFPPQSCGYATVIDAEAVIVQV | |
| | | | | | TPHHVLGMLDSDLHLSSKAQVFEHPHIQD | |
| | | | | | AASQLPDDESLFFGDTGLSKNPIELVEGW | |
| | | | | | FSSWKSSIASFFFIIGLIIGLFLVLRVGIHLCI | |
| | | | | | KLKHTKKRQIYTDIEMNRLGK | |
| mu_ | murine | ⁶D4 | VM⁴ | — | MTFVFTAVLCLGLNLGQETSMSEGNPHK | 83 |
| LILRA5/ | LILRA5 | | | | PTLSVQPGLVVAKGKQVTISCEVTTGARE | |
| D4 | ectodomain | | | | YRLYKEGGPHPWRTRNTPKTTNKAQFLIP | |
| | | | | | SIEQRSGGIYRCYYKTPTGWSEHSDPLEL | |
| | | | | | AVTGLYSKPSLSTQPSNVVNSGETVTLQC | |
| | | | | | VSTLGFNRFVLTKEGEQKWSLIQESEFINS | |
| | | | | | TGQFQGLFTVGPVTPSQRWIFRCYGYHV | |
| | | | | | NSPQVWSEPSDLLEIHVSEADQPLRPSPNI | |
| | | | | | SDPKTVSQPQNYTMENLIRMGASILVLVL | |
| | | | | | LGVLLFEAQHSQRQTQHAAGRESSASFM | |
| | | | | | VADTWEIQADGWMCHASKWVTTCDFR | |
| | | | | | WYGPKYITHSIRSFTPSVEQCKESIEQTKQ | |
| | | | | | GTWLNPGFPPQSCGYATVIDAEAVIVQV | |
| | | | | | TPHHVLVDEYTGEWVDSQFINGKCSNYIC | |
| | | | | | PTVHNSTTWHSDYKVKGLCDSNLGMLDS | |
| | | | | | DLHLSSKAQVFEHPHIQDAASQLPDDESL | |
| | | | | | FFGDTGLSKNPIELVEGWFSSWKSSIASFF | |
| | | | | | FIIGLIIGLFLVLRVGIHLCIKLKHTKKRQI | |
| | | | | | YPYDVPDYA | |
| mu_ | murine | ⁶D4 | VM⁴ | — | MSCTFTALLRLGLTLSLWIPVLTGSLPKPI | 84 |
| LILRB3/ | LILRB3 | | | | LRVQPDSVVSRRTKVTFLCEETIGANEYR | |
| D4 | ectodomain | | | | LYKDGKLYKTVTKNKQKPENKAEFSFSN | |
| | | | | | VDLSNAGQYRCSYSTQYKSSGYSDLLEL | |
| | | | | | VVTGHYWTPSLLAQASPVVTSGGYVTLQ | |
| | | | | | CESWHNDHKFILTVEGPQKLSWTQDSQY | |
| | | | | | NYSTRKYHALFSVGPVTPNQRWICRCYS | |
| | | | | | YDRNRPYVWSPPSESVELLVSGNLQKPTI | |
| | | | | | KAEPGSVITSKRAMTIWCQGNLDAEVYF | |
| | | | | | LHNEKSQKTQSTQTLQEPGNKGKFFIPSV | |
| | | | | | TLQHAGQYRCYCYGSAGWSQPSDTLELV | |
| | | | | | VTGIYEYYEPRLSVLPSPVVTAGGNMTLH | |
| | | | | | CASDFPYDKFILTKEDKKFGNSLDTEHISS | |
| | | | | | SGQYRALFIIGPTTPTHTGAFRCYGYYKN | |
| | | | | | APQLWSVPSALQQILISGLSKKPSLLTHQG | |
| | | | | | HILDPGMTLTLQCFSDINYDRFALHKVGG | |
| | | | | | ADIMQHSSQQTDTGFSVANFTLGYVSSST | |
| | | | | | GGQYRCYGAHNLSSEWSASSEPLDILITG | |
| | | | | | QLPLTPSLSVQPNHTVHSGETVSLLCWSM | |
| | | | | | DSVDTFILSKEGSAQQPLRLKSKSHDQQS | |
| | | | | | QAEFSMSAVTSHLSGTYRCYGAQDSSFY | |
| | | | | | LLSSASAPVELTVSGPIETSTPPPTMSMPL | |
| | | | | | GGLHMYLKIQADGWMCHASKWVTTCDF | |
| | | | | | RWYGPKYITHSIRSFTPSVEQCKESIEQTK | |
| | | | | | QGTWLNPGFPPQSCGYATVTDAEAVIVQ | |
| | | | | | VTPHHVLVDEYTGEWVDSQFINGKCSNY | |
| | | | | | ICPTVHNSTTWHSDYKVKGLCDSNLGML | |
| | | | | | DSDLHLSSKAQVFEHPHIQDAASQLPDDE | |
| | | | | | SLFFGDTGLSKNPIELVEGWFSSWKSSIAS | |
| | | | | | FFFIIGLIIGLFLVLRVGIHLCIKLKHTKKR | |
| | | | | | QIYPYDVPDYA | |
| mu_ | murine | ⁶D4 | VM4 | — | MIAMLTVLLYLGLILEPRTAVQAGHLPKP | 85 |
| LILRB4/ | LILRB4 | | | | IIWAEPGSVIAAYTSVITWCQGSWEAQYY | |
| D4 | ectodomain | | | | HLYKEKSVNPWDTQVPLETRNKAKFNIPS | |
| | | | | | MTTSYAGIYKCYYESAAGFSEHSDAMEL | |
| | | | | | VMTGAYENPSLSVYPSSNVTSGVSISFSCS | |
| | | | | | SSIVFGRFILIQEGKHGLSWTLDSQHQAN | |
| | | | | | QPSYATFVLDAVTPNHNGTFRCYGYFRN | |
| | | | | | EPQVWSKPSNSLDLMISETKDQSSTPTED | |
| | | | | | GLETYQKILIGVLVSFLLLFFLLLFLILIGY | |
| | | | | | QYGHKKKANASVKNTQSENNAELNSWN | |
| | | | | | PQNEDPQGIVYAQVKPSRLQKDTACKET | |
| | | | | | QDVTYAQLCIRTQEQNNSIQADGWMCHA | |
| | | | | | SKWVTTCDFRWYGPKYITHSIRSFTPSVE | |
| | | | | | QCKESIEQTKQGTWLNPGFPPQSCGYATV | |
| | | | | | TDAEAVIVQVTPHHVLVDEYTGEWVDSQ | |
| | | | | | FINGKCSNYICPTVHNSTTWHSDYKVKGL | |
| | | | | | CDSNLGMLDSDLHLSSKAQVFEHPHIQD | |
| | | | | | AASQLPDDESLFFGDTGLSKNPIELVEGW | |
| | | | | | FSSWKSSIASFFFIIGLIIGLFLVLRVGIHLCI | |
| | | | | | KLKHTKKRQIYPYDVPDYA | |
| mu_ | murine | NA¹⁴ | — | — | MYTDIEMNRLGKNPNQKIITIGSICMVTGI | 86 |
| BAFF/ | BAFF | | | | VSLMLQIGNMISIWVSHSIHTGNQHQSEPI | |
| NA | ectodomain | | | | SNTNFLTEKAVASVKLAGNSSLCPINDQL | |
| | | | | | AALQADLMNLRMELQSYRGSATPAAAG | |
| | | | | | APELTAGVKLLTPAAPRPHNSSRGHRNRR | |
| | | | | | AFQGPEETEQDVDLSAPPAPCLPGCRHSQ | |
| | | | | | HDDNGMNLRNIIQDCLQLIADSDTPTIRK | |
| | | | | | GTYTFVPWLLSFKRGNALEEKENKIVVR | |
| | | | | | QTGYFFIYSQVLYTDPIFAMGHVIQRKKV | |
| | | | | | HVFGDELSLVTLFRCIQNMPKTLPNNSCY | |
| | | | | | SAGIARLEEGDEIQLAIPRENAQISRNGDD | |
| | | | | | TFFGALKLL | |
| mu_ | murine | ⁶D4 | VM⁴ | — | MGARRLRVRSQRSRDSSVPTQCNQTECF | 87 |
| BAFF- | BAFF-R | | | | DPLVRNCVSCELFHTPDTGHTSSLEPGTA | |
| R/D4 | ectodomain | | | | LQPQEGSALRPDVAIIQADGWMCHASKW | |
| | | | | | VTTCDFRWYGPKYITHSIRSFTPSVEQCKE | |
| | | | | | SIEQTKQGTWLNPGFPPQSCGYATVIDAE | |
| | | | | | AVIVQVTPHHVLVDEYTGEWVDSQFING | |
| | | | | | KCSNYICPTVHNSTTWHSDYKVKGLCDS | |
| | | | | | NLGMLDSDLHLSSKAQVFEHPHIQDAAS | |
| | | | | | QLPDDESLFFGDTGLSKNPIELVEGWFSS | |
| | | | | | WKSSIASFFFIIGLIIGLFLVLRVGIHLCIKL | |
| | | | | | KHTKKRQIYTDIEMNRLGK | |
| mu_ | murine | ⁶D4 | VM⁴ | — | MAQQCFHSEYFDSLLHACKPCHLRCSNPP | 88 |
| BCMA/ | BCMA | | | | ATCQPYCDPSVTSSVKGTYTIIQADGWM | |
| D4 | ectodomain | | | | CHASKWVTTCDFRWYGPKYITHSIRSFTP | |
| | | | | | SVEQCKESIEQTKQGTWLNPGFPPQSCGY | |
| | | | | | ATVTDAEAVIVQVTPHHVLVDEYTGEWV | |
| | | | | | DSQFINGKCSNYICPTVHNSTTWHSDYKV | |
| | | | | | KGLCDSNLGMLDSDLHLSSKAQVFEHPHI | |
| | | | | | QDAASQLPDDESLFFGDTGLSKNPIELVE | |
| | | | | | GWFSSWKSSIASFFFIIGLIIGLFLVLRVGIH | |
| | | | | | LCIKLKHTKKRQIYTDIEMNRLGK | |
| mu_ | murine | ⁶D4 | VM⁴ | — | MAMAFCPKDQYWDSSRKSCVSCALTCS | 89 |
| TACI/ | TACI | | | | QRSQRTCTDFCKFINCRKEQGRYYDHLL | |
| D4 | ectodomain | | | | GACVSCDSTCTQHPQQCAHFCEKRPRSQ | |
| | | | | | ANLQPELGRPQAGEVEVRSDNSGRHQGS | |
| | | | | | EHGPGLRLSSDQLTLYCIIQADGWMCHAS | |
| | | | | | KWVTTCDFRWYGPKYITHSIRSFTPSVEQ | |
| | | | | | CKESIEQTKQGTWLNPGFPPQSCGYATVT | |
| | | | | | DAEAVIVQVTPHHVLVDEYTGEWVDSQF | |
| | | | | | INGKCSNYICPTVHNSTTWHSDYKVKGL | |
| | | | | | CDSNLGMLDSDLHLSSKAQVFEHPHIQD | |
| | | | | | AASQLPDDESLFFGDTGLSKNPIELVEGW | |
| | | | | | FSSWKSSIASFFFIIGLIIGLFLVLRVGIHLCI | |
| | | | | | KLKHTKKRQIYTDIEMNRLGK | |
| mu_ | murine | ⁶D4 | VM⁴ | — | MASTRAKPTLPLLLALVTVVIPGPGDAQV | 90 |
| ICAM-1/ | ICAM-1 | | | | SIHPREAFLPQGGSVQVNCSSSCKEDLSLG | |
| D4 | ectodomain | | | | LETQWLKDELESGPNWKLFELSEIGEDSS | |
| | | | | | PLCFENCGTVQSSASATITVYSFPESVELR | |
| | | | | | PLPAWQQVGKDLTLRCHVDGGAPRTQLS | |
| | | | | | AVLLRGEEILSRQPVGGHPKDPKEITFTVL | |
| | | | | | ASRGDHGANFSCRTELDLRPQGLALFSNV | |
| | | | | | SEARSLRTFDLPATIPKLDTPDLLEVGTQQ | |
| | | | | | KLFCSLEGLFPASEARIYLELGGQMPTQES | |
| | | | | | TNSSDSVSATALVEVTEEFDRTLPLRCVL | |
| | | | | | ELADQILETQRTLTVYNFSAPVLTLSQLE | |
| | | | | | VSEGSQVTVKCEAHSGSKVVLLSGVEPRP | |
| | | | | | PTPQVQFTLNASSEDHKRSFFCSAALEVA | |
| | | | | | GKFLFKNQTLELHVLYGPRLDETDCLGN | |
| | | | | | WTWQEGSQQTLKCQAWGNPSPKMTCRR | |
| | | | | | KADGALLPIGVVKSVKQEMNGTYVCHAF | |
| | | | | | SSHGNVTRNVYLTVLYHSQNNDIIQADG | |
| | | | | | WMCHASKWVTTCDFRWYGPKYITHSIRS | |
| | | | | | FTPSVEQCKESIEQTKQGTWLNPGFPPQS | |
| | | | | | CGYATVTDAEAVIVQVTPHHVLVDEYTG | |
| | | | | | EWVDSQFINGKCSNYICPTVHNSTTWHSD | |
| | | | | | YKVKGLCDSNLGMLDSDLHLSSKAQVFE | |
| | | | | | HPHIQDAASQLPDDESLFFGDTGLSKNPIE | |
| | | | | | LVEGWFSSWKSSIASFFFIIGLIIGLFLVLR | |
| | | | | | VGIHLCIKLKHTKKRQIYTDIEMNRLGK | |
| mu_ | murine | ⁶D4 | VM⁴ | — | MDAKVVAVLALVLAALCISDGKPVSLSY | 91 |
| CXCL- | CXCL-12 | | | | RCPCRFFESHIARANVKHLKILNTPNCAL | |
| 12/D4 | | | | | QIVARLKNNNRQVCIDPKLKWIQEYLEK | |
| | | | | | ALNKRLKMIIQADGWMCHASKWVTTCD | |
| | | | | | FRWYGPKYITHSIRSFTPSVEQCKESIEQT | |
| | | | | | KQGTWLNPGFPPQSCGYATVIDAEAVIV | |
| | | | | | QVTPHHVLVDEYTGEWVDSQFINGKCSN | |
| | | | | | YICPTVHNSTTWHSDYKVKGLCDSNLGM | |
| | | | | | LDSDLHLSSKAQVFEHPHIQDAASQLPDD | |
| | | | | | ESLFFGDTGLSKNPIELVEGWFSSWKSSIA | |
| | | | | | SFFFIIGLIIGLFLVLRVGIHLCIKLKHTKKR | |
| | | | | | QIYTDIEMNRLGK | |
| mu_IL- | murine IL-4 | ⁶D4 | VM⁴ | — | MGLNPQLVVILLFFLECTRSHIHGCDKNH | 92 |
| 4/D4 | | | | | LREIIGILNEVTGEGTPCTEMDVPNVLTAT | |
| | | | | | KNTTESELVCRASKVLRIFYLKHGKTPCL | |
| | | | | | KKNSSVLMELQRLFRAFRCLDSSISCTMN | |
| | | | | | ESKSTSLKDFLESLKSIMQMDYSIIQADG | |
| | | | | | WMCHASKWVTTCDFRWYGPKYITHSIRS | |
| | | | | | FTPSVEQCKESIEQTKQGTWLNPGFPPQS | |
| | | | | | CGYATVTDAEAVIVQVTPHHVLVDEYTG | |
| | | | | | EWVDSQFINGKCSNYICPTVHNSTTWHSD | |
| | | | | | YKVKGLCDSNLGMLDSDLHLSSKAQVFE | |
| | | | | | HPHIQDAASQLPDDESLFFGDTGLSKNPIE | |
| | | | | | LVEGWFSSWKSSIASFFFIIGLIIGLFLVLR | |
| | | | | | VGIHLCIKLKHTKKRQIYTDIEMNRLGK | |
| mu_IL- | murine IL-6 | ⁶D4 | VM⁴ | — | MKFLSARDFHPVAFLGLMLVTTTAFPTSQ | 93 |
| 6/D4 | | | | | VRRGDFTEDTTPNRPVYTTSQVGGLITHV | |
| | | | | | LWEIVEMRKELCNGNSDCMNNDDALAE | |
| | | | | | NNLKLPEIQRNDGCYQTGYNQEICLLKISS | |
| | | | | | GLLEYHSYLEYMKNNLKDNKKDKARVL | |
| | | | | | QRDTETLIHIFNQEVKDLHKIVLPTPISNA | |
| | | | | | LLTDKLESQKEWLRTKTIQFILKSLEEFLK | |
| | | | | | VTLRSTRQTIIQADGWMCHASKWVTTCD | |
| | | | | | FRWYGPKYITHSIRSFTPSVEQCKESIEQT | |
| | | | | | KQGTWLNPGFPPQSCGYATVTDAEAVIV | |
| | | | | | QVTPHHVLVDEYTGEWVDSQFINGKCSN | |
| | | | | | YICPTVHNSTTWHSDYKVKGLCDSNLGM | |
| | | | | | LDSDLHLSSKAQVFEHPHIQDAASQLPDD | |
| | | | | | ESLFFGDTGLSKNPIELVEGWFSSWKSSIA | |
| | | | | | SFFFIIGLIIGLFLVLRVGIHLCIKLKHTKKR | |
| | | | | | QIYTDIEMNRLGK | |
| hu_Wnt2/ | human | ⁶D4 | VM⁴ | — | MNAPLGGIWLWLPLLLTWLTPEVNSSW | 94 |
| D4 | Wnt2 | | | | WYMRATGGSSRVMCDNVPGLVSSQRQL | |
| | | | | | CHRHPDVMRAISQGVAEWTAECQHQFR | |
| | | | | | QHRWNCNTLDRDHSLFGRVLLRSSRESA | |
| | | | | | FVYAISSAGVVFAITRACSQGEVKSCSCD | |
| | | | | | PKKMGSAKDSKGIFDWGGCSDNIDYGIK | |
| | | | | | FARAFVDAKERKGKDARALMNLHNNRA | |
| | | | | | GRKAVKRFLKQECKCHGVSGSCTLRTCW | |
| | | | | | LAMADFRKTGDYLWRKYNGAIQVVMNQ | |
| | | | | | DGTGFTVANERFKKPTKNDLVYFENSPD | |
| | | | | | YCIRDREAGSLGTAGRVCNLTSRGMDSC | |
| | | | | | EVMCCGRGYDTSHVTRMTKCGCKFHWC | |
| | | | | | CAVRCQDCLEALDVHTCKAPKNADWTT | |
| | | | | | ATIQADGWMCHASKWVTTCDFRWYGPK | |
| | | | | | YITHSIRSFTPSVEQCKESIEQTKQGTWLN | |
| | | | | | PGFPPQSCGYATVIDAEAVIVQVTPHHVL | |
| | | | | | VDEYTGEWVDSQFINGKCSNYICPTVHNS | |
| | | | | | TTWHSDYKVKGLCDSNLGMLDSDLHLSS | |
| | | | | | KAQVFEHPHIQDAASQLPDDESLFFGDTG | |
| | | | | | LSKNPIELVEGWFSSWKSSIASFFFIIGLIIG | |
| | | | | | LFLVLRVGIHLCIKLKHTKKRQIYTDIEMN | |
| | | | | | RLGK | |
| aCD19/VM | anti-CD19 | None | VM⁴ | — | MALPVTALLLPLALLLHAARPDIQMTQTT | 95 |
| | scFv | | | | SSLSASLGDRVTISCRASQDISKYLNWYQ | |
| | | | | | QKPDGTVKLLIYHTSRLHSGVPSRFSGSG | |
| | | | | | SGTDYSLTISNLEQEDIATYFCQQGNTLPY | |
| | | | | | TFGGGTKLEITGGGGSGGGGSGGGGSEV | |
| | | | | | KLQESGPGLVAPSQSLSVTCTVSGVSLPD | |
| | | | | | YGVSWIRQPPRKGLEWLGVIWGSETTYY | |
| | | | | | NSALKSRLTIIKDNSKSQVFLKMNSLQTD | |
| | | | | | DTAIYYCAKHYYYGGSYAMDYWGQGTS | |
| | | | | | VTVSSTTTPAPRPPTPAPTIASQPLSLRPEA | |
| | | | | | CRPAAGGAVHTRGLDFACDRSGMLDSDL | |
| | | | | | HLSSKAQVFEHPHIQDAASQLPDDESLFF | |
| | | | | | GDTGLSKNPIELVEGWFSSWKSSIASFFFII | |
| | | | | | GLIIGLFLVLRVGIHLCIKLKHTKKRQIYT | |
| | | | | | DIEMNRLGK | |
| αCD19/ | anti-CD19 | ⁶D4 | VM⁴ | — | MALPVTALLLPLALLLHAARPDIQMTQTT | 96 |
| D4 | scFv | | | | SSLSASLGDRVTISCRASQDISKYLNWYQ | |
| | | | | | QKPDGTVKLLIYHTSRLHSGVPSRFSGSG | |
| | | | | | SGTDYSLTISNLEQEDIATYFCQQGNTLPY | |
| | | | | | TFGGGTKLEITGGGGSGGGGSGGGGSEV | |
| | | | | | KLQESGPGLVAPSQSLSVTCTVSGVSLPD | |
| | | | | | YGVSWIRQPPRKGLEWLGVIWGSETTYY | |
| | | | | | NSALKSRLTIIKDNSKSQVFLKMNSLQTD | |
| | | | | | DTAIYYCAKHYYYGGSYAMDYWGQGTS | |
| | | | | | VTVSSTTTPAPRPPTPAPTIASQPLSLRPEA | |
| | | | | | CRPAAGGAVHTRGLDFACDDIIQADGWM | |
| | | | | | CHASKWVTTCDFRWYGPKYITHSIRSFTP | |
| | | | | | SVEQCKESIEQTKQGTWLNPGFPPQSCGY | |
| | | | | | ATVTDAEAVIVQVTPHHVLVDEYTGEWV | |
| | | | | | DSQFINGKCSNYICPTVHNSTTWHSDYKV | |
| | | | | | KGLCDSNLGMLDSDLHLSSKAQVFEHPHI | |
| | | | | | QDAASQLPDDESLFFGDTGLSKNPIELVE | |
| | | | | | GWFSSWKSSIASFFFIIGLIIGLFLVLR VGIH | |
| | | | | | LCIKLKHTKKRQIYTDIEMNRLGK | |
| hu_CD86/ | human | ⁶D4 | VM⁴ | — | MGLSNILFVMAFLLSGAAPLKIQAYFNET | 97 |
| D4 | CD86 | | | | ADLPCQFANSQNQSLSELVVFWQDQENL | |
| | ectodomain | | | | VLNEVYLGKEKFDSVHSKYMGRTSFDSD | |
| | | | | | SWTLRLHNLQIKDKGLYQCIIHHKKPTGM | |
| | | | | | IRIHQMNSELSVLANFSQPEIVPISNITENV | |
| | | | | | YINLTCSSIHGYPEPKKMSVLLRTKNSTIE | |
| | | | | | YDGIMQKSQDNVTELYDVSISLSVSFPDV | |
| | | | | | TSNMTIFCILETDKTRLLSSPFSIELEDPQP | |
| | | | | | PPDHIPWITAVLDIIQADGWMCHASKWV | |
| | | | | | TTCDFRWYGPKYITHSIRSFTPSVEQCKES | |
| | | | | | IEQTKQGTWLNPGFPPQSCGYATVTDAE | |
| | | | | | AVIVQVTPHHVLVDEYTGEWVDSQFING | |
| | | | | | KCSNYICPTVHNSTTWHSDYKVKGLCDS | |
| | | | | | NLGMLDSDLHLSSKAQVFEHPHIQDAAS | |
| | | | | | QLPDDESLFFGDTGLSKNPIELVEGWFSS | |
| | | | | | WKSSIASFFFIIGLIIGLFLVLRVGIHLCIKL | |
| | | | | | KHTKKRQIYTDIEMNRLGK | |
| αCD3/ | anti-CD3 | ⁶D4 | VM⁴ | — | MPMGSLQPLATLYLLGMLVASVLADIKL | 98 |
| D4 | scFv | | | | QQSGAELARPGASVKMSCKTSGYTFTRY | |
| | | | | | TMHWVKQRPGQGLEWIGYINPSRGYTNY | |
| | | | | | NQKFKDKATLTTDKSSSTAYMQLSSLTSE | |
| | | | | | DSAVYYCARYYDDHYCLDYWGQGTTLT | |
| | | | | | VSSVEGGSGGSGGSGGSGGVDDIQLTQSP | |
| | | | | | AIMSASPGEKVTMTCRASSSVSYMNWYQ | |
| | | | | | QKSGTSPKRWIYDTSKVASGVPYRFSGSG | |
| | | | | | SGTSYSLTISSMEAEDAATYYCQQWSSNP | |
| | | | | | LTFGAGTKLELKVIQADGWMCHASKWV | |
| | | | | | TTCDFRWYGPKYITHSIRSFTPSVEQCKES | |
| | | | | | IEQTKQGTWLNPGFPPQSCGYATVIDAE | |
| | | | | | AVIVQVTPHHVLVDEYTGEWVDSQFING | |
| | | | | | KCSNYICPTVHNSTTWHSDYKVKGLCDS | |
| | | | | | NLGMLDSDLHLSSKAQVFEHPHIQDAAS | |
| | | | | | QLPDDESLFFGDTGLSKNPIELVEGWFSS | |
| | | | | | WKSSIASFFFIIGLIIGLFLVLRVGIHLCIKL | |
| | | | | | KHTKKRQIYPYDVPDYA | |
| hu_ | OX40L | NA¹⁴ | — | — | MYTDIEMNRLGKNPNQKIITIGSICMVTGI | 99 |
| OX40L/ | ectodomain | | | | VSLMLQIGNMISIWVSHSIHTGNQHQSEPI | |
| NA | | | | | SNTNFLTEKAVASVKLAGNSSLCPINDMV | |
| | | | | | SHRYPRIQSIKVQFTEYKKEKGFILTSQKE | |
| | | | | | DEIMKVQNNSVIINCDGFYLISLKGYFSQE | |
| | | | | | VNISLHYQKDEEPLFQLKKVRSVNSLMV | |
| | | | | | ASLTYKDKVYLNVTTDNTSLDDFHVNGG | |
| | | | | | ELILIHQNPGEFCVL | |
| hu_4- | 4-1BBL | NA¹⁴ | — | — | MYTDIEMNRLGKNPNQKIITIGSICMVTGI | 100 |
| 1BBL/ | ectodomain | | | | VSLMLQIGNMISIWVSHSIHTGNQHQSEPI | |
| NA | | | | | SNTNFLTEKAVASVKLAGNSSLCPINDAC | |
| | | | | | PWAVSGARASPGSAASPRLRGPELSPDD | |
| | | | | | PAGLLDLRQGMFAQLVAQNVLLIDGPLS | |
| | | | | | WYSDPGLAGVSLTGGLSYKEDTKELVVA | |
| | | | | | KAGVYYVFFQLELRRVVAGEGSGSVSLA | |
| | | | | | LHLQPLRSAAGAAALALTVDLPPASSEAR | |
| | | | | | NSAFGFQGRLLHLSAGQRLGVHLHTEAR | |
| | | | | | ARHAWQLTQGATVLGLFRVTPEIPAGLPS | |
| | | | | | PRSE | |

OD¹: Oligomerization Domain
TM²: Transmembrane Domain
CD³: Cytosolic Domain
VM⁴: VSV-G protein transmembrane region and cytosolic tail
LZ⁵: GCN4 Leucine zipper domain
⁶D4: VSV-G D4 post-fusion trimerization domain
DE⁷: Dengue E protein post-fusion trimerization domain
Fold⁸: T4 Phage Fibritin Foldon
⁹NA-V1: Neuraminidase Stem (NA) version 1
¹⁰NA-V2: Neuraminidase Stem (NA) version 2
D4_Cyt¹¹: Cytoplasmic D4 domain
¹²D4-V4: Truncated 30 AA D4
¹³D4-V5: Truncated 45 AA D4
NA¹⁴: Neuraminidase stem

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EMBODIMENTS

The following non-limiting embodiments provide illustrative examples of the invention, but do not limit the scope of the invention.

Embodiment 1. A recombinant fusion protein comprising a transmembrane domain, a display polypeptide, and an oligomerization domain wherein when the recombinant fusion protein is expressed on the surface of an enveloped particle, the recombinant fusion protein is displayed in an oligomeric format.

Embodiment 2. The recombinant fusion protein of embodiment 1, wherein the oligomerization domain is a dimerization domain.

Embodiment 3. The recombinant fusion protein of embodiment 2, wherein the dimerization domain comprises a leucine zipper dimerization domain.

Embodiment 4. The recombinant fusion protein of embodiment 1, wherein the oligomerization domain is a trimerization domain.

Embodiment 5. The recombinant fusion protein of embodiment 4, wherein the trimerization domain comprises a post-fusion oligomerization domain of viral surface protein.

Embodiment 6. The recombinant fusion protein of embodiment 4, wherein the trimerization domain comprises a D4 post-fusion trimerization domain of VSV-G protein.

Embodiment 7. The recombinant fusion protein of embodiment 4, wherein the trimerization domain comprises a Dengue E protein post-fusion trimerization domain.

Embodiment 8. The recombinant fusion protein of embodiment 4, wherein the trimerization domain comprises a foldon trimerization domain.

Embodiment 9. The recombinant fusion protein of embodiment 1, wherein the oligomerization domain is a tetramerization domain.

Embodiment 10. The recombinant fusion protein of embodiment 9, wherein the tetramerization domain comprises an influenza neuraminidase stem domain.

Embodiment 11. The recombinant fusion protein of any of embodiments 1-10, wherein the oligomerization domain comprises an amino acid sequence that has at least 95% sequence identity to an amino acid sequence according to SEQ ID NOs: 6-19.

Embodiment 12. The recombinant fusion protein of any of embodiments 1-11, wherein when the recombinant fusion protein is expressed on the surface of the enveloped particle, the oligomerization domain is outside of the enveloped particle.

Embodiment 13. The recombinant fusion protein of any of embodiments 1-12, wherein when the recombinant fusion protein is expressed on the surface of the enveloped particle, the oligomerization domain is outside of the enveloped particle and adjacent to a signal peptide.

Embodiment 14. The recombinant fusion protein of any of embodiments 1-11, wherein when the recombinant fusion protein is expressed on the surface of the enveloped particle, the oligomerization domain is inside of the enveloped particle.

Embodiment 15. The recombinant fusion protein of embodiment 14, wherein when the recombinant fusion protein is expressed on the surface of the enveloped particle, the oligomerization domain is inside of the enveloped particle and adjacent to the transmembrane domain.

Embodiment 16. The recombinant fusion protein of any of embodiments 1-15, wherein the recombinant fusion protein comprises a signal peptide.

Embodiment 17. The recombinant fusion protein of embodiment 16, wherein domains of the recombinant fusion protein are arranged from the N-terminus to the C-terminus in the following orders:
(a) signal peptide, display polypeptide, oligomerization domain, and transmembrane domain;
(b) signal peptide, display polypeptide, transmembrane domain, and oligomerization domain: or
(c) signal peptide, oligomerization domain, display peptide, and transmembrane domain.

Embodiment 18. The recombinant fusion protein of any of embodiments 1-17, wherein the recombinant fusion protein further comprises a cytosolic domain.

Embodiment 19. The recombinant fusion protein of embodiment 18, wherein domains of the recombinant fusion protein are arranged from the N-terminus to the C-terminus in the following orders:
(a) signal peptide, display polypeptide, oligomerization domain, transmembrane domain, and cytosolic domain;
(b) signal peptide, display polypeptide, transmembrane domain, oligomerization domain, and cytosolic domain: or
(c) signal peptide, oligomerization domain, display peptide, transmembrane domain, and cytosolic domain.

Embodiment 20. The recombinant fusion protein of any of embodiments 1-19, wherein the enveloped particle comprises an enveloped viral-like particle, an enveloped virus, an extracellular vesicle.

Embodiment 21. The recombinant fusion protein of embodiment 20, wherein the extracellular vesicle comprises an exosome or an ectosome.

Embodiment 22. The recombinant fusion protein of any of embodiments 1-21, wherein the display polypeptide targets a cell surface protein that is oligomerized on a target cell for agonistic or antagonistic function.

Embodiment 23. The recombinant fusion protein of embodiment 22, wherein the cell surface protein is dimerized and the oligomerization domain is a dimerization domain.

Embodiment 24. The recombinant fusion protein of embodiment 23, wherein the cell surface protein is trimerized and the oligomerization domain is a trimerization domain.

Embodiment 25. The recombinant fusion protein of embodiment 23, wherein the cell surface protein is tetramerized and the oligomerization domain is a tetramerization domain.

Embodiment 26. The recombinant fusion protein of any of embodiments 1-25, wherein the display polypeptide comprises a single chain variable fragment (scFv), a type I transmembrane polypeptide, a type II transmembrane polypeptide, a type III transmembrane polypeptide, a GPI-anchored polypeptide, a secreted polypeptide, or a multi-pass transmembrane polypeptide.

Embodiment 27. The recombinant fusion protein of embodiment 26, wherein the scFv comprises an anti-BCMA, an anti-NTD scFv polypeptide, an anti-RBD scFv polypeptide, or an anti-CD19 scFv polypeptide.

Embodiment 28. The recombinant fusion protein of embodiment 26, wherein the type I transmembrane polypeptide comprises ACE2, ICAM-1, LILRA5, LILRB3, or LILRB4.

Embodiment 29. The recombinant fusion protein of embodiment 26, wherein the type II transmembrane polypeptide comprises DPP4, or BAFF.

Embodiment 30. The recombinant fusion protein of embodiment 26, wherein the type III transmembrane polypeptide comprises BCMA, TACI, or BAFF Receptor.

Embodiment 31. The recombinant fusion protein of embodiment 26, wherein the GPI-anchored polypeptide comprises CD24, CD48, CD59, or CD55.

Embodiment 32. The recombinant fusion protein of embodiment 26, wherein the secreted polypeptide comprises IL-4, or CXCL-12.

Embodiment 33. The recombinant fusion protein of embodiment 26, wherein the multi-pass transmembrane polypeptide comprises NTCP, or FCεRI beta.

Embodiment 34. The recombinant fusion protein of any of embodiments 1-25, wherein the display polypeptide comprises an antibody, a receptor, a cytokine, an immune checkpoint protein, a chemokine, an adhesion factor, or a mitogen.

Embodiment 35. The recombinant fusion protein of embodiment 34, wherein the cytokine comprises IL-4, or IL-6.

Embodiment 36. The recombinant fusion protein of embodiment 34, wherein the chemokine comprises CXCL-12.

Embodiment 37. The recombinant fusion protein of embodiment 34, wherein the adhesion factor comprises ICAM-1, or LFA-1.

Embodiment 38. The recombinant fusion protein of embodiment 34, wherein the mitogen comprises Wnt.

Embodiment 39. The recombinant fusion protein of any of embodiments 1-38, wherein the display polypeptide comprises an amino acid sequence that has at least 95% sequence identity to an amino acid sequence according to SEQ ID NOs: 20-51, 56-70.

Embodiment 40. The recombinant fusion protein of any of embodiments 1-39, wherein the transmembrane domain anchors the fusion protein to a bilayer of the enveloped particle.

Embodiment 41. The recombinant fusion protein of any of embodiments 1-40, wherein the transmembrane domain comprises the transmembrane domain of a Vesicular Stomatitis virus glycoprotein (VSV-G).

Embodiment 42. The recombinant fusion protein of embodiment 41, wherein the recombinant fusion protein comprises the transmembrane domain and cytosolic domain of a Vesicular Stomatitis virus glycoprotein (VSV-G).

Embodiment 43. The recombinant fusion protein of any of embodiments 1-40, wherein the transmembrane domain comprises the transmembrane domain of a Dengue E protein.

Embodiment 44. The recombinant fusion protein of embodiment 43, wherein the recombinant fusion protein comprises the transmembrane domain and cytosolic domain of a Dengue E protein.

Embodiment 45. The recombinant fusion protein of any of embodiments 1-40, wherein the transmembrane domain comprises the transmembrane domain of influenza Hemagglutinin (HA).

Embodiment 46. The recombinant fusion protein of embodiment 45, wherein the recombinant fusion protein comprises the transmembrane domain and cytosolic domain of influenza Hemagglutinin (HA).

Embodiment 47. The recombinant fusion protein of any of embodiments 1-40, wherein the transmembrane domain comprises the transmembrane domain of HIV surface glycoprotein GP120 or GP41.

Embodiment 48. The recombinant fusion protein of embodiment 47, wherein the recombinant fusion protein comprises the transmembrane domain and cytosolic domain of HIV surface glycoprotein GP120 or GP41.

Embodiment 49. The recombinant fusion protein of any of embodiments 1-40, wherein the transmembrane domain comprises the transmembrane domain of measles virus surface glycoprotein hamagglutinin (H) protein.

Embodiment 50. The recombinant fusion protein of embodiment 49, wherein the recombinant fusion protein comprises the transmembrane domain and cytosolic domain of measles virus surface glycoprotein hamagglutinin (H) protein.

Embodiment 51. The recombinant fusion protein of any of embodiments 1-40, wherein the transmembrane domain comprises the transmembrane domain of influenza Neuraminidase (NA).

Embodiment 52. The recombinant fusion protein of embodiment 51, wherein the recombinant fusion protein comprises the transmembrane domain and cytosolic domain of influenza Neuraminidase (NA).

Embodiment 53. The recombinant fusion protein of any of embodiments 1-52, wherein the transmembrane domain comprises an amino acid sequence that has at least 95% sequence identity to an amino acid sequence according to SEQ ID NOs: 1-4, 52-55.

Embodiment 54. The recombinant fusion protein of any of embodiments 1-53, wherein when the recombinant fusion protein is expressed on the surface of the enveloped particle the recombinant fusion protein is expressed at a valency of about 10 copies on the surface of the enveloped particle.

Embodiment 55. The recombinant fusion protein of any of embodiments 1-53, wherein when the recombinant fusion protein is expressed on the surface of the enveloped particle the recombinant fusion protein is expressed at a valency of about 10 to 15 copies on the surface of the enveloped particle.

Embodiment 56. The recombinant fusion protein of any of embodiments 1-53, wherein when the recombinant fusion protein is expressed on the surface of the enveloped particle the recombinant fusion protein is expressed at a valency of at least about 25 copies on the surface of the enveloped particle.

Embodiment 57. The recombinant fusion protein of any of embodiments 1-53, wherein when the recombinant fusion protein is expressed on the surface of the enveloped particle the recombinant fusion protein is expressed at a valency of at least about 50 copies on the surface of the enveloped particle.

Embodiment 58. The recombinant fusion protein of any of embodiments 1-53, wherein when the recombinant fusion protein is expressed on the surface of the enveloped particle the recombinant fusion protein is expressed at a valency of at least about 100 copies on the surface of the enveloped particle.

Embodiment 59. The recombinant fusion protein of any of embodiments 1-53, wherein when the recombinant fusion protein is expressed on the surface of the enveloped particle the recombinant fusion protein is expressed at a valency of at least about 200 copies on the surface of the enveloped particle.

Embodiment 60. The recombinant fusion protein of any of embodiments 1-53, wherein when the recombinant fusion protein is expressed on the surface of the enveloped particle the recombinant fusion protein is expressed at a valency of at least about 400 copies on the surface of the enveloped particle.

Embodiment 61. The recombinant fusion protein of any of embodiments 1-53, wherein when the recombinant fusion protein is expressed on the surface of the enveloped particle the recombinant fusion protein is expressed at a valency of at least about 600 copies on the surface of the enveloped particle.

Embodiment 62. The recombinant fusion protein of any of embodiments 1-53, wherein when the recombinant fusion protein is expressed on the surface of the enveloped particle the recombinant fusion protein is expressed at least about 1000 copies on the surface of the enveloped particle.

Embodiment 63. An enveloped particle comprising the recombinant fusion protein according to any one of embodiments 1-62.

Embodiment 64. An enveloped particle comprising:
(a) a first recombinant fusion protein that is displayed in an oligomeric format on the surface of the enveloped particle wherein the first recombinant fusion protein comprises a transmembrane domain, a display polypeptide, and an oligomerization domain; and
(b) a second recombinant fusion protein that is displayed in a monomeric format on the surface of the enveloped particle wherein the second recombinant fusion protein comprises a transmembrane domain and a display polypeptide.

Embodiment 65. The enveloped particle of embodiment 64, wherein the oligomerization domain is a dimerization domain.

Embodiment 66. The enveloped particle of embodiment 65, wherein the dimerization domain comprises a leucine zipper dimerization domain.

Embodiment 67. The enveloped particle of embodiment 64, wherein the oligomerization domain is a trimerization domain.

Embodiment 68. The enveloped particle of embodiment 67, wherein the trimerization domain comprises a post-fusion oligomerization domain of viral surface protein.

Embodiment 69. The enveloped particle of embodiment 68, wherein the trimerization domain comprises a D4 post-fusion trimerization domain of VSV-G protein.

Embodiment 70. The enveloped particle of embodiment 67, wherein the trimerization domain comprises a Dengue E protein post-fusion trimerization domain.

Embodiment 71. The enveloped particle of embodiment 67, wherein the trimerization domain comprises a foldon trimerization domain.

Embodiment 72. The enveloped particle of embodiment 64, wherein the oligomerization domain is a tetramerization domain.

Embodiment 73. The enveloped particle of embodiment 72, wherein the tetramerization domain comprises an influenza neuraminidase stem domain.

Embodiment 74. The enveloped particle of any of embodiments 64-73, wherein the oligomerization domain comprises an amino acid sequence that has at least 95% sequence identity to an amino acid sequence according to SEQ ID NOs: 6-19.

Embodiment 75. The enveloped particle of any of embodiments 64-74, wherein when the first recombinant fusion protein is expressed on the surface of the enveloped particle, the oligomerization domain is outside of the enveloped particle.

Embodiment 76. The enveloped particle of embodiment 75, wherein when the first recombinant fusion protein is expressed on the surface of the enveloped particle, the oligomerization domain is outside of the enveloped particle and adjacent to a signal peptide.

Embodiment 77. The enveloped particle of any of embodiments 64-74, wherein when the first recombinant fusion protein is expressed on the surface of the enveloped particle, the oligomerization domain is inside of the enveloped particle.

Embodiment 78. The enveloped particle of embodiment 77, wherein when the first recombinant fusion protein is expressed on the surface of the enveloped particle, the oligomerization domain is inside of the enveloped particle and adjacent to the transmembrane domain.

Embodiment 79. The enveloped particle of any of embodiments 64-78, wherein the first recombinant fusion protein comprises a signal peptide.

Embodiment 80. The enveloped particle of embodiment 79, wherein domains of the first recombinant fusion protein are arranged from the N-terminus to the C-terminus in the following orders:
(a) signal peptide, display polypeptide, oligomerization domain, and transmembrane domain;
(b) signal peptide, display polypeptide, transmembrane domain, and oligomerization domain: or
(c) signal peptide, oligomerization domain, display peptide, and transmembrane domain.

Embodiment 81. The enveloped particle of any of embodiments 64-80, wherein the first recombinant fusion protein further comprises a cytosolic domain.

Embodiment 82. The enveloped particle of embodiment 81, wherein domains of the first recombinant fusion protein are arranged from the N-terminus to the C-terminus in the following orders:
(a) signal peptide, display polypeptide, oligomerization domain, transmembrane domain, and cytosolic domain;
(b) signal peptide, display polypeptide, transmembrane domain, oligomerization domain, and cytosolic domain; or
(c) signal peptide, oligomerization domain, display peptide, transmembrane domain, and cytosolic domain.

Embodiment 83. The enveloped particle of any of embodiments 64-82, wherein the enveloped particle comprises an enveloped viral-like particle, an enveloped virus, an extracellular vesicle.

Embodiment 84. The enveloped particle of embodiment 83, wherein the extracellular vesicle comprises an exosome or an ectosome.

Embodiment 85. The enveloped particle of any of embodiments 64-84, wherein the first recombinant fusion protein and the second recombinant fusion protein have the same display polypeptide.

Embodiment 86. The enveloped particle of any of embodiments 64-84, wherein the first recombinant fusion protein and the second recombinant fusion protein have different display polypeptides.

Embodiment 87. The enveloped particle of any of embodiments 64-86, wherein the display polypeptide targets a cell surface protein that is oligomerized on a target cell for agonistic or antagonistic function.

Embodiment 88. The enveloped particle of embodiment 87, wherein the cell surface protein is dimerized and the oligomerization domain is a dimerization domain.

Embodiment 89. The enveloped particle of embodiment 87, wherein the cell surface protein is trimerized and the oligomerization domain is a trimerization domain.

Embodiment 90. The enveloped particle of embodiment 87, wherein the cell surface protein is tetramerized and the oligomerization domain is a tetramerization domain.

Embodiment 91. The enveloped particle of any of embodiments 64-90, wherein the display polypeptide comprises a single chain variable fragment (scFv), a type I transmembrane polypeptide, a type II transmembrane polypeptide, a type III transmembrane polypeptide, a GPI-anchored polypeptide, a secreted polypeptide, or a multi-pass transmembrane polypeptide.

Embodiment 92. The enveloped particle of embodiment 91, wherein the scFv comprises an anti-BCMA scFv polypeptide, an anti-NTD scFv polypeptide, an anti-RBD scFv polypeptide, or an anti-CD19 scFv polypeptide.

Embodiment 93. The enveloped particle of embodiment 91, wherein the type I transmembrane polypeptide comprises ACE2, ICAM-1, LILRA5, LILRB3, or LILRB4.

Embodiment 94. The enveloped particle of embodiment 91, wherein the type II transmembrane polypeptide comprises DPP4, or BAFF.

Embodiment 95. The enveloped particle of embodiment 91, wherein the type III transmembrane polypeptide comprises BCMA, TACI, or BAFF Receptor.

Embodiment 96. The enveloped particle of embodiment 91, wherein the GPI-anchored polypeptide comprises CD24, CD48, CD59, or CD55.

Embodiment 97. The enveloped particle of embodiment 91, wherein the secreted polypeptide comprises IL-4, or CXCL-12.

Embodiment 98. The enveloped particle of embodiment 91, wherein the multi-pass transmembrane polypeptide comprises NTCP, or FCεRI beta.

Embodiment 99. The enveloped particle of any of embodiment s 64-90, wherein the display polypeptide comprises an antibody, a receptor, a cytokine, an immune checkpoint protein, a chemokine, an adhesion factor, or a mitogen.

Embodiment 100. The enveloped particle of embodiment 99, wherein the cytokine comprises IL-4, or IL-6.

Embodiment 101. The enveloped particle of embodiment 99, wherein the chemokine comprises CXCL-12.

Embodiment 102. The enveloped particle of embodiment 99, wherein the adhesion factor comprises ICAM-1 or LFA-1.

Embodiment 103. The enveloped particle of embodiment 99, wherein the mitogen comprises Wnt.

Embodiment 104. The enveloped particle of embodiment 99, wherein the display polypeptide comprises an amino acid sequence that has at least 95% sequence identity to an amino acid sequence according to SEQ ID NOs: 20-51, 56-70.

Embodiment 105. The enveloped particle of any of embodiments 64-104, wherein the first recombinant fusion protein and the second recombinant fusion protein have the same transmembrane domains.

Embodiment 106. The enveloped particle of any of embodiments 64-104, wherein the first recombinant fusion protein and the second recombinant fusion protein have different transmembrane domains.

Embodiment 107. The enveloped particle of any of embodiments 64-106, wherein the transmembrane domain anchors the fusion protein to a bilayer of the enveloped particle.

Embodiment 108. The enveloped particle of any of embodiments 64-107, wherein the transmembrane domain comprises the transmembrane domain of a Vesicular Stomatitis virus glycoprotein (VSV-G).

Embodiment 109. The enveloped particle of embodiment 108, wherein the first recombinant fusion protein comprises the transmembrane domain and cytosolic domain of a Vesicular Stomatitis virus glycoprotein (VSV-G).

Embodiment 110. The enveloped particle of any of embodiments 64-107, wherein the transmembrane domain comprises the transmembrane domain of a Dengue E protein.

Embodiment 111. The enveloped particle of embodiments 110, wherein the first recombinant fusion protein comprises the transmembrane domain and cytosolic domain of a Dengue E protein.

Embodiment 112. The enveloped particle of any of embodiments 64-107, wherein the transmembrane domain comprises the transmembrane domain of influenza Hemagglutinin (HA).

Embodiment 113. The enveloped particle of embodiment 112, wherein the first recombinant fusion protein comprises the transmembrane domain and cytosolic domain of influenza Hemagglutinin (HA).

Embodiment 114. The enveloped particle of any of embodiments 64-107, wherein the transmembrane domain comprises the transmembrane domain of HIV surface glycoprotein GP120 or GP41.

Embodiment 115. The enveloped particle of embodiment 114, wherein the first recombinant fusion protein comprises the transmembrane domain and cytosolic domain of HIV surface glycoprotein GP120 or GP41.

Embodiment 116. The enveloped particle of any of claims 64-107, wherein the transmembrane domain comprises the transmembrane domain of measles virus surface glycoprotein hamagglutinin (H) protein.

Embodiment 117. The enveloped particle of embodiment 116, wherein the first recombinant fusion protein comprises the transmembrane domain and cytosolic domain of measles virus surface glycoprotein hamagglutinin (H) protein.

Embodiment 118. The enveloped particle of any of embodiments 64-107, wherein the transmembrane domain comprises the transmembrane domain of influenza Neuraminidase (NA).

Embodiment 119. The enveloped particle of embodiment 118, wherein the first recombinant fusion protein comprises the transmembrane domain and cytosolic domain of influenza Neuraminidase (NA).

Embodiment 120. The enveloped particle of any of embodiments 64-119, wherein the transmembrane domain comprises an amino acid sequence that has at least 95% sequence identity to an amino acid sequence according to SEQ ID NOs: 1-4, 52-55.

Embodiment 121. The enveloped particle of any of embodiments 64-120, wherein when the first recombinant fusion protein is expressed on the surface of the enveloped particle the first recombinant fusion protein is expressed at a valency of about 10 copies on the surface of the enveloped particle.

Embodiment 122. The enveloped particle of any of embodiments 64-120, wherein when the first recombinant fusion protein is expressed on the surface of the enveloped particle the first recombinant fusion protein is expressed at a valency of about 10 to 15 copies on the surface of the enveloped particle.

Embodiment 123. The enveloped particle of any of embodiments 64-120, wherein when the first recombinant fusion protein is expressed on the surface of the enveloped particle the first recombinant fusion protein is expressed at a valency of at least about 25 copies on the surface of the enveloped particle.

Embodiment 124. The enveloped particle of any of embodiments 64-120, wherein when the first recombinant fusion protein is expressed on the surface of the enveloped particle the first recombinant fusion protein is expressed at a valency of at least about 50 copies on the surface of the enveloped particle.

Embodiment 125. The enveloped particle of any of embodiments 64-120, wherein when the first recombinant fusion protein is expressed on the surface of the enveloped particle the first recombinant fusion protein is expressed at a valency of at least about 100 copies on the surface of the enveloped particle.

Embodiment 126. The enveloped particle of any of embodiments 64-120, wherein when the first recombinant fusion protein is expressed on the surface of the enveloped particle the first recombinant fusion protein is expressed at a valency of at least about 200 copies on the surface of the enveloped particle.

Embodiment 127. The enveloped particle of any of embodiments 64-120, wherein when the first recombinant fusion protein is expressed on the surface of the enveloped particle the first recombinant fusion protein is expressed at a valency of at least about 400 copies on the surface of the enveloped particle.

Embodiment 128. The enveloped particle of any of embodiments 64-120, wherein when the first recombinant fusion protein is expressed on the surface of the enveloped particle the first recombinant fusion protein is expressed at a valency of at least about 600 copies on the surface of the enveloped particle.

Embodiment 129. The enveloped particle of any of embodiments 64-120, wherein when the first recombinant fusion protein is expressed on the surface of the enveloped particle the first recombinant fusion protein is expressed on the surface of the enveloped particle at a valency of at least about 1000 copies on the surface of the enveloped particle.

Embodiment 130. The enveloped particle of any of embodiments 64-120, wherein when the second recombinant fusion protein is expressed on the surface of the enveloped particle the second recombinant fusion protein is expressed at a valency of about 10 copies on the surface of the enveloped particle.

Embodiment 131. The enveloped particle of any of embodiments 64-120, wherein when the second recombinant fusion protein is expressed on the surface of the enveloped particle the second recombinant fusion protein is expressed at a valency of about 10 to 15 copies on the surface of the enveloped particle.

Embodiment 132. The enveloped particle of any of embodiments 64-120, wherein when the second recombinant fusion protein is expressed on the surface of the enveloped particle the second recombinant fusion protein is expressed at a valency of at least about 25 copies on the surface of the enveloped particle.

Embodiment 133. The enveloped particle of any of embodiments 64-120, wherein when the second recombinant fusion protein is expressed on the surface of the enveloped particle the second recombinant fusion protein is expressed at a valency of at least about 50 copies on the surface of the enveloped particle.

Embodiment 134. The enveloped particle of any of embodiments 64-120, wherein when the second recombinant fusion protein is expressed on the surface of the enveloped particle the second recombinant fusion protein is expressed at a valency of at least about 100 copies on the surface of the enveloped particle.

Embodiment 135. The enveloped particle of any of embodiments 64-120, wherein when the second recombinant fusion protein is expressed on the surface of the enveloped particle the second recombinant fusion protein is expressed at a valency of at least about 200 copies on the surface of the enveloped particle.

Embodiment 136. The enveloped particle of any of embodiments 64-120, wherein when the second recombinant fusion protein is expressed on the surface of the enveloped particle the second recombinant fusion protein is expressed at a valency of at least about 400 copies on the surface of the enveloped particle.

Embodiment 137. The enveloped particle of any of embodiments 64-120, wherein when the second recombinant fusion protein is expressed on the surface of the enveloped particle the second recombinant fusion protein is expressed at a valency of at least about 600 copies on the surface of the enveloped particle.

Embodiment 138. The enveloped particle of any of embodiments 64-120, wherein when the second recombinant fusion protein is expressed on the surface of the enveloped particle the second recombinant fusion protein is expressed on the surface of the enveloped particle at a valency of at least about 1000 copies on the surface of the enveloped particle.

Embodiment 139. A composition comprising a nucleic acid sequence that encodes the recombinant fusion protein of any of embodiments 1-62.

Embodiment 140. A composition comprising a nucleic acid sequence that encodes the first recombinant fusion protein of any one of embodiments 64-138 and the second recombinant fusion protein of any one of embodiments 64-138.

Embodiment 141. The composition of any one of embodiments 139 or 140, wherein the composition further comprises a second nucleic acid sequence that encodes one or more packaging viral proteins.

Embodiment 142. The composition of embodiment 141, wherein the one or more packaging viral proteins is a lentiviral protein, a retroviral protein, an adenoviral protein, or combinations thereof.

Embodiment 143. The composition of embodiment 141, wherein the one or more packaging viral proteins comprises gag, pol, pre, tat, rev, or combinations thereof.

Embodiment 144. The composition of any one of embodiments 139-143, further comprising a third nucleic acid sequence that encodes a reporter, a therapeutic molecule, or combinations thereof.

Embodiment 145. The composition of embodiment 144, wherein the reporter is a fluorescent protein or luciferase.

Embodiment 146. The composition of embodiment 145, wherein the fluorescent protein is green fluorescent protein.

Embodiment 147. The composition of embodiment 144, wherein the therapeutic molecule is an immune modulating protein, a cellular signal modulating molecule, a proliferation modulating molecule, a cell death modulating molecule, or combinations thereof.

Embodiment 148. The composition of any of embodiments 144-147, wherein the nucleic acid sequence that encodes the recombinant fusion protein and the second nucleic acid sequence and the third nucleic acid sequence are within a same vector.

Embodiment 149. The composition of any of embodiments 144-147, wherein the nucleic acid sequence that encodes the first recombinant fusion protein and the second recombinant fusion protein and the second nucleic acid sequence and the third nucleic acid sequence are within a same vector.

Embodiment 150. The composition of any of embodiments 144-147, wherein the nucleic acid sequence that encodes the recombinant fusion protein and the second nucleic acid sequence and the third nucleic acid sequence are within different vectors

Embodiment 151. The composition of any of embodiments 144-147, wherein the nucleic acid sequence that encodes the first recombinant fusion protein and the second recombinant fusion protein and the second nucleic acid sequence and the third nucleic acid sequence are within different vectors.

Embodiment 152. The composition of any of embodiments 148-151, wherein the vector is a lentivirus vector, an adenovirus vector, or an adeno-associated virus vector.

Embodiment 153. A method of agonizing or antagonizing a cell that has an oligomerized cell surface protein comprising contacting the cell with an enveloped particle comprising a first recombinant fusion protein that is displayed in an oligomeric format on the surface of the enveloped particle wherein the first recombinant fusion protein comprises a transmembrane domain, a display polypeptide, and an oligomerization domain.

Embodiment 154. The method of embodiment 153, wherein the enveloped particle further comprises a second recombinant fusion protein that is displayed in a monomeric format on the surface of the enveloped particle wherein the second recombinant fusion protein comprises a transmembrane domain and a display polypeptide.

Embodiment 155. The method of embodiment 153 or 154, wherein the oligomerization domain is a dimerization domain.

Embodiment 156. The method of embodiment 155, wherein the dimerization domain comprises a leucine zipper dimerization domain.

Embodiment 157. The method of embodiment 153 or 154, wherein the oligomerization domain is a trimerization domain.

Embodiment 158. The method of embodiment 157, wherein the trimerization domain comprises a post-fusion oligomerization domain of viral surface protein.

Embodiment 159. The method of embodiment 158, wherein the trimerization domain comprises a D4 post-fusion trimerization domain of VSV-G protein.

Embodiment 160. The method of embodiment 157, wherein the trimerization domain comprises a Dengue E protein post-fusion trimerization domain.

Embodiment 161. The method of embodiment 157, wherein the trimerization domain comprises a foldon trimerization domain.

Embodiment 162. The method of embodiment 153 or 154, wherein the oligomerization domain is a tetramerization domain.

Embodiment 163. The method of embodiment 162, wherein the tetramerization domain comprises an influenza neuraminidase stem domain.

Embodiment 164. The method of any of embodiments 153-163, wherein the oligomerization domain comprises an amino acid sequence that has at least 95% sequence identity to an amino acid sequence according to SEQ ID NOs: 6-19.

Embodiment 165. The method of any of embodiments 153-163, wherein when the first recombinant fusion protein is expressed on the surface of the enveloped particle, the oligomerization domain is outside of the enveloped particle.

Embodiment 166. The method of embodiment 165, wherein when the first recombinant fusion protein is expressed on the surface of the enveloped particle, the oligomerization domain is outside of the enveloped particle and adjacent to a signal peptide.

Embodiment 167. The method of any of embodiments 153-163, wherein when the first recombinant fusion protein is expressed on the surface of the enveloped particle, the oligomerization domain is inside of the enveloped particle.

Embodiment 168. The method of embodiment 167, wherein when the first recombinant fusion protein is expressed on the surface of the enveloped particle, the oligomerization domain is inside of the enveloped particle and adjacent to the transmembrane domain.

Embodiment 169. The method of any of embodiments 153-168, wherein the first recombinant fusion protein comprises a signal peptide.

Embodiment 170. The method of any of embodiments 153-169, wherein domains of the first recombinant fusion protein are arranged from the N-terminus to the C-terminus in the following orders:
(a) signal peptide, display polypeptide, oligomerization domain, and transmembrane domain;
(b) signal peptide, display polypeptide, transmembrane domain, and oligomerization domain: or
(c) signal peptide, oligomerization domain, display peptide, and transmembrane domain.

Embodiment 171. The method of embodiment 170, wherein the first recombinant fusion protein further comprises a cytosolic domain.

Embodiment 172. The method of any of embodiments 153-169, wherein domains of the first recombinant fusion protein are arranged from the N-terminus to the C-terminus in the following orders:
(a) signal peptide, display polypeptide, oligomerization domain, transmembrane domain, and cytosolic domain;
(b) signal peptide, display polypeptide, transmembrane domain, oligomerization domain, and cytosolic domain: or
(c) signal peptide, oligomerization domain, display peptide, transmembrane domain, and cytosolic domain.

Embodiment 173. The method of embodiment 172, wherein the enveloped particle comprises an enveloped viral-like particle, an enveloped virus, an extracellular vesicle.

Embodiment 174. The method of embodiment 173, wherein the extracellular vesicle comprises an exosome or an ectosome.

Embodiment 175. The method of any of embodiment s 153-174, wherein the first recombinant fusion protein and the second recombinant fusion protein have the same display polypeptide.

Embodiment 176. The method of any of embodiment s 153-174, wherein the first recombinant fusion protein and the second recombinant fusion protein have different display polypeptides.

Embodiment 177. The method of any of embodiments 153-176, wherein the display polypeptide targets the cell surface protein that is oligomerized on a target cell for agonistic or antagonistic function.

Embodiment 178. The method of embodiment 177, wherein the cell surface protein is dimerized and the oligomerization domain is a dimerization domain.

Embodiment 179. The method of embodiment 177, wherein the cell surface protein is trimerized and the oligomerization domain is a trimerization domain.

Embodiment 180. The method of embodiment 177, wherein the cell surface protein is tetramerized and the oligomerization domain is a tetramerization domain.

Embodiment 181. The method of any of embodiments 153-180, wherein the display polypeptide comprises a single chain variable fragment (scFv), a type I transmembrane polypeptide, a type II transmembrane polypeptide, a type III transmembrane polypeptide, a GPI-anchored polypeptide, a secreted polypeptide, or a multi-pass transmembrane polypeptide.

Embodiment 182. The method of embodiment 181, wherein the scFv comprises an anti-BCMA scFv polypeptide, an anti-NTD scFv polypeptide, an anti-RBD scFv polypeptide, or an anti-CD19 scFv polypeptide.

Embodiment 183. The method of embodiment 181, wherein the type I transmembrane polypeptide comprises ACE2, ICAM-1, LILRA5, LILRB3, or LILRB4.

Embodiment 184. The method of embodiment 181, wherein the type II transmembrane polypeptide comprises DPP4, or BAFF.

Embodiment 185. The method of embodiment 181, wherein the type III transmembrane polypeptide comprises BCMA, TACI, or BAFF Receptor.

Embodiment 186. The method of embodiment 181, wherein the GPI-anchored polypeptide comprises CD24, CD48, CD59, or CD55.

Embodiment 187. The method of embodiment 181, wherein the secreted polypeptide comprises IL-4, or CXCL-12.

Embodiment 188. The method of embodiment 181, wherein the multi-pass transmembrane polypeptide comprises NTCP, or FCεRI beta.

Embodiment 189. The method of embodiment 181, wherein the display polypeptide comprises an antibody, a receptor, a cytokine, an immune checkpoint protein, a chemokine, an adhesion factor, or a mitogen.

Embodiment 190. The method of embodiment 189, wherein the cytokine comprises IL-4, or IL-6.

Embodiment 191. The method of embodiment 189, wherein the chemokine comprises CXCL-12.

Embodiment 192. The method of embodiment 189, wherein the adhesion factor comprises ICAM-1, or LFA-1.

Embodiment 193. The method of embodiment 189, wherein the mitogen comprises Wnt.

Embodiment 194. The method of any of embodiments 153-193, wherein the display polypeptide comprises an amino acid sequence that has at least 95% sequence identity to an amino acid sequence according to SEQ ID NOs: 20-51, 56-70.

Embodiment 195. The method of any of embodiments 153-193, wherein the first recombinant fusion protein and the second recombinant fusion protein have the same transmembrane domains.

Embodiment 196. The method of any of embodiments 153-193, wherein the first recombinant fusion protein and the second recombinant fusion protein have different transmembrane domains.

Embodiment 197. The method of any of embodiments 153-196, wherein the transmembrane domain anchors the fusion protein to a bilayer of the enveloped particle.

Embodiment 198. The method of any of embodiment 153-197, wherein the transmembrane domain comprises the transmembrane domain of a Vesicular Stomatitis virus glycoprotein (VSV-G).

Embodiment 199. The method of embodiment 198, wherein the transmembrane domain comprises the transmembrane domain and cytosolic domain of a Vesicular Stomatitis virus glycoprotein (VSV-G).

Embodiment 200. The method of any of embodiment s 153-197, wherein the transmembrane domain comprises the transmembrane domain of a Dengue E protein.

Embodiment 201. The method of embodiment 200, wherein the transmembrane domain comprises the transmembrane domain and cytosolic domain of a Dengue E protein.

Embodiment 202. The method of any of embodiments 153-197, wherein the transmembrane domain comprises the transmembrane domain of influenza Hemagglutinin (HA).

Embodiment 203. The method of embodiment 202, wherein the transmembrane domain comprises the transmembrane domain and cytosolic domain of influenza Hemagglutinin (HA).

Embodiment 204. The method of any of embodiment s 153-197, wherein the transmembrane domain comprises the transmembrane domain of HIV surface glycoprotein GP120 or GP41.

Embodiment 205. The method of embodiment 204, wherein the transmembrane domain comprises the transmembrane domain and cytosolic domain of HIV surface glycoprotein GP120 or GP41.

Embodiment 206. The method of any of embodiments 153-197, wherein the transmembrane domain comprises the transmembrane domain of measles virus surface glycoprotein hamagglutinin (H) protein.

Embodiment 207. The method of embodiment 206, wherein the transmembrane domain comprises the transmembrane domain and cytosolic domain of measles virus surface glycoprotein hamagglutinin (H) protein.

Embodiment 208. The method of any of embodiments 153-197, wherein the transmembrane domain comprises the transmembrane domain of influenza Neuraminidase (NA).

Embodiment 209. The method of embodiment 208, wherein the transmembrane domain comprises the transmembrane domain and cytosolic domain of influenza Neuraminidase (NA).

Embodiment 210. The method of any of embodiments 153-209, wherein the transmembrane domain comprises an amino acid sequence that has at least 95% sequence identity to an amino acid sequence according to SEQ ID NOs: 1-4, 52-55.

Embodiment 211. The recombinant fusion protein of embodiment 34, wherein the antibody comprises a non-neutralizing antibody, a neutralizing antibody, or an antibody of multiple formats.

Embodiment 212. The recombinant fusion protein of embodiment 34, wherein the receptor comprises ACE2, or DPP4.

Embodiment 213. The recombinant fusion protein of embodiment 34, wherein the immune checkpoint protein comprises CTLA-4, or PD-1.

Embodiment 214. The enveloped particle of embodiment 99, wherein the antibody comprises a non-neutralizing antibody, a neutralizing antibody, or an antibody of multiple formats.

Embodiment 215. The enveloped particle of embodiment 99, wherein the receptor comprises ACE2, or DPP4.

Embodiment 216. The enveloped particle of embodiment 99, wherein the immune checkpoint protein comprises CTLA-4, or PD-1.

Embodiment 217. The method of embodiment 189, wherein the antibody comprises a non-neutralizing antibody, a neutralizing antibody, or an antibody of multiple formats.

Embodiment 218. The method of embodiment 189, wherein the receptor comprises ACE2, or DPP4.

Embodiment 219. The method of embodiment 189, wherein the immune checkpoint protein comprises CTLA-4, or PD-1.

Embodiment 220. The method of embodiment 171, wherein the cytosolic domain comprises an amino acid that has at least 95% sequence identity to an amino acid sequence set forth in SEQ ID NO: 5.

Embodiment 221. The enveloped particle of embodiment 81, wherein the cytosolic domain comprises an amino acid that has at least 95% sequence identity to an amino acid sequence set forth in SEQ ID NO: 5.

Embodiment 222. The recombinant fusion protein of embodiment 18, wherein the cytosolic domain comprises an amino acid that has at least 95% sequence identity to an amino acid sequence set forth in SEQ ID NO: 5.

Embodiment 223. The recombinant fusion protein of embodiment 1, wherein the display peptide comprise an anti-BCMA scFv polypeptide, an anti-CD19 scFv polypeptide, an anti-NTD scFv polypeptide, an anti-RBD scFv polypeptide, ACE2, ICAM-1, LILRA5, LILRB3, LILRB4, DPP4, BAFF, BCMA, TACI, BAFF Receptor, CD24, CD48, CD59, CD55, IL-4, CXCL-12, NTCP, FCεRI beta, a non-neutralizing antibody, a neutralizing antibody, an antibody of multiple formats, IL-6, CTLA-4, PD-1, LFA-1, Wnt, CD80, CD86, OX40L, CD3, or 4-1BBL.

Embodiment 224. The enveloped particle of embodiment 64, wherein the display polypeptide of the first or second recombinant fusion protein comprises an anti-BCMA scFv polypeptide, an anti-CD19 scFv polypeptide, an anti-NTD scFv polypeptide, an anti-RBD scFv polypeptide, ACE2, ICAM-1, LILRA5, LILRB3, LILRB4, DPP4, BAFF, BCMA, TACI, BAFF Receptor, CD24, CD48, CD59, CD55, IL-4, CXCL-12, NTCP, FCεRI beta, a non-neutralizing antibody, a neutralizing antibody, an antibody of multiple formats, IL-6, CTLA-4, PD-1, LFA-1, Wnt, CD80, CD86, OX40L, CD3, or 4-1BBL.

Embodiment 225. The method of embodiment 153, wherein the display polypeptide of the first or second recombinant fusion protein comprises an anti-BCMA scFv polypeptide, an anti-CD19 scFv polypeptide, an anti-NTD scFv polypeptide, an anti-RBD scFv polypeptide, ACE2, ICAM-1, LILRA5, LILRB3, LILRB4, DPP4, BAFF, BCMA, TACI, BAFF Receptor, CD24, CD48, CD59, CD55, IL-4, CXCL-12, NTCP, FCεRI beta, a non-neutralizing antibody, a neutralizing antibody, an antibody of multiple formats, IL-6, CTLA-4, PD-1, LFA-1, Wnt, CD80, CD86, OX40L, CD3, or 4-1BBL.

Embodiment 226. The recombinant fusion protein of embodiment 1, wherein the recombinant fusion protein comprises an amino acid sequence having at least about 75%, 80%, 85%, 90%, 95%, or 99% sequence identity to any of SEQ ID NOs: 72-78, 81-94, 96-100.

Embodiment 227. The enveloped particle of embodiment 64, wherein the first recombinant fusion protein comprises an amino acid sequence having at least about 75%, 80%, 85%, 90%, 95%, or 99% sequence identity to any of SEQ ID NOs: 72-78, 81-94, 96-100.

Embodiment 228. The enveloped particle of embodiment 64, wherein the second recombinant fusion protein comprises an amino acid sequence having at least about 75%, 80%, 85%, 90%, 95%, or 99% sequence identity to any of SEQ ID NOs: 71-100.

Embodiment 229. The composition of embodiment 139, wherein the recombinant fusion protein comprises an amino acid sequence having at least about 75%, 80%, 85%, 90%, 95%, or 99% sequence identity to any of SEQ ID NOs: 72-78, 81-94, 96-100.

Embodiment 230. The composition of embodiment 140, wherein the first recombinant fusion protein comprises an amino acid sequence having at least about 75%, 80%, 85%, 90%, 95%, or 99% sequence identity to any of SEQ ID NOs: 72-78, 81-94, 96-100.

Embodiment 231. The composition of embodiment 140, wherein the second recombinant fusion protein comprises an amino acid sequence having at least about 75%, 80%, 85%, 90%, 95%, or 99% sequence identity to any of SEQ ID NOs: 71-100.

What is claimed is:

1. A recombinant fusion protein comprising a transmembrane domain, a display polypeptide, and an oligomerization domain wherein when the recombinant fusion protein is expressed on the surface of an enveloped particle, the recombinant fusion protein is displayed in an oligomeric format.

2. The recombinant fusion protein of claim 1, wherein the oligomerization domain comprises a dimerization domain, a trimerization domain, or a tetramerization domain.

3. The recombinant fusion protein of claim 2, wherein the dimerization domain comprises a leucine zipper dimerization domain.

4. The recombinant fusion protein of claim 2, wherein the trimerization domain comprises a post-fusion oligomerization domain of viral surface protein.

5. The recombinant fusion protein of claim 2, wherein the trimerization domain comprises a D4 post-fusion trimerization domain of VSV-G protein.

6. The recombinant fusion protein of claim 2, wherein the trimerization domain comprises a Dengue E protein post-fusion trimerization domain, or a foldon trimerization domain.

7. The recombinant fusion protein of claim 2, wherein the tetramerization domain comprises an influenza neuraminidase stem domain.

8. The recombinant fusion protein of claim 1, wherein the oligomerization domain comprises an amino acid sequence that has at least about 80%, 85%, 90%, 95%, or 99% sequence identity to an amino acid sequence of SEQ ID NOs: 6-19.

9. The recombinant fusion protein of claim 1, wherein when the recombinant fusion protein is expressed on the surface of the enveloped particle, the oligomerization domain is outside of the enveloped particle.

10. The recombinant fusion protein of claim 1, wherein when the recombinant fusion protein is expressed on the surface of the enveloped particle, the oligomerization domain is outside of the enveloped particle and adjacent to a signal peptide.

11. The recombinant fusion protein of claim 1, wherein when the recombinant fusion protein is expressed on the surface of the enveloped particle, the oligomerization domain is inside of the enveloped particle.

12. The recombinant fusion protein of claim 1, wherein when the recombinant fusion protein is expressed on the surface of the enveloped particle, the oligomerization domain is inside of the enveloped particle and adjacent to the transmembrane domain.

13. The recombinant fusion protein of claim 1, wherein the recombinant fusion protein comprises a signal peptide.

14. The recombinant fusion protein of claim 13, wherein domains of the recombinant fusion protein are arranged from the N-terminus to the C-terminus in the following orders:
(a) signal peptide, display polypeptide, oligomerization domain, and transmembrane domain;
(b) signal peptide, display polypeptide, transmembrane domain, and oligomerization domain: or
(c) signal peptide, oligomerization domain, display peptide, and transmembrane domain.

15. The recombinant fusion protein of claim 13, wherein the recombinant fusion protein further comprises a cytosolic domain.

16. The recombinant fusion protein of claim 15, wherein domains of the recombinant fusion protein are arranged from the N-terminus to the C-terminus in the following orders:
(a) signal peptide, display polypeptide, oligomerization domain, transmembrane domain, and cytosolic domain;
(b) signal peptide, display polypeptide, transmembrane domain, oligomerization domain, and cytosolic domain; or
(c) signal peptide, oligomerization domain, display peptide, transmembrane domain, and cytosolic domain.

17. The recombinant fusion protein of claim 1, wherein the enveloped particle comprises an enveloped viral-like particle, an enveloped virus, an extracellular vesicle.

18. The recombinant fusion protein of claim 17, wherein the extracellular vesicle comprises an exosome or an ectosome.

19. The recombinant fusion protein of claim 1, wherein the display polypeptide targets a cell surface protein that is oligomerized on a target cell for agonistic or antagonistic function.

20. The recombinant fusion protein of claim 19, wherein the cell surface protein is dimerized and the oligomerization domain is a dimerization domain.

21. The recombinant fusion protein of claim 19, wherein the cell surface protein is trimerized and the oligomerization domain is a trimerization domain.

22. The recombinant fusion protein of claim 19, wherein the cell surface protein is tetramerized and the oligomerization domain is a tetramerization domain.

23. The recombinant fusion protein of claim 1, wherein the display polypeptide comprises a single chain variable fragment (scFv), a type I transmembrane polypeptide, a type II transmembrane polypeptide, a type III transmembrane polypeptide, a GPI-anchored polypeptide, a secreted polypeptide, or a multi-pass transmembrane polypeptide.

24. The recombinant fusion protein of claim 23, wherein the scFv comprises an anti-BCMA scFv polypeptide, an anti-CD19 scFv polypeptide, an anti-NTD scFv polypeptide, or an anti-RBD scFv polypeptide.

25. The recombinant fusion protein of claim 23, wherein the type I transmembrane polypeptide comprises ACE2, ICAM-1, LILRA5, LILRB3, or LILRB4.

26. The recombinant fusion protein of claim 23, wherein the type II transmembrane polypeptide comprises DPP4, or BAFF.

27. The recombinant fusion protein of claim 23, wherein the type III transmembrane polypeptide comprises BCMA, TACI, or BAFF Receptor.

28. The recombinant fusion protein of claim 23, wherein the GPI-anchored polypeptide comprises CD24, CD48, CD59 or CD55.

29. The recombinant fusion protein of claim 23, wherein the secreted polypeptide comprises IL-4, or CXCL-12.

30. The recombinant fusion protein of claim 23, wherein the multi-pass transmembrane polypeptide comprises NTCP, or FCεRI beta.

31. The recombinant fusion protein of claim 1, wherein the display polypeptide comprises an antibody, a receptor, a cytokine, an immune checkpoint protein, a chemokine, an adhesion factor, or a mitogen.

32. The recombinant fusion protein of claim 31, wherein the antibody comprises a non-neutralizing antibody, a neutralizing antibody, or an antibody of multiple formats.

33. The recombinant fusion protein of claim 31, wherein the receptor comprises ACE2, or DPP4.

34. The recombinant fusion protein of claim 31, wherein the cytokine comprises IL-4, or IL-6.

35. The recombinant fusion protein of claim 31, wherein the immune checkpoint protein comprises CTLA-4, or PD-1.

36. The recombinant fusion protein of claim 31, wherein the chemokine comprises CXCL-12.

37. The recombinant fusion protein of claim 31, wherein the adhesion factor comprises ICAM-1, or LFA-1.

38. The recombinant fusion protein of claim 31, wherein the mitogen comprises Wnt.

39. The recombinant fusion protein of claim 1, wherein the display polypeptide comprises an amino acid sequence that has at least about 80%, 85%, 90%, 95% or 99% sequence identity to an amino acid sequence according to SEQ ID NOs: 20-51, 56-70.

40. The recombinant fusion protein of claim 1, wherein the transmembrane domain anchors the fusion protein to a bilayer of the enveloped particle.

41. The recombinant fusion protein of claim 1, wherein the transmembrane domain comprises:
(a) the transmembrane domain of a Vesicular Stomatitis virus glycoprotein (VSV-G);
(b) the transmembrane domain of a Dengue E protein;
(c) the transmembrane domain of influenza Hemagglutinin (HA);
(d) the transmembrane domain of HIV surface glycoprotein GP120 or GP41;
(e) the transmembrane domain of measles virus surface glycoprotein hamagglutinin (H) protein: or
(f) the transmembrane domain of influenza Neuraminidase (NA).

42. The recombinant fusion protein of claim 1, wherein the recombinant fusion protein comprises a cytosolic domain.

43. The recombinant fusion protein of claim 42, wherein the recombinant fusion protein comprises:
(a) the transmembrane domain and cytosolic domain of a Vesicular Stomatitis virus glycoprotein (VSV-G);
(b) the transmembrane domain and cytosolic domain of a Dengue E protein;
(c) the transmembrane domain and cytosolic domain of influenza Hemagglutinin (HA)
(d) the transmembrane domain and cytosolic domain of HIV surface glycoprotein GP120 or GP41;
(e) the transmembrane domain and cytosolic domain of measles virus surface glycoprotein hamagglutinin (H) protein: or
(f) the transmembrane domain and cytosolic domain of influenza Neuraminidase (NA).

44. The recombinant fusion protein of claim 1, wherein:
(a) the display polypeptide comprises a single chain variable fragment (scFv), a type I transmembrane polypeptide, a type II transmembrane polypeptide, a type III transmembrane polypeptide, a GPI-anchored polypeptide, a secreted polypeptide, a multi-pass transmembrane polypeptide, an antibody, a receptor, a cytokine, an immune checkpoint protein, a chemokine, an adhesion factor, or a mitogen;
(b) the transmembrane domain comprises the transmembrane domain of a Vesicular Stomatitis virus glycoprotein (VSV-G), the transmembrane domain of a Dengue E protein, the transmembrane domain of influenza Hemagglutinin (HA), the transmembrane domain of HIV surface glycoprotein GP120 or GP41, the transmembrane domain of measles virus surface glycoprotein hamagglutinin (H) protein, or the transmembrane domain of influenza Neuraminidase (NA); and
(c) the oligomerization domain comprises a leucine zipper dimerization domain, D4 post-fusion trimerization domain of VSV-G protein, a Dengue E protein post-fusion trimerization domain, a foldon trimerization domain, or an influenza neuraminidase stem domain.

45. The recombinant fusion protein of claim 1, wherein:
(a) the display polypeptide comprises an anti-BCMA scFv polypeptide, an anti-CD19 scFv polypeptide, an anti-NTD scFv polypeptide, an anti-RBD scFv polypeptide, ACE2, ICAM-1, LILRA5, LILRB3, LILRB4, DPP4, BAFF, BCMA, TACI, BAFF Receptor, CD24, CD48, CD59, CD55, IL-4, CXCL-12, NTCP, FCεRI beta, a non-neutralizing antibody, a neutralizing antibody, an antibody of multiple formats, IL-6, CTLA-4, PD-1, LFA-1, Wnt, CD80, CD86, OX40L, CD3, or 4-1BBL;
(b) the transmembrane domain comprises the transmembrane domain of a Vesicular Stomatitis virus glycoprotein (VSV-G), the transmembrane domain of a Dengue E protein, the transmembrane domain of influenza Hemagglutinin (HA), the transmembrane domain of HIV surface glycoprotein GP120 or GP41, the transmembrane domain of measles virus surface glycoprotein hamagglutinin (H) protein, or the transmembrane domain of influenza Neuraminidase (NA); and
(c) the oligomerization domain comprises a leucine zipper dimerization domain, D4 post-fusion trimerization domain of VSV-G protein, a Dengue E protein post-fusion trimerization domain, a foldon trimerization domain, or an influenza neuraminidase stem domain.

46. The recombinant fusion protein of claim 1, wherein:
(a) the display polypeptide comprises an amino acid sequence that has at least about 80%, 85%, 90%, 95% or 99% sequence identity to an amino acid sequence according to SEQ ID NOs: 20-51, 56-70;
(b) the transmembrane domain comprises the transmembrane domain of a Vesicular Stomatitis virus glycoprotein (VSV-G), the transmembrane domain of a Dengue E protein, the transmembrane domain of influenza Hemagglutinin (HA), the transmembrane domain of HIV surface glycoprotein GP120 or GP41, the transmembrane domain of measles virus surface glycoprotein hamagglutinin (H) protein, or the transmembrane domain of influenza Neuraminidase (NA); and
(c) the oligomerization domain comprises a leucine zipper dimerization domain, D4 post-fusion trimerization domain of VSV-G protein, a Dengue E protein post-fusion trimerization domain, a foldon trimerization domain, or an influenza neuraminidase stem domain.

47. The recombinant fusion protein of claim 1, wherein:
(a) the display polypeptide comprises an amino acid sequence that has at least about 80%, 85%, 90%, 95% or 99% sequence identity to an amino acid sequence according to SEQ ID NOs: 20-51, 56-70;
(b) the transmembrane domain comprises an amino acid sequence that has at least about 80%, 85%, 90%, 95% or 99% sequence identity to an amino acid sequence according to SEQ ID NOs: 1-4, 52-55; and
(c) the oligomerization domain comprises an amino acid sequence that has at least about 80%, 85%, 90%, 95% or 99% sequence identity to an amino acid sequence of SEQ ID NOs: 6-19.

48. The recombinant fusion protein of claim 42, wherein the cytosolic domain comprises an amino acid sequence that has at least about 80%, 85%, 90%, 95% or 99% sequence identity to SEQ ID NO: 5.

49. The recombinant fusion protein of claim 1, wherein the transmembrane domain comprises an amino acid sequence that has at least about 80%, 85%, 90%, 95% or 99% sequence identity to an amino acid sequence according to SEQ ID NOs: 1-4, 52-55.

50. The recombinant fusion protein of claim 1, wherein when the recombinant fusion protein is expressed on the surface of the enveloped particle the recombinant fusion protein is expressed at a valency of about 10 copies on the surface of the enveloped particle.

51. The recombinant fusion protein of claim 1, wherein when the recombinant fusion protein is expressed on the surface of the enveloped particle the recombinant fusion protein is expressed at a valency of about 10 to 15 copies on the surface of the enveloped particle.

52. The recombinant fusion protein of claim 1, wherein when the recombinant fusion protein is expressed on the surface of the enveloped particle the recombinant fusion protein is expressed at a valency of at least about 25 copies on the surface of the enveloped particle.

53. The recombinant fusion protein of claim 1, wherein when the recombinant fusion protein is expressed on the surface of the enveloped particle the recombinant fusion protein is expressed at a valency of at least about 50 copies on the surface of the enveloped particle.

54. The recombinant fusion protein of claim 1, wherein when the recombinant fusion protein is expressed on the surface of the enveloped particle the recombinant fusion protein is expressed at a valency of at least about 100 copies on the surface of the enveloped particle.

55. The recombinant fusion protein of claim 1, wherein when the recombinant fusion protein is expressed on the surface of the enveloped particle the recombinant fusion protein is expressed at a valency of at least about 200 copies on the surface of the enveloped particle.

56. The recombinant fusion protein of claim 1, wherein when the recombinant fusion protein is expressed on the surface of the enveloped particle the recombinant fusion protein is expressed at a valency of at least about 400 copies on the surface of the enveloped particle.

57. The recombinant fusion protein of claim 1, wherein when the recombinant fusion protein is expressed on the surface of the enveloped particle the recombinant fusion protein is expressed at a valency of at least about 600 copies on the surface of the enveloped particle.

58. The recombinant fusion protein of claim 1, wherein when the recombinant fusion protein is expressed on the surface of the enveloped particle the recombinant fusion protein is expressed at least about 1000 copies on the surface of the enveloped particle.

59. An enveloped particle comprising the recombinant fusion protein of claim 1.

60. An enveloped particle comprising:
(a) a first recombinant fusion protein that is displayed in an oligomeric format on the surface of the enveloped particle wherein the first recombinant fusion protein comprises a transmembrane domain, a display polypeptide, and an oligomerization domain; and
(b) a second recombinant fusion protein that is displayed in a monomeric format on the surface of the enveloped particle wherein the second recombinant fusion protein comprises a transmembrane domain and a display polypeptide.

61. The enveloped particle of claim 60, wherein the oligomerization domain comprises a dimerization domain, a trimerization domain, or a tetramerization domain.

62. The enveloped particle of claim 61, wherein the dimerization domain comprises a leucine zipper dimerization domain.

63. The enveloped particle of claim 61, wherein the trimerization domain comprises a post-fusion oligomerization domain of viral surface protein.

64. The enveloped particle of claim 61, wherein the trimerization domain comprises a D4 post-fusion trimerization domain of VSV-G protein.

65. The enveloped particle of claim 61, wherein the trimerization domain comprises a Dengue E protein post-fusion trimerization domain, or a foldon trimerization domain.

66. The enveloped particle of claim 61, wherein the tetramerization domain comprises an influenza neuraminidase stem domain.

67. The enveloped particle of claim 60, wherein the oligomerization domain comprises an amino acid sequence that has at least about 80%, 85%, 90%, 95% or 99% sequence identity to an amino acid sequence of SEQ ID NOs: 6-19.

68. The enveloped particle of claim 60, wherein when the first recombinant fusion protein is expressed on the surface of the enveloped particle, the oligomerization domain is outside of the enveloped particle.

69. The enveloped particle of claim 60, wherein when the first recombinant fusion protein is expressed on the surface of the enveloped particle, the oligomerization domain is outside of the enveloped particle and adjacent to a signal peptide.

70. The enveloped particle of claim 60, wherein when the first recombinant fusion protein is expressed on the surface of the enveloped particle, the oligomerization domain is inside of the enveloped particle.

71. The enveloped particle of claim 60, wherein when the first recombinant fusion protein is expressed on the surface of the enveloped particle, the oligomerization domain is inside of the enveloped particle and adjacent to the transmembrane domain.

72. The enveloped particle of claim 60, wherein the first recombinant fusion protein comprises a signal peptide.

73. The enveloped particle of claim 72, wherein domains of the first recombinant fusion protein are arranged from the N-terminus to the C-terminus in the following orders:
(a) signal peptide, display polypeptide, oligomerization domain, and transmembrane domain;
(b) signal peptide, display polypeptide, transmembrane domain, and oligomerization domain: or
(c) signal peptide, oligomerization domain, display peptide, and transmembrane domain.

74. The enveloped particle of claim 60, wherein the first or second recombinant fusion protein further comprises a cytosolic domain.

75. The enveloped particle of claim 74, wherein domains of the first recombinant fusion protein are arranged from the N-terminus to the C-terminus in the following orders:
(a) signal peptide, display polypeptide, oligomerization domain, transmembrane domain, and cytosolic domain;
(b) signal peptide, display polypeptide, transmembrane domain, oligomerization domain, and cytosolic domain; or
(c) signal peptide, oligomerization domain, display peptide, transmembrane domain, and cytosolic domain.

76. The enveloped particle of claim 60, wherein the enveloped particle comprises an enveloped viral-like particle, an enveloped virus, an extracellular vesicle.

77. The enveloped particle of claim 76, wherein the extracellular vesicle comprises an exosome or an ectosome.

78. The enveloped particle of claim 60, wherein the first recombinant fusion protein and the second recombinant fusion protein have the same display polypeptide.

79. The enveloped particle of claim 60, wherein the first recombinant fusion protein and the second recombinant fusion protein have different display polypeptides.

80. The enveloped particle of claim 60, wherein the display polypeptide of the first or second recombinant fusion protein targets a cell surface protein that is oligomerized on a target cell for agonistic or antagonistic function.

81. The enveloped particle of claim 80, wherein the cell surface protein is dimerized and the oligomerization domain of the first recombinant fusion protein comprises a dimerization domain.

82. The enveloped particle of claim 80, wherein the cell surface protein is trimerized and the oligomerization domain of the first recombinant fusion protein comprises a trimerization domain.

83. The enveloped particle of claim 80, wherein the cell surface protein is tetramerized and the oligomerization domain of the first recombinant fusion protein comprises a tetramerization domain.

84. The enveloped particle of claim 60, wherein the display polypeptide of the first or second recombinant fusion protein comprises a single chain variable fragment (scFv), a type I transmembrane polypeptide, a type II transmembrane polypeptide, a type III transmembrane polypeptide, a GPI-anchored polypeptide, a secreted polypeptide, or a multi-pass transmembrane polypeptide.

85. The enveloped particle of claim 84, wherein the scFv comprises an anti-BCMA scFv polypeptide, an anti-NTD scFv polypeptide, an anti-RBD scFv polypeptide, or an anti-CD19 scFv polypeptide.

86. The enveloped particle of claim 84, wherein the type I transmembrane polypeptide comprises ACE2, ICAM-1, LILRA5, LILRB3, or LILRB4.

87. The enveloped particle of claim 84, wherein the type II transmembrane polypeptide comprises DPP4, or BAFF.

88. The enveloped particle of claim 84, wherein the type III transmembrane polypeptide comprises BCMA, TACI, or BAFF Receptor.

89. The enveloped particle of claim 84, wherein the GPI-anchored polypeptide comprises CD24, CD48, CD59 or CD55.

90. The enveloped particle of claim 84, wherein the secreted polypeptide comprises IL-4, or CXCL-12.

91. The enveloped particle of claim 84, wherein the multi-pass transmembrane polypeptide comprises NTCP, or FCεRI beta.

92. The enveloped particle of claim 60, wherein the display polypeptide of the first or second recombinant fusion protein comprises an antibody, a receptor, a cytokine, an immune checkpoint protein, a chemokine, an adhesion factor, or a mitogen.

93. The enveloped particle of claim 92, wherein the antibody comprises a non-neutralizing antibody, a neutralizing antibody, or an antibody of multiple formats.

94. The enveloped particle of claim 92, wherein the receptor comprises ACE2, or DPP4.

95. The enveloped particle of claim 92, wherein the cytokine comprises IL-4, or IL-6.

96. The enveloped particle of claim 92, wherein the immune checkpoint protein comprises CTLA-4, or PD-1.

97. The enveloped particle of claim 92, wherein the chemokine comprises CXCL-12.

98. The enveloped particle of claim 92, wherein the adhesion factor comprises ICAM-1, or LFA-1.

99. The enveloped particle of claim 92, wherein the mitogen comprises Wnt.

100. The enveloped particle of claim 60, wherein the display polypeptide of the first or second recombinant fusion protein comprises an amino acid sequence that has at least about 80%, 85%, 90%, 95% or 99% sequence identity to an amino acid sequence according to SEQ ID NOs: 20-51, 56-70.

101. The enveloped particle of claim 60, wherein the first recombinant fusion protein and the second recombinant fusion protein have the same transmembrane domains.

102. The enveloped particle of claim 60, wherein the first recombinant fusion protein and the second recombinant fusion protein have different transmembrane domains.

103. The enveloped particle of claim 60, wherein the transmembrane domain anchors the fusion protein to a bilayer of the enveloped particle.

104. The enveloped particle of claim 60, wherein the first or second recombinant fusion protein comprises:
(a) the transmembrane domain of a Vesicular Stomatitis virus glycoprotein (VSV-G);
(b) the transmembrane domain of a Dengue E protein;
(c) the transmembrane domain of influenza Hemagglutinin (HA);
(d) the transmembrane domain of HIV surface glycoprotein GP120 or GP41;
(e) the transmembrane domain of measles virus surface glycoprotein hamagglutinin (H) protein: or
(f) the transmembrane domain of influenza Neuraminidase (NA).

105. The enveloped particle of claim 74, wherein the first or second recombinant fusion protein comprises:
(a) the transmembrane domain and cytosolic domain of a Vesicular Stomatitis virus glycoprotein (VSV-G);
(b) the transmembrane domain and cytosolic domain of a Dengue E protein;
(c) the transmembrane domain and cytosolic domain of influenza Hemagglutinin (HA)
(d) the transmembrane domain and cytosolic domain of HIV surface glycoprotein GP120 or GP41;
(e) the transmembrane domain and cytosolic domain of measles virus surface glycoprotein hamagglutinin (H) protein: or
(f) the transmembrane domain and cytosolic domain of influenza Neuraminidase (NA).

106. The enveloped particle of claim 60, wherein:
(a) the display polypeptide of the first or second recombinant fusion protein comprises a single chain variable fragment (scFv), a type I transmembrane polypeptide, a type II transmembrane polypeptide, a type III transmembrane polypeptide, a GPI-anchored polypeptide, a secreted polypeptide, a multi-pass transmembrane polypeptide, an antibody, a receptor, a cytokine, an immune checkpoint protein, a chemokine, an adhesion factor, or a mitogen;
(b) the transmembrane domain of the first or second recombinant fusion comprises the transmembrane domain of a Vesicular Stomatitis virus glycoprotein (VSV-G), the transmembrane domain of a Dengue E protein, the transmembrane domain of influenza Hemagglutinin (HA), the transmembrane domain of HIV surface glycoprotein GP120 or GP41, the transmembrane domain of measles virus surface glycoprotein hamagglutinin (H) protein, or the transmembrane domain of influenza Neuraminidase (NA); and
(c) the oligomerization domain of the first recombinant fusion protein comprises a leucine zipper dimerization domain, D4 post-fusion trimerization domain of VSV-G protein, a Dengue E protein post-fusion trimerization domain, a foldon trimerization domain, or an influenza neuraminidase stem domain.

107. The enveloped particle of claim 60, wherein:
(a) the display polypeptide of the first or second recombinant fusion protein comprises an anti-BCMA scFv polypeptide, an anti-CD19 scFv polypeptide, an anti-NTD scFv polypeptide, an anti-RBD scFv polypeptide, ACE2, ICAM-1, LILRA5, LILRB3, LILRB4, DPP4, BAFF, BCMA, TACI, BAFF Receptor, CD24, CD48, CD59, CD55, IL-4, CXCL-12, NTCP, FCεRI beta, a non-neutralizing antibody, a neutralizing antibody, an antibody of multiple formats, IL-6, CTLA-4, PD-1, LFA-1, Wnt, CD80, CD86, OX40L, CD3, or 4-1BBL;
(b) the transmembrane domain of the first or second recombinant fusion comprises the transmembrane domain of a Vesicular Stomatitis virus glycoprotein (VSV-G), the transmembrane domain of a Dengue E protein, the transmembrane domain of influenza Hemagglutinin (HA), the transmembrane domain of HIV surface glycoprotein GP120 or GP41, the transmembrane domain of measles virus surface glycoprotein hamagglutinin (H) protein, or the transmembrane domain of influenza Neuraminidase (NA); and
(c) the oligomerization domain of the first recombinant fusion protein comprises a leucine zipper dimerization domain, D4 post-fusion trimerization domain of VSV-G protein, a Dengue E protein post-fusion trimerization domain, a foldon trimerization domain, or an influenza neuraminidase stem domain.

108. The enveloped particle of claim 60, wherein:
(a) the display polypeptide of the first or second recombinant fusion protein comprises an amino acid sequence that has at least about 80%, 85%, 90%, 95% or 99% sequence identity to an amino acid sequence according to SEQ ID NOs: 20-51, 56-70;
(b) the transmembrane domain of the first or second recombinant fusion comprises the transmembrane domain of a Vesicular Stomatitis virus glycoprotein (VSV-G), the transmembrane domain of a Dengue E protein, the transmembrane domain of influenza Hemagglutinin (HA), the transmembrane domain of HIV surface glycoprotein GP120 or GP41, the transmembrane domain of measles virus surface glycoprotein hamagglutinin (H) protein, or the transmembrane domain of influenza Neuraminidase (NA); and
(c) the oligomerization domain of the first recombinant fusion protein comprises a leucine zipper dimerization domain, D4 post-fusion trimerization domain of VSV-G protein, a Dengue E protein post-fusion trimerization domain, a foldon trimerization domain, or an influenza neuraminidase stem domain.

109. The enveloped particle of claim 60, wherein:
(a) the display polypeptide of the first or second recombinant fusion protein comprises an amino acid sequence that has at least about 80%, 85%, 90%, 95% or 99% sequence identity to an amino acid sequence according to SEQ ID NOs: 20-51, 56-70;
(b) the transmembrane domain of the first or second recombinant fusion comprises an amino acid sequence that has at least about 80%, 85%, 90%, 95% or 99% sequence identity to an amino acid sequence according to SEQ ID NOs: 1-4, 52-55; and
(c) the oligomerization domain of the first recombinant fusion protein comprises an amino acid sequence that has at least about 80%, 85%, 90%, 95% or 99% sequence identity to an amino acid sequence of SEQ ID NOs: 6-19.

110. The enveloped particle of claim 74, wherein the cytosolic domain comprises an amino acid sequence that has at least about 80%, 85%, 90%, 95% or 99% sequence identity to SEQ ID No: 5.

111. The enveloped particle of claim 60, wherein the transmembrane domain comprises an amino acid sequence that has at least about 80%, 85%, 90%, 95% or 99% sequence identity to an amino acid sequence according to SEQ ID NOs: 1-4, 52-55.

112. The enveloped particle of claim 60, wherein when the first or second recombinant fusion protein is expressed on the surface of the enveloped particle the first or second recombinant fusion protein is expressed at a valency of about 10 copies on the surface of the enveloped particle.

113. The enveloped particle of claim 60, wherein when the first or second recombinant fusion protein is expressed on the surface of the enveloped particle the first or second recombinant fusion protein is expressed at a valency of about 10 to 15 copies on the surface of the enveloped particle.

114. The enveloped particle of claim 60, wherein when the first or second recombinant fusion protein is expressed on the surface of the enveloped particle the first or second recombinant fusion protein is expressed at a valency of at least about 25 copies on the surface of the enveloped particle.

115. The enveloped particle of claim 60, wherein when the first or second recombinant fusion protein is expressed on the surface of the enveloped particle the first or second recombinant fusion protein is expressed at a valency of at least about 50 copies on the surface of the enveloped particle.

116. The enveloped particle of claim 60, wherein when the first or second recombinant fusion protein is expressed on the surface of the enveloped particle the first or second recombinant fusion protein is expressed at a valency of at least about 100 copies on the surface of the enveloped particle.

117. The enveloped particle of claim 60, wherein when the first or second recombinant fusion protein is expressed on the surface of the enveloped particle the first or second recombinant fusion protein is expressed at a valency of at least about 200 copies on the surface of the enveloped particle.

118. The enveloped particle of claim 60, wherein when the first or second recombinant fusion protein is expressed on the surface of the enveloped particle the first or second recombinant fusion protein is expressed at a valency of at least about 400 copies on the surface of the enveloped particle.

119. The enveloped particle of claim 60, wherein when the first or second recombinant fusion protein is expressed on the surface of the enveloped particle the first or second recombinant fusion protein is expressed at a valency of at least about 600 copies on the surface of the enveloped particle.

120. The enveloped particle of claim 60, wherein when the first or second recombinant fusion protein is expressed on the surface of the enveloped particle the first or second recombinant fusion protein is expressed on the surface of the enveloped particle at a valency of at least about 1000 copies on the surface of the enveloped particle.

121. The recombinant fusion protein of claim 1, wherein the recombinant fusion protein comprises an amino acid sequence having at least about 75%, 80%, 85%, 90%, 95%, or 99% sequence identity to any of SEQ ID NOs: 72-78, 81-94, 96-100.

122. The enveloped particle of claim 60, wherein the first recombinant fusion protein comprises an amino acid sequence having at least about 75%, 80%, 85%, 90%, 95%, or 99% sequence identity to any of SEQ ID NOs: 72-78, 81-94, 96-100.

123. The enveloped particle of claim 60, wherein the second recombinant fusion protein comprises an amino acid sequence having at least about 75%, 80%, 85%, 90%, 95%, or 99% sequence identity to any of SEQ ID NOs: 71-100.
